SEQUENCE LISTING (539 sequences)

SEQ ID NO: 1 - protein, 10 aa, Artificial Sequence
  Synthetic Glycine Serine Linker
GSGSGSGSGG

SEQ ID NO: 2 - protein, 10 aa, Artificial Sequence
  Synthetic Glycine Serine Linker
GGSGSGSGSG

SEQ ID NO: 3 - protein, 15 aa, Artificial Sequence
  Synthetic Glycine Serine Linker
GGGGSGGGGSGGGGS

SEQ ID NO: 4 - protein, 4 aa, Artificial Sequence
  Synthetic Glycine Serine Linker
GGGS

SEQ ID NO: 5 - protein, 5 aa, Artificial Sequence
  Synthetic Glycine Serine Linker
GGGGS

SEQ ID NO: 6 - protein, 9 aa, Artificial Sequence
  Synthetic Glycine Serine Linker
GGSGGSGGS

SEQ ID NO: 7 - protein, 10 aa, Artificial Sequence
  Synthetic Glycine Serine Linker
GGGGSGGGGS

SEQ ID NO: 8 - protein, 20 aa, Artificial Sequence
  Synthetic Glycine Serine Linker
GGGGSGGGGSGGGGSGGGGS

SEQ ID NO: 9 - protein, 25 aa, Artificial Sequence
  Synthetic Glycine Serine Linker
GGGGSGGGGSGGGGSGGGGSGGGGS

SEQ ID NO: 10 - protein, 30 aa, Artificial Sequence
  Synthetic Glycine Serine Linker
GGGGSGGGGSGGGGSGGGGSGGGGSGGGGS

SEQ ID NO: 11 - protein, 35 aa, Artificial Sequence
  Synthetic Glycine Serine Linker
GGGGSGGGGSGGGGSGGGGSGGGGSGGGGSGGGGS

SEQ ID NO: 12 - protein, 40 aa, Artificial Sequence
  Synthetic Glycine Serine Linker
GGGGSGGGGSGGGGSGGGGSGGGGSGGGGSGGGGSGGGGS

SEQ ID NO: 13 - protein, 45 aa, Artificial Sequence
  Synthetic Glycine Serine Linker
GGGGSGGGGSGGGGSGGGGSGGGGSGGGGSGGGGSGGGGSGGGGS

SEQ ID NO: 14 - protein, 50 aa, Artificial Sequence
  Synthetic Glycine Serine Linker
GGGGSGGGGSGGGGSGGGGSGGGGSGGGGSGGGGSGGGGSGGGGSGGGGS

SEQ ID NO: 15 - protein, 55 aa, Artificial Sequence
  Synthetic Glycine Serine Linker
GGGGSGGGGSGGGGSGGGGSGGGGSGGGGSGGGGSGGGGSGGGGSGGGGSGGGGS

SEQ ID NO: 16 - protein, 60 aa, Artificial Sequence
  Synthetic Glycine Serine Linker
GGGGSGGGGSGGGGSGGGGSGGGGSGGGGSGGGGSGGGGSGGGGSGGGGSGGGGSGGGGS

SEQ ID NO: 17 - protein, 32 aa, Artificial Sequence
  Synthetic Linker
LEPGEKPYKCPECGKSFSQSGALTRHQRTHTR

SEQ ID NO: 18 - protein, 20 aa, Artificial Sequence
  Synthetic Linker
PKKKRKVEASSPKKRKVEAS

SEQ ID NO: 19 - DNA, 9 bp, Artificial Sequence
  Synthetic
ggtggtagt

SEQ ID NO: 20 - DNA, 27 bp, Artificial Sequence
  Synthetic
ggtggtagtggagggagcggcggttca

SEQ ID NO: 21 - DNA, 72 bp, Artificial Sequence
  Synthetic
ggtggaggaggctctggtggaggcggtagcggaggcggagggtcgggtggtagtggagggagcggcggttca

SEQ ID NO: 22 - DNA, 48 bp, Artificial Sequence
  Synthetic
tcgggatctgagacgcctgggacctcggaatcggctacgcccgaaagt

SEQ ID NO: 23 - DNA, 192 bp, Artificial Sequence
  Synthetic
gtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaaaccggcggtggttctggt

SEQ ID NO: 24 - DNA, 108 bp, Artificial Sequence
  Synthetic
ggtggttctgccggtggctccggttctggctccagcggtggcagctctggtgcgtccggcacgggtactgcgggtggcactggcagcggttccggtactggctctggc

SEQ ID NO: 25 - protein, 7 aa, Simian virus 40
PKKKRKV

SEQ ID NO: 26 - protein, 16 aa, Artificial Sequence
  Synthetic
KRPAATKKAGQAKKKK

SEQ ID NO: 27 - protein, 9 aa, Homo sapiens
PAAKRVKLD

SEQ ID NO: 28 - protein, 11 aa, Homo sapiens
RQRRNELKRSP

SEQ ID NO: 29 - protein, 38 aa, Homo sapiens
NQSSNFGPMKGGNFGGRSSGPYGGGGQYFAKPRNQGGY

SEQ ID NO: 30 - protein, 42 aa, Homo sapiens
RMRIZFKNKGKDTAELRRRRVEVSVELRKAKKDEQILKRRNV

SEQ ID NO: 31 - protein, 8 aa, Homo sapiens
VSRKRPRP

SEQ ID NO: 32 - protein, 8 aa, Homo sapiens
PPKKARED

SEQ ID NO: 33 - protein, 8 aa, Homo sapiens
PQPKKKPL

SEQ ID NO: 34 - protein, 12 aa, Mus sp.
SALIKKKKKMAP

SEQ ID NO: 35 - protein, 5 aa, Influenza virus
DRLRR

SEQ ID NO: 36 - protein, 7 aa, Influenza virus
PKQKKRK

SEQ ID NO: 37 - protein, 10 aa, Hepatitis D virus
RKLKKKIKKL

SEQ ID NO: 38 - protein, 10 aa, Mus sp.
REKKKFLKRR

SEQ ID NO: 39 - protein, 20 aa, Homo sapiens
KRKGDEVDGVDEVAKKKSKK

SEQ ID NO: 40 - protein, 17 aa, Homo sapiens
RKCLQAGMNLEARKTKK

SEQ ID NO: 41 - DNA, 240 bp, Artificial Sequence
  Synthetic
ggctcaggtagcggaagcggatcaggtggattcaatgtactgatggtccataaacggagtcacactggcgagcgcccgctccaatgtgaaatctgcgggttcacgtgtcggcagaagggcaacctcctccggcatatcaagctgcacacgggtgaaaaaccgtttaagtgccatctctgcaattacgcctgtcagagaagagatgctttgggtggatctggatctggcagcgggtctggc

SEQ ID NO: 42 - protein, 80 aa, Artificial Sequence
  Synthetic
GSGSGSGSGGFNVLMVHKRSHTGERPLQCEICGFTCRQKGNLLRHIKLHTGEKPFKCHLCNYACQRRDALGGSGSGSGSG

SEQ ID NO: 43 - DNA, 129 bp, Artificial Sequence
  Synthetic
ggctctgggagtgggtccggctctggaggtctccagtgcgagatctgtggcttcacctgtagacagaaaggtaacttgcttcgacatatcaaactccatggggggtcagggtctggtagtggaagcggc

SEQ ID NO: 44 - protein, 43 aa, Artificial Sequence
  Synthetic
GSGSGSGSGGLQCEICGFTCRQKGNLLRHIKLHGGSGSGSGSG

SEQ ID NO: 45 - DNA, 4509 bp, Artificial Sequence
  Synthetic
gactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcggctcaggtagcggaagcggatcaggtggattcaatgtactgatggtccataaacggagtcacactggcgagcgcccgctccaatgtgaaatctgcgggttcacgtgtcggcagaagggcaacctcctccggcatatcaagctgcacacgggtgaaaaaccgtttaagtgccatctctgcaattacgcctgtcagagaagagatgctttgggtggatctggatctggcagcgggtctggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaa

SEQ ID NO: 46 - protein, 11 aa, Artificial Sequence
  Synthetic
FQCNQCGASFT

SEQ ID NO: 47 - protein, 12 aa, Artificial Sequence
  Synthetic
TKGSLIRHHRRH

SEQ ID NO: 48 - protein, 23 aa, Artificial Sequence
  Synthetic
FQCNQCGASFTTKGSLIRHHRRH

SEQ ID NO: 49 - protein, 11 aa, Artificial Sequence
  Synthetic
FICGQCGLAFK

SEQ ID NO: 50 - protein, 23 aa, Artificial Sequence
  Synthetic
FICGQCGLAFKTKGSLIRHHRRH

SEQ ID NO: 51 - protein, 11 aa, Artificial Sequence
  Synthetic
YSCPVCGLRFK

SEQ ID NO: 52 - protein, 23 aa, Artificial Sequence
  Synthetic
YSCPVCGLRFKTKGSLIRHHRRH

SEQ ID NO: 53 - protein, 11 aa, Artificial Sequence
  Synthetic
FSCPQCGIDFN

SEQ ID NO: 54 - protein, 23 aa, Artificial Sequence
  Synthetic
FSCPQCGIDFNTKGSLIRHHRRH

SEQ ID NO: 55 - protein, 11 aa, Artificial Sequence
  Synthetic
FACVICGRKFR

SEQ ID NO: 56 - protein, 23 aa, Artificial Sequence
  Synthetic
FACVICGRKFRTKGSLIRHHRRH

SEQ ID NO: 57 - protein, 11 aa, Artificial Sequence
  Synthetic
FQCPICGLVIK

SEQ ID NO: 58 - protein, 23 aa, Artificial Sequence
  Synthetic
FQCPICGLVIKTKGSLIRHHRRH

SEQ ID NO: 59 - protein, 11 aa, Artificial Sequence
  Synthetic
LQCPDCDMTFP

SEQ ID NO: 60 - protein, 23 aa, Artificial Sequence
  Synthetic
LQCPDCDMTFPTKGSLIRHHRRH

SEQ ID NO: 61 - protein, 11 aa, Artificial Sequence
  Synthetic
YKCQECDKAFK

SEQ ID NO: 62 - protein, 23 aa, Artificial Sequence
  Synthetic
YKCQECDKAFKTKGSLIRHHRRH

SEQ ID NO: 63 - protein, 11 aa, Artificial Sequence
  Synthetic
YKCLECGKSFS

SEQ ID NO: 64 - protein, 23 aa, Artificial Sequence
  Synthetic
YKCLECGKSFSTKGSLIRHHRRH

SEQ ID NO: 65 - protein, 11 aa, Artificial Sequence
  Synthetic
LQCEICGYQCR

SEQ ID NO: 66 - protein, 23 aa, Artificial Sequence
  Synthetic
LQCEICGYQCRTKGSLIRHHRRH

SEQ ID NO: 67 - protein, 11 aa, Artificial Sequence
  Synthetic
LQCEICGFTCR

SEQ ID NO: 68 - protein, 23 aa, Artificial Sequence
  Synthetic
LQCEICGYQCRTKGSLIRHHRRH

SEQ ID NO: 69 - protein, 11 aa, Artificial Sequence
  Synthetic
FHCNQCGASFT

SEQ ID NO: 70 - protein, 23 aa, Artificial Sequence
  Synthetic
FHCNQCGASFTTKGSLIRHHRRH

SEQ ID NO: 71 - protein, 11 aa, Artificial Sequence
  Synthetic
LQCEVCGFQCR

SEQ ID NO: 72 - protein, 23 aa, Artificial Sequence
  Synthetic
LQCEVCGFQCRTKGSLIRHHRRH

SEQ ID NO: 73 - protein, 11 aa, Artificial Sequence
  Synthetic
YGCNECGKNFG

SEQ ID NO: 74 - protein, 23 aa, Artificial Sequence
  Synthetic
YGCNECGKNFGTKGSLIRHHRRH

SEQ ID NO: 75 - protein, 11 aa, Artificial Sequence
  Synthetic
FKCADCGKGFS

SEQ ID NO: 76 - protein, 23 aa, Artificial Sequence
  Synthetic
FKCADCGKGFSTKGSLIRHHRRH

SEQ ID NO: 77 - protein, 11 aa, Artificial Sequence
  Synthetic
YQCKVCGRAFK

SEQ ID NO: 78 - protein, 23 aa, Artificial Sequence
  Synthetic
YQCKVCGRAFKTKGSLIRHHRRH

SEQ ID NO: 79 - protein, 11 aa, Artificial Sequence
  Synthetic
FVCPRCGRGFS

SEQ ID NO: 80 - protein, 23 aa, Artificial Sequence
  Synthetic
FVCPRCGRGFSTKGSLIRHHRRH

SEQ ID NO: 81 - protein, 11 aa, Artificial Sequence
  Synthetic
HECKLCGASFR

SEQ ID NO: 82 - protein, 23 aa, Artificial Sequence
  Synthetic
HECKLCGASFRTKGSLIRHHRRH

SEQ ID NO: 83 - protein, 11 aa, Artificial Sequence
  Synthetic
YRCRACGRACS

SEQ ID NO: 84 - protein, 23 aa, Artificial Sequence
  Synthetic
YRCRACGRACSTKGSLIRHHRRH

SEQ ID NO: 85 - protein, 11 aa, Artificial Sequence
  Synthetic
FQCNTCVKVFS

SEQ ID NO: 86 - protein, 23 aa, Artificial Sequence
  Synthetic
FQCNTCVKVFSTKGSLIRHHRRH

SEQ ID NO: 87 - protein, 11 aa, Artificial Sequence
  Synthetic
YICNVCSKTFS

SEQ ID NO: 88 - protein, 23 aa, Artificial Sequence
  Synthetic
YICNVCSKTFSTKGSLIRHHRRH

SEQ ID NO: 89 - protein, 12 aa, Artificial Sequence
  Synthetic
QKGNLLRHIKLH

SEQ ID NO: 90 - protein, 23 aa, Artificial Sequence
  Synthetic
FKCADCGKGFSQKGNLLRHIKLH

SEQ ID NO: 91 - protein, 23 aa, Artificial Sequence
  Synthetic
HECKLCGASFRQKGNLLRHIKLH

SEQ ID NO: 92 - protein, 23 aa, Artificial Sequence
  Synthetic
YQCKVCGRAFKQKGNLLRHIKLH

SEQ ID NO: 93 - protein, 23 aa, Artificial Sequence
  Synthetic
YKCQECDKAFKQKGNLLRHIKLH

SEQ ID NO: 94 - protein, 23 aa, Artificial Sequence
  Synthetic
LQCEICGYQCRQKGNLLRHIKLH

SEQ ID NO: 95 - protein, 23 aa, Artificial Sequence
  Synthetic
FQCNTCVKVFSQKGNLLRHIKLH

SEQ ID NO: 96 - protein, 23 aa, Artificial Sequence
  Synthetic
YICNVCSKTFSQKGNLLRHIKLH

SEQ ID NO: 97 - protein, 23 aa, Artificial Sequence
  Synthetic
LQCPDCDMTFPQKGNLLRHIKLH

SEQ ID NO: 98 - protein, 23 aa, Artificial Sequence
  Synthetic
FHCNQCGASFTQKGNLLRHIKLH

SEQ ID NO: 99 - protein, 23 aa, Artificial Sequence
  Synthetic
FHCNQCGASFTQKGNLLRHIKLH

SEQ ID NO: 100 - protein, 23 aa, Artificial Sequence
  Synthetic
FICGQCGLAFKQKGNLLRHIKLH

SEQ ID NO: 101 - protein, 23 aa, Artificial Sequence
  Synthetic
LQCEVCGFQCRQKGNLLRHIKLH

SEQ ID NO: 102 - protein, 23 aa, Artificial Sequence
  Synthetic
FQCNQCGASFTQKGNLLRHIKLH

SEQ ID NO: 103 - protein, 23 aa, Artificial Sequence
  Synthetic
FSCPQCGIDFNQKGNLLRHIKLH

SEQ ID NO: 104 - protein, 23 aa, Artificial Sequence
  Synthetic
FACVICGRKFRQKGNLLRHIKLH

SEQ ID NO: 105 - protein, 23 aa, Artificial Sequence
  Synthetic
YKCLECGKSFSQKGNLLRHIKLH

SEQ ID NO: 106 - protein, 23 aa, Artificial Sequence
  Synthetic
FQCPICGLVIKQKGNLLRHIKLH

SEQ ID NO: 107 - protein, 23 aa, Artificial Sequence
  Synthetic
YGCNECGKNFGQKGNLLRHIKLH

SEQ ID NO: 108 - protein, 23 aa, Artificial Sequence
  Synthetic
YSCPVCGLRFKQKGNLLRHIKLH

SEQ ID NO: 109 - protein, 23 aa, Artificial Sequence
  Synthetic
FVCPRCGRGFSQKGNLLRHIKLH

SEQ ID NO: 110 - protein, 23 aa, Artificial Sequence
  Synthetic
YRCRACGRACSQKGNLLRHIKLH

SEQ ID NO: 111 - protein, 12 aa, Artificial Sequence
  Synthetic
RKDRMSYHVRSH

SEQ ID NO: 112 - protein, 23 aa, Artificial Sequence
  Synthetic
YGCNECGKNFGRKDRMSYHVRSH

SEQ ID NO: 113 - protein, 23 aa, Artificial Sequence
  Synthetic
YQCKVCGRAFKRKDRMSYHVRSH

SEQ ID NO: 114 - protein, 23 aa, Artificial Sequence
  Synthetic
LQCEICGFTCRRKDRMSYHVRSH

SEQ ID NO: 115 - protein, 23 aa, Artificial Sequence
  Synthetic
FVCPRCGRGFSRKDRMSYHVRSH

SEQ ID NO: 116 - protein, 23 aa, Artificial Sequence
  Synthetic
YICNVCSKTFSRKDRMSYHVRSH

SEQ ID NO: 117 - protein, 23 aa, Artificial Sequence
  Synthetic
FSCPQCGIDFNRKDRMSYHVRSH

SEQ ID NO: 118 - protein, 23 aa, Artificial Sequence
  Synthetic
YRCRACGRACSRKDRMSYHVRSH

SEQ ID NO: 119 - protein, 23 aa, Artificial Sequence
  Synthetic
YSCPVCGLRFKRKDRMSYHVRSH

SEQ ID NO: 120 - protein, 23 aa, Artificial Sequence
  Synthetic
LQCEVCGFQCRRKDRMSYHVRSH

SEQ ID NO: 121 - protein, 23 aa, Artificial Sequence
  Synthetic
YKCLECGKSFSRKDRMSYHVRSH

SEQ ID NO: 122 - protein, 23 aa, Artificial Sequence
  Synthetic
FKCADCGKGFSRKDRMSYHVRSH

SEQ ID NO: 123 - protein, 23 aa, Artificial Sequence
  Synthetic
FHCNQCGASFTRKDRMSYHVRSH

SEQ ID NO: 124 - protein, 23 aa, Artificial Sequence
  Synthetic
HECKLCGASFRRKDRMSYHVRSH

SEQ ID NO: 125 - protein, 23 aa, Artificial Sequence
  Synthetic
FICGQCGLAFKRKDRMSYHVRSH

SEQ ID NO: 126 - protein, 23 aa, Artificial Sequence
  Synthetic
FQCNQCGASFTRKDRMSYHVRSH

SEQ ID NO: 127 - protein, 23 aa, Artificial Sequence
  Synthetic
FQCPICGLVIKRKDRMSYHVRSH

SEQ ID NO: 128 - protein, 23 aa, Artificial Sequence
  Synthetic
FACVICGRKFRRKDRMSYHVRSH

SEQ ID NO: 129 - protein, 23 aa, Artificial Sequence
  Synthetic
LQCPDCDMTFPRKDRMSYHVRSH

SEQ ID NO: 130 - protein, 23 aa, Artificial Sequence
  Synthetic
LQCEICGYQCRRKDRMSYHVRSH

SEQ ID NO: 131 - protein, 23 aa, Artificial Sequence
  Synthetic
YKCQECDKAFKRKDRMSYHVRSH

SEQ ID NO: 132 - protein, 23 aa, Artificial Sequence
  Synthetic
FQCNTCVKVFSRKDRMSYHVRSH

SEQ ID NO: 133 - protein, 12 aa, Artificial Sequence
  Synthetic
HSTYLTQHQRTH

SEQ ID NO: 134 - protein, 23 aa, Artificial Sequence
  Synthetic
FICGQCGLAFKHSTYLTQHQRTH

SEQ ID NO: 135 - protein, 23 aa, Artificial Sequence
  Synthetic
HECKLCGASFRHSTYLTQHQRTH

SEQ ID NO: 136 - protein, 23 aa, Artificial Sequence
  Synthetic
FVCPRCGRGFSHSTYLTQHQRTH

SEQ ID NO: 137 - protein, 23 aa, Artificial Sequence
  Synthetic
YQCKVCGRAFKHSTYLTQHQRTH

SEQ ID NO: 138 - protein, 23 aa, Artificial Sequence
  Synthetic
YKCQECDKAFKHSTYLTQHQRTH

SEQ ID NO: 139 - protein, 23 aa, Artificial Sequence
  Synthetic
FQCNQCGASFTHSTYLTQHQRTH

SEQ ID NO: 140 - protein, 23 aa, Artificial Sequence
  Synthetic
LQCEVCGFQCRHSTYLTQHQRTH

SEQ ID NO: 141 - protein, 23 aa, Artificial Sequence
  Synthetic
FQCNTCVKVFSHSTYLTQHQRTH

SEQ ID NO: 142 - protein, 23 aa, Artificial Sequence
  Synthetic
FSCPQCGIDFNHSTYLTQHQRTH

SEQ ID NO: 143 - protein, 23 aa, Artificial Sequence
  Synthetic
LQCEICGFTCRHSTYLTQHQRTH

SEQ ID NO: 144 - protein, 23 aa, Artificial Sequence
  Synthetic
FACVICGRKFRHSTYLTQHQRTH

SEQ ID NO: 145 - protein, 23 aa, Artificial Sequence
  Synthetic
FHCNQCGASFTHSTYLTQHQRTH

SEQ ID NO: 146 - protein, 23 aa, Artificial Sequence
  Synthetic
YSCPVCGLRFKHSTYLTQHQRTH

SEQ ID NO: 147 - protein, 23 aa, Artificial Sequence
  Synthetic
YICNVCSKTFSHSTYLTQHQRTH

SEQ ID NO: 148 - protein, 23 aa, Artificial Sequence
  Synthetic
LQCEICGYQCRHSTYLTQHQRTH

SEQ ID NO: 149 - protein, 23 aa, Artificial Sequence
  Synthetic
YGCNECGKNFGHSTYLTQHQRTH

SEQ ID NO: 150 - protein, 23 aa, Artificial Sequence
  Synthetic
LQCPDCDMTFPHSTYLTQHQRTH

SEQ ID NO: 151 - protein, 23 aa, Artificial Sequence
  Synthetic
YKCLECGKSFSHSTYLTQHQRTH

SEQ ID NO: 152 - protein, 23 aa, Artificial Sequence
  Synthetic
FKCADCGKGFSHSTYLTQHQRTH

SEQ ID NO: 153 - protein, 23 aa, Artificial Sequence
  Synthetic
YRCRACGRACSHSTYLTQHQRTH

SEQ ID NO: 154 - protein, 23 aa, Artificial Sequence
  Synthetic
FQCPICGLVIKHSTYLTQHQRTH

SEQ ID NO: 155 - protein, 12 aa, Artificial Sequence
  Synthetic
QKASLNWHMKKH

SEQ ID NO: 156 - protein, 23 aa, Artificial Sequence
  Synthetic
YQCKVCGRAFKQKASLNWHMKKH

SEQ ID NO: 157 - protein, 23 aa, Artificial Sequence
  Synthetic
FSCPQCGIDFNQKASLNWHMKKH

SEQ ID NO: 158 - protein, 23 aa, Artificial Sequence
  Synthetic
YICNVCSKTFSQKASLNWHMKKH

SEQ ID NO: 159 - protein, 23 aa, Artificial Sequence
  Synthetic
FQCPICGLVIKQKASLNWHMKKH

SEQ ID NO: 160 - protein, 23 aa, Artificial Sequence
  Synthetic
YSCPVCGLRFKQKASLNWHMKKH

SEQ ID NO: 161 - protein, 23 aa, Artificial Sequence
  Synthetic
LQCEICGYQCRQKASLNWHMKKH

SEQ ID NO: 162 - protein, 23 aa, Artificial Sequence
  Synthetic
LQCEVCGFQCRQKASLNWHMKKH

SEQ ID NO: 163 - protein, 23 aa, Artificial Sequence
  Synthetic
FVCPRCGRGFSQKASLNWHMKKH

SEQ ID NO: 164 - protein, 23 aa, Artificial Sequence
  Synthetic
FACVICGRKFRQKASLNWHMKKH

SEQ ID NO: 165 - protein, 23 aa, Artificial Sequence
  Synthetic
FICGQCGLAFKQKASLNWHMKKH

SEQ ID NO: 166 - protein, 23 aa, Artificial Sequence
  Synthetic
FHCNQCGASFTQKASLNWHMKKH

SEQ ID NO: 167 - protein, 23 aa, Artificial Sequence
  Synthetic
LQCPDCDMTFPQKASLNWHMKKH

SEQ ID NO: 168 - protein, 23 aa, Artificial Sequence
  Synthetic
FQCNQCGASFTQKASLNWHMKKH

SEQ ID NO: 169 - protein, 23 aa, Artificial Sequence
  Synthetic
FKCADCGKGFSQKASLNWHMKKH

SEQ ID NO: 170 - protein, 23 aa, Artificial Sequence
  Synthetic
HECKLCGASFRQKASLNWHMKKH

SEQ ID NO: 171 - protein, 23 aa, Artificial Sequence
  Synthetic
YGCNECGKNFGQKASLNWHMKKH

SEQ ID NO: 172 - protein, 23 aa, Artificial Sequence
  Synthetic
LQCEICGFTCRQKASLNWHMKKH

SEQ ID NO: 173 - protein, 23 aa, Artificial Sequence
  Synthetic
YKCLECGKSFSQKASLNWHMKKH

SEQ ID NO: 174 - protein, 23 aa, Artificial Sequence
  Synthetic
YKCQECDKAFKQKASLNWHMKKH

SEQ ID NO: 175 - protein, 23 aa, Artificial Sequence
  Synthetic
YRCRACGRACSQKASLNWHMKKH

SEQ ID NO: 176 - protein, 23 aa, Artificial Sequence
  Synthetic
FQCNTCVKVFSQKASLNWHMKKH

SEQ ID NO: 177 - protein, 12 aa, Artificial Sequence
  Synthetic
RHSHLIEHLKRH

SEQ ID NO: 178 - protein, 23 aa, Artificial Sequence
  Synthetic
LQCPDCDMTFPRHSHLIEHLKRH

SEQ ID NO: 179 - protein, 23 aa, Artificial Sequence
  Synthetic
YKCQECDKAFKRHSHLIEHLKRH

SEQ ID NO: 180 - protein, 23 aa, Artificial Sequence
  Synthetic
FSCPQCGIDFNRHSHLIEHLKRH

SEQ ID NO: 181 - protein, 23 aa, Artificial Sequence
  Synthetic
FHCNQCGASFTRHSHLIEHLKRH

SEQ ID NO: 182 - protein, 23 aa, Artificial Sequence
  Synthetic
YGCNECGKNFGRHSHLIEHLKRH

SEQ ID NO: 183 - protein, 23 aa, Artificial Sequence
  Synthetic
FICGQCGLAFKRHSHLIEHLKRH

SEQ ID NO: 184 - protein, 23 aa, Artificial Sequence
  Synthetic
YSCPVCGLRFKRHSHLIEHLKRH

SEQ ID NO: 185 - protein, 23 aa, Artificial Sequence
  Synthetic
FACVICGRKFRRHSHLIEHLKRH

SEQ ID NO: 186 - protein, 23 aa, Artificial Sequence
  Synthetic
YKCLECGKSFSRHSHLIEHLKRH

SEQ ID NO: 187 - protein, 23 aa, Artificial Sequence
  Synthetic
YQCKVCGRAFKRHSHLIEHLKRH

SEQ ID NO: 188 - protein, 23 aa, Artificial Sequence
  Synthetic
FVCPRCGRGFSRHSHLIEHLKRH

SEQ ID NO: 189 - protein, 23 aa, Artificial Sequence
  Synthetic
LQCEVCGFQCRRHSHLIEHLKRH

SEQ ID NO: 190 - protein, 23 aa, Artificial Sequence
  Synthetic
YICNVCSKTFSRHSHLIEHLKRH

SEQ ID NO: 191 - protein, 23 aa, Artificial Sequence
  Synthetic
FQCNTCVKVFSRHSHLIEHLKRH

SEQ ID NO: 192 - protein, 23 aa, Artificial Sequence
  Synthetic
LQCEICGYQCRRHSHLIEHLKRH

SEQ ID NO: 193 - protein, 23 aa, Artificial Sequence
  Synthetic
HECKLCGASFRRHSHLIEHLKRH

SEQ ID NO: 194 - protein, 23 aa, Artificial Sequence
  Synthetic
YRCRACGRACSRHSHLIEHLKRH

SEQ ID NO: 195 - protein, 23 aa, Artificial Sequence
  Synthetic
FKCADCGKGFSRHSHLIEHLKRH

SEQ ID NO: 196 - protein, 23 aa, Artificial Sequence
  Synthetic
LQCEICGFTCRRHSHLIEHLKRH

SEQ ID NO: 197 - protein, 23 aa, Artificial Sequence
  Synthetic
FQCNQCGASFTRHSHLIEHLKRH

SEQ ID NO: 198 - protein, 23 aa, Artificial Sequence
  Synthetic
FQCPICGLVIKRHSHLIEHLKRH

SEQ ID NO: 199 - protein, 12 aa, Artificial Sequence
  Synthetic
QRASLKYHMTKH

SEQ ID NO: 200 - protein, 23 aa, Artificial Sequence
  Synthetic
YICNVCSKTFSQRASLKYHMTKH

SEQ ID NO: 201 - protein, 23 aa, Artificial Sequence
  Synthetic
YRCRACGRACSQRASLKYHMTKH

SEQ ID NO: 202 - protein, 23 aa, Artificial Sequence
  Synthetic
FHCNQCGASFTQRASLKYHMTKH

SEQ ID NO: 203 - protein, 23 aa, Artificial Sequence
  Synthetic
FQCNQCGASFTQRASLKYHMTKH

SEQ ID NO: 204 - protein, 23 aa, Artificial Sequence
  Synthetic
FICGQCGLAFKQRASLKYHMTKH

SEQ ID NO: 205 - protein, 23 aa, Artificial Sequence
  Synthetic
FSCPQCGIDFNQRASLKYHMTKH

SEQ ID NO: 206 - protein, 23 aa, Artificial Sequence
  Synthetic
LQCPDCDMTFPQRASLKYHMTKH

SEQ ID NO: 207 - protein, 23 aa, Artificial Sequence
  Synthetic
HECKLCGASFRQRASLKYHMTKH

SEQ ID NO: 208 - protein, 23 aa, Artificial Sequence
  Synthetic
FQCPICGLVIKQRASLKYHMTKH

SEQ ID NO: 209 - protein, 23 aa, Artificial Sequence
  Synthetic
FVCPRCGRGFSQRASLKYHMTKH

SEQ ID NO: 210 - protein, 23 aa, Artificial Sequence
  Synthetic
YKCQECDKAFKQRASLKYHMTKH

SEQ ID NO: 211 - protein, 23 aa, Artificial Sequence
  Synthetic
YKCLECGKSFSQRASLKYHMTKH

SEQ ID NO: 212 - protein, 23 aa, Artificial Sequence
  Synthetic
YSCPVCGLRFKQRASLKYHMTKH

SEQ ID NO: 213 - protein, 23 aa, Artificial Sequence
  Synthetic
YQCKVCGRAFKQRASLKYHMTKH

SEQ ID NO: 214 - protein, 23 aa, Artificial Sequence
  Synthetic
LQCEICGYQCRQRASLKYHMTKH

SEQ ID NO: 215 - protein, 23 aa, Artificial Sequence
  Synthetic
LQCEVCGFQCRQRASLKYHMTKH

SEQ ID NO: 216 - protein, 23 aa, Artificial Sequence
  Synthetic
LQCEICGFTCRQRASLKYHMTKH

SEQ ID NO: 217 - protein, 23 aa, Artificial Sequence
  Synthetic
FKCADCGKGFSQRASLKYHMTKH

SEQ ID NO: 218 - protein, 23 aa, Artificial Sequence
  Synthetic
YGCNECGKNFGQRASLKYHMTKH

SEQ ID NO: 219 - protein, 23 aa, Artificial Sequence
  Synthetic
FACVICGRKFRQRASLKYHMTKH

SEQ ID NO: 220 - protein, 23 aa, Artificial Sequence
  Synthetic
FQCNTCVKVFSQRASLKYHMTKH

SEQ ID NO: 221 - protein, 12 aa, Artificial Sequence
  Synthetic
GHSALIRHQMIH

SEQ ID NO: 222 - protein, 23 aa, Artificial Sequence
  Synthetic
YSCPVCGLRFKGHSALIRHQMIH

SEQ ID NO: 223 - protein, 23 aa, Artificial Sequence
  Synthetic
FKCADCGKGFSGHSALIRHQMIH

SEQ ID NO: 224 - protein, 23 aa, Artificial Sequence
  Synthetic
YGCNECGKNFGGHSALIRHQMIH

SEQ ID NO: 225 - protein, 23 aa, Artificial Sequence
  Synthetic
LQCEVCGFQCRGHSALIRHQMIH

SEQ ID NO: 226 - protein, 23 aa, Artificial Sequence
  Synthetic
YRCRACGRACSGHSALIRHQMIH

SEQ ID NO: 227 - protein, 23 aa, Artificial Sequence
  Synthetic
FQCPICGLVIKGHSALIRHQMIH

SEQ ID NO: 228 - protein, 23 aa, Artificial Sequence
  Synthetic
FHCNQCGASFTGHSALIRHQMIH

SEQ ID NO: 229 - protein, 23 aa, Artificial Sequence
  Synthetic
FSCPQCGIDFNGHSALIRHQMIH

SEQ ID NO: 230 - protein, 23 aa, Artificial Sequence
  Synthetic
YICNVCSKTFSGHSALIRHQMIH

SEQ ID NO: 231 - protein, 23 aa, Artificial Sequence
  Synthetic
HECKLCGASFRGHSALIRHQMIH

SEQ ID NO: 232 - protein, 23 aa, Artificial Sequence
  Synthetic
FACVICGRKFRGHSALIRHQMIH

SEQ ID NO: 233 - protein, 23 aa, Artificial Sequence
  Synthetic
FICGQCGLAFKGHSALIRHQMIH

SEQ ID NO: 234 - protein, 23 aa, Artificial Sequence
  Synthetic
FQCNTCVKVFSGHSALIRHQMIH

SEQ ID NO: 235 - protein, 23 aa, Artificial Sequence
  Synthetic
FQCNQCGASFTGHSALIRHQMIH

SEQ ID NO: 236 - protein, 23 aa, Artificial Sequence
  Synthetic
YQCKVCGRAFKGHSALIRHQMIH

SEQ ID NO: 237 - protein, 23 aa, Artificial Sequence
  Synthetic
YKCQECDKAFKGHSALIRHQMIH

SEQ ID NO: 238 - protein, 23 aa, Artificial Sequence
  Synthetic
FVCPRCGRGFSGHSALIRHQMIH

SEQ ID NO: 239 - protein, 23 aa, Artificial Sequence
  Synthetic
YKCLECGKSFSGHSALIRHQMIH

SEQ ID NO: 240 - protein, 23 aa, Artificial Sequence
  Synthetic
LQCEICGFTCRGHSALIRHQMIH

SEQ ID NO: 241 - protein, 23 aa, Artificial Sequence
  Synthetic
LQCPDCDMTFPGHSALIRHQMIH

SEQ ID NO: 242 - protein, 23 aa, Artificial Sequence
  Synthetic
LQCEICGYQCRGHSALIRHQMIH

SEQ ID NO: 243 - protein, 12 aa, Artificial Sequence
  Synthetic
RLSTLIQHQKVH

SEQ ID NO: 244 - protein, 23 aa, Artificial Sequence
  Synthetic
FICGQCGLAFKRLSTLIQHQKVH

SEQ ID NO: 245 - protein, 23 aa, Artificial Sequence
  Synthetic
FQCNQCGASFTRLSTLIQHQKVH

SEQ ID NO: 246 - protein, 23 aa, Artificial Sequence
  Synthetic
YRCRACGRACSRLSTLIQHQKVH

SEQ ID NO: 247 - protein, 23 aa, Artificial Sequence
  Synthetic
LQCEICGYQCRRLSTLIQHQKVH

SEQ ID NO: 248 - protein, 23 aa, Artificial Sequence
  Synthetic
YSCPVCGLRFKRLSTLIQHQKVH

SEQ ID NO: 249 - protein, 23 aa, Artificial Sequence
  Synthetic
FQCNTCVKVFSRLSTLIQHQKVH

SEQ ID NO: 250 - protein, 23 aa, Artificial Sequence
  Synthetic
LQCPDCDMTFPRLSTLIQHQKVH

SEQ ID NO: 251 - protein, 23 aa, Artificial Sequence
  Synthetic
YKCLECGKSFSRLSTLIQHQKVH

SEQ ID NO: 252 - protein, 23 aa, Artificial Sequence
  Synthetic
LQCEICGFTCRRLSTLIQHQKVH

SEQ ID NO: 253 - protein, 23 aa, Artificial Sequence
  Synthetic
YKCQECDKAFKRLSTLIQHQKVH

SEQ ID NO: 254 - protein, 23 aa, Artificial Sequence
  Synthetic
FACVICGRKFRRLSTLIQHQKVH

SEQ ID NO: 255 - protein, 23 aa, Artificial Sequence
  Synthetic
FSCPQCGIDFNRLSTLIQHQKVH

SEQ ID NO: 256 - protein, 23 aa, Artificial Sequence
  Synthetic
LQCEVCGFQCRRLSTLIQHQKVH

SEQ ID NO: 257 - protein, 23 aa, Artificial Sequence
  Synthetic
FHCNQCGASFTRLSTLIQHQKVH

SEQ ID NO: 258 - protein, 23 aa, Artificial Sequence
  Synthetic
YGCNECGKNFGRLSTLIQHQKVH

SEQ ID NO: 259 - protein, 23 aa, Artificial Sequence
  Synthetic
FVCPRCGRGFSRLSTLIQHQKVH

SEQ ID NO: 260 - protein, 23 aa, Artificial Sequence
  Synthetic
YICNVCSKTFSRLSTLIQHQKVH

SEQ ID NO: 261 - protein, 23 aa, Artificial Sequence
  Synthetic
FQCPICGLVIKRLSTLIQHQKVH

SEQ ID NO: 262 - protein, 23 aa, Artificial Sequence
  Synthetic
FKCADCGKGFSRLSTLIQHQKVH

SEQ ID NO: 263 - protein, 23 aa, Artificial Sequence
  Synthetic
YQCKVCGRAFKRLSTLIQHQKVH

SEQ ID NO: 264 - protein, 23 aa, Artificial Sequence
  Synthetic
HECKLCGASFRRLSTLIQHQKVH

SEQ ID NO: 265 - protein, 12 aa, Artificial Sequence
  Synthetic
RVSHLTVHYRIH

SEQ ID NO: 266 - protein, 23 aa, Artificial Sequence
  Synthetic
FQCNTCVKVFSRVSHLTVHYRIH

SEQ ID NO: 267 - protein, 23 aa, Artificial Sequence
  Synthetic
FHCNQCGASFTRVSHLTVHYRIH

SEQ ID NO: 268 - protein, 23 aa, Artificial Sequence
  Synthetic
YQCKVCGRAFKRVSHLTVHYRIH

SEQ ID NO: 269 - protein, 23 aa, Artificial Sequence
  Synthetic
YRCRACGRACSRVSHLTVHYRIH

SEQ ID NO: 270 - protein, 23 aa, Artificial Sequence
  Synthetic
FICGQCGLAFKRVSHLTVHYRIH

SEQ ID NO: 271 - protein, 23 aa, Artificial Sequence
  Synthetic
FACVICGRKFRRVSHLTVHYRIH

SEQ ID NO: 272 - protein, 23 aa, Artificial Sequence
  Synthetic
LQCPDCDMTFPRVSHLTVHYRIH

SEQ ID NO: 273 - protein, 23 aa, Artificial Sequence
  Synthetic
YICNVCSKTFSRVSHLTVHYRIH

SEQ ID NO: 274 - protein, 23 aa, Artificial Sequence
  Synthetic
FKCADCGKGFSRVSHLTVHYRIH

SEQ ID NO: 275 - protein, 23 aa, Artificial Sequence
  Synthetic
YKCQECDKAFKRVSHLTVHYRIH

SEQ ID NO: 276 - protein, 23 aa, Artificial Sequence
  Synthetic
FSCPQCGIDFNRVSHLTVHYRIH

SEQ ID NO: 277 - protein, 23 aa, Artificial Sequence
  Synthetic
YGCNECGKNFGRVSHLTVHYRIH

SEQ ID NO: 278 - protein, 23 aa, Artificial Sequence
  Synthetic
FQCNQCGASFTRVSHLTVHYRIH

SEQ ID NO: 279 - protein, 23 aa, Artificial Sequence
  Synthetic
LQCEVCGFQCRRVSHLTVHYRIH

SEQ ID NO: 280 - protein, 23 aa, Artificial Sequence
  Synthetic
YKCLECGKSFSRVSHLTVHYRIH

SEQ ID NO: 281 - protein, 23 aa, Artificial Sequence
  Synthetic
HECKLCGASFRRVSHLTVHYRIH

SEQ ID NO: 282 - protein, 23 aa, Artificial Sequence
  Synthetic
YSCPVCGLRFKRVSHLTVHYRIH

SEQ ID NO: 283 - protein, 23 aa, Artificial Sequence
  Synthetic
LQCEICGYQCRRVSHLTVHYRIH

SEQ ID NO: 284 - protein, 23 aa, Artificial Sequence
  Synthetic
LQCEICGFTCRRVSHLTVHYRIH

SEQ ID NO: 285 - protein, 23 aa, Artificial Sequence
  Synthetic
FVCPRCGRGFSRVSHLTVHYRIH

SEQ ID NO: 286 - protein, 23 aa, Artificial Sequence
  Synthetic
FQCPICGLVIKRVSHLTVHYRIH

SEQ ID NO: 287 - protein, 12 aa, Artificial Sequence
  Synthetic
KFSNSNKHKIRH

SEQ ID NO: 288 - protein, 23 aa, Artificial Sequence
  Synthetic
YKCLECGKSFSKFSNSNKHKIRH

SEQ ID NO: 289 - protein, 23 aa, Artificial Sequence
  Synthetic
YQCKVCGRAFKKFSNSNKHKIRH

SEQ ID NO: 290 - protein, 23 aa, Artificial Sequence
  Synthetic
FSCPQCGIDFNKFSNSNKHKIRH

SEQ ID NO: 291 - protein, 23 aa, Artificial Sequence
  Synthetic
YSCPVCGLRFKKFSNSNKHKIRH

SEQ ID NO: 292 - protein, 23 aa, Artificial Sequence
  Synthetic
FVCPRCGRGFSKFSNSNKHKIRH

SEQ ID NO: 293 - protein, 23 aa, Artificial Sequence
  Synthetic
YGCNECGKNFGKFSNSNKHKIRH

SEQ ID NO: 294 - protein, 23 aa, Artificial Sequence
  Synthetic
YKCQECDKAFKKFSNSNKHKIRH

SEQ ID NO: 295 - protein, 23 aa, Artificial Sequence
  Synthetic
LQCPDCDMTFPKFSNSNKHKIRH

SEQ ID NO: 296 - protein, 23 aa, Artificial Sequence
  Synthetic
FQCPICGLVIKKFSNSNKHKIRH

SEQ ID NO: 297 - protein, 23 aa, Artificial Sequence
  Synthetic
FQCNQCGASFTKFSNSNKHKIRH

SEQ ID NO: 298 - protein, 23 aa, Artificial Sequence
  Synthetic
FQCNTCVKVFSKFSNSNKHKIRH

SEQ ID NO: 299 - protein, 23 aa, Artificial Sequence
  Synthetic
LQCEVCGFQCRKFSNSNKHKIRH

SEQ ID NO: 300 - protein, 23 aa, Artificial Sequence
  Synthetic
FKCADCGKGFSKFSNSNKHKIRH

SEQ ID NO: 301 - protein, 23 aa, Artificial Sequence
  Synthetic
FICGQCGLAFKKFSNSNKHKIRH

SEQ ID NO: 302 - protein, 23 aa, Artificial Sequence
  Synthetic
YICNVCSKTFSKFSNSNKHKIRH

SEQ ID NO: 303 - protein, 23 aa, Artificial Sequence
  Synthetic
LQCEICGFTCRKFSNSNKHKIRH

SEQ ID NO: 304 - protein, 23 aa, Artificial Sequence
  Synthetic
FACVICGRKFRKFSNSNKHKIRH

SEQ ID NO: 305 - protein, 23 aa, Artificial Sequence
  Synthetic
LQCEICGYQCRKFSNSNKHKIRH

SEQ ID NO: 306 - protein, 23 aa, Artificial Sequence
  Synthetic
YRCRACGRACSKFSNSNKHKIRH

SEQ ID NO: 307 - protein, 23 aa, Artificial Sequence
  Synthetic
HECKLCGASFRKFSNSNKHKIRH

SEQ ID NO: 308 - protein, 23 aa, Artificial Sequence
  Synthetic
FHCNQCGASFTKFSNSNKHKIRH

SEQ ID NO: 309 - protein, 12 aa, Artificial Sequence
  Synthetic
CFSELISHQNIH

SEQ ID NO: 310 - protein, 23 aa, Artificial Sequence
  Synthetic
HECKLCGASFRCFSELISHQNIH

SEQ ID NO: 311 - protein, 23 aa, Artificial Sequence
  Synthetic
FQCPICGLVIKCFSELISHQNIH

SEQ ID NO: 312 - protein, 23 aa, Artificial Sequence
  Synthetic
FKCADCGKGFSCFSELISHQNIH

SEQ ID NO: 313 - protein, 23 aa, Artificial Sequence
  Synthetic
YKCQECDKAFKCFSELISHQNIH

SEQ ID NO: 314 - protein, 23 aa, Artificial Sequence
  Synthetic
FVCPRCGRGFSCFSELISHQNIH

SEQ ID NO: 315 - protein, 23 aa, Artificial Sequence
  Synthetic
FQCNQCGASFTCFSELISHQNIH

SEQ ID NO: 316 - protein, 23 aa, Artificial Sequence
  Synthetic
YQCKVCGRAFKCFSELISHQNIH

SEQ ID NO: 317 - protein, 23 aa, Artificial Sequence
  Synthetic
FACVICGRKFRCFSELISHQNIH

SEQ ID NO: 318 - protein, 23 aa, Artificial Sequence
  Synthetic
LQCPDCDMTFPCFSELISHQNIH

SEQ ID NO: 319 - protein, 23 aa, Artificial Sequence
  Synthetic
FQCNTCVKVFSCFSELISHQNIH

SEQ ID NO: 320 - protein, 23 aa, Artificial Sequence
  Synthetic
FICGQCGLAFKCFSELISHQNIH

SEQ ID NO: 321 - protein, 23 aa, Artificial Sequence
  Synthetic
FSCPQCGIDFNCFSELISHQNIH

SEQ ID NO: 322 - protein, 23 aa, Artificial Sequence
  Synthetic
YRCRACGRACSCFSELISHQNIH

SEQ ID NO: 323 - protein, 23 aa, Artificial Sequence
  Synthetic
YICNVCSKTFSCFSELISHQNIH

SEQ ID NO: 324 - protein, 23 aa, Artificial Sequence
  Synthetic
YSCPVCGLRFKCFSELISHQNIH

SEQ ID NO: 325 - protein, 23 aa, Artificial Sequence
  Synthetic
LQCEICGYQCRCFSELISHQNIH

SEQ ID NO: 326 - protein, 23 aa, Artificial Sequence
  Synthetic
YGCNECGKNFGCFSELISHQNIH

SEQ ID NO: 327 - protein, 23 aa, Artificial Sequence
  Synthetic
FHCNQCGASFTCFSELISHQNIH

SEQ ID NO: 328 - protein, 23 aa, Artificial Sequence
  Synthetic
YKCLECGKSFSCFSELISHQNIH

SEQ ID NO: 329 - protein, 23 aa, Artificial Sequence
  Synthetic
LQCEICGFTCRCFSELISHQNIH

SEQ ID NO: 330 - protein, 23 aa, Artificial Sequence
  Synthetic
LQCEVCGFQCRCFSELISHQNIH

SEQ ID NO: 331 - protein, 12 aa, Artificial Sequence
  Synthetic
WSSHYQYHLRQH

SEQ ID NO: 332 - protein, 23 aa, Artificial Sequence
  Synthetic
YSCPVCGLRFKWSSHYQYHLRQH

SEQ ID NO: 333 - protein, 23 aa, Artificial Sequence
  Synthetic
FSCPQCGIDFNWSSHYQYHLRQH

SEQ ID NO: 334 - protein, 23 aa, Artificial Sequence
  Synthetic
FQCPICGLVIKWSSHYQYHLRQH

SEQ ID NO: 335 - protein, 23 aa, Artificial Sequence
  Synthetic
FVCPRCGRGFSWSSHYQYHLRQH

SEQ ID NO: 336 - protein, 23 aa, Artificial Sequence
  Synthetic
LQCEVCGFQCRWSSHYQYHLRQH

SEQ ID NO: 337 - protein, 23 aa, Artificial Sequence
  Synthetic
LQCEICGFTCRWSSHYQYHLRQH

SEQ ID NO: 338 - protein, 23 aa, Artificial Sequence
  Synthetic
FKCADCGKGFSWSSHYQYHLRQH

SEQ ID NO: 339 - protein, 23 aa, Artificial Sequence
  Synthetic
FQCNTCVKVFSWSSHYQYHLRQH

SEQ ID NO: 340 - protein, 23 aa, Artificial Sequence
  Synthetic
LQCEICGYQCRWSSHYQYHLRQH

SEQ ID NO: 341 - protein, 23 aa, Artificial Sequence
  Synthetic
YGCNECGKNFGWSSHYQYHLRQH

SEQ ID NO: 342 - protein, 23 aa, Artificial Sequence
  Synthetic
HECKLCGASFRWSSHYQYHLRQH

SEQ ID NO: 343 - protein, 23 aa, Artificial Sequence
  Synthetic
YKCQECDKAFKWSSHYQYHLRQH

SEQ ID NO: 344 - protein, 23 aa, Artificial Sequence
  Synthetic
FICGQCGLAFKWSSHYQYHLRQH

SEQ ID NO: 345 - protein, 23 aa, Artificial Sequence
  Synthetic
YKCLECGKSFSWSSHYQYHLRQH

SEQ ID NO: 346 - protein, 23 aa, Artificial Sequence
  Synthetic
LQCPDCDMTFPWSSHYQYHLRQH

SEQ ID NO: 347 - protein, 23 aa, Artificial Sequence
  Synthetic
FACVICGRKFRWSSHYQYHLRQH

SEQ ID NO: 348 - protein, 23 aa, Artificial Sequence
  Synthetic
YRCRACGRACSWSSHYQYHLRQH

SEQ ID NO: 349 - protein, 23 aa, Artificial Sequence
  Synthetic
FQCNQCGASFTWSSHYQYHLRQH

SEQ ID NO: 350 - protein, 23 aa, Artificial Sequence
  Synthetic
FHCNQCGASFTWSSHYQYHLRQH

SEQ ID NO: 351 - protein, 23 aa, Artificial Sequence
  Synthetic
YICNVCSKTFSWSSHYQYHLRQH

SEQ ID NO: 352 - protein, 23 aa, Artificial Sequence
  Synthetic
YQCKVCGRAFKWSSHYQYHLRQH

SEQ ID NO: 353 - protein, 12 aa, Artificial Sequence
  Synthetic
QRASLNWHMKKH

SEQ ID NO: 354 - protein, 23 aa, Artificial Sequence
  Synthetic
FACVICGRKFRQRASLNWHMKKH

SEQ ID NO: 355 - protein, 23 aa, Artificial Sequence
  Synthetic
YKCQECDKAFKQRASLNWHMKKH

SEQ ID NO: 356 - protein, 23 aa, Artificial Sequence
  Synthetic
FQCNTCVKVFSQRASLNWHMKKH

SEQ ID NO: 357 - protein, 23 aa, Artificial Sequence
  Synthetic
YQCKVCGRAFKQRASLNWHMKKH

SEQ ID NO: 358 - protein, 23 aa, Artificial Sequence
  Synthetic
FQCPICGLVIKQRASLNWHMKKH

SEQ ID NO: 359 - protein, 23 aa, Artificial Sequence
  Synthetic
FQCNQCGASFTQRASLNWHMKKH

SEQ ID NO: 360 - protein, 23 aa, Artificial Sequence
  Synthetic
FVCPRCGRGFSQRASLNWHMKKH

SEQ ID NO: 361 - protein, 23 aa, Artificial Sequence
  Synthetic
YRCRACGRACSQRASLNWHMKKH

SEQ ID NO: 362 - protein, 23 aa, Artificial Sequence
  Synthetic
FHCNQCGASFTQRASLNWHMKKH

SEQ ID NO: 363 - protein, 23 aa, Artificial Sequence
  Synthetic
FKCADCGKGFSQRASLNWHMKKH

SEQ ID NO: 364 - protein, 23 aa, Artificial Sequence
  Synthetic
LQCEICGFTCRQRASLNWHMKKH

SEQ ID NO: 365 - protein, 23 aa, Artificial Sequence
  Synthetic
HECKLCGASFRQRASLNWHMKKH

SEQ ID NO: 366 - protein, 23 aa, Artificial Sequence
  Synthetic
LQCEICGYQCRQRASLNWHMKKH

SEQ ID NO: 367 - protein, 23 aa, Artificial Sequence
  Synthetic
YGCNECGKNFGQRASLNWHMKKH

SEQ ID NO: 368 - protein, 23 aa, Artificial Sequence
  Synthetic
FSCPQCGIDFNQRASLNWHMKKH

SEQ ID NO: 369 - protein, 23 aa, Artificial Sequence
  Synthetic
LQCPDCDMTFPQRASLNWHMKKH

SEQ ID NO: 370 - protein, 23 aa, Artificial Sequence
  Synthetic
FICGQCGLAFKQRASLNWHMKKH

SEQ ID NO: 371 - protein, 23 aa, Artificial Sequence
  Synthetic
LQCEVCGFQCRQRASLNWHMKKH

SEQ ID NO: 372 - protein, 23 aa, Artificial Sequence
  Synthetic
YICNVCSKTFSQRASLNWHMKKH

SEQ ID NO: 373 - protein, 23 aa, Artificial Sequence
  Synthetic
YSCPVCGLRFKQRASLNWHMKKH

SEQ ID NO: 374 - protein, 23 aa, Artificial Sequence
  Synthetic
YKCLECGKSFSQRASLNWHMKKH

SEQ ID NO: 375 - protein, 12 aa, Artificial Sequence
  Synthetic
NRGLMQKHLKNH

SEQ ID NO: 376 - protein, 23 aa, Artificial Sequence
  Synthetic
LQCEVCGFQCRNRGLMQKHLKNH

SEQ ID NO: 377 - protein, 23 aa, Artificial Sequence
  Synthetic
FQCNTCVKVFSNRGLMQKHLKNH

SEQ ID NO: 378 - protein, 23 aa, Artificial Sequence
  Synthetic
LQCEICGFTCRNRGLMQKHLKNH

SEQ ID NO: 379 - protein, 23 aa, Artificial Sequence
  Synthetic
LQCPDCDMTFPNRGLMQKHLKNH

SEQ ID NO: 380 - protein, 23 aa, Artificial Sequence
  Synthetic
HECKLCGASFRNRGLMQKHLKNH

SEQ ID NO: 381 - protein, 23 aa, Artificial Sequence
  Synthetic
FICGQCGLAFKNRGLMQKHLKNH

SEQ ID NO: 382 - protein, 23 aa, Artificial Sequence
  Synthetic
FSCPQCGIDFNNRGLMQKHLKNH

SEQ ID NO: 383 - protein, 23 aa, Artificial Sequence
  Synthetic
YGCNECGKNFGNRGLMQKHLKNH

SEQ ID NO: 384 - protein, 23 aa, Artificial Sequence
  Synthetic
YRCRACGRACSNRGLMQKHLKNH

SEQ ID NO: 385 - protein, 23 aa, Artificial Sequence
  Synthetic
YKCLECGKSFSNRGLMQKHLKNH

SEQ ID NO: 386 - protein, 23 aa, Artificial Sequence
  Synthetic
FKCADCGKGFSNRGLMQKHLKNH

SEQ ID NO: 387 - protein, 23 aa, Artificial Sequence
  Synthetic
FQCPICGLVIKNRGLMQKHLKNH

SEQ ID NO: 388 - protein, 23 aa, Artificial Sequence
  Synthetic
YQCKVCGRAFKNRGLMQKHLKNH

SEQ ID NO: 389 - protein, 23 aa, Artificial Sequence
  Synthetic
FVCPRCGRGFSNRGLMQKHLKNH

SEQ ID NO: 390 - protein, 23 aa, Artificial Sequence
  Synthetic
FQCNQCGASFTNRGLMQKHLKNH

SEQ ID NO: 391 - protein, 23 aa, Artificial Sequence
  Synthetic
FHCNQCGASFTNRGLMQKHLKNH

SEQ ID NO: 392 - protein, 23 aa, Artificial Sequence
  Synthetic
LQCEICGYQCRNRGLMQKHLKNH

SEQ ID NO: 393 - protein, 23 aa, Artificial Sequence
  Synthetic
YICNVCSKTFSNRGLMQKHLKNH

SEQ ID NO: 394 - protein, 23 aa, Artificial Sequence
  Synthetic
FACVICGRKFRNRGLMQKHLKNH

SEQ ID NO: 395 - protein, 23 aa, Artificial Sequence
  Synthetic
YSCPVCGLRFKNRGLMQKHLKNH

SEQ ID NO: 396 - protein, 23 aa, Artificial Sequence
  Synthetic
YKCQECDKAFKNRGLMQKHLKNH

SEQ ID NO: 397 - protein, 12 aa, Artificial Sequence
  Synthetic
QKASLNWHQRKH

SEQ ID NO: 398 - protein, 23 aa, Artificial Sequence
  Synthetic
FQCNQCGASFTQKASLNWHQRKH

SEQ ID NO: 399 - protein, 23 aa, Artificial Sequence
  Synthetic
LQCEVCGFQCRQKASLNWHQRKH

SEQ ID NO: 400 - protein, 23 aa, Artificial Sequence
  Synthetic
FKCADCGKGFSQKASLNWHQRKH

SEQ ID NO: 401 - protein, 23 aa, Artificial Sequence
  Synthetic
FACVICGRKFRQKASLNWHQRKH

SEQ ID NO: 402 - protein, 23 aa, Artificial Sequence
  Synthetic
FVCPRCGRGFSQKASLNWHQRKH

SEQ ID NO: 403 - protein, 23 aa, Artificial Sequence
  Synthetic
LQCEICGFTCRQKASLNWHQRKH

SEQ ID NO: 404 - protein, 23 aa, Artificial Sequence
  Synthetic
FICGQCGLAFKQKASLNWHQRKH

SEQ ID NO: 405 - protein, 23 aa, Artificial Sequence
  Synthetic
LQCEICGYQCRQKASLNWHQRKH

SEQ ID NO: 406 - protein, 23 aa, Artificial Sequence
  Synthetic
YICNVCSKTFSQKASLNWHQRKH

SEQ ID NO: 407 - protein, 23 aa, Artificial Sequence
  Synthetic
HECKLCGASFRQKASLNWHQRKH

SEQ ID NO: 408 - protein, 23 aa, Artificial Sequence
  Synthetic
LQCPDCDMTFPQKASLNWHQRKH

SEQ ID NO: 409 - protein, 23 aa, Artificial Sequence
  Synthetic
YKCLECGKSFSQKASLNWHQRKH

SEQ ID NO: 410 - protein, 23 aa, Artificial Sequence
  Synthetic
YGCNECGKNFGQKASLNWHQRKH

SEQ ID NO: 411 - protein, 23 aa, Artificial Sequence
  Synthetic
YKCQECDKAFKQKASLNWHQRKH

SEQ ID NO: 412 - protein, 23 aa, Artificial Sequence
  Synthetic
YSCPVCGLRFKQKASLNWHQRKH

SEQ ID NO: 413 - protein, 23 aa, Artificial Sequence
  Synthetic
FQCNTCVKVFSQKASLNWHQRKH

SEQ ID NO: 414 - protein, 23 aa, Artificial Sequence
  Synthetic
YRCRACGRACSQKASLNWHQRKH

SEQ ID NO: 415 - protein, 23 aa, Artificial Sequence
  Synthetic
YQCKVCGRAFKQKASLNWHQRKH

SEQ ID NO: 416 - protein, 23 aa, Artificial Sequence
  Synthetic
FSCPQCGIDFNQKASLNWHQRKH

SEQ ID NO: 417 - protein, 23 aa, Artificial Sequence
  Synthetic
FQCPICGLVIKQKASLNWHQRKH

SEQ ID NO: 418 - protein, 23 aa, Artificial Sequence
  Synthetic
FHCNQCGASFTQKASLNWHQRKH

SEQ ID NO: 419 - protein, 12 aa, Artificial Sequence
  Synthetic
QPKSLARHLRLH

SEQ ID NO: 420 - protein, 23 aa, Artificial Sequence
  Synthetic
YQCKVCGRAFKQPKSLARHLRLH

SEQ ID NO: 421 - protein, 23 aa, Artificial Sequence
  Synthetic
FQCNQCGASFTQPKSLARHLRLH

SEQ ID NO: 422 - protein, 23 aa, Artificial Sequence
  Synthetic
FICGQCGLAFKQPKSLARHLRLH

SEQ ID NO: 423 - protein, 23 aa, Artificial Sequence
  Synthetic
YRCRACGRACSQPKSLARHLRLH

SEQ ID NO: 424 - protein, 23 aa, Artificial Sequence
  Synthetic
FQCPICGLVIKQPKSLARHLRLH

SEQ ID NO: 425 - protein, 23 aa, Artificial Sequence
  Synthetic
FSCPQCGIDFNQPKSLARHLRLH

SEQ ID NO: 426 - protein, 23 aa, Artificial Sequence
  Synthetic
LQCEICGFTCRQPKSLARHLRLH

SEQ ID NO: 427 - protein, 23 aa, Artificial Sequence
  Synthetic
FHCNQCGASFTQPKSLARHLRLH

SEQ ID NO: 428 - protein, 23 aa, Artificial Sequence
  Synthetic
YSCPVCGLRFKQPKSLARHLRLH

SEQ ID NO: 429 - protein, 23 aa, Artificial Sequence
  Synthetic
HECKLCGASFRQPKSLARHLRLH

SEQ ID NO: 430 - protein, 23 aa, Artificial Sequence
  Synthetic
YKCLECGKSFSQPKSLARHLRLH

SEQ ID NO: 431 - protein, 23 aa, Artificial Sequence
  Synthetic
LQCEICGYQCRQPKSLARHLRLH

SEQ ID NO: 432 - protein, 23 aa, Artificial Sequence
  Synthetic
FKCADCGKGFSQPKSLARHLRLH

SEQ ID NO: 433 - protein, 23 aa, Artificial Sequence
  Synthetic
YICNVCSKTFSQPKSLARHLRLH

SEQ ID NO: 434 - protein, 23 aa, Artificial Sequence
  Synthetic
FQCNTCVKVFSQPKSLARHLRLH

SEQ ID NO: 435 - protein, 23 aa, Artificial Sequence
  Synthetic
LQCEVCGFQCRQPKSLARHLRLH

SEQ ID NO: 436 - protein, 23 aa, Artificial Sequence
  Synthetic
YGCNECGKNFGQPKSLARHLRLH

SEQ ID NO: 437 - protein, 23 aa, Artificial Sequence
  Synthetic
YKCQECDKAFKQPKSLARHLRLH

SEQ ID NO: 438 - protein, 23 aa, Artificial Sequence
  Synthetic
LQCPDCDMTFPQPKSLARHLRLH

SEQ ID NO: 439 - protein, 23 aa, Artificial Sequence
  Synthetic
FACVICGRKFRQPKSLARHLRLH

SEQ ID NO: 440 - protein, 23 aa, Artificial Sequence
  Synthetic
FVCPRCGRGFSQPKSLARHLRLH

SEQ ID NO: 441 - protein, 12 aa, Artificial Sequence
  Synthetic
RKSYWKRHMVIH

SEQ ID NO: 442 - protein, 23 aa, Artificial Sequence
  Synthetic
YKCLECGKSFSRKSYWKRHMVIH

SEQ ID NO: 443 - protein, 23 aa, Artificial Sequence
  Synthetic
LQCEICGYQCRRKSYWKRHMVIH

SEQ ID NO: 444 - protein, 23 aa, Artificial Sequence
  Synthetic
FICGQCGLAFKRKSYWKRHMVIH

SEQ ID NO: 445 - protein, 23 aa, Artificial Sequence
  Synthetic
YGCNECGKNFGRKSYWKRHMVIH

SEQ ID NO: 446 - protein, 23 aa, Artificial Sequence
  Synthetic
LQCEVCGFQCRRKSYWKRHMVIH

SEQ ID NO: 447 - protein, 23 aa, Artificial Sequence
  Synthetic
FSCPQCGIDFNRKSYWKRHMVIH

SEQ ID NO: 448 - protein, 23 aa, Artificial Sequence
  Synthetic
FQCNQCGASFTRKSYWKRHMVIH

SEQ ID NO: 449 - protein, 23 aa, Artificial Sequence
  Synthetic
YSCPVCGLRFKRKSYWKRHMVIH

SEQ ID NO: 450 - protein, 23 aa, Artificial Sequence
  Synthetic
FVCPRCGRGFSRKSYWKRHMVIH

SEQ ID NO: 451 - protein, 23 aa, Artificial Sequence
  Synthetic
LQCEICGFTCRRKSYWKRHMVIH

SEQ ID NO: 452 - protein, 23 aa, Artificial Sequence
  Synthetic
FACVICGRKFRRKSYWKRHMVIH

SEQ ID NO: 453 - protein, 23 aa, Artificial Sequence
  Synthetic
FKCADCGKGFSRKSYWKRHMVIH

SEQ ID NO: 454 - protein, 23 aa, Artificial Sequence
  Synthetic
YICNVCSKTFSRKSYWKRHMVIH

SEQ ID NO: 455 - protein, 23 aa, Artificial Sequence
  Synthetic
HECKLCGASFRRKSYWKRHMVIH

SEQ ID NO: 456 - protein, 23 aa, Artificial Sequence
  Synthetic
FQCPICGLVIKRKSYWKRHMVIH

SEQ ID NO: 457 - protein, 23 aa, Artificial Sequence
  Synthetic
YRCRACGRACSRKSYWKRHMVIH

SEQ ID NO: 458 - protein, 23 aa, Artificial Sequence
  Synthetic
YQCKVCGRAFKRKSYWKRHMVIH

SEQ ID NO: 459 - protein, 23 aa, Artificial Sequence
  Synthetic
FHCNQCGASFTRKSYWKRHMVIH

SEQ ID NO: 460 - protein, 23 aa, Artificial Sequence
  Synthetic
LQCPDCDMTFPRKSYWKRHMVIH

SEQ ID NO: 461 - protein, 23 aa, Artificial Sequence
  Synthetic
FQCNTCVKVFSRKSYWKRHMVIH

SEQ ID NO: 462 - protein, 12 aa, Artificial Sequence
  Synthetic
CHAYLLVHRRIH

SEQ ID NO: 463 - protein, 23 aa, Artificial Sequence
  Synthetic
HECKLCGASFRCHAYLLVHRRIH

SEQ ID NO: 464 - protein, 23 aa, Artificial Sequence
  Synthetic
FQCPICGLVIKCHAYLLVHRRIH

SEQ ID NO: 465 - protein, 23 aa, Artificial Sequence
  Synthetic
FICGQCGLAFKCHAYLLVHRRIH

SEQ ID NO: 466 - protein, 23 aa, Artificial Sequence
  Synthetic
FQCNTCVKVFSCHAYLLVHRRIH

SEQ ID NO: 467 - protein, 23 aa, Artificial Sequence
  Synthetic
FSCPQCGIDFNCHAYLLVHRRIH

SEQ ID NO: 468 - protein, 23 aa, Artificial Sequence
  Synthetic
LQCEICGYQCRCHAYLLVHRRIH

SEQ ID NO: 469 - protein, 23 aa, Artificial Sequence
  Synthetic
FQCNQCGASFTCHAYLLVHRRIH

SEQ ID NO: 470 - protein, 23 aa, Artificial Sequence
  Synthetic
YICNVCSKTFSCHAYLLVHRRIH

SEQ ID NO: 471 - protein, 23 aa, Artificial Sequence
  Synthetic
YSCPVCGLRFKCHAYLLVHRRIH

SEQ ID NO: 472 - protein, 23 aa, Artificial Sequence
  Synthetic
FKCADCGKGFSCHAYLLVHRRIH

SEQ ID NO: 473 - protein, 23 aa, Artificial Sequence
  Synthetic
YKCLECGKSFSCHAYLLVHRRIH

SEQ ID NO: 474 - protein, 23 aa, Artificial Sequence
  Synthetic
YKCQECDKAFKCHAYLLVHRRIH

SEQ ID NO: 475 - protein, 23 aa, Artificial Sequence
  Synthetic
YGCNECGKNFGCHAYLLVHRRIH

SEQ ID NO: 476 - protein, 23 aa, Artificial Sequence
  Synthetic
FHCNQCGASFTCHAYLLVHRRIH

SEQ ID NO: 477 - protein, 23 aa, Artificial Sequence
  Synthetic
LQCPDCDMTFPCHAYLLVHRRIH

SEQ ID NO: 478 - protein, 23 aa, Artificial Sequence
  Synthetic
LQCEVCGFQCRCHAYLLVHRRIH

SEQ ID NO: 479 - protein, 23 aa, Artificial Sequence
  Synthetic
YQCKVCGRAFKCHAYLLVHRRIH

SEQ ID NO: 480 - protein, 23 aa, Artificial Sequence
  Synthetic
YRCRACGRACSCHAYLLVHRRIH

SEQ ID NO: 481 - protein, 23 aa, Artificial Sequence
  Synthetic
FVCPRCGRGFSCHAYLLVHRRIH

SEQ ID NO: 482 - protein, 23 aa, Artificial Sequence
  Synthetic
LQCEICGFTCRCHAYLLVHRRIH

SEQ ID NO: 483 - protein, 23 aa, Artificial Sequence
  Synthetic
FACVICGRKFRCHAYLLVHRRIH

SEQ ID NO: 484 - protein, 12 aa, Artificial Sequence
  Synthetic
YSSALSTHKIIH

SEQ ID NO: 485 - protein, 23 aa, Artificial Sequence
  Synthetic
YICNVCSKTFSYSSALSTHKIIH

SEQ ID NO: 486 - protein, 23 aa, Artificial Sequence
  Synthetic
YGCNECGKNFGYSSALSTHKIIH

SEQ ID NO: 487 - protein, 23 aa, Artificial Sequence
  Synthetic
LQCEICGFTCRYSSALSTHKIIH

SEQ ID NO: 488 - protein, 23 aa, Artificial Sequence
  Synthetic
FQCNTCVKVFSYSSALSTHKIIH

SEQ ID NO: 489 - protein, 23 aa, Artificial Sequence
  Synthetic
LQCPDCDMTFPYSSALSTHKIIH

SEQ ID NO: 490 - protein, 23 aa, Artificial Sequence
  Synthetic
LQCEICGYQCRYSSALSTHKIIH

SEQ ID NO: 491 - protein, 23 aa, Artificial Sequence
  Synthetic
FVCPRCGRGFSYSSALSTHKIIH

SEQ ID NO: 492 - protein, 23 aa, Artificial Sequence
  Synthetic
FQCPICGLVIKYSSALSTHKIIH

SEQ ID NO: 493 - protein, 23 aa, Artificial Sequence
  Synthetic
YQCKVCGRAFKYSSALSTHKIIH

SEQ ID NO: 494 - protein, 23 aa, Artificial Sequence
  Synthetic
LQCEVCGFQCRYSSALSTHKIIH

SEQ ID NO: 495 - protein, 23 aa, Artificial Sequence
  Synthetic
FQCNQCGASFTYSSALSTHKIIH

SEQ ID NO: 496 - protein, 23 aa, Artificial Sequence
  Synthetic
FACVICGRKFRYSSALSTHKIIH

SEQ ID NO: 497 - protein, 23 aa, Artificial Sequence
  Synthetic
FICGQCGLAFKYSSALSTHKIIH

SEQ ID NO: 498 - protein, 23 aa, Artificial Sequence
  Synthetic
FKCADCGKGFSYSSALSTHKIIH

SEQ ID NO: 499 - protein, 23 aa, Artificial Sequence
  Synthetic
YKCLECGKSFSYSSALSTHKIIH

SEQ ID NO: 500 - protein, 23 aa, Artificial Sequence
  Synthetic
YRCRACGRACSYSSALSTHKIIH

SEQ ID NO: 501 - protein, 23 aa, Artificial Sequence
  Synthetic
YKCQECDKAFKYSSALSTHKIIH

SEQ ID NO: 502 - protein, 23 aa, Artificial Sequence
  Synthetic
HECKLCGASFRYSSALSTHKIIH

SEQ ID NO: 503 - protein, 23 aa, Artificial Sequence
  Synthetic
FSCPQCGIDFNYSSALSTHKIIH

SEQ ID NO: 504 - protein, 23 aa, Artificial Sequence
  Synthetic
FHCNQCGASFTYSSALSTHKIIH

SEQ ID NO: 505 - protein, 23 aa, Artificial Sequence
  Synthetic
YSCPVCGLRFKYSSALSTHKIIH

SEQ ID NO: 506 - protein, 12 aa, Artificial Sequence
  Synthetic
DHSNLITHQRIH

SEQ ID NO: 507 - protein, 23 aa, Artificial Sequence
  Synthetic
YKCQECDKAFKDHSNLITHQRIH

SEQ ID NO: 508 - protein, 23 aa, Artificial Sequence
  Synthetic
FQCNTCVKVFSDHSNLITHQRIH

SEQ ID NO: 509 - protein, 23 aa, Artificial Sequence
  Synthetic
FQCNQCGASFTDHSNLITHQRIH

SEQ ID NO: 510 - protein, 23 aa, Artificial Sequence
  Synthetic
FQCPICGLVIKDHSNLITHQRIH

SEQ ID NO: 511 - protein, 23 aa, Artificial Sequence
  Synthetic
LQCEVCGFQCRDHSNLITHQRIH

SEQ ID NO: 512 - protein, 23 aa, Artificial Sequence
  Synthetic
YGCNECGKNFGDHSNLITHQRIH

SEQ ID NO: 513 - protein, 23 aa, Artificial Sequence
  Synthetic
FICGQCGLAFKDHSNLITHQRIH

SEQ ID NO: 514 - protein, 23 aa, Artificial Sequence
  Synthetic
LQCEICGYQCRDHSNLITHQRIH

SEQ ID NO: 515 - protein, 23 aa, Artificial Sequence
  Synthetic
YRCRACGRACSDHSNLITHQRIH

SEQ ID NO: 516 - protein, 23 aa, Artificial Sequence
  Synthetic
FSCPQCGIDFNDHSNLITHQRIH

SEQ ID NO: 517 - protein, 23 aa, Artificial Sequence
  Synthetic
YQCKVCGRAFKDHSNLITHQRIH

SEQ ID NO: 518 - protein, 23 aa, Artificial Sequence
  Synthetic
YICNVCSKTFSDHSNLITHQRIH

SEQ ID NO: 519 - protein, 23 aa, Artificial Sequence
  Synthetic
FVCPRCGRGFSDHSNLITHQRIH

SEQ ID NO: 520 - protein, 23 aa, Artificial Sequence
  Synthetic
FACVICGRKFRDHSNLITHQRIH

SEQ ID NO: 521 - protein, 23 aa, Artificial Sequence
  Synthetic
HECKLCGASFRDHSNLITHQRIH

SEQ ID NO: 522 - protein, 23 aa, Artificial Sequence
  Synthetic
FHCNQCGASFTDHSNLITHQRIH

SEQ ID NO: 523 - protein, 23 aa, Artificial Sequence
  Synthetic
FKCADCGKGFSDHSNLITHQRIH

SEQ ID NO: 524 - protein, 23 aa, Artificial Sequence
  Synthetic
YKCLECGKSFSDHSNLITHQRIH

SEQ ID NO: 525 - protein, 23 aa, Artificial Sequence
  Synthetic
LQCPDCDMTFPDHSNLITHQRIH

SEQ ID NO: 526 - protein, 23 aa, Artificial Sequence
  Synthetic
YSCPVCGLRFKDHSNLITHQRIH

SEQ ID NO: 527 - protein, 23 aa, Artificial Sequence
  Synthetic
LQCEICGFTCRDHSNLITHQRIH

SEQ ID NO: 528 - protein, 11 aa, Artificial Sequence
  Synthetic
FQCNQCGASFT

SEQ ID NO: 529 - protein, 11 aa, Artificial Sequence
  Synthetic
FQCPICGLVIK

SEQ ID NO: 530 - protein, 11 aa, Artificial Sequence
  Synthetic
LQCEICGFTCR

SEQ ID NO: 531 - protein, 11 aa, Artificial Sequence
  Synthetic
LQCEICGYQCR

SEQ ID NO: 532 - protein, 11 aa, Artificial Sequence
  Synthetic
LQCEVCGFQCR

SEQ ID NO: 533 - protein, 12 aa, Artificial Sequence
  Synthetic
QKGNLLRHIKLH

SEQ ID NO: 534 - protein, 12 aa, Artificial Sequence
  Synthetic
QPKSLARHLRLH

SEQ ID NO: 535 - protein, 12 aa, Artificial Sequence
  Synthetic
RKDRMSYHVRSH

SEQ ID NO: 536 - protein, 12 aa, Artificial Sequence
  Synthetic
RLSTLIQHQKVH

SEQ ID NO: 537 - protein, 12 aa, Artificial Sequence
  Synthetic
TKGSLIRHHRRH

SEQ ID NO: 538 - protein, 12 aa, Artificial Sequence
  Synthetic
NRGLMQKHLKNH

SEQ ID NO: 539 - protein, 12 aa, Artificial Sequence
  Synthetic
RHSHLIEHLKRH

(19) United States
(12) Patent Application Publication
(10) Pub. No.: US 2023/0151342 A1
(43) Pub. Date: May 18, 2023

(54) ZINC FINGER DEGRADATION DOMAINS

(71) Applicants: THE BROAD INSTITUTE, INC, CAMBRIDGE, MA (US); THE BRIGHAM AND WOMEN’S HOSPITAL, INC., BOSTON, MA (US); THE GENERAL HOSPITAL CORPORATION, BOSTON, MA (US); DANA-FARBER CANCER INSTITUTE, INC., BOSTON, MA (US)

(72) Inventors: Amit CHOUDHARY, Boston, MA (US); Donghyun LIM, Cambridge, MA (US); Sreekanth VEDAGOPURAM, Boston, MA (US); Benjamin EBERT, Boston, MA (US); Max JAN, Boston, MA (US)

(21) Appl. No.: 17/802,932

(22) PCT Filed: Feb. 26, 2021
(86) PCT No.: PCT/US2021/020106

§ 371 Date: Aug. 26, 2022

Related U.S. Application Data
(60) Provisional application No. 62/983,448, filed on Feb. 28, 2020.

(51) Int. Cl. C12N 9/22; C12N 15/11; C12N 9/78; C12N 15/63; C12N 15/90; C07K 14/00

(52) U.S. Cl. CPC C12N 9/22; C12N 15/11; C12N 9/78; C12N 15/635; C12N 15/907; C07K 14/00; C12N 2310/20; C12N 2800/80; C07K 2319/95

(57) ABSTRACT

The disclosure includes compositions comprising synthetic zinc finger degrons, and their use with non-naturally occurring or engineered programmable nucleases. Compositions specifically targeting the engineered programmable nucleases for control of gene editing outcomes, and compositions, systems and method of use are further detailed.

### CROSS-REFERENCE TO RELATED APPLICATIONS

This application claims the benefit of U.S. Provisional Application No. 62/983,448 filed Feb. 28, 2020. The entire contents of the above-identified applications are hereby fully incorporated herein by reference.

### STATEMENT REGARDING FEDERALLY SPONSORED RESEARCH

This invention was made with government support under Grant No. N66001-17-2-4055 granted by the Defense Advanced Research Projects Agency; Grant No. AI126239 granted by the National Institutes of Health; and Grant No. W911NF1610586 granted by the Army Research Office. The government has certain rights in the invention.

### REFERENCE TO AN ELECTRONIC SEQUENCE LISTING

The contents of the electronic sequence listing (“BROD-5040WP_ST25.txt”; Size is 165,151 bytes and it was created on Feb. 25, 2021) is herein incorporated by reference in its entirety.

### TECHNICAL FIELD

The subject matter disclosed herein is generally directed to systems for target-specific protein degradation, controlled gene editing and methods of their use.

### BACKGROUND

Recent advances in genome sequencing techniques and analysis methods have significantly accelerated the ability to catalog and map genetic factors associated with a diverse range of biological functions and diseases. Precise genome targeting technologies are needed to enable systematic reverse engineering of causal genetic variations by allowing selective perturbation of individual genetic elements, as well as to advance synthetic biology, biotechnological, and medical applications. Although genome-editing techniques are available for producing targeted genome perturbations, there remains a need for new genome engineering technologies that employ novel strategies and molecular mechanisms and are affordable, easy to set up, scalable, and amenable to targeting multiple positions within the eukaryotic genome.

RNA-guided endonucleases, such as Cas9, are easily targeted to any desired DNA or RNA locus using guide RNAs (gRNA), which has provided new transformative technologies. For example, Cas9 has enabled facile and efficient induction of genomic alterations in cells and multiple organisms, and Cas9-based gene drives permit super-Mendelian self-propagation of such modifications (3). Furthermore, catalytically inactive CRISPR effectors, such as Cas9 (dCas9) can be fused to a wide range of effectors, including fluorescent proteins for genome imaging (4), enzymes that modify DNA or histones for epigenome editing (5), and transcription regulating domains for controlling endogenous gene expression (6). Streptococcus pyogenes and Staphylococcus aureus provide naturally occurring SpCas9 and SaCas9, respectively, that are commonly used in CRISPR approaches.

Despite such advances, a critical need still exists for methods to precisely and switchably regulate CRISPR effector activities across multiple dimensions, including dose, target, and time (7). Finely-tuned control of CRISPR effector proteins levels is important, as high concentrations result in elevated off-target DNA cleavage. Rapidly disabling activity after a desired genomic modification is also essential (8). However, the ability to control such systems is still needed. One method of control would be degradation of the Cas effector protein to effectively shut down systems after use. Typically, once proteins are no longer needed in a cell, they are tagged in the cell with ubiquitin utilizing an E3 ligase to designate the protein for degradation in the proteasome. Exploitation of a mechanism to target proteins for degradation in the proteasome would be one approach to degrade Cas effector protein after its use and provide a means of control after desired genomic modification or other uses of CRISPR Cas systems has been effected.

### SUMMARY

In exemplary embodiments, hybrid zinc finger polypeptides are provided. In embodiments, the hybrid zinc finger polypeptide comprises a sequence selected from Table 2, 3A or 3B. In one embodiment, In certain embodiments, the hybrid zinc finger polypeptide comprises an N-terminal bet hairpin subdomain selected from SEQ ID NOs: 46, 49, 51, 53, 55, 57, 59, 61, 63, 65, 67, 69, 71, 73, 75, 77, 79, 81, 83, 85, 87; and a C-terminal alpha-helix subdomain selected from SEQ ID NOs: 47, 89, 111, 133, 155, 177, 199, 221, 243, 265, 287, 309, 331, 353, 375, 397, 419, 441, 462, 484, and 506. In an aspect, the hybrid zinc finger polypeptide comprises a sequence selected from SEQ ID NOs: 48, 50, 52, 54, 56, 58, 60, 62, 64, 66, 68, 70, 72, 74, 76, 78, 80, 82, 84, 86, 88, 90, 91, 92, 93, 94, 95, 96, 97, 98, 99, 100, 101, 102, 103, 104, 105, 106, 107, 108, 109, 110, 112, 113, 114, 115, 116, 117, 118, 119, 120, 121, 122, 123, 124, 125, 126, 127, 128, 129, 130, 131, 132, 134, 135, 136, 137, 138, 139, 140, 141, 142, 143, 144, 145, 146, 147, 148, 149, 150, 151, 152, 153, 154, 156, 157, 158, 159, 160, 161, 162, 163, 164, 165, 166, 167, 168, 169, 170, 171, 172, 173, 174, 175, 176, 178, 179, 180, 181, 182, 183, 184, 185, 186, 187, 188, 189, 190, 191, 192, 193, 194, 195, 196, 197, 198, 200, 201, 202, 203, 204, 205, 206, 207, 208, 209, 210, 211, 212, 213, 214, 215, 216, 217, 218, 219, 220, 222, 223, 224, 225, 226, 227, 228, 229, 230, 231, 232, 233, 234, 235, 236, 237, 238, 239, 240, 241, 242, 244, 245, 246, 247, 248, 249, 250, 251, 252, 253, 254, 255, 256, 257, 258, 259, 260, 261, 262, 263, 264, 266, 267, 268, 269, 270, 271, 272, 273, 274, 275, 276, 277, 278, 279, 280, 281, 282, 283, 284, 285, 286, 288, 289, 290, 291, 292, 293, 294, 295, 296, 297, 298, 299, 300, 301, 302, 303, 304, 305, 306, 307, 308, 310, 311, 312, 313, 314, 315, 316, 317, 318, 319, 320, 321, 322, 323, 324, 325, 326, 327, 328, 329, 330, 332, 333, 334, 335, 336, 337, 338, 339, 340, 341, 342, 343, 344, 345, 346, 347, 348, 349, 350, 351, 352, 354, 355, 356, 357, 358, 359, 360, 361, 362, 363, 364, 365, 366, 367, 368, 369, 370, 371, 372, 373, 374, 376, 377, 378, 379, 380, 381, 382, 383, 384, 385, 386, 387, 388, 389, 390, 391, 392, 393, 394, 395, 396, 398, 399, 400, 401, 402, 403, 404, 405, 406, 407, 408, 409, 410, 411, 412, 413, 414, 415, 416, 417, 418, 420, 421, 422, 423, 424, 425, 426, 427, 428, 429, 430, 431, 432, 433, 434, 435, 436, 437, 438, 439, 440, 442, 443, 444, 445, 446, 447, 448, 449, 450, 451, 452, 453, 454, 455, 456, 457, 458, 459, 460, 461, 463, 464, 465, 466, 467, 468, 469, 470, 471, 472, 473, 474, 475, 476, 477, 478, 479, 480, 481, 482, 483, 485, 486, 487, 488, 489, 490, 491, 492, 493, 494, 495, 496, 497, 498, 499, 500, 501, 502, 503, 504, 505, 507, 508, 509, 510, 511, 512, 513, 514, 515, 516, 517, 518, 519, 520, 521, 522, 523, 524, 525, 526, or 527. In an aspect, the hybrid zinc finger polypeptide is optimized for degradation by pomalidomide, avadomide, lenalidomide, iberomide, or another thalidomide analog.

In one embodiment, the hybrid zinc finger polypeptide is optimized for degradation by pomalidomide, and the zinc finger polypeptide comprises a sequence selected from SEQ ID NOs: 175, 361, 201, 457, 269, 110, 84, 246, 168, 359, 203, 448, 278, 102, 48, 209, 450, 285, 109, 440, 171, 367, 218, 277, 107, 161, 366, 214, 443, 283, 172, 364, 216, 451, 284, 162, 371, 165, 370, 444, 452, 170, 91, 82, 373, and 156.

In one embodiment, the hybrid zinc finger polypeptide is optimized for degradation by avadomide, and the zinc finger polypeptide comprises a sequence selected from SEQ ID NOs: 175, 361, 457, 201, 269, 110, 84, 246, 168, 359, 448, 203, 278, 102, 171, 367, 445, 277, 107, 182, 163, 360, 450, 209, 109, 164, 354, 452, 219, 271, 161, 366, 443, 283, 162, 371, 446, 170, 365, 91, 172, 364, 451, 373, 156, 357, and 444.

In one embodiment the hybrid zinc finger polypeptide is optimized for degradation by iberomide, and the zinc finger polypeptide comprises a sequence selected from SEQ ID NOs: 360, 209, 405, 109, 440, 359, 203, 448, 48, 102, 278, 367, 171, 218, 445, 74, 107, 361, 175, 201, 84, 371, 162, 215, 446, 443, 354, 164, 219, 452, 170, 82, 91, 364, 172, 216, 373, 212, 165, and 156.

In one embodiment, the hybrid zinc finger polypeptide is optimized for degradation by lenalidomide, and the zinc finger polypeptide comprises a sequence selected from SEQ ID NOs: 445, 455, 91, 373, 449, 160, 212, 354, 452, 164, 219, 359, 448, 168, 102, 361, 457, 175, 201, 360, 450, 163, 209, and 109.

In one embodiment, a programmable nuclease is provided comprising one or more hybrid zinc finger polypeptides introduced into the nuclease at one or more insertion sites. In an embodiment, the hybrid zinc finger peptides can be utilized as a degradation domains in a modified programmable nuclease, which may be a CRISPR-Cas protein, a Zinc finger nuclease, a TALEN or a meganuclease. CRISPR-Cas proteins and other programmable nucleases which may be further comprise fusion domains and used as base editors, transposases or in other applications can be utilized with the hybrid zinc finger polypeptides without loss of function. In an aspect a programmable nuclease, for example, a CRISPR-Cas protein comprising one or more zinc finger degradation domains introduced into the CRISPR-Cas protein at one or more insertion sites is provided. The variant CRISPR-Cas protein may comprise a Type II, Type V or Type VI Cas protein, in an aspect, wherein the CRISPR-Cas protein is a Cas9, a Cas12a, Cas12b, Cas12c, Cas12d, Cas13a, Cas13b, Cas13c, or Cas13d protein. The variant CRISPR-Cas polypeptide may be codon optimized for expression in eukaryotes.

In certain embodiments, the variant CRISPR-Cas protein comprising a zinc finger degradation domain may comprise one or more insertion sites at the N-terminal (Nt), C-terminal (Ct) or at a position corresponding to as position on the loop of a SpCas9 protein. In an aspect, the variant CRISPR-Cas protein comprises SEQ ID NO: 45.

A ribonucleoprotein comprising the variant CRISPR-Cas protein that comprises a degradation domain is disclosed herein. Embodiments include a plasmid comprising the variant CRISPR-Cas protein and a cell transfected with the ribonucleoprotein or the plasmid comprising the variant CRISPR-Cas protein.

A method of inducing degradation of a variant CRISPR-Cas protein is provided, comprising: exposing a cell comprising or expressing a variant CRISPR-Cas protein with an immunomodulatory imide drug (IMiD) or a pharmaceutically acceptable salt thereof, in embodiments, the IMiD is selected from thalidomide, lenalidomide, pomalidomide, avadomide, iberdomide, and analogs thereof. Exposing the cell with the IMiD is in certain embodiments performed about 3 to 6 hours after the cell is transfected. In an aspect, exposing comprises incubating the cell with the compound or pharmaceutically acceptable salt thereof, wherein the compound is provided at a concentration of about 10 nM to about 10 μM. In certain embodiments, the cell is a germline cell. In embodiments, the cell is in an organism.

The methods disclosed herein can utilize CRISPR-Cas proteins with degradation domains optimized for particular immunomodulatory inducing drugs, for example pomalidomide, avadomide, iberomide or lenalidomide.

A method of controlling CRISPR-Cas protein editing outcomes can comprise administering an immunomodulatory imide drug (IMiD) or a pharmaceutically acceptable salt thereof to a cell or a population of cells comprising or expressing a variant CRISPR-Cas protein according to the embodiments disclosed herein.

The method may be performed in vitro or in vivo. The step of exposing or administering of the IMiD to the cell can be performed at a time to encourage microhomology repair or single base insertion outcomes, or to promote HDR repair pathways over NHEJ repair pathways.

The methods disclosed include embodiments wherein the variant CRISPR-Cas protein comprises degradation domains, at one or more insertion sites are at the N-terminal (Nt), C-terminal (Ct) or at a position corresponding to the loop on a Cas protein, preferably position 231 (Lp) of a SpCas9 protein. In embodiments, the variant CRISPR-Cas protein insertion sites are selected from: Nt and Ct; Nt and Lp; Lp and Ct; and Nt, Lp and Ct.

In embodiments, the variant CRISPR Cas protein is a Cas9, a Cas12a, Cas12b, Cas12c, Cas12d, Cas13a, Cas13b, Cas13c, or Cas13d protein, in one aspect preferably CRISPR Cas 9. In certain embodiments of the method, the cell is exposed to the compound or pharmaceutically acceptable salt thereof at a concentration of about 10 nM to about 10 μM. In some methods, the step of exposing comprises incubating the cell with the compound or pharmaceutically acceptable salt thereof.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A shows Non-homologous End Joining (Non-MH deletions outcomes predominate early on after Cas9 treatment, with 1 bp insertions increasing the longer Cas9 is present; FIG. 1B charts observed CRISPR phenotypes increasing relative to wildtype observation the longer Cas9 is present.

FIG. 2 charts the % of 1 bp insertions based on the 3 categories of the 48 gRNA library, namely, control, insertion, and microhomology precision libraries.

FIG. 3 shows that in both insertion and microhomology precision libraries, microhomology deletions events require longer presence of Cas9.

FIG. 4 depicts Cys2His2 (C2H2) zinc finger degron-Cas9 example embodiment constructs along with proteasomal degradation in the presence of thalidomide and/or its analogues such as lenalidomide and pomalidomide.

FIG. 5A-5B—Activity of example embodiment single degron-Cas9 constructs, super-degron (FIG. 5A) and minimal degron (FIG. 5B) in an eGFP disruption assay, N is degron insertion at N-terminal of Cas9, L is degron insertion at the Cas9 loop, and C is degron insertion at C-terminal of Cas9 construct.

FIG. 6—Imaging of activity of single degron-Cas9 exemplary constructs (eGFP disruption assay)

FIG. 7—Dose curves for exemplary single Super Degron-Cas9 constructs (eGFP disruption assay)

FIG. 8—shows exemplary L-SD-Cas9 degradation in HEK293T cells

FIG. 9A-9B dose curve for exemplary super degron constructs in eGFP disruption assay (9A) and dose curves for exemplary minimal degron constructs eGFP disruption assay (R1)(9B).

FIG. 10A-10D—Engineering example embodiment lenalidomide ON- and OFF-switch controllable CAR T cells. (FIG. 10A) Degradable CARs can be depleted from the cell surface upon addition of lenalidomide or other thalidomide analogs via recruitment to the CRL4^{CRBN} E3 ubiquitin ligase, ubiquitination, and proteasomal degradation. (FIG. 10B) Jurkat cells were engineered to express an anti-CD19 CAR or the same with addition of an example embodiment zinc finger degron from IKZF3 (19BBz-dIKZF3), exposed to 1 μM lenalidomide or vehicle control overnight, and analyzed by flow cytometry for CAR expression. UTD, untransduced. (FIG. 10C) Split CARs incorporating an exemplary lenalidomide-inducible dimerization domain composed of fragments of CRBN (left) and IKZF3 (right) are licensed by lenalidomide for antigen-dependent activation. (FIG. 10D) Jurkat cells were engineered to express an anti-CD19 CAR (1928z) or a split CAR, co-cultured overnight with the indicated target cells and 1 μM lenalidomide or vehicle control, and analyzed by flow cytometry to quantify the percentage of CD69+ cells. Experiments were performed in duplicate (10B) or triplicate (10D); Error bars indicate standard deviation.

FIG. 11A-11H—A screen of 440 hybrid zinc fingers identifies example embodiment “super-degrons” targeted by sub-nanomolar concentrations of thalidomide analogs (FIG. 11A) Schematic for the design and screening of a hybrid zinc finger library encoded in a GFP-tagged protein degradation reporter lentivector. Jurkat cells were transduced with this lentivirus library, and then exposed to various thalidomide analogs or vehicle control. FACS sorting was used to isolate GFP^{low} cells, and next-generation sequencing was then used to quantify the relative abundance of each sequence with and without drug treatment. Flow plot for Jurkat cells transduced with the GFP-tagged zinc finger library of example embodiment, which also expresses mCherry as a control for lentivector transgene expression (FIG. 11B), after overnight incubation with 1 μM lenalidomide or vehicle control. (FIG. 11C) Fold-enrichment of sequencing read counts (lenalidomide/DMSO) and corresponding P values. (FIG. 11D) Sequence features for N- and C-terminal domains present in example embodiment top candidate super-degrons. Amino acid positions with prior crystallographic evidence of side-chain interactions with pomalidomide (open circle) or CRBN (open circle) are noted. (FIG. 11E) Vehicle control-normalized eGFP/mCherry fluorescence ratios measured by flow cytometry for Jurkat cells expressing the indicated zinc finger constructs after treatment with lenalidomide or iberdomide (FIG. 11F). IC50 values for the indicated endogenous and exemplary hybrid zinc fingers calculated from single reporter degradation experiments. (FIG. 11G). EC50 values for the indicated endogenous and hybrid zinc fingers calculated from single reporter degradation experiments. Experiments were performed in triplicate and error bars indicate standard deviation (FIG. 11H).

FIG. 12A-12D—ON-switch split CARs only function in the presence of lenalidomide. (FIG. 12A) Schematic of split CAR constructs. Each split CAR is composed of the indicated antigen-binding part A and the ITAM-containing part B. The lenalidomide-induced dimerization module is encoded by zinc fingers from IKZF3 or the engineered 913 zinc finger and a fragment of CRBN (CRBNΔ3). The intracellular domains of each split CAR part A is protected from CRL4^{CRBN} ubiquitination by K>R “K0” substitutions. The control second generation CAR FMC63-CD28-CD3z was also used. sCAR, split CAR. (FIG. 12B) CAR-Jurkat cells were co-cultured with K562 or K562-CD19 cells and lenalidomide or vehicle control and then analyzed by flow cytometry to quantify the percentage of CD69+ cells. EC₅₀ values for the sCAR-IKZF3 and sCAR-91.3 are 206.2 and 29.3 nM lenalidomide, respectively. (FIG. 12C) Primary T cells were infected with lentiviruses encoding parts A and B of split CAR 913. Untransduced cells and cells expressing components A only, B only, and both A+B were purified by FACS. Cytotoxic activity of each sorted cell population was measured after overnight co-culture with NALM6 target cells and lenalidomide or vehicle control at the indicated effector:target ratios. The maximum plasma concentration for once daily 25 mg lenalidomide in multiple myeloma patients is indicated. (FIG. 12D) Scatterplots showing the production of cytokines after co-culture (1:1 CAR T:NALM6 ratio) in the presence of 1000 nM lenalidomide versus vehicle control. Experiments were performed in triplicate and error bars indicate standard deviation.

FIG. 13A-13H—Functional control of degradable CAR T cell activation. (FIG. 13A) Schematic of CAR constructs with or without degron tags. CAR-Jurkat cells were treated with lenalidomide or vehicle control and then (FIG. 13B) analyzed by western blot for the specified targets or (FIG. 13C) analyzed by flow cytometry to quantify the CAR protein abundance normalized to vehicle control (anti-Myc tag). (FIG. 13D) CAR-Jurkat cells were co-cultured with K562-CD19 cells and lenalidomide or vehicle control and then analyzed by flow cytometry for the percentage of CD69+ cells. (FIG. 13E) The concentration of IL2 in supernatants from FIG. 13D was measured by ELISA. (FIG. 13F) IC50 values and 95% confidence intervals calculated from dose response experiments described in FIG. 13C-FIG. 13E. (FIG. 13G) Time course of CAR depletion upon addition of lenalidomide (t½=0.33 h, 95% CI 0.29-0.38). (FIG. 13H) Time course of CAR re-expression following lenalidomide treatment and drug washout (t½=3.57 h, 95% CI 1.88-13.6). All experiments were performed in triplicate. Error bars indicate standard deviation.

FIG. 14A-14I—OFF-switch degradable CARs can be transiently depleted with pomalidomide and enforce tumor control in vivo. (FIG. 14A) Schematic of luciferase-tagged CAR constructs. (FIG. 14B) Experimental design for in vivo CAR depletion model: NSG mice were injected intravenously with 5e6 Jurkat cells expression 19BBz-FLuc-d91.3 or 19BBz-FLuc-d91.3*; after allowing for engraftment, bioluminescent imaging (BLI) was performed before and after one dose of 10 mg/kg pomalidomide administered by oral gavage. (FIG. 14C) Summary of BLI 24 hours before, 6 hours after, and 24 hours after pomalidomide. Comparing the d91.3 and d91.3* CARs across each timepoint using two-tailed t-tests yielded p-values of 0.35, 0.003, and 0.14, respectively. (FIG. 14D) BLI representing CAR abundance over time. (FIG. 14E) Experimental design for in vivo tumor control model: NSG mice were injected intravenously with 1e6 GFP+/luciferase+ JeKo-1 tumor cells. At day 0, mice were randomly assigned on the basis of tumor burden to receive 1e6 control T cells (UTD), 19BBz, or 19BBz-d91.3. (FIG. 14F) Average luminescence of whole mice in the 3 groups over time. (FIG. 14G) Representative BLI demonstrating tumor burden over time. The percentage of JeKo-1 cells (FIG. 14H) and human T cell (FIG. 14I) among mononuclear cells in the bone marrow or spleen at day 35.

FIG. 15A-15E—OFF-switch degradable CAR T cell cytotoxicity and cytokine production can be inhibited in vitro and in vivo. (FIG. 15A) Cytotoxic activity of 19BBz and 19BBz-d91.3 CAR T cells measured after overnight co-culture with NALM6 target cells and lenalidomide or vehicle control. The cytotoxicity assay is representative of 3 independent experiments conducted with different healthy donors. (FIG. 15B) Scatterplots showing the concentration of cytokines in pg/mL after co-culture (9:1 CAR T:NALM6 ratio) in the presence of 100 nM lenalidomide versus vehicle control by 19BBz or 19BBz-d91.3 CART cells. UTD=untransduced. experiments were performed in triplicate. Error bars indicate standard deviation. (FIG. 15C) Experimental design for in vivo CAR T cell cytokine release model: NSG mice were injected intravenously with 1e6 NALM6 cells. At day 0, mice were randomly assigned on the basis of tumor burden to receive 2e6 control T cells (UTD), 19BBz, or 19BBz-d91.3. From days 3-5, mice received no treatment, once daily, or twice daily 30 mg/kg pomalidomide by oral gavage. On the afternoon of day 5, serum was collected for cytokine analysis. (FIG. 15D) Serum IFN-gamma concentration on day 5. (FIG. 15E) Serum IL-2 concentration on day 5.

FIG. 16A-16D—Engineering of a lenalidomide-inducible dimerization system and ON-switch split CAR. (FIG. 16A) Schema for the discrete steps in receptor engineering. For experiments FIG. 16B-FIG. 16D, NanoBRET was used to measure the association between proteins bearing Nanoluc luciferase and HaloTag in 293T cells. 2 hours after addition of MG132 and lenalidomide or vehicle control, the Nanoluc substrate was added and BRET signal was assessed using a plate reader. (FIG. 16B) NanoBRET analysis of dIKZF3 interaction with CRBN deletion variants. (FIG. 16C) NanoBRET analysis of dIKZF3-CRBNΔ3 incorporated into cell surface-localized fusion proteins. 1928=FMC63 scFv—CD28 costimulatory domain. CD8-CD28=CD8 hinge and transmembrane domain and CD28 co-stimulatory domain. PD1=PD1 transmembrane and cytoplasmic domain. Myr-CD28=LYN myristoylation and palmitoylation motif—CD28 costimulatory domain. (FIG. 16D) NanoBRET analysis of CD8-CD28-CRBNΔ3 and 1928dIKZF3 with or without intracellular K->R mutations (iK0).

FIG. 17A-17E Hybrid C2H2 zinc finger library screen. (FIG. 17A)—Hybrid C2H2 zinc finger library screen for pomalidomide-induced degrons. Average fold-enrichment of sequencing read counts (pomalidomide/DMSO) and corresponding P values; (FIG. 17B)—Hybrid C2H2 zinc finger library screen for avadomide-induced degrons. Average fold-enrichment of sequencing read counts (avadomide/DMSO) and corresponding P values; (FIG. 17C)—Hybrid C2H2 zinc finger library screen for iberomide-induced degrons. Average fold-enrichment of sequencing read counts (iberomide/DMSO) and corresponding P values; (FIG. 17D) Fold enrichment and significance of sequences enriched with lenalidomide versus vehicle control, ordered by cumulative enrichment of N- and C-terminal domains for lenalidomide-induced degrons; (FIG. 17E) Fold enrichment and significance of sequences enriched with lenalidomide versus vehicle control, ordered by cumulative enrichment of N- and C-terminal domains. Inset demonstrates subset of N- and C-terminal domains that combine to generate the majority of top hits.

FIG. 18A-18B Validation of individual hybrid zinc finger degrons. (FIG. 18A) Vehicle control-normalized eGFP/mCherry fluorescence ratios measured by flow cytometry for Jurkat cells expressing the indicated minimal 23 amino acid zinc finger degron constructs after treatment with pomalidomide or vehicle control. Experiments were performed in triplicate and error bars indicate standard deviation. IC₅₀ values for PATZ1 (32.4 nM), ZN653 (5.17 nM), ZN653-PATZ1 (0.160 nM). (FIG. 18B) IC₅₀ values for lenalidomide- or pomalidomide-induced degradation of endogenous and hybrid zinc fingers calculated from single reporter degradation experiments. (FIG. 18C) Jurkat cells expressing the 19BBz-d91.3 CAR were treated overnight with lenalidomide and the E1 inhibitor MLN7243 (500 nM), the Neddylation inhibitor MLN4294 (5000 nM), the lysosomal acidification inhibitor Chloroquine (50,000 nM), or the lysosomal acidification inhibitor Bafilomycin A (100 nM). CAR degradation requires ubiquitin ligase and Cullin-RING ligase function, and is insensitive to inhibition of autophagy.

FIG. 19A-19B OFF-switch degradable CAR gated by lenalidomide. (FIG. 19A) CAR-Jurkat cells were treated with pomalidomide or vehicle control and then analyzed by flow cytometry to quantify the CAR protein abundance normalized to vehicle control (anti-Myc tag). (FIG. 19B) CAR-Jurkat cells were co-cultured with K562-CD19 cells and pomalidomide or vehicle control and then analyzed by flow cytometry for the percentage of CD69+ cells. (FIG. 19C) Luciferase-tagged degradable CAR abundance can be monitored by bioluminescence. Normalized luminescence of firefly luciferase-tagged degradable CAR Jurkat cells following overnight exposure to lenalidomide or vehicle control.

FIG. 20. Schema for the functional genomic screening of a hybrid zinc finger library for sequences that are efficiently degraded with the indicated thalidomide analogs.

FIG. 21. Scheme to sort cells with low GFP expression. The gate is unchanged across each drug concentration. The increase in the fraction of GFP low cells in the various drug concentrations is indicative of drug-dependent degradation of a subset of sequences in the library. Concentrations used in screen: 1 uM lenalidomide, 1 uM pomalidomide, 1 uM CC-122 aka iberdomide, 0.05 uM CC-220 aka avadomide.

FIG. 22. Waterfall plot of significance versus fold-enrichment in the sorted population (GFP low), lenalidomide versus vehicle control. Endogenous ZF domains are highlighted orange. Select candidate super-degrons are colored blue and labeled.

FIG. 23. Validation of individual hybrid zinc finger degrons. Individual 23 amino acid zinc finger domains were cloned into the Cilantro 2 protein degradation reporter lentivector. Jurkat cells were transduced with each of these viruses. The GFP/mCherry ratio was calculated in the presence of various thalidomide analogs, indicative of drug-dependent degradation. The EC50 for degradation of each sequence is also presented in table format. Dark Gray=hybrid zinc fingers. Light Gray=endogenous zinc fingers. Dotted line=ZFP91-IKZF3.

FIG. 24 Validation of lenalidomide-OFF-switch control of CAR T cell activation, as assessed by expression of the early activation marker CD69, in Jurkat T cells expressing various super-degron tagged chimeric antigen receptors. Regulation of CAR T cell activation with the indicated super-degrons, in comparison to the previously described degron d913. CARs with dZFP91-ZN787 and dZN653-PATZ1 degrons are more efficiently inhibited with lenalidomide than the 1928z-d913 degradable CAR.

FIG. 25A-25H Demonstration of Cas9 degradation using exemplary zinc finger degrons. (FIG. 25A) Schematic showing the proteasomal degradation of Cas9 using exemplary C21-12 zinc finger based chimeric degron (super degron) and pomalidomide. (FIG. 25B) Exemplary embodiment fusions of Cas9 with single super degron tag at N-terminal (NSD-Cas9), Loop-231 (LSD-Cas9), and C-terminal (CSD-Cas9) regions and investigated for pomalidomide-induced proteasomal degradation. (FIG. 25C) Dose-dependent and pomalidomide-induced Cas9 degradation in HEK293T cells, transiently transfected with N-terminal HiBiT fused exemplary Cas9-super degron, WT-Cas9 constructs. Post 24 h of transfection and pomalidomide treatment, cell lysates were complemented with LgBiT, luminescence measured was normalized with total protein present in the lysate. (FIG. 250, FIG. 25E) Pomalidomide dose-dependent degradation (FIG. 25D) of exemplary super degron-Cas9 constructs in U2OS.eGFP.PEST cells measured by analyzing the images (FIG. 25E) in the eGFP disruption assay. (FIG. 25F) Pomalidomide-induced degradation of N-HiBiT fused LSD-Cas9 in transiently transfected HEK293T cells. (FIG. 25G, FIG. 25H) Pomalidomide-induced degradation of an example embodiment N-HiBiT fused LSD-Cas9 in transiently transfected HEK293T CRB−/− and CRBN+/+ cell lines, measured by HiBiT Luminescence (FIG. 25G), and immunoblot (FIG. 25H).

FIG. 26A-26E Cas9 lifetime can impact targeting specificity and DNA repair outcome. (FIG. 26A) U2OS cell line with stable Reduced Library genomic integration was transfected with an exemplary LSD-Cas9 transposon plasmid, followed by treatment with 1 pomalidomide at different time points after transfection (0-48 h) before genomic DNA was extracted at 120 h post-transfection. HTS sequencing was performed to analyse the +1 bp insertions, MH deletions and Non-MH deletions. (FIG. 26B) ddPCR quantification of single-nucleotide exchange at the RBM20 locus in HEK293T cells following templated DNA repair. For this, an exemplary LSD-Cas9 plasmid, RBM20 gRNA plasmid, and ssODN template were transfected in HEK293T cells followed by addition of pomalidomide at different time points after transfection. Cells were harvested at 72 h post-transfection, and percentages of HDR and NHEJ in the genomic DNA were analyzed by ddPCR analysis. (FIG. 26C) Luminescence-based quantification of HiBiT knock-in at the GAPDH locus in HEK293T cells following templated DNA repair. An example embodiment LSD-Cas9 plasmid, GAPDH gRNA plasmid, and ssODN template were transfected in HEK293T cells followed by addition of pomalidomide at different time points after transfection. Cells were lysed at 72 h post-transfection and complemented with LgBiT protein to measure the luminescence. (FIG. 26D, FIG. 26E) Cas9 lifetime can impact Cas9 targeting specificity. Pomalidomide dose-dependent control of on-target versus off-target activity of an example embodiment LSD-Cas9 targeting EMX1. VEGFA (FIG. 26D). Pomalidomide induced lifetime-dependent control of on-target versus off-target activity of an example embodiment LSD-Cas9 targeting EMX1, VEGFA (FIG. 26E).

FIG. 27A-27C—Demonstration of dCas9 based CRISPR system degradation using example embodiment zinc finger degrons. (FIG. 27A) dCas9-KRAB repressor is fused with an exemplary single super degron tag at Loop-231 (LSD-dCas9-BFP-KRAB) in a Citrate Lyase Beta Like (CLYBL) safe harbor targeting donor vector and knock-in using Cas9 in human iPSCs. iPSCs stably expressing an exemplary embodiment LSD-dCas9-BFP-KRAB were selected by neomycin selection. (FIG. 27B, FIG. 27C) Pomalidomide dose-induced (FIG. 27B) and time dependent (FIG. 27C) dCas9 degradation in iPSCs according to an example embodiment were monitored by immunoblots.

FIG. 28A-28F—Demonstration of an example embodiment base editor degradation using zinc finger degrons. (FIG. 284) Adenine base editor (ABE8e) is fused with an example embodiment single super degron tag at N-terminal (ABE-SD1), C-terminal (ABE-SD2) of TadA deaminase, at the linker region (ABE-SD3, ABE-SD4), and N-terminal (ABE-SD5), Loop-231 (ABE-SD6). and C-terminal (ABE-SD7) of the Cas9 nickase regions. (FIG. 28B) Pomalidomide-dose induced base editor degradation in HEK293T cells, transiently transfected with ABE8e and ABE-super degron constructs according to exemplary embodiments. Post 72 h of transfection and pomalidomide treatment, genomic DNA extracted was analyzed by NGS for the conversion of A.T to G.C. (FIG. 28C, FIG. 28D) Pomalidomide dose-induced (FIG. 28C) and time dependent (FIG. 280) ABE-SD6 degradation according to an example embodiment in transiently transfected 1-IEK293T cells was monitored by immunoblots. (FIG. 28E, FIG. 28F) Base editor lifetime can impact editing specificity. Pomalidomide dose-dependent control of on-target versus off-target activity of an example embodiment ABE-SD6 targeting HBG2 (FIG. 28E). Pomalidomide induced lifetime-dependent control of on-target versus off-target activity of an example embodiment ABE-SD6 targeting HBG2 (FIG. 28F).

FIG. 29A-29D—Kinetics of base editing activity of an example embodiment AAV based split ABE-SD6 in mice model. (FIG. 29A) An exemplary intein reconstitution strategy uses two fragments of protein fused to split-intein halves that splice to reconstitute a full-length protein following co-expression in host cells. (FIG. 29B-29D) Schematic showing injection of two doses (FIG. 29C: 5×10¹⁰), (29D: 5×10¹¹) of example embodiment AAVs in C57Bl6/J mice (FIG. 29B). These mice were harvested at different time points (3 days. 1 week, 3 weeks post injection) for the editing efficiency (FIG. 29C, FIG. 29D).

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboraotry Manual (1988) (Harlow and Lane, eds.): Antibodies A Laboraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may be. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Compositions are used herein that modulates the activity of a protein or polypeptide. The compositions can modulate the nucleic acid editing of the CRISPR-Cas protein. In some instances, these compositions for modulating activity target a variant CRISPR Cas protein.

All publications, published patent documents, and patent applications cited herein are hereby incorporated by reference to the same extent as though each individual publication, published patent document, or patent application was specifically and individually indicated as being incorporated by reference.

### Overview

The presently disclosed subject matter provides hybrid zinc finger polypeptides comprising a sequence selected from Table 3, Table 4A or Table 4B. In particular embodiments, the zinc finger comprises a Cys2His2 (C2H2) domain. The hybrid zinc finger polypeptides can be utilized in compounds, systems and methods for controlling or modulating CRISPR-Cas protein editing outcomes. In particular, the currently disclosed system can be provided with small molecules such as immunomodulatory inducing drugs (IMiDs) that can control or modulate Cas variant proteins that comprise one or more hybrid zinc fingers, also referred to herein as a zinc finger degradation domains or zinc finger degrons.

In some embodiments, the CRISPR Cas variants comprise one or more degrons. In embodiments, the degron is a zinc finger degron that can be controlled with thalidomide, lenalidomide, pomalidomide, and/or analogs thereof. In particular embodiments, the zinc finger comprises a Cys2His2 (C2H2) domain. The CRISPR Cas variant may comprise two or more zinc finger degradation domains

The compositions of the current system are utilized for controlling CRISPR-Cas editing outcomes. In one aspect, the protein is a Cas effector protein. The CRISPR Cas protein may comprise a Type II, V, or VI protein. In some embodiments, the Cas effector protein is a Cas9, a Cas12a, Cas12b, Cas12c, Cas12d, Cas13a, Cas13b, Cas13c, or Cas13d system. In one embodiment the Cas protein is a Cas9 or Cas 12 protein, in a particular embodiment, the Cas protein is a SpCas9 protein. The Cas effector protein can be provided as a variant which can also be disposed to degrade upon contact with the compositions disclosed herein. Use of zinc finger base editing degradation with improved control of the kinetics of base editing activity is also detailed herein.

In one aspect, the invention provides an engineered, non-naturally occurring CRISPR-Cas system comprising a variant CRISPR Cas protein, and a guide RNA (or guide DNA) that targets a DNA or RNA molecule encoding a gene product in a cell, whereby the guide RNA/DNA targets the DNA/RNA molecule encoding the gene product and the Cas cleaves the DNA or RNA molecule encoding the gene product, whereby expression of the gene product is altered; and, wherein the Cas protein and the guide RNA (or DNA) do not naturally occur together. The Cas variant protein of the specific invention can be engineered to contain insertions to which a degrader molecule of the instant invention targets. Such Cas variant proteins can also be controlled to effect editing outcomes. In one manner, the compositions disclosed herein can be administered subsequent to administration of a CRIPSR-Cas system, for example to a cell, to allow the CRISPR-Cas protein to edit nucleic acid. In embodiments, a compound or pharmaceutically acceptable salt thereof is administered more than 4 hours, more than 12 hours, or more than 24 hours after administering the CRISPR Cas protein-RNA complex. In embodiments, 1 bp insertions and/or microhomology end-joining is allowed is accomplished prior to administration of the compound or pharmaceutically acceptable salt thereof. In certain instances, the compositions can be administered so that CRISPR/Cas expression in that cell can be discontinued. Indeed, sustained expression could be undesirable in case of off-target effects at unintended genomic sites, etc. Accordingly, in one aspect, the compounds can target the Cas variant protein at the insertions to degrade the Cas variant protein. In this manner, the degrader molecule will alter or decrease the enzymatic activity of the variant CRISPR Cas protein. Delay of the compound's administration can be utilized to control or modulate the editing of the CRISPR-Cas system.

### Zinc Finger Polypeptide

The compositions of the current system may comprise a zinc finger degron. Generally, a degron is a peptide sequence or protein element that confers metabolic instability. A degron may refer to a portion of a protein involved in regulating the degradation rate of a protein. Degrons may include short amino acid sequences, structural motifs, and exposed amino acids (e.g., lysine or arginine). In particular, the currently disclosed system provides Cas variant proteins and other programmable nucleases that comprise one or more degrons. In embodiments, the degron is a zinc finger degron that can be controlled with thalidomide, lenalidomide, pomalidomide, and/or analogs thereof. In particular embodiments, the one or more degrons comprise a zinc finger polypeptide. In particular embodiments, the zinc finger comprises a Cys2 His2 (C2H2) domain. The programmable nuclease, e.g. Cas polypeptide, may be engineered to comprise two or more zinc finger degron domains. Each zinc finger domain may comprise a hybrid zinc finger, comprising two or more subdomains, each subdomain from a different wild type zinc finger.

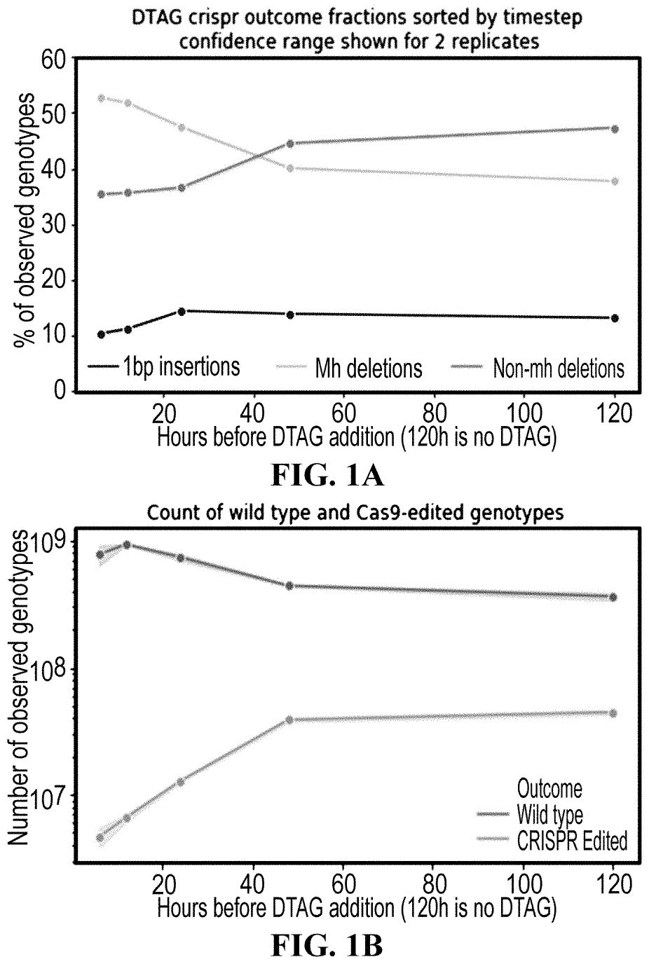

The C2H2 zinc finger domain shape has been found to be an important binding determinant, which can be a more important determining factor than the primary amino acid sequence. See, e.g. Sievers et al. 2018, “Defining the human C2H2 zinc-finger degrome targeted by thalidomide analogs through CRBN” Science 2018 Nov. 2:326(6414): eeat0572; doi: 10.1126/science.aat0572, incorporated herein by reference. Cys2-His2 (C2H2) zinc fingers have emerged as a recurrent degron motif mediating drug-dependent interactions with CRL4^{CRB}. See, e.g. An et al., Nat Commun. 8:15398 (2017), doi: 10.1038/ncomms15398 (showing ZFP91 harbors a zinc finger motif, and is related to the IKZF1/3 ZnF), incorporated herein by reference; Koduri et al., PNAS 116(7) 2539-2544 (2019), doi:10.1073/pnas.1818109116 (finding an IKZF3-derived 25mer constitutes a modular degron that can be used to target heterologous proteins for destruction by IMiDs) incorporated herein by reference, see, e.g. FIG. 1A-1L; see also, International Patent Publication No. WO 2019/089592, incorporated herein by reference. The C2H2 zinc fingers comprise beta-hairpin and alpha-helix subdomains; a domain typically consisting of about 28 to 30 amino acids comprising an N-terminal beta-hairpin followed by an alpha helix comprising two conserved histidine residues at its C-terminus. See, e.g. Fedotova et al., Acta Naturae, 2017 April-Jim; 9(2): 47-58. Applicants leveraged this modularity of beta-hairpin and alpha-helix subdomains to build a library of hybrid (also referred to alternately herein as synthetic) zinc fingers. As detailed herein, the hybrid zinc finger degron is a fusion protein comprising an N-terminal beta hairpin subdomain from one C2H2 zinc finger domain, and a C-terminal alpha helix subdomain from a different zinc finger domain from a library of identified C2H2 zinc finger domains identified. In an aspect, the hybrid zinc finger degron has enhanced or increased sensitivity to an IMiD molecule, e.g. thalidomide analog relative to a wild-type zinc finger domain.

Variants of the zinc finger degrons can be identified using methods such as, for example, phage assisted continuous evolution (PACE), see, e.g. Esvelt et al. 2011; doi: 10.1038/nature09929. PACE is a system that enables the continuous directed evolution of gene-encoded molecules that can be linked to protein production in Escherichia coli. Other methods of continuous directed evolution can be utilized in the identification of variants. In this manner, variants with increased sensitivity to small molecules other than thalidomide and/or its analogues.

In an aspect, the hybrid zinc finger has enhanced or increased sensitivity to one or more IMiD molecules relative to the wild-type zinc finger domain from which the beta-hairpin and/or the alpha helix subdomain are derived. In one embodiment, the enhances or increased sensitivity to one or more IMiD molecules allows for a reduction in the amount of IMiD molecule administered to induce degradation by 5%, 10%, 15%, 20%, 25%, 30%, 35%, 40%, 45%, 50%, 55%, 60%, 65%, 70%, 75%, 80% or more. In an aspect, the amount of small molecule, e.g. IMiD molecule, administered is reduced by a factor of 2, 3, 4, 5, 6, 7, 8, 9, 10, 20, 50, 100, 110, 120, 130, 140, 150 or more.

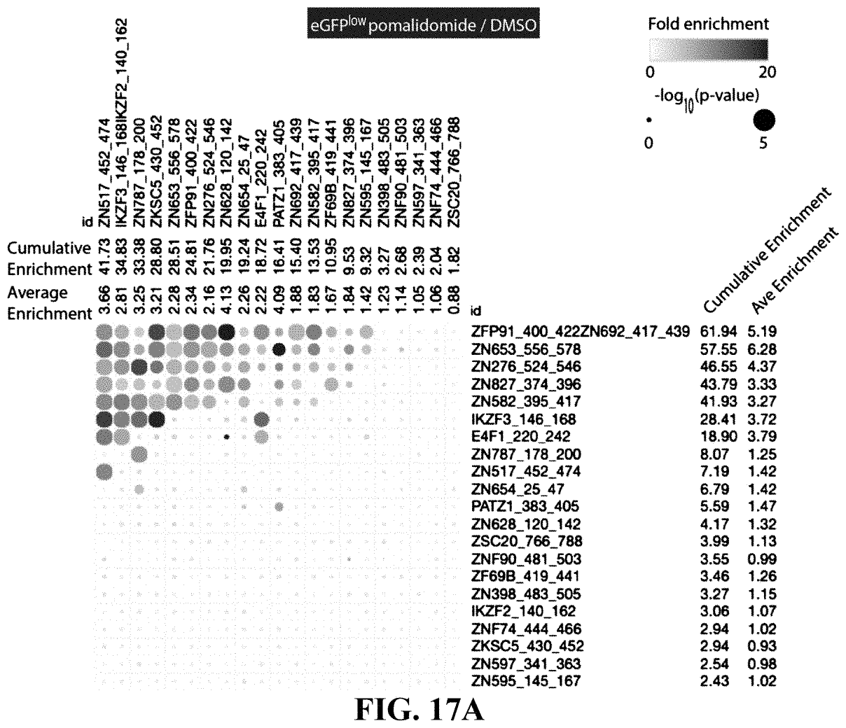

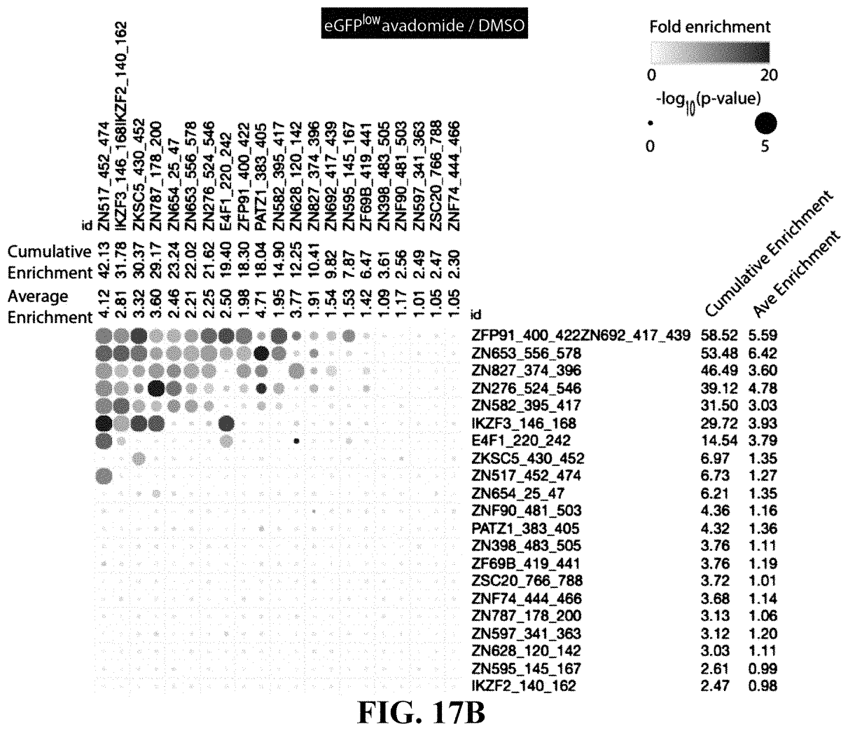

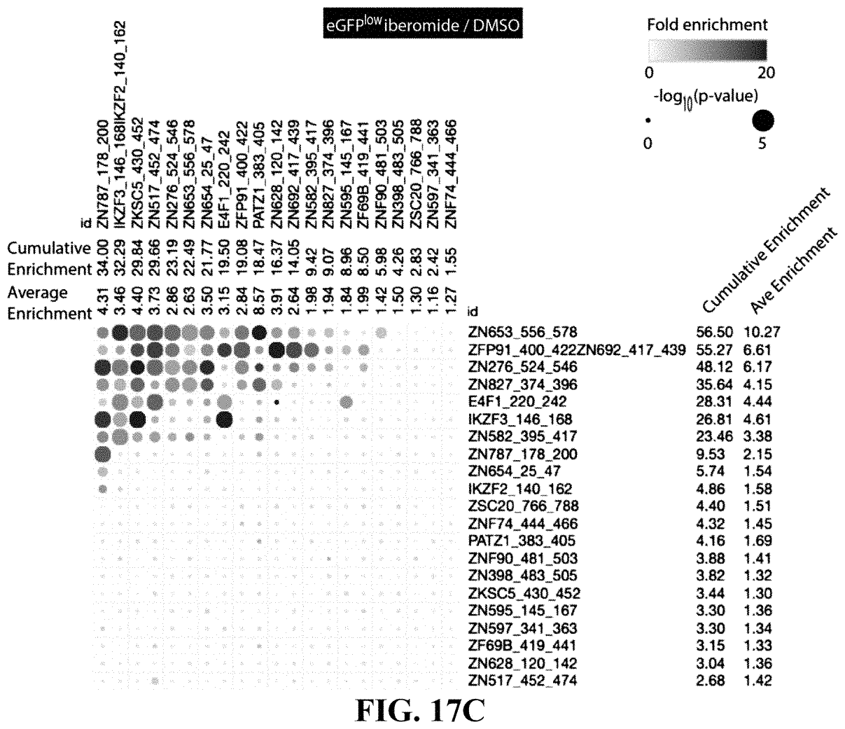

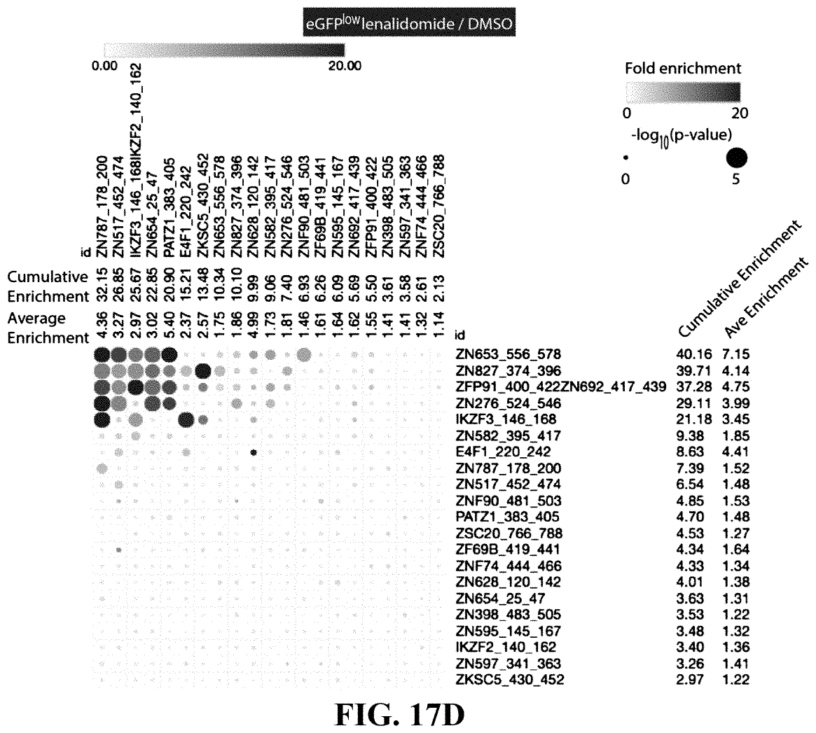

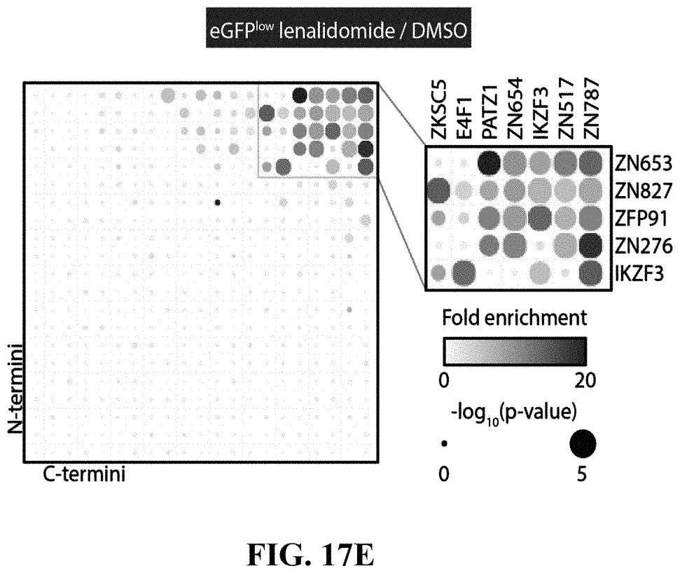

In particular aspects, the hybrid zinc finger degron comprises a sequence from Table 3, 4A, 4B. In an aspect, the beta hairpin and alpha-helix of two different zinc fingers a beta-hairpin and alpha-helix from a can be utilized to create a synthetic zinc finger. Optimization of the zinc finger can be based on screening methods described herein. The zinc finger may be tailored for use with a desired IMiD or small molecule. Exemplary screening of combinations of zinc finger domains best utilized for particular small molecules were identified for pomalidomide (FIG. 17A), avadomide (FIG. 17B), iberomide (FIG. 17C) and lenalidomide (FIGS. 17D-17E). By way of example, FIG. 17E provides screening results for combination of N-terminus and C-terminus synthetic zinc fingers utilized with lenalidomide. One can select, based on the fold-enrichment screening results, synthetic zinc fingers comprising a C-terminus selected from ZN787, ZN517, IKZF3, ZN654, PATZ1, E4F1, and ZKSC5 and a N-terminus selected from ZN653, ZN827, ZFP91, ZN276, and IKZF3 for components of a synthetic zinc finger optimized for use with lenalidomide. Similar identification from FIGS. 17A-17C can be derived for the small molecule.

In preferred embodiments, the synthetic zinc finger mediates drug-dependent degradation more efficiently, either at a more rapid pace of degradation, more complete degradation, or utilization of a lower dose of drug than that of a zinc finger of a human proteome. In an aspect, the zinc finger comprises at the N-terminus one of ZN653, ZN827, ZFP91, ZN276, E4F1, ZN582, ZN787, or IKZF3. In an aspect, the zinc finger comprises at the C-terminus one of ZN787, ZN517, IKZF3, ZN654, PATZ1, E4F1, ZN276, ZN268, ZN692, ZN582, ZN827, ZN653, ZN628, or ZKSC5. In embodiments, the combination of beta-hairpin and alpha-helix varies according to the IMiD, for example pomalidomide, avadomide, iberdomide, lenalidomide or thalidomide.

### Zinc Finger Screening

Methods of screening for zinc finger degrons optimized for use with CRISPR-Cas systems is also provided. In an exemplary embodiment, a library composed of all possible beta-hairpin and alpha-helix combinations from a set of C2H2 zinc fingers destabilized by various thalidomide derivatives, IMiDs, is generated. The library may be encoded into a degradation reporter vector, an exemplary vector is described in example 3, with cells of interest transduced with the vector. Cells can then be treated with destabilizing compositions, such as an IMiD, with subsequent identification and/or isolation of cells showing enhanced degradation in IMiD treated versus control-treated cell populations. In embodiments, the zinc finger is a hybrid form, comprised of an N-termini of one zinc finger, and the C-termini of a different zinc finger. Screening may be accomplished to find and optimize engineered zinc fingers showing enhanced drug-dependent degradation, as well as specific compositions that can be used for degradation. Isolation of transduced and treated cells can be according to known methods in the art, for example by cell sorting methods such as fluorescence-activated cell sorting (FACS). A control for such screening methods can include use of a wild-type zinc finger or no zinc finger.

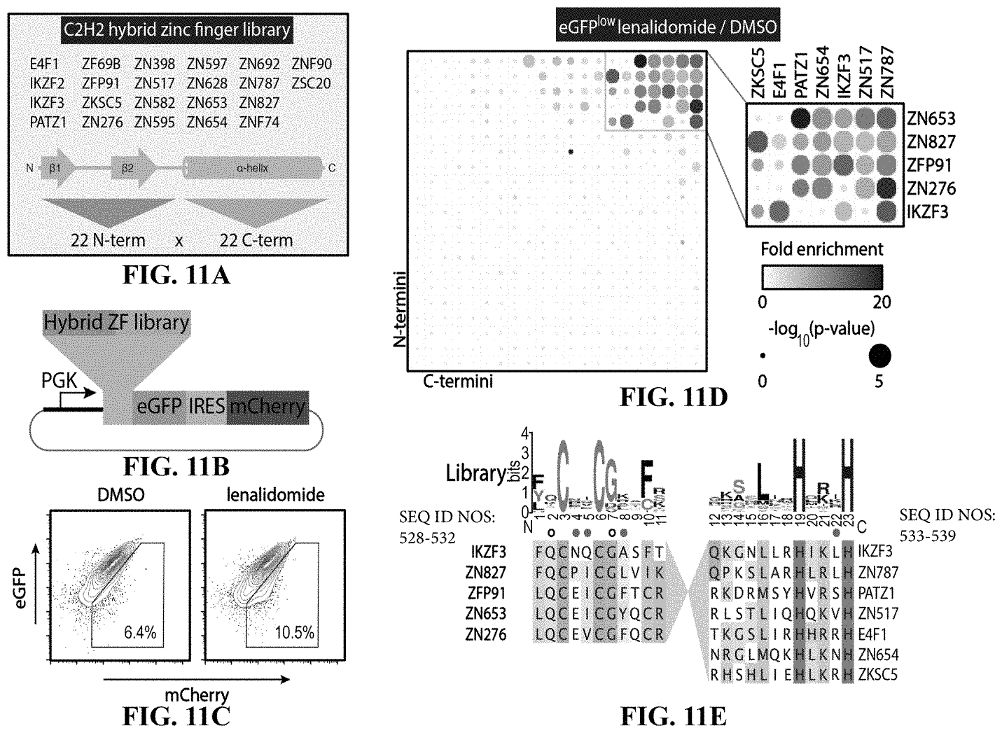

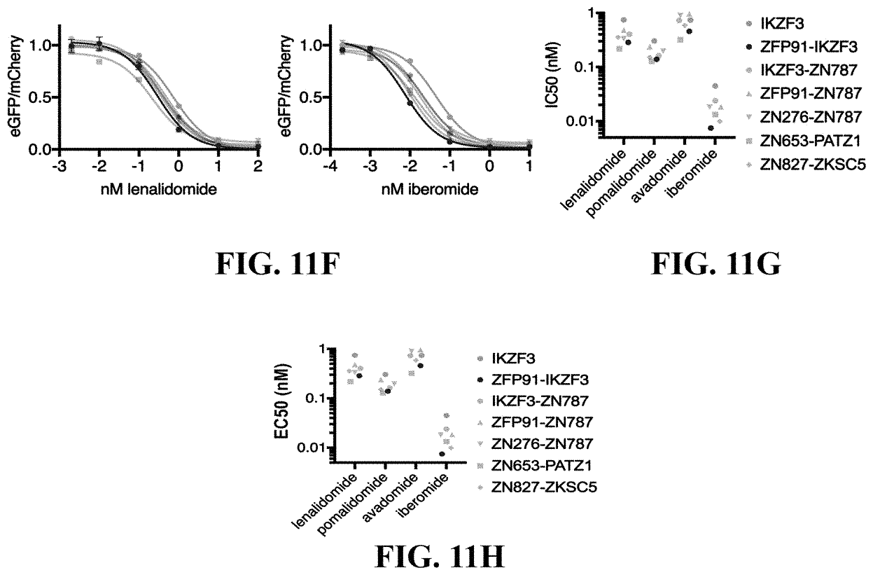

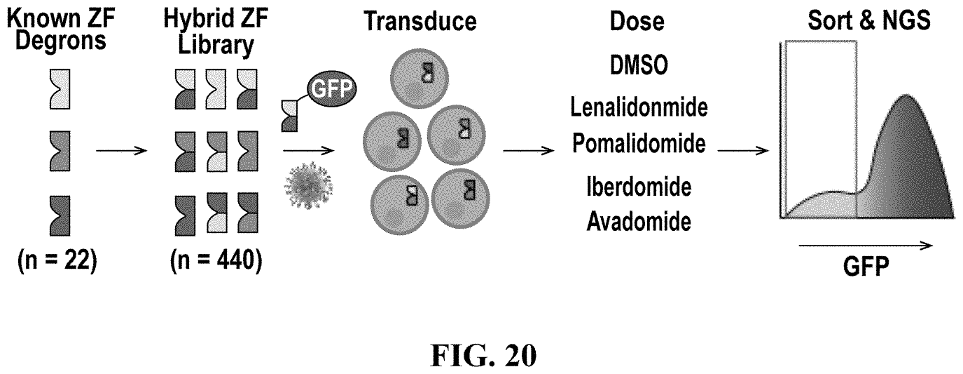

Subsequent to creation of the hybrid zinc finger library, the zinc fingers can be cloned into a protein degradation reporter, as detailed in FIGS. 11A and 11B. Transduction of the cloned reporter followed by dosing with one or more IMiDs, as shown in FIG. 20, for example, allows for the functional genomic screening for sequences that are efficiently degraded by one or more IMiDs.

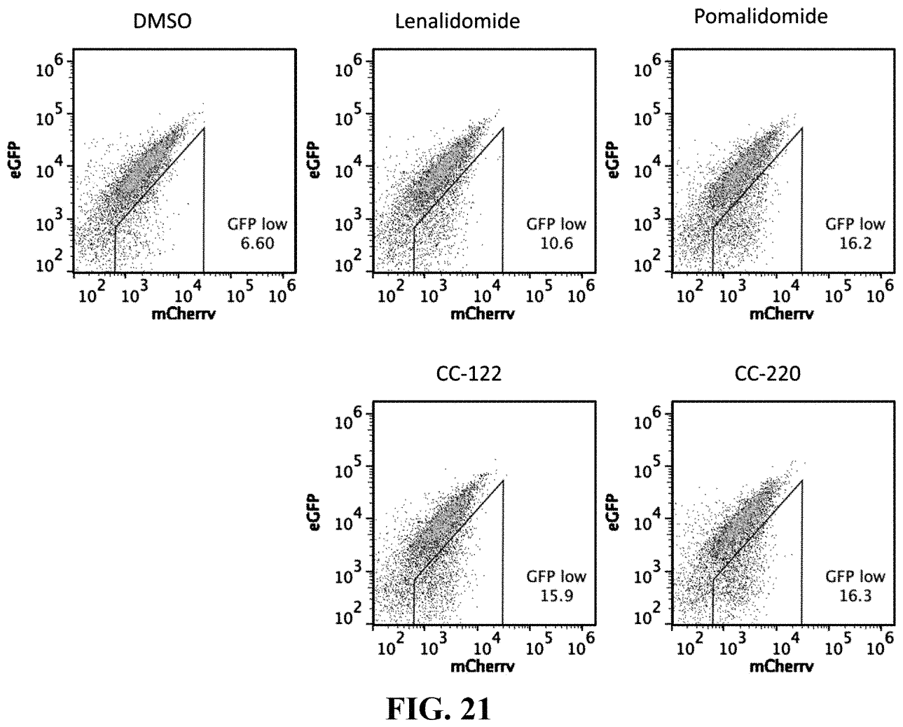

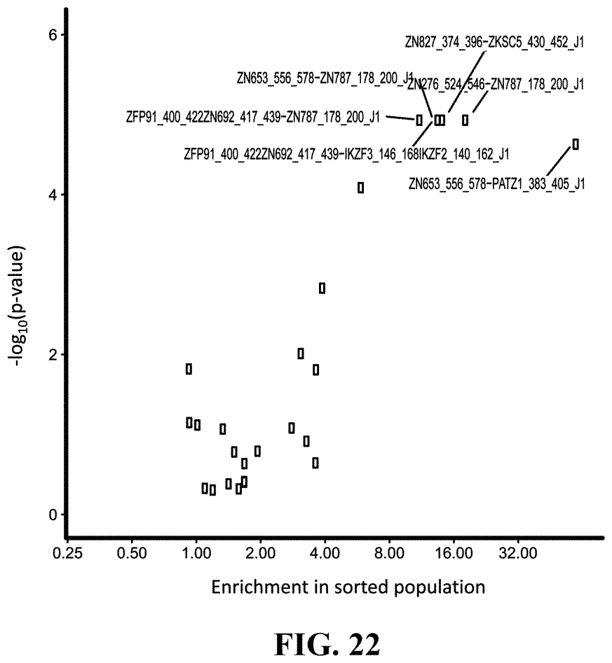

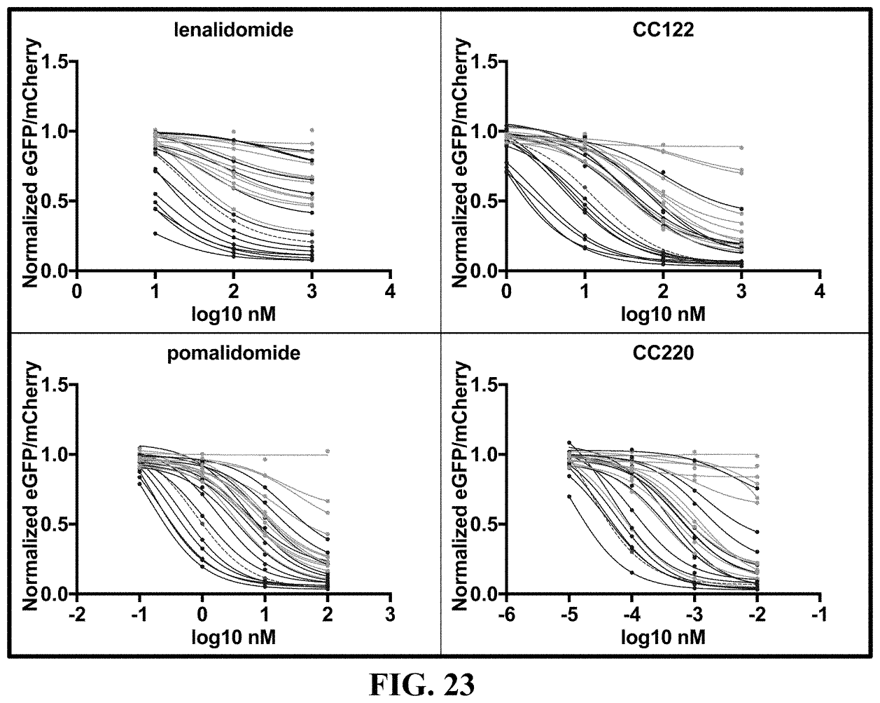

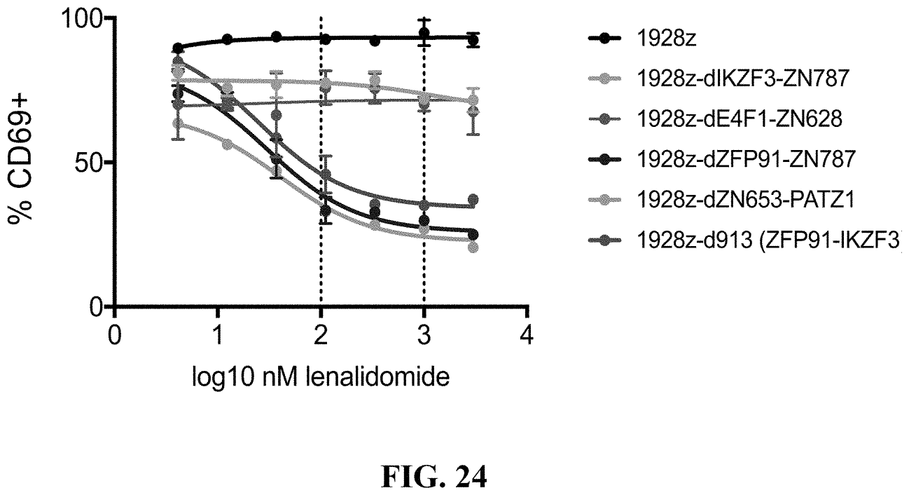

ZFs demonstrating drug-dependent degradation were significantly enriched in drug-treated versus control-treated mCherry⁺eGFP^{low} populations. Sorting cells with low GFP expression can comprise a scheme as described in FIG. 21. Briefly, the gate remains unchanged across each drug concentration, an increase in the fraction of low GFP cells in the various drug concentrations is indicative of drug-dependent degradation of a sequence from the library.

In certain embodiments, the hybrid zinc finger comprises enhanced lenalidomide-sensitive degradation, which may comprise an N-termini selected from ZN653, ZN827, ZFP91, ZN276, IKZF3, a C-termini selected from ZN787, ZN517, IKZF3, ZN654, PATZ1, E4F1, and ZKSC5, or a combination thereof (FIG. 11D). Similar findings were identified for pomalidomide, avadomide, and iberdomide (FIG. 17A-17C). The preferred N-terminal beta-hairpins converge on a similar sequence at residues with crystallographic evidence of side chain-drug interactions (15), but are otherwise molecularly diverse (FIG. 11E). The screening approach and data provided herein identify a group of ZF subdomains that can promiscuously combine to form lenalidomide-dependent hybrid super degrons, and other IMiD dependent hybrid degrons that are more efficiently degraded than their parent ZFs. The presently described screening can also be used to determine and optimize zinc finger degrons for use with other degraders and/or particular Cas peptides.

In an aspect, the degron is selected for its ability to be induced by a particular small molecule. In an aspect, the degron is induced by an immunomodulatory inducing drug. (IMiD). In one aspect, the IMiD is a thalidomide or one of its analogues, in an aspect, lenalidomide, pomalidomide, avadomide, or iberomide.

### Modified Programmable Nucleases Comprising a Hybrid Zn Finger Polypeptide

In embodiments, a modified programmable nuclease is provided comprising a hybrid Zn finger degron according to the present disclosure. Programmable nuclease can be, for example, components of transcription activator-like effector nuclease (TALEN), Zn finger nucleases, meganucleases, RNA-guided nucleases, for example, Class 1 or Class 2 CRISPR-Cas systems, a functional fragment thereof, a variant thereof, of any combination thereof. In some these embodiments, the other nucleotide targeting and/or binding molecule or components thereof can be in place of the CRISPR-Cas system components described herein. Also described herein are polynucleotides capable of encoding the other nucleotide binding and/or targeting molecules described herein. In particular embodiments, the modified programmable nuclease comprises at least one zinc finger degron inserted on an external portion of the modified programmable nuclease, which can be identified using known protein modeling techniques. In embodiments, the degron is attached to an N-terminal or C-terminal of the modified programmable nuclease.

Screening of hybrid zinc fingers for use in the current systems can identify optimized modified programmable nucleases comprising one or more hybrid zinc fingers, as well as identify IMiDs or other degradation inducing molecules for the modified programmable nucleases comprising one or more zinc finger degrons.

The degradation of the zinc finger modified Cas or other programmable nuclease is controlled through the use of a small molecule, which may be thalidomide, lenalidomide, pomalidomide, or any analog thereof (Immunomodulatory inducing drugs (IMiDs)). Advantageously, the control of the half-life of the programmable nuclease by degradation control such as via zinc finger degrons, aids in controlling or enhancing homology-directed repair (HDR) outcomes, over non-homologous end joining (NHEJ) outcomes in Cas-mediated genome editing, which may include temporal and lifetime control of the programmable nucleases detailed herein.

### CRISPR-Cas

In particular embodiments, the modified programmable nuclease is a Cas polypeptide. The Cas polypeptide comprises at least one zinc finger degron inserted on an external portion of the Cas polypeptide, which can be identified using known protein modeling techniques. In particular instances, the external portion of the Cas polypeptide is the loop of the Cas polypeptide. In an embodiment, the modified programmable nuclease comprises A cas protein, for example a Cas9 protein comprising a full-length IKZF3, IKZF1, or a fragment or variant thereof comprising a degron, which may include a C2H2 Zinc finger.

In particular embodiments, the Cas 9 polypeptide is an SpCas9 polypeptide comprising at least one zinc finger degron inserted in the loop of the SpCas9 polypeptide. The degron is preferably attached to the external portion of any Cas polypeptide. In embodiments, the degron is attached to an N-terminal, C-terminal or loop of the Cas polypeptide. In particular embodiments, the zinc finger is inserted in a loop of the Cas polypeptide.

In embodiments, the Cas9 protein comprises a full-length IKZF3, IKZF1, or a fragment or variant thereof comprising a degron, which may include a C2H2 Zinc finger.

In particular embodiments, the Cas polypeptide comprises at least one zinc finger degron inserted on an external portion of the Cas polypeptide, which can be identified using known protein modeling techniques. In particular instances, the external portion of the Cas polypeptide is the loop of the Cas polypeptide. In particular embodiments, the Cas 9 polypeptide is an SpCas9 polypeptide comprising at least one zinc finger degron inserted in the loop of the SpCas9 polypeptide. The degron is preferably attached to the external portion of any Cas polypeptide. In embodiments, the degron is attached to an N-terminal, C-terminal or loop of the Cas polypeptide. In particular embodiments, the zinc finger is inserted in a loop of the Cas polypeptide.

In embodiments, the Cas polypeptide comprises a CRBN polypeptide substrate domain capable of binding CRBN in response to thalidomide or one of its analogs, thereby promoting ubiquitin pathway-mediated degradation, which can be as described, for example, in Sievers et al., Science v. 362, no. 6414 (2018). Further embodiments comprise use of the hybrid zinc fingers in embodiments with CAR-T cells such as those described in International Patent Publication WO 2019, 089592, incorporated herein by reference for its teachings of zinc finger degron application with chimeric antigen receptor cellular therapy, at Example 2-5.

The Cas polypeptide may comprise one or more zinc finger degrons. Insertion of the degrons may further comprise a linker on one or both ends of the degron connected to the Cas polypeptide. The linker in some embodiments is a glycine serine linker. The linker may comprise about 5 to about 15 amino acids. In embodiments, the linker comprises: GSGSGSGSGG (SEQ ID NO: 1) or GGSGSGSGSG (SEQ ID NO: 2).

In an aspect, the Cas polypeptide is modified with a zinc finger degron. The modified Cas polypeptide can be any polypeptide described herein, including a Type II, Type V, or Type VI Cas polypeptide. In one aspect, the Cas polypeptide is a Cas 9 polypeptide comprising a zinc finger degron. In particular embodiments, the Cas 9 polypeptide is an SpCas9 polypeptide comprising at least one zinc finger degron inserted in the loop of the SpCas9 polypeptide. The degradation of the zinc finger modified Cas9 is controlled through the use of a small molecule, which may be thalidomide, lenalidomide, pomalidomide, or any analog thereof (Immunomodulatory inducing drugs (IMiDs)). Advantageously, the control of the half-life of the Cas9 by degradation control such as via zinc finger degrons, aids in controlling or enhancing homology-directed repair (HDR) outcomes, over non-homologous end joining (NHEJ) outcomes in Cas-mediated genome editing.

In embodiments, the Cas polypeptide comprises a CRBN polypeptide substrate domain capable of binding CRBN in response to thalidomide or one of its analogs, thereby promoting ubiquitin pathway-mediated degradation, which can be as described, for example, in Sievers et al., Science v. 362, no. 6414 (2018).

In an aspect, the Cas polypeptide is modified with a zinc finger degron. The modified Cas polypeptide can be any polypeptide described herein, including a Type II, Type V, or Type VI Cas polypeptide. In one aspect, the Cas polypeptide is a Cas 9 polypeptide comprising a zinc finger degron. In particular embodiments, the Cas 9 polypeptide is an SpCas9 polypeptide comprising at least one zinc finger degron inserted in the loop of the SpCas9 polypeptide.

In general, a CRISPR-Cas or CRISPR system as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008. When the CRISPR protein is a Cpf1 protein, a tracrRNA is not required.

In certain embodiments, the CRISPR-Cas system is a class 2 CRISPR system, including Type II, Type V and Type VI systems. In certain example embodiments, the CRISPR system is a Cas9, a Cas12a, Cas12b, Cas12c, Cas12d, Cas13a, Cas13b, Cas13c, or Cas13d system.

As used herein, the term “Cas” can refer to a (modified) effector protein of the CRISPR/Cas system or complex, and can be without limitation a (modified) Cas9 or a (modified) Cas12 (e.g. Cas12a “Cpf1”, Cas12b “C2c1,” Cas12c “C2c3”), or, can be any other class 2 CRISPR system, for example, Cas 13a, Cas13b, Cas13c or Cas13d. The term “Cas” may be used herein interchangeably with the terms “CRISPR” protein, “CRISPR/Cas protein”, “CRISPR effector”, “CRISPR/Cas effector”, “CRISPR enzyme”, “CRISPR/Cas enzyme” and the like, unless otherwise apparent, such as by specific and exclusive reference to Cas9. It is to be understood that the term “CRISPR protein” may be used interchangeably with “CRISPR enzyme”, irrespective of whether the CRISPR protein has altered, such as increased or decreased (or no) enzymatic activity, compared to the wild type CRISPR protein.

In some embodiments, the CRISPR Cas variant is based on a Type-II CRISPR effector protein such as Cas9. In some embodiments, the CRISPR Cas variant is based on a Type-V CRISPR effector protein such as Cas12a, Cas12b, or Cas12c. In some embodiments the CRISPR Cas variant is based on a Type-VI CRISPR effector protein such as Cas13a, Cas13b, Cas13c or Cas13d.

In some embodiments, the CRISPR Cas variant protein is a Cas9 CRISPR Cas variant, for instance SaCas9, SpCas9, StCas9, CjCas9 and so forth—any ortholog is envisaged. In some embodiments, the CRISPR Cas variant is a Cpf1 CRISPR Cas variant, for instance AsCpf1, LbCpf1, FnCpf1 and so forth—any ortholog is envisaged. Modifications to the location of insertion sites can be made according to the Cas effector protein, with structural features such as loops and other accessible locations available for fusions, for example with the hybrid zinc finger domains detailed herein.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”. In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects, the invention involves ribonucleoprotein comprising the variant CRISPR-Cas proteins disclosed herein. Pre-formed RNP comprising the variant CRISPR-Cas proteins can be used for nucleofection of cells.

The present invention also contemplates use of the systems described herein to control RNA-guided gene drives, for example in systems analogous to gene drives described in PCT Patent Publication WO 2015/105928. Further reference can be found for instance in Esvelt et al. (eLife 2014; 3:e03401; DOI: 10.7554/eLife.03401.001); Webber et al. (PNAS; 2015; 112(34):10565-10567); DeFrancesco (Nature Biotechnology, 2015, 33(10):1019-1021); DiCarlo et al. (Nature Biotechnology, 2015; 33: 1250-1255); Gantz et al. (PNAS; 2015; 112(49):E6736-E6743). Systems of this kind may for example provide methods for altering eukaryotic germline cells, by introducing into the germline cell a nucleic acid sequence encoding an RNA or DNA-guided DNA or RNA nuclease and one or more guide RNAs or guide DNAs, control of the germline cell can be accomplished when utilizing the Cas variant proteins of the current invention by exposing the cell to an IMiD or other drug designed to degrade the Cas-variant protein. Exposing the cell may occur after about 1, 2, 3, 4, 5, 6, 7, 8, 9, 10, 11, 12, 13, 14, 15, 16, 17, 18, 19, 20, 21, 22, 23, 24, 18, 32, 36, 40, 44, or 48 hours. The guide RNAs/DNAs may be designed to be complementary to one or more target locations on (genomic) DNA or RNA of the germline cell. The nucleic acid sequence encoding the DNA/RNA guided DNA/RNA nuclease and the nucleic acid sequence encoding the guide RNAs/DNAs may be provided on constructs between flanking sequences, with promoters arranged such that the germline cell may express the nuclease and the guides, together with any desired cargo-encoding sequences that are also situated between the flanking sequences. The flanking sequences will typically include a sequence which is identical to a corresponding sequence on a selected target chromosome, so that the flanking sequences work with the components encoded by the construct to facilitate insertion of the foreign nucleic acid construct sequences into RNA or DNA at a target cut site by mechanisms such as homologous recombination, to render the germline cell homozygous for the foreign nucleic acid sequence. In this way, gene-drive systems are capable of introgressing desired cargo genes throughout a breeding population (Gantz et al., 2015, Highly efficient Cas9-mediated gene drive for population modification of the malaria vector mosquito Anopheles stephensi, PNAS 2015, published ahead of print Nov. 23, 2015, doi:10.1073/pnas.1521077112; Esvelt et al., 2014, Concerning DNA- or RNA-guided gene drives for the alteration of wild populations eLife 2014; 3:e03401). In select embodiments, target sequences may be selected which have few potential off-target sites in a genome. Targeting multiple sites within a target locus, using multiple guide RNAs, may increase the cutting frequency and hinder the evolution of drive resistant alleles. Truncated guide RNAs may reduce off-target cutting. Paired nickases may be used instead of a single nuclease, to further increase specificity. Gene drive constructs may include cargo sequences encoding transcriptional regulators, for example to activate homologous recombination genes and/or repress non-homologous end-joining. Target sites may be chosen within an essential gene, so that non-homologous end-joining events may cause lethality rather than creating a drive-resistant allele. The gene drive constructs can be engineered to function in a range of hosts at a range of temperatures (Cho et al. 2013, Rapid and Tunable Control of Protein Stability in Caenorhabditis elegans Using a Small Molecule, PLoS ONE 8(8): e72393. doi:10.1371/journal.pone.0072393). Degrading the Cas protein, or other programmable nuclease, comprising the hybrid zinc fingers according to the current invention allows for control of the gene drive, as well as editing outcomes.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

### Destabilized Cas and Fusion Proteins

In certain embodiments, the Cas protein according to the invention as described herein is associated with or fused to a destabilization domain (DD). In some embodiments, the DD is ER50. A corresponding stabilizing ligand for this DD is, in some embodiments, 4HT. As such, in some embodiments, one of the at least one DDs is ER50 and a stabilizing ligand therefor is 4HT or CMP8. In some embodiments, the DD is DHFR50. A corresponding stabilizing ligand for this DD is, in some embodiments, TMP. As such, in some embodiments, one of the at least one DDs is DHFR50 and a stabilizing ligand therefor is TMP. In some embodiments, the DD is ER50. A corresponding stabilizing ligand for this DD is, in some embodiments, CMP8. CMP8 may therefore be an alternative stabilizing ligand to 4HT in the ER50 system. While it may be possible that CMP8 and 4HT can/should be used in a competitive matter, some cell types may be more susceptible to one or the other of these two ligands, and from this disclosure and the knowledge in the art the skilled person can use CMP8 and/or 4HT.

In some embodiments, one or two DDs may be fused to the N-terminal end of the Cas with one or two DDs fused to the C-terminal of the Cas. In some embodiments, the at least two DDs are associated with the Cas and the DDs are the same DD, i.e. the DDs are homologous. Thus, both (or two or more) of the DDs could be ER50 DDs. This is preferred in some embodiments. Alternatively, both (or two or more) of the DDs could be DHFR50 DDs. This is also preferred in some embodiments. In some embodiments, the at least two DDs are associated with the Cas and the DDs are different DDs, i.e. the DDs are heterologous. Thus, one of the DDS could be ER50 while one or more of the DDs or any other DDs could be DHFR50. Having two or more DDs which are heterologous may be advantageous as it would provide a greater level of degradation control. A tandem fusion of more than one DD at the N or C-term may enhance degradation; and such a tandem fusion can be, for example ER50-ER50-Cas or DHFR-DHFR-Cas It is envisaged that high levels of degradation would occur in the absence of either stabilizing ligand, intermediate levels of degradation would occur in the absence of one stabilizing ligand and the presence of the other (or another) stabilizing ligand, while low levels of degradation would occur in the presence of both (or two of more) of the stabilizing ligands. Control may also be imparted by having an N-terminal ER50 DD and a C-terminal DHFR50 DD.

In some embodiments, the fusion of the Cas with the DD comprises a linker between the DD and the Cas. In some embodiments, the linker is a GlySer linker. In some embodiments, the DD-Cas further comprises at least one Nuclear Export Signal (NES). In some embodiments, the DD-Cas comprises two or more NESs. In some embodiments, the DD-Cas comprises at least one Nuclear Localization Signal (NLS). This may be in addition to an NES. In some embodiments, the Cas comprises or consists essentially of or consists of a localization (nuclear import or export) signal as, or as part of, the linker between the Cas and the DD. HA or Flag tags are also within the ambit of the invention as linkers. Applicants use NLS and/or NES as linker and also use Glycine Serine linkers as short as GS up to (GGGGS)₃.

Destabilizing domains have general utility to confer instability to a wide range of proteins; see, e.g., Miyazaki, J Am Chem Soc. Mar. 7, 2012; 134(9): 3942-3945, incorporated herein by reference. CMP8 or 4-hydroxytamoxifen can be destabilizing domains. More generally, A temperature-sensitive mutant of mammalian DHFR (DHFRts), a destabilizing residue by the N-end rule, was found to be stable at a permissive temperature but unstable at 37° C. The addition of methotrexate, a high-affinity ligand for mammalian DHFR, to cells expressing DHFRts inhibited degradation of the protein partially. This was an important demonstration that a small molecule ligand can stabilize a protein otherwise targeted for degradation in cells. A rapamycin derivative was used to stabilize an unstable mutant of the FRB domain of mTOR (FRB*) and restore the function of the fused kinase, GSK-3β.6,7 This system demonstrated that ligand-dependent stability represented an attractive strategy to regulate the function of a specific protein in a complex biological environment. A system to control protein activity can involve the DD becoming functional when the ubiquitin complementation occurs by rapamycin induced dimerization of FK506-binding protein and FKBP12. Mutants of human FKBP12 or ecDHFR protein can be engineered to be metabolically unstable in the absence of their high-affinity ligands, Shield-1 or trimethoprim (TMP), respectively. These mutants are some of the possible destabilizing domains (DDs) useful in the practice of the invention and instability of a DD as a fusion with a Cas confers to the Cas degradation of the entire fusion protein by the proteasome. Shield-1 and TMP bind to and stabilize the DD in a dose-dependent manner. The estrogen receptor ligand binding domain (ERLBD, residues 305-549 of ERS1) can also be engineered as a destabilizing domain. Since the estrogen receptor signaling pathway is involved in a variety of diseases such as breast cancer, the pathway has been widely studied and numerous agonist and antagonists of estrogen receptor have been developed. Thus, compatible pairs of ERLBD and drugs are known. There are ligands that bind to mutant but not wild-type forms of the ERLBD. By using one of these mutant domains encoding three mutations (L384M, M421G, G521R)12, it is possible to regulate the stability of an ERLBD-derived DD using a ligand that does not perturb endogenous estrogen-sensitive networks. An additional mutation (Y537S) can be introduced to further destabilize the ERLBD and to configure it as a potential DD candidate. This tetra-mutant is an advantageous DD development. The mutant ERLBD can be fused to a Cas and its stability can be regulated or perturbed using a ligand, whereby the Cas has a DD. Another DD can be a 12-kDa (107-amino-acid) tag based on a mutated FKBP protein, stabilized by Shieldl ligand; see, e.g., Nature Methods 5, (2008). For instance a DD can be a modified FK506 binding protein 12 (FKBP12) that binds to and is reversibly stabilized by a synthetic, biologically inert small molecule, Shield-1; see, e.g., Banaszynski L A, Chen L C, Maynard-Smith L A, Ooi A G, Wandless T J. A rapid, reversible, and tunable method to regulate protein function in living cells using synthetic small molecules. Cell. 2006; 126:995-1004; Banaszynski L A, Sellmyer M A, Contag C H, Wandless T J, Thorne S H. Chemical control of protein stability and function in living mice. Nat Med. 2008; 14:1123-1127; Maynard-Smith L A, Chen L C, Banaszynski L A, Ooi A G, Wandless T J. A directed approach for engineering conditional protein stability using biologically silent small molecules. The Journal of biological chemistry. 2007; 282:24866-24872; and Rodriguez, Chem Biol. Mar. 23, 2012; 19(3): 391-398—all of which are incorporated herein by reference and may be employed in the practice of the invention in selected a DD to associate with a Cas in the practice of this invention. As can be seen, the knowledge in the art includes a number of DDs, and the DD can be associated with, e.g., fused to, advantageously with a linker, to a Cas, whereby the DD can be stabilized in the presence of a ligand and when there is the absence thereof the DD can become destabilized, whereby the Cas is entirely destabilized, or the DD can be stabilized in the absence of a ligand and when the ligand is present the DD can become destabilized; the DD allows the Cas and hence the CRISPR-Cas complex or system to be regulated or controlled—turned on or off so to speak, to thereby provide means for regulation or control of the system, e.g., in an in vivo or in vitro environment. For instance, when a protein of interest is expressed as a fusion with the DD tag, it is destabilized and rapidly degraded in the cell, e.g., by proteasomes. Thus, absence of stabilizing ligand leads to a D associated Cas being degraded. When a new DD is fused to a protein of interest, its instability is conferred to the protein of interest, resulting in the rapid degradation of the entire fusion protein. Peak activity for Cas is sometimes beneficial to reduce off-target effects. Thus, short bursts of high activity are preferred. The present invention is able to provide such peaks. In some senses the system is inducible. In some other senses, the system repressed in the absence of stabilizing ligand and de-repressed in the presence of stabilizing ligand.

### Deactivated/Inactivated/Dead Cas Proteins

In certain embodiments, the Cas protein herein is a catalytically inactive or dead Cas protein. In some cases, Cas protein herein is a catalytically inactive or dead Cas protein (dCas). In some cases, a dead Cas protein, e.g., a dead Cas protein has nickase activity. In some embodiments, the dCas protein comprises mutations in the nuclease domain. In some embodiments, the dCas protein has been truncated. In some cases, the dead Cas proteins may be fused with a deaminase herein, e.g., an adenosine deaminase.

Where the Cas protein has nuclease activity, the Cas protein may be modified to have diminished nuclease activity e.g., nuclease inactivation of at least 70%, at least 80%, at least 90%, at least 95%, at least 97%, or 100% as compared with the wild type enzyme; or to put in another way, a Cas enzyme having advantageously about 0% of the nuclease activity of the non-mutated or wild type Cas, or no more than about 3% or about 5% or about 10% of the nuclease activity of the non-mutated or wild type Cas. This is possible by introducing mutations into the nuclease domains of the Cas and orthologs thereof.

The inactivated Cas CRISPR enzyme may have associated (e.g., via fusion protein) one or more functional domains, including for example, one or more domains from the group comprising, consisting essentially of, or consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g., light inducible). Preferred domains are Fok1, VP64, P65, HSF1, MyoD1. In the event that Fok1 is provided, it is advantageous that multiple Fok1 functional domains are provided to allow for a functional dimer and that gRNAs are designed to provide proper spacing for functional use (Fok1) as specifically described in Tsai et al. Nature Biotechnology, Vol. 32, Number 6, June 2014). The adaptor protein may utilize known linkers to attach such functional domains. In some cases it is advantageous that additionally at least one NLS is provided. In some instances, it is advantageous to position the NLS at the N terminus. When more than one functional domain is included, the functional domains may be the same or different.

In general, the positioning of the one or more functional domain on the inactivated Cas enzyme is one which allows for correct spatial orientation for the functional domain to affect the target with the attributed functional effect. For example, if the functional domain is a transcription activator (e.g., VP64 or p65), the transcription activator is placed in a spatial orientation which allows it to affect the transcription of the target. Likewise, a transcription repressor will be advantageously positioned to affect the transcription of the target, and a nuclease (e.g., Fok1) will be advantageously positioned to cleave or partially cleave the target. This may include positions other than the N-/C-terminus of the CRISPR enzyme.

The dead or deactivated Cas proteins may be used as target-binding proteins, (e.g., DNA binding proteins). In these cases, the dead or deactivated Cas proteins may be fused with one or more functional domains.

### Nickases

In embodiments, the nucleic acid binding enzyme is a nickase. A nickase may be designed as disclosed in the art and in accordance with the site specific nucleases disclosed herein, for example, a TnpB nickase.

In some embodiments, the Cas protein or polypeptide may be a nickase. The Cas proteins with nickase activity may be a mutated form of a wildtype Cas protein. Mutations can also be made at neighboring residues at amino acids that participate in the nuclease activity. In some embodiments, only the RuvC domain is inactivated, and in other embodiments, another putative nuclease domain is inactivated, wherein the effector protein complex functions as a nickase and cleaves only one DNA strand. In some embodiments, two Cas variants (each a different nickase) are used to increase specificity, two nickase variants are used to cleave DNA at a target (where both nickases cleave a DNA strand, while minimizing or eliminating off-target modifications where only one DNA strand is cleaved and subsequently repaired). In preferred embodiments the Cas protein cleaves sequences associated with or at a target locus of interest as a homodimer comprising two Cas protein molecules. In a preferred embodiment the homodimer may comprise two Cas protein molecules comprising a different mutation in their respective RuvC domains.

The Cas protein may be mutated with respect to a corresponding wild-type enzyme such that the mutated Cas protein lacks the ability to cleave one or both DNA strands of a target locus containing a target sequence. In particular embodiments, one or more catalytic domains of the Cas protein are mutated to produce a mutated Cas protein which cleaves only one DNA strand of a target sequence.

In an embodiment, the CRISPR enzyme is a Cas9 enzyme that comprises one or more mutations in one of the catalytic domains, wherein the one or more mutations is selected from the group consisting of D10A, E762A, and D986A in the RuvC domain or the one or more mutations is selected from the group consisting of H840A, N854A and N863A in the HNH domain. In an embodiment, the Cas protein comprises multiple mutations in the CRISPR enzyme or the Cas protein. In an aspect, a Cas9 D10A nickase may include the mutations D10A, E762A and D986A (or some subset of these) and a Cas9 H840A nickase may include the mutations H840A, N854A and N863A (or some subset of these). In an aspect, the nickase is a modified Cas9 comprising a mutation at N863A (according to the numbering found in SpCas9 from S. pyogenes) or at N580 (according to the numbering found in SaCas9 from S. aureus) or at a residue which is equivalent or corresponding to those residues in orthologs of S. pyogenes or S. aureus. In particular, mutation of the residue to A (alanine) is preferred in some embodiments, but any catalytically inactive mutation at these residues should suffice. In an aspect, and without being bound by theory, the mutation may have the advantage of being a more predictable mutation for protein function than a H840A nickase equivalent, which may change binding behavior. Thus, the Cas9 enzyme comprises a mutation and may be used as a generic DNA binding protein (e.g. the mutated Cas9 may or may not function as a double stranded nuclease or as a single stranded nickase; can function as merely a binding protein; but advantageously, the Cas9 is a nickase); and the so-mutated Cas9 may be with or without fusion to a functional domain or protein domain. The mutation concerns the catalytic domain HNH at residue N863; the Cas9 enzyme is, a SpCas9 protein comprising the mutation N863A, or any mutated ortholog having a mutation corresponding to SpCas9N863A. In one aspect of the invention, the mutated Cas9 enzyme may be fused to a protein domain or functional domain, e.g., such as a transcriptional activation domain. In one aspect, the transcriptional activation domain may be VP64. In another aspect the protein domain or functional domain can be, for example, a FokI domain. In an aspect, the nickase mutation may allow for an improved HDR efficiency is considered a higher frequency of HDR events (and/or reduced indel formation) as a result of double nickase activity resulting from either the use of SpCas9N863A mutant or an ortholog having a mutation corresponding to SpCas9N863A (e.g., S. aureus N580A) as compared to double nickase activity resulting from a SpCas9 which does not comprise the N863A mutation or an ortholog not comprising a corresponding mutation to SpCas9N863A (e.g., S. aureus N580A). Further description of such nickases are as described in International Patent Publication WO 2014/204725, filed Jun. 10, 2014 and entitled “Optimized Crispr-Cas Double Nickase Systems, Methods And Compositions For Sequence Manipulation” and International Patent Publication WO 2016/028682, filed Aug. 17, 2015 and entitled “Genome Editing using Cas9 Nickases” both incorporated herein by reference in their entirety.

In certain embodiments of the methods provided herein the Cas protein is a mutated Cas protein which cleaves only one DNA strand, i.e. a nickase. More particularly, in the context of the present invention, the nickase ensures cleavage within the non-target sequence, i.e. the sequence which is on the opposite DNA strand of the target sequence and which is 3′ of the PAM sequence. By means of further guidance, and without limitation, an arginine-to-alanine substitution (R911A) in the Nuc domain of C2c1 from Alicyclobacillus acidoterrestris converts C2c1 from a nuclease that cleaves both strands to a nickase (cleaves a single strand). It will be understood by the skilled person that where the enzyme is not AacC2c1, a mutation may be made at a residue in a corresponding position.

In certain embodiments, the Cas protein may be a C2c1 nickase which comprises a mutation in the Nuc domain. In some embodiments, the C2c1 nickase comprises a mutation corresponding to amino acid positions R911, R1000, or R1015 in Alicyclobacillus acidoterrestris C2c1. In some embodiments, the C2c1 nickase comprises a mutation corresponding to R911A, R1000A, or R1015A in Alicyclobacillus acidoterrestris C2c1. In some embodiments, the C2c1 nickase comprises a mutation corresponding to R894A in Bacillus sp. V3-13 C2c1. In certain embodiments, the C2c1 protein recognizes PAMs with increased or decreased specificity as compared with an unmutated or unmodified form of the protein. In some embodiments, the C2c1 protein recognizes altered PAMs as compared with an unmutated or unmodified form of the protein.

In some embodiments, to minimize the level of toxicity and off-target effect, a Cas nickase can be used with a pair of guide RNAs targeting a site of interest. Guide sequences and strategies to minimize toxicity and off-target effects can be as in WO 2014/093622 (PCT/US2013/074667); or, via mutation as described herein.

In some examples, the system may comprise two or more nickases, in particular a dual or double nickase approach. In some aspects and embodiments, a single type Cas nickase may be delivered, for example a modified Cas or a modified Cas nickase as described herein. This results in the target DNA being bound by two Cas nickases. In addition, it is also envisaged that different orthologs may be used, e.g., a Cas nickase on one strand (e.g., the coding strand) of the DNA and an ortholog on the non-coding or opposite DNA strand. The ortholog can be, but is not limited to, a Cas nickase. It may be advantageous to use two different orthologs that require different PAMs and may also have different guide requirements, thus allowing a greater deal of control for the user. In certain embodiments, DNA cleavage will involve at least four types of nickases, wherein each type is guided to a different sequence of target DNA, wherein each pair introduces a first nick into one DNA strand and the second introduces a nick into the second DNA strand. In such methods, at least two pairs of single stranded breaks are introduced into the target DNA wherein upon introduction of first and second pairs of single-strand breaks, target sequences between the first and second pairs of single-strand breaks are excised. In certain embodiments, one or both of the orthologs is controllable, i.e. inducible.

### Dead Cas

In certain embodiments, the Cas protein is a catalytically inactive or dead Cas protein (dCas). For example, the Cas protein or polypeptide may lack nuclease activity. In some embodiments, the dCas comprises mutations in the nuclease domain. In some embodiments, the dCas effector protein has been truncated. In some cases, the dead Cas proteins may be fused with one or more functional domains.

The Cas protein or its variant (e.g., dCas) may be associated (e.g., fused) to one or more functional domains. The association can be by direct linkage of the Cas protein to the functional domain, or by association with the crRNA. In a non-limiting example, the crRNA comprises an added or inserted sequence that can be associated with a functional domain of interest, including, for example, an aptamer or a nucleotide that binds to a nucleic acid binding adapter protein. The functional domain may be a functional heterologous domain.

The functional domain may cleave a DNA sequence or modify transcription or translation of a gene. Examples of functional domains include domains that have methylase activity, demethylase activity, transcription activation activity, transcription repression activity, transcription release factor activity, histone modification activity, RNA cleavage activity, DNA cleavage activity, nucleic acid binding activity, and molecular switches (e.g., light inducible). Preferred domains are Fok1, VP64, P65, HSF1, MyoD1. In the event that Fok1 is provided, multiple Fok1 functional domains may be provided to allow for a functional dimer and that gRNAs are designed to provide proper spacing for functional use (Fok1).

In some cases, the functional domains may be heterologous functional domains. For example, the one or more heterologous functional domains may comprise one or more nuclear localization signal (NLS) domains. The one or more heterologous functional domains may comprise at least two or more NLS domains. The one or more NLS domain(s) may be positioned at or near or in proximity to a terminus of the Cas protein and if two or more NLSs, each of the two may be positioned at or near or in proximity to a terminus of the Cas protein. The one or more heterologous functional domains may comprise one or more transcriptional activation domains. In a preferred embodiment the transcriptional activation domain may comprise VP64. The one or more heterologous functional domains may comprise one or more transcriptional repression domains. In a preferred embodiment the transcriptional repression domain comprises a KRAB domain or a SID domain (e.g. SID4X). The one or more heterologous functional domains may comprise one or more nuclease domains. In a preferred embodiment a nuclease domain comprises Fok1. Other examples of functional domains include translational initiator, translational activator, translational repressor, nucleases, in particular ribonucleases, a spliceosome, beads, a light inducible/controllable domain or a chemically inducible/controllable domain.

The positioning of the one or more functional domain on Cas or dCas protein is one which allows for correct spatial orientation for the functional domain to affect the target with the attributed functional effect. For example, if the functional domain is a transcription activator (e.g., VP64 or p65), the transcription activator is placed in a spatial orientation which allows it to affect the transcription of the target. Likewise, a transcription repressor may be positioned to affect the transcription of the target, and a nuclease (e.g., Fok1) will be advantageously positioned to cleave or partially cleave the target. This may include positions other than the N-/C-terminus of the Cas protein.

The Cas or dCas protein may be associated with the one or more functional domains through one or more adaptor proteins. The adaptor protein may utilize known linkers to attach such functional domains.

The fusion between the adaptor protein and the activator or repressor may include a linker.

### Functional Domains

The systems and compositions provided herein may comprise one or more of the Cas proteins associated with one or more functional domains. In certain embodiments, the systems and compositions comprise fusion proteins comprising the Cas proteins(s)/subunit(s) associated with the functional domain(s).

In some embodiments, one or more functional domains are associated with an adaptor protein, for example as used with the modified guides of Konnerman et al. (Nature 517, 583-588, 29 Jan. 2015). In some embodiments, one or more functional domains are associated with a dead gRNA (dRNA). In some embodiments, a dRNA complex with active Cas system/protein subunit(s) directs gene regulation by a functional domain at on gene locus while an gRNA directs DNA cleavage by the active Cas protein at another locus, for example as described analogously in CRISPR-Cas systems by Dahlman et al., ‘Orthogonal gene control with a catalytically active Cas9 nuclease’. In some embodiments, dRNAs are selected to maximize selectivity of regulation for a gene locus of interest compared to off-target regulation. In some embodiments, dRNAs are selected to maximize target gene regulation and minimize target cleavage.

For the purposes of the following discussion, reference to a functional domain could be a functional domain associated with one or more Cas protein of the Cas system, the zinc finger, or a functional domain associated with the adaptor protein.

In the practice of the invention, loops of the gRNA may be extended, without colliding with the Cas protein by the insertion of distinct RNA loop(s) or distinct sequence(s) that may recruit adaptor proteins that can bind to the distinct RNA loop(s) or distinct sequence(s). The adaptor proteins may include but are not limited to orthogonal RNA-binding protein/aptamer combinations that exist within the diversity of bacteriophage coat proteins. A list of such coat proteins includes, but is not limited to: Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. These adaptor proteins or orthogonal RNA binding proteins can further recruit effector proteins or fusions which comprise one or more functional domains.

In some embodiments, the functional domain may be selected from the group consisting of: transposase domain, integrase domain, recombinase domain, resolvase domain, invertase domain, protease domain, DNA methyltransferase domain, DNA hydroxylmethylase domain, ligase domain, polymerase domain, helicase domain, resolvase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease. In some preferred embodiments, the functional domain is a transcriptional activation domain, such as, without limitation, VP64, p65, MyoD1, HSF1, RTA, SET7/9 or a histone acetyltransferase. In some embodiments, the functional domain is a transcription repression domain, preferably KRAB. In some embodiments, the transcription repression domain is SID, or concatemers of SID (eg SID4X). In some embodiments, the functional domain is an epigenetic modifying domain, such that an epigenetic modifying enzyme is provided. In some embodiments, the functional domain is an activation domain, which may be the P65 activation domain.

In some examples, the Cas is associated with a ligase or functional fragment thereof. The ligase may ligate a single-strand break (a nick) generated by the Cas. In certain cases, the ligase may ligate a double-strand break generated by the Cas. In certain examples, the Cas is associated with a reverse transcriptase or functional fragment thereof.

In some embodiments, the one or more functional domains is an NLS (Nuclear Localization Sequence) or an NES (Nuclear Export Signal). In some embodiments, the one or more functional domains is a transcriptional activation domain comprises VP64, p65, MyoD1, HSF1, RTA, SET7/9 and a histone acetyltransferase. Other references herein to activation (or activator) domains in respect of those associated with the CRISPR enzyme include any known transcriptional activation domain and specifically VP64, p65, MyoD1, HSF1, RTA, SET7/9 or a histone acetyltransferase.

In some embodiments, the one or more functional domains is a transcriptional repressor domain. In some embodiments, the transcriptional repressor domain is a KRAB domain. In some embodiments, the transcriptional repressor domain is a NuE domain, NcoR domain, SID domain or a SID4X domain.

In some embodiments, the one or more functional domains have one or more activities comprising methylase activity, demethylase activity, transcription activation activity, transcription repression activity, transcription release factor activity, histone modification activity, RNA cleavage activity, DNA cleavage activity, DNA integration activity or nucleic acid binding activity.

Histone modifying domains are also preferred in some embodiments. Exemplary histone modifying domains are discussed below. Transposase domains, HR (Homologous Recombination) machinery domains, recombinase domains, and/or integrase domains are also preferred as the present functional domains. In some embodiments, DNA integration activity includes HR machinery domains, integrase domains, recombinase domains and/or transposase domains. Histone acetyltransferases are preferred in some embodiments.

In some embodiments, the DNA cleavage activity is due to a nuclease. In some embodiments, the nuclease comprises a Fok1 nuclease. See, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

In some embodiments, the one or more functional domains is attached to the Cas protein so that upon binding to the sgRNA and target the functional domain is in a spatial orientation allowing for the functional domain to function in its attributed function.

Functional domains may be used to regulate transcription, e.g., transcriptional repression. Transcriptional repression is often mediated by chromatin modifying enzymes such as histone methyltransferases (HMTs) and deacetylases (HDACs). Repressive histone effector domains are known and an exemplary list is provided below. In the exemplary table, preference was given to proteins and functional truncations of small size to facilitate efficient viral packaging (for instance via AAV). In general, however, the domains may include HDACs, histone methyltransferases (HMTs), and histone acetyltransferase (HAT) inhibitors, as well as HDAC and HMT recruiting proteins. The functional domain may be or include, in some embodiments, HDAC Effector Domains, HDAC Recruiter Effector Domains, Histone Methyltransferase (HMT) Effector Domains, Histone Methyltransferase (HMT) Recruiter Effector Domains, or Histone Acetyltransferase Inhibitor Effector Domains.

It is also preferred to target endogenous (regulatory) control elements (such as enhancers and silencers) in addition to a promoter or promoter-proximal elements. Thus, the invention can also be used to target endogenous control elements (including enhancers and silencers) in addition to targeting of the promoter. These control elements can be located upstream and downstream of the transcriptional start site (TSS), starting from 200 bp from the TSS to 100 kb away. Targeting of known control elements can be used to activate or repress the gene of interest. In some cases, a single control element can influence the transcription of multiple target genes. Targeting of a single control element could therefore be used to control the transcription of multiple genes simultaneously.

Targeting of putative control elements on the other hand (e.g. by tiling the region of the putative control element as well as 200 bp up to 100 kB around the element) can be used as a means to verify such elements (by measuring the transcription of the gene of interest) or to detect novel control elements (e.g. by tiling 100 kb upstream and downstream of the TSS of the gene of interest). In addition, targeting of putative control elements can be useful in the context of understanding genetic causes of disease. Many mutations and common SNP variants associated with disease phenotypes are located outside coding regions. Targeting of such regions with either the activation or repression systems described herein can be followed by readout of transcription of either a) a set of putative targets (e.g. a set of genes located in closest proximity to the control element) or b) whole-transcriptome readout by e.g. RNAseq or microarray. This would allow for the identification of likely candidate genes involved in the disease phenotype. Such candidate genes could be useful as novel drug targets.

Histone acetyltransferase (HAT) inhibitors are mentioned herein. However, an alternative in some embodiments is for the one or more functional domains to comprise an acetyltransferase, preferably a histone acetyltransferase. These are useful in the field of epigenomics, for example in methods of interrogating the epigenome. Methods of interrogating the epigenome may include, for example, targeting epigenomic sequences. Targeting epigenomic sequences may include the guide being directed to an epigenomic target sequence. Epigenomic target sequence may include, in some embodiments, include a promoter, silencer or an enhancer sequence.

Examples of acetyltransferases are known but may include, in some embodiments, histone acetyltransferases. In some embodiments, the histone acetyltransferase may comprise the catalytic core of the human acetyltransferase p300 (Gerbasch & Reddy, Nature Biotech 6 Apr. 2015).

### Linkers

The term “linker” as used in reference to a fusion protein refers to a molecule which joins the proteins to form a fusion protein. Generally, such molecules have no specific biological activity other than to join or to preserve some minimum distance or other spatial relationship between the proteins. However, in certain embodiments, the linker may be selected to influence some property of the linker and/or the fusion protein such as the folding, net charge, or hydrophobicity of the linker.

Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the Cas protein and the nucleotide deaminase by a distance sufficient to ensure that each protein retains its required functional property. Preferred peptide linker sequences adopt a flexible extended conformation and do not exhibit a propensity for developing an ordered secondary structure. In certain embodiments, the linker can be a chemical moiety which can be monomeric, dimeric, multimeric or polymeric. Preferably, the linker comprises amino acids. Typical amino acids in flexible linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180. For example, GlySer linkers GlySer linkers GGS, GGGS (SEQ ID NO: 4) or GSG can be used. GGS, GSG, GGGS (SEQ ID NO: 4) or GGGGS (SEQ ID NO: 5) linkers can be used in repeats of 3 (such as (GGS)₃ (SEQ ID NO: 6), (GGGGS)₃ (SEQ ID NO: 3)) or 5, 6, 7, 9 or even 12 or more, to provide suitable lengths. In some cases, the linker may be (GGGGS)₃₋₁₅, For example, in some cases, the linker may be (GGGGS)₃₋₁₁, e.g., GGGGS (SEQ ID NO: 5), (GGGGS)₂ (SEQ ID NO: 7), (GGGGS)₃ (SEQ ID NO: 3), (GGGGS)₄ (SEQ ID NO: 8), (GGGGS)₅ (SEQ ID NO: 9), (GGGGS)₆ (SEQ ID NO: 10), (GGGGS)₇ (SEQ ID NO: 11), (GGGGS)₈ (SEQ ID NO: 12), (GGGGS)₉ (SEQ ID NO: 13), (GGGGS)₁₀ (SEQ ID NO: 14), or (GGGGS)₁₁ (SEQ ID NO: 15).

In particular embodiments, linkers such as (GGGGS)₃ (SEQ ID NO: 3) are preferably used herein. (GGGGS)₆ (SEQ ID NO: 10), (GGGGS)₉ (SEQ ID NO: 13) or (GGGGS)₁₂ (SEQ ID NO: 16) may preferably be used as alternatives. Other preferred alternatives are (GGGGS)₁ (SEQ ID NO: 5), (GGGGS)₂ (SEQ ID NO: 7), (GGGGS)₄ (SEQ ID NO: 8), (GGGGS)₅ (SEQ ID NO: 9, (GGGGS)₇ (SEQ ID NO: 11), (GGGGS)₈ (SEQ ID NO: 12), (GGGGS)₁₀ (SEQ ID NO: 14), or (GGGGS)₁₁ (SEQ ID NO: 15). In yet a further embodiment, LEPGEKPYKCPECGKSFSQSGALTRHQRTHTR (SEQ ID NO: 17) is used as a linker. In yet an additional embodiment, the linker is an XTEN linker. In particular embodiments, the Cas protein is linked to the deaminase protein or its catalytic domain by means of an LEPGEKPYKCPECGKSFSQSGALTRHQRTHTR (SEQ ID NO: 17) linker. In further particular embodiments, the Cas protein is linked C-terminally to the N-terminus of a deaminase protein or its catalytic domain by means of an LEPGEKPYKCPECGKSFSQSGALTRHQRTHTR (SEQ ID NO: 17) linker. In addition, N- and C-terminal NLSs can also function as linker (e.g., PKKKRKVEASSPKKRKVEAS (SEQ ID NO: 18)). Examples of suitable linkers are shown in Table 1.

TABLE 1
Examples of suitable linkers as disclosed herein.

| | |
|---|---|
| GGS | GGTGGTAGT (SEQ ID NO: 19) |
| GGSx3 | GGTGGTAGTGGAGGGAGCGGCGGTTCA |
| (9) | (SEQ ID NO: 20) |
| GGSx7 | ggtggaggaggctctggtggaggcggtagcggaggcggag |
| (21) | ggtcgGGTGGTAGTGGAGGGAGCGGCGGTTCA |
| | (SEQ ID NO: 21) |
| XTEN | TCGGGATCTGAGACGCCTGGGACCTCGGAATCGGCTACGC |
| | CCGAAAGT (SEQ ID NO: 22) |
| Z- | Gtggataacaaatttaacaaagaaatgtgggcggcgtggg |
| EGFR_ | aagaaattcgtaacctgccgaacctgaacggc |
| Short | tggcagatgaccgcgtttattgcgagcctggtggatgatc |
| | cgagccagagcgcgaacctgctggcggaagcgaaaaaact |
| | gaacgatgcgcaggcgccgaaaaccggcggtggttctggt |
| | (SEQ ID NO: 23) |
| GSAT | Ggtggttctgccggtggctccggttctggctccagcggtg |
| | gcagctctggtgcgtccggcacgggtactgcgggtggcac |
| | tggcagcggttccggtactggctctggc |
| | (SEQ ID NO: 24) |

Linkers may be used between the guide RNAs and the functional domain (activator or repressor), or between the Cas protein and the functional domain. The linkers may be used to engineer appropriate amounts of “mechanical flexibility”.

In certain embodiments, the one or more functional domains are controllable, i.e. inducible.

### Split Proteins

It is noted that in this context, and more generally for the various applications as described herein, the use of a split version of the Cas protein can be envisaged. Indeed, this may not only allow increased specificity but may also be advantageous for delivery. The Cas is split in the sense that the two parts of the Cas enzyme substantially comprise a functioning Cas. The split may be so that the catalytic domain(s) are unaffected. That Cas may function as a nuclease or it may be a dead-Cas which is essentially an RNA-binding protein with very little or no catalytic activity, due to typically mutation(s) in its catalytic domains.

Each half of the split Cas may be fused to a dimerization partner. By means of example, and without limitation, employing rapamycin sensitive dimerization domains, allows to generate a chemically inducible split Cas for temporal control of Cas activity. Cas can thus be rendered chemically inducible by being split into two fragments and that rapamycin-sensitive dimerization domains may be used for controlled reassembly of the Cas. The two parts of the split Cas can be thought of as the N′ terminal part and the C′ terminal part of the split Cas. The fusion is typically at the split point of the Cas. In other words, the C′ terminal of the N′ terminal part of the split Cas is fused to one of the dimer halves, whilst the N′ terminal of the C′ terminal part is fused to the other dimer half.

The Cas does not have to be split in the sense that the break is newly created. The split point is typically designed in silico and cloned into the constructs. Together, the two parts of the split Cas, the N′ terminal and C′ terminal parts, form a full Cas, comprising preferably at least 70% or more of the wildtype amino acids (or nucleotides encoding them), preferably at least 80% or more, preferably at least 90% or more, preferably at least 95% or more, and most preferably at least 99% or more of the wildtype amino acids (or nucleotides encoding them). Some trimming may be possible, and mutants are envisaged. Non-functional domains may be removed entirely. What is important is that the two parts may be brought together and that the desired Cas function is restored or reconstituted. The dimer may be a homodimer or a heterodimer.

The effector protein can moreover be fused to another functional RNase domain, such as a non-specific RNase or Argonaute 2, which acts in synergy to increase the RNase activity or to ensure further degradation of the message.

The term “pharmaceutically acceptable salt” refers to those salts that are within the scope of proper medicinal assessment, suitable for use in contact with human tissues and organs and those of lower animals, without undue toxicity, irritation, allergic response or similar and are consistent with a reasonable benefit/risk ratio. In some embodiments, pharmaceutically acceptable salts can be formed by the reaction of a disclosed compound with an equimolar or excess amount of acid. Alternatively, hemi-salts can be formed by the reaction of a compound with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., about one hour to about ten days and can be isolated by filtration or other conventional methods.

### Guide Molecules

The methods described herein may be used to modulate and/or screen modulation of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62). In some embodiments, it is of interest to reduce the susceptibility of the guide molecule to RNA cleavage, such as to cleavage by Cas13. Accordingly, in particular embodiments, the guide molecule is adjusted to avoid cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′ phosphorothioate (MS), S-constrained ethyl(cEt), or 2′-O-methyl 3′ thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J Biotech. 233:74-83). In certain embodiments, a guide comprises ribonucleotides in a region that binds to a target RNA and one or more deoxyribonucleotides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine(5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of th guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-Cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the complementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In a particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928). Aptamers may also be constructed that function as molecular switches, responding to a que by changing properties, such as RNA aptamers that bind fluorophores to mimic the activity of green flourescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, 02 concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB 1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the CRISPR-Cas system or complex function. The invention can involve applying the chemical source or energy so as to have the guide function and the CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans;4/164/rs2), 2. FKBP-FRB based system inducible by rapamycin (or related chemicals based on rapamycin) (see, e.g., nature.com/nmeth/journal/v2/n6/full/nmeth763.html), 3. GID1-GAI based system inducible by Gibberellin (GA) (see, e.g., nature.com/nchembio/journal/v8/n5/full/nchembio.922.html).

A chemical inducible system can be an estrogen receptor (ER) based system inducible by 4-hydroxytamoxifen (4 OHT) (see, e.g. pnas.org/content/104/3/1027. abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel-based system inducible by energy, heat or radio-wave (see, e.g., sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the CRISPR-Cas complex or system, and the binding will induce the change of sub-cellular localization of the polypeptide, leading to the entire polypeptide entering the nucleus of cells. Once inside the nucleus, the guide protein and the other components of the CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100.mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et. al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

The present invention may be further illustrated and extended based on aspects of CRISPR-Cas development and use as set forth in the following articles and particularly as relates to delivery of a CRISPR protein complex and uses of an RNA guided endonuclease in cells and organisms as described in any of the publications of International Publication WO2018035250 at [0027] specifically incorporated herein by reference.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cast. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US 2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17-Jun-15, PROTECTED GUIDE RNAS (PGRNAS); U.S. application 62/091,455, filed, 12-Dec-14, PROTECTED GUIDE RNAS (PGRNAS); U.S. application 62/096,708, 24-Dec-14, PROTECTED GUIDE RNAS (PGRNAS); U.S. applications 62/091,462, 12-Dec-14, 62/096,324, 23-Dec-14, 62/180,681, 17 Jun. 2015, and 62/237,496, 5 Oct. 2015, DEAD GUIDES FOR CRISPR TRANSCRIPTION FACTORS; U.S. application 62/091,456, 12-Dec-14 and 62/180,692, 17 Jun. 2015, ESCORTED AND FUNCTIONALIZED GUIDES FOR CRISPR-CAS SYSTEMS; U.S. application 62/091,461, 12-Dec-14, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19-Dec-14, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24-Dec-14, ENGINEERING OF SYSTEMS, METHODS AND OPTIMIZED ENZYME AND GUIDE SCAFFOLDS FOR SEQUENCE MANIPULATION; U.S. application 62/098,059, 30-Dec-14, 62/181,641, 18 Jun. 2015, and 62/181,667, 18 Jun. 2015, RNA-TARGETING SYSTEM; U.S. application 62/096,656, 24-Dec-14 and 62/181,151, 17 Jun. 2015, CRISPR HAVING OR ASSOCIATED WITH DESTABILIZATION DOMAINS; U.S. application 62/096,697, 24-Dec-14, CRISPR HAVING OR ASSOCIATED WITH AAV; U.S. application 62/098,158, 30-Dec-14, ENGINEERED CRISPR COMPLEX INSERTIONAL TARGETING SYSTEMS; U.S. application 62/151,052, 22-Apr-15, CELLULAR TARGETING FOR EXTRACELLULAR EXOSOMAL REPORTING; U.S. application 62/054,490, 24-Sep-14, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12-FEB-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25-Sep-14, SYSTEMS, METHODS AND COMPOSITIONS FOR SEQUENCE MANIPULATION

WITH OPTIMIZED FUNCTIONAL CRISPR-CAS SYSTEMS; U.S. application 62/087,537, 4-Dec-14, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24-Sep-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23-Oct-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24-Sep-14 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24-Sep-14, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25-Sep-14, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25-Sep-14, MULTIFUNCTIONAL-CRISPR COMPLEXES AND/OR OPTIMIZED ENZYME LINKED FUNCTIONAL-CRISPR COMPLEXES; U.S. application 62/087,475, 4-Dec-14 and 62/181,690, 18 Jun. 2015, FUNCTIONAL SCREENING WITH OPTIMIZED FUNCTIONAL CRISPR-CAS SYSTEMS; U.S. application 62/055,487, 25-Sep-14, FUNCTIONAL SCREENING WITH OPTIMIZED FUNCTIONAL CRISPR-CAS SYSTEMS; U.S. application 62/087,546, 4-Dec-14 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30-Dec-14, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

### Nuclear Localization Sequences

In some embodiments, the Cas sequence is fused to one or more nuclear localization sequences (NLSs), such as about or more than about 1, 2, 3, 4, 5, 6, 7, 8, 9, 10, or more NLSs. In some embodiments, the Cas comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the Cas protein comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 25); the NLS from nucleoplasmin (e.g. the nucleoplasmin bipartite NLS with the sequence KRPAATKKAGQAKKKK (SEQ ID NO: 26); the c-myc NLS having the amino acid sequence PAAKRVKLD (SEQ ID NO: 27) or RQRRNELKRSP (SEQ ID NO: 28); the hRNPA1 M9 NLS having the sequence NQSSNFGPMKGGNFGGRSSGPYGGGGQYFAKPRNQGGY (SEQ ID NO: 29); the sequence RMRIZFKNKGKDTAELRRRRVEVSVELRKAKKDEQILKRRNV (SEQ ID NO: 30) of the IBB domain from importin-alpha; the sequences VSRKRPRP (SEQ ID NO: 31) and PPKKARED (SEQ ID NO: 32) of the myoma T protein; the sequence PQPKKKPL (SEQ ID NO: 33) of human p53; the sequence SALIKKKKKMAP (SEQ ID NO: 34) of mouse c-abl IV; the sequences DRLRR (SEQ ID NO: 35) and PKQKKRK (SEQ ID NO: 36) of the influenza virus NS1; the sequence RKLKKKIKKL (SEQ ID NO: 37) of the Hepatitis virus delta antigen; the sequence REKKKFLKRR (SEQ ID NO: 38) of the mouse Mx1 protein; the sequence KRKGDEVDGVDEVAKKKSKK (SEQ ID NO: 39) of the human poly(ADP-ribose) polymerase; and the sequence RKCLQAGMNLEARKTKK (SEQ ID NO: 40)) of the steroid hormone receptors (human) glucocorticoid. In general, the one or more NLSs are of sufficient strength to drive accumulation of the Cas in a detectable amount in the nucleus of a eukaryotic cell. In general, strength of nuclear localization activity may derive from the number of NLSs in the Cas, the particular NLS(s) used, or a combination of these factors. Detection of accumulation in the nucleus may be performed by any suitable technique. For example, a detectable marker may be fused to the Cas, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or Cas enzyme activity), as compared to a control no exposed to the Cas or complex, or exposed to a Cas lacking the one or more NLSs. In certain embodiments, other localization tags may be fused to the Cas protein, such as without limitation for localizing the Cas to particular sites in a cell, such as organells, such mitochondria, plastids, chloroplast, vesicles, golgi, (nuclear or cellular) membranes, ribosomes, nucleoluse, ER, cytoskeleton, vacuoles, centrosome, nucleosome, granules, centrioles, etc.

In certain embodiments of the invention, at least one nuclear localization signal (NLS) is attached to the nucleic acid sequences encoding the Cas proteins. In preferred embodiments at least one or more C-terminal or N-terminal NLSs are attached (and hence nucleic acid molecule(s) coding for the Cas protein can include coding for NLS(s) so that the expressed product has the NLS(s) attached or connected). In a preferred embodiment a C-terminal NLS is attached for optimal expression and nuclear targeting in eukaryotic cells, preferably human cells. The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

### Multiplex Targeting Approach

The Cas proteins herein can employ more than one RNA guide without losing activity. This may enable the use of the Cas proteins, CRISPR-Cas systems or complexes as defined herein for targeting multiple targets (e.g., DNA targets), genes or gene loci, with a single enzyme, system or complex as defined herein. The guide RNAs may be tandemly arranged, optionally separated by a nucleotide sequence such as a direct repeat as defined herein. The position of the different guide RNAs is the tandem does not influence the activity.

In any of the described methods the complex may be delivered with multiple guides for multiplexed use. In any of the described methods more than one protein(s) may be used. In some examples, one Cas protein may be delivered with multiple guides, e.g., at least 2, at least 5, at least 10, at least 15, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 350, at least 400, or at least 500 guides. In some examples, a system herein may comprise a Cas protein and multiple guides, e.g., at least 2, at least 5, at least 10, at least 15, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 350, at least 400, or at least 500 guides.

The Cas enzyme may form part of a CRISPR system or complex, which further comprises tandemly arranged guide RNAs (gRNAs) comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In some embodiments, the functional Cas CRISPR system or complex binds to the multiple target sequences. In some embodiments, the functional CRISPR system or complex may edit the multiple target sequences, e.g., the target sequences may comprise a genomic locus, and in some embodiments there may be an alteration of gene expression. In some embodiments, the functional CRISPR system or complex may comprise further functional domains. In some embodiments, the invention provides a method for altering or modifying expression of multiple gene products. The method may comprise introducing into a cell containing said target nucleic acids, e.g., DNA molecules, or containing and expressing target nucleic acid, e.g., DNA molecules; for instance, the target nucleic acids may encode gene products or provide for expression of gene products (e.g., regulatory sequences). In some general embodiments, the Cas enzyme used for multiplex targeting is associated with one or more functional domains. In some more specific embodiments, the CRISPR enzyme used for multiplex targeting is a deadCas as defined herein elsewhere. In some embodiments, each of the guide sequence is at least 16, 17, 18, 19, 20, 25 nucleotides, or between 16-30, or between 16-25, or between 16-20 nucleotides in length. Examples of multiplex genome engineering using CRISPR effector proteins are provided in Cong et al. (Science February 15; 339(6121):819-23 (2013) and other publications cited herein.

In any of the described methods the strand break may be a single strand break or a double strand break. In preferred embodiments the double strand break may refer to the breakage of two sections of RNA, such as the two sections of RNA formed when a single strand RNA molecule has folded onto itself or putative double helices that are formed with an RNA molecule which contains self-complementary sequences allows parts of the RNA to fold and pair with itself.

### Base Editing

The present disclosure also provides for a base editing system that can be utilized with the synthetic zinc fingers detailed herein. In general, such a system may comprise a deaminase (e.g., an adenosine deaminase or cytidine deaminase) fused with a Cas protein (e.g., a Type IV Cas protein herein). The Cas protein may be a dead Cas protein or a Cas nickase protein. In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities. In one embodiment, the base editor (is fused with a single super degron tag at N-terminal, C-terminal of the deaminase, at the linker region, N-terminal, loop (e.g. Loop-231), or C- of the CRISPR Cas protein (e.g. Cas9 nickase).

In one aspect, the present disclosure provides an engineered adenosine deaminase. The engineered adenosine deaminase may comprise one or more mutations herein. In some embodiments, the engineered adenosine deaminase has cytidine deaminase activity. In certain examples, the engineered adenosine deaminase has both cytidine deaminase activity and adenosine deaminase. In some cases, the modifications by base editors herein may be used for targeting post-translational signaling or catalysis. In some embodiments, compositions herein comprise nucleotide sequence comprising encoding sequences for one or more components of a base editing system. A base-editing system may comprise a deaminase (e.g., an adenosine deaminase or cytidine deaminase) fused with a Cas protein or a variant thereof.

In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities. In some embodiments, the adenosine deaminase may comprise one or more of the mutations: E488Q based on amino acid sequence positions of hADAR2-D, and mutations in a homologous ADAR protein corresponding to the above. In some embodiments, the adenosine deaminase may comprise one or more of the mutations: E488Q, V351G, based on amino acid sequence positions of hADAR2-D, and mutations in a homologous ADAR protein corresponding to the above. In some embodiments, the adenosine deaminase may comprise one or more of the mutations: E488Q, V351G, S486A, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N, K418E based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N, K418E, S661T based on amino acid sequence positions of hADAR2-D, and mutations in a homologous ADAR protein corresponding to the above. In some examples, provided herein includes a mutated adenosine deaminase e.g., an adenosine deaminase comprising one or more mutations of E488Q, V351G, S486A, T375S, S370C, P462A, N597I, L332I, I398V, K350I, M383L, D619G, S582T, V440I, S495N, K418E, S661T, fused with a dead CRISPR-Cas protein or CRISPR-Cas nickase. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, and S661T, fused with a dead CRISPR-Cas protein or a CRISPR-Cas nickase. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, S661T, and S375N fused with a dead CRISPR-Cas protein or a CRISPR-Cas nickase.

In some embodiments, the adenosine deaminase may be a tRNA-specific adenosine deaminase or a variant thereof. In some embodiments, the adenosine deaminase may comprise one or more of the mutations: W23L, W23R, R26G, H36L, N37S, P48S, P48T, P48A, I49V, R51L, N72D, L84F, S97C, A106V, D108N, H123Y, G125A, A142N, S146C, D147Y, R152H, R152P, E155V, I156F, K157N, K161T, based on amino acid sequence positions of E. coli TadA, and mutations in a homologous deaminase protein corresponding to the above. In some embodiments, the adenosine deaminase may comprise one or more of the mutations: D108N based on amino acid sequence positions of E. coli TadA, and mutations in a homologous deaminase protein corresponding to the above. In some embodiments, the adenosine deaminase may comprise one or more of the mutations: A106V, D108N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, based on amino acid sequence positions of E. coli TadA, and mutations in a homologous deaminase protein corresponding to the above. In some embodiments, the adenosine deaminase may comprise one or more of the mutations: A106V, D108N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, R152P, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, R152P, A142N, based on amino acid sequence positions of E. coli TadA, and mutations in a homologous deaminase protein corresponding to the above.

In some examples, the base editing systems may comprise an intein-mediated trans-splicing system that enables in vivo delivery of a base editor, e.g., a split-intein cytidine base editors (CBE) or adenine base editor (ABE) engineered to trans-splice. Examples of the such base editing systems include those described in Colin K. W. Lim et al., Treatment of a Mouse Model of ALS by In Vivo Base Editing, Mol Ther. 2020 Jan. 14. pii: S1525-0016(20)30011-3. doi: 10.1016/j.ymthe.2020.01.005; and Jonathan M. Levy et al., Cytosine and adenine base editing of the brain, liver, retina, heart and skeletal muscle of mice via adeno-associated viruses, Nature Biomedical Engineering volume 4, pages 97-110(2020), which are incorporated by reference herein in their entireties.

Examples of base editing systems include those described in WO2019071048 (e.g. paragraphs [0933]-0938]), WO2019084063 (e.g., paragraphs [0173]-[0186], [0323]-[0475], [0893]-[1094]), WO2019126716 (e.g., paragraphs [0290]-[0425], [1077]-[1084]), WO2019126709 (e.g., paragraphs [0294]-[0453]), WO2019126762 (e.g., paragraphs [0309]-[0438]), WO2019126774 (e.g., paragraphs [0511]-[0670]), Cox D B T, et al., RNA editing with CRISPR-Cas13, Science. 2017 Nov. 24; 358(6366):1019-1027; Abudayyeh 00, et al., A cytosine deaminase for programmable single-base RNA editing, Science 26 Jul. 2019: Vol. 365, Issue 6451, pp. 382-386; Gaudelli N M et al., Programmable base editing of AT to GC in genomic DNA without DNA cleavage, Nature volume 551, pages 464-471 (23 Nov. 2017); Komor A C, et al., Programmable editing of a target base in genomic DNA without double-stranded DNA cleavage. Nature. 2016 May 19; 533(7603):420-4; Jordan L. Doman et al., Evaluation and minimization of Cas9-independent off-target DNA editing by cytosine base editors, Nat Biotechnol (2020). https://doi.org/10.1038/s41587-020-0414-6, which are incorporated by reference herein in their entireties.

### Prime Editing

In some embodiments, the Cas protein herein may be used for prime editing. In some cases, the Cas protein may be a nickase, e.g., a DNA nickase. The Cas may be a dCas. In some cases, the Cas has one or more mutations.

The Cas protein may be associated with a reverse transcriptase. The reverse transcriptase may be fused to the C-terminus of a Cas protein. Alternatively or additionally, the reverse transcriptase may be fused to the N-terminus of a Cas protein. The fusion may be via a linker and/or an adaptor protein. In some examples, the reverse transcriptase may be an M-MLV reverse transcriptase or variant thereof. The M-MLV reverse transcriptase variant may comprise one or more mutations. For the examples, the M-MLV reverse transcriptase may comprise D200N, L603W, and T330P. In another example, the M-MLV reverse transcriptase may comprise D200N, L603W, T330P, T306K, and W313F. In a particular example, the fusion of Cas and reverse transcriptase is Cas (H840A) fused with M-MLV reverse transcriptase (D200N+L603W+T330P+T306K+W313F).

In some embodiments, the Cas protein herein may target DNA using a guide RNA containing a binding sequence that hybridizes to the target sequence on the DNA. The guide RNA may further comprise an editing sequence that contains new genetic information that replaces target DNA nucleotides.

A single-strand break (a nick) may be generated on the target DNA by the Cas protein at the target site to expose a 3′-hydroxyl group, thus priming the reverse transcription of an edit-encoding extension on the guide directly into the target site. These steps may result in a branched intermediate with two redundant single-stranded DNA flaps: a 5′ flap that contains the unedited DNA sequence, and a 3′ flap that contains the edited sequence copied from the guide RNA. The 5′ flaps may be removed by a structure-specific endonuclease, e.g., FEN122, which excises 5′ flaps generated during lagging-strand DNA synthesis and long-patch base excision repair. The non-edited DNA strand may be nicked to induce bias DNA repair to preferentially replace the non-edited strand. Examples of prime editing systems and methods include those described in Anzalone A V et al., Search-and-replace genome editing without double-strand breaks or donor DNA, Nature. 2019 Oct 21. doi: 10.1038/s41586-019-1711-4, which is incorporated by reference herein in its entirety.

The Cas proteins may be used to prime-edit a single nucleotide on a target DNA. Alternatively or additionally, the Cas proteins may be used to prime-edit at least 2, at least 3, at least 4, at least 5, at least 6, at least 7, at least 8, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 200, at least 300, at least 400, at least 500, at least 600, at least 700, at least 800, at least 900, or at least 10000 nucleotides on a target DNA.

### TALE Systems

In some embodiments, the programmable nuclease, e.g. nucleotide-binding molecule in the systems comprising a zinc finger hybrid polypeptide may be a transcription activator-like effector nuclease, a functional fragment thereof, or a variant thereof. The present disclosure also includes nucleotide sequences that are or encode one or more components of a TALE system. As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In some embodiments, provided herein include isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### Zn-Finger Nucleases

In some embodiment, the programmable nuclease, e.g. nucleotide-binding molecule, of the systems may be a Zn-finger nuclease, a functional fragment thereof, or a variant thereof. The composition may comprise one or more Zn-finger nucleases or nucleic acids encoding thereof. In some cases, the nucleotide sequences may comprise coding sequences for Zn-Finger nucleases. Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to FokI cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiments, the programmable nuclease, e.g. nucleotide-binding domain, may be a meganuclease, a functional fragment thereof, or a variant thereof. The composition may comprise one or more meganucleases or nucleic acids encoding thereof. As disclosed herein editing can be made by way of meganucleases, which are endodeoxyribonucleases characterized by a large recognition site (double-stranded DNA sequences of 12 to 40 base pairs). In some cases, the nucleotide sequences may comprise coding sequences for meganucleases. Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, any of the nucleases, including the modified nucleases as described herein, may be used in the methods, compositions, and kits according to the invention. In particular embodiments, nuclease activity of an unmodified nuclease may be compared with nuclease activity of any of the modified nucleases as described herein, e.g. to compare for instance off-target or on-target effects. Alternatively, nuclease activity (or a modified activity as described herein) of different modified nucleases may be compared, e.g. to compare for instance off-target or on-target effects.

### Cells and Organisms

In a further aspect, the invention provides a eukaryotic cell comprising a modified target locus of interest, wherein the target locus of interest has been modified according to in any of the herein described methods. A further aspect provides a cell line of said cell. Another aspect provides a multicellular organism comprising one or more said cells. The cells, cell lines and/or organism comprising said cells advantageously allow for control and/or degradation of the CRISPR-Cas system comprised therein.

The present disclosure provides cells, tissues, organisms comprising the engineered Cas protein, the CRISPR-Cas systems, the polynucleotides encoding one or more components of the CRISPR-Cas systems, and/or vectors comprising the polynucleotides. The invention also provides for the nucleotide sequence encoding the effector protein being codon optimized for expression in a eukaryote or eukaryotic cell in any of the herein described methods or compositions. In an embodiment of the invention, the codon optimized effector protein is any Cas protein discussed herein and is codon optimized for operability in a eukaryotic cell or organism, e.g., such cell or organism as elsewhere herein mentioned, for instance, without limitation, a yeast cell, or a mammalian cell or organism, including a mouse cell, a rat cell, and a human cell or non-human eukaryote organism, e.g., plant.

In certain embodiments, the modification of the target locus of interest may result in: the eukaryotic cell comprising altered expression of at least one gene product; the eukaryotic cell comprising altered expression of at least one gene product, wherein the expression of the at least one gene product is increased; the eukaryotic cell comprising altered expression of at least one gene product, wherein the expression of the at least one gene product is decreased; or the eukaryotic cell comprising an edited genome.

In certain embodiments, the eukaryotic cell may be a mammalian cell or a human cell.

In further embodiments, the non-naturally occurring or engineered compositions, the vector systems, or the delivery systems as described in the present specification may be used for: site-specific gene knockout; site-specific genome editing; RNA sequence-specific interference; or multiplexed genome engineering.

Also provided is a gene product from the cell, the cell line, or the organism as described herein. In certain embodiments, the amount of gene product expressed may be greater than or less than the amount of gene product from a cell that does not have altered expression or edited genome. In certain embodiments, the gene product may be altered in comparison with the gene product from a cell that does not have altered expression or edited genome.

### Cargos

The delivery systems may comprise one or more cargos. The cargos may comprise one or more components of the systems and compositions herein. A cargo may comprise one or more of the following: i) a plasmid encoding one or more Cas proteins; ii) a plasmid encoding one or more guide RNAs, iii) mRNA of one or more Cas proteins; iv) one or more guide RNAs; v) one or more Cas proteins; vi) any combination thereof. In some examples, a cargo may comprise a plasmid encoding one or more Cas protein and one or more (e.g., a plurality of) guide RNAs. In some embodiments, a cargo may comprise mRNA encoding one or more Cas proteins and one or more guide RNAs.

In some examples, a cargo may comprise one or more Cas proteins and one or more guide RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516.

### Physical Delivery

In some embodiments, the cargos may be introduced to cells by physical delivery methods. Examples of physical methods include microinjection, electroporation, and hydrodynamic delivery.

### Microinjection

Microinjection of the cargo directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for Cas proteins and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas-encoding mRNA to the cytoplasm, e.g., facilitating translation and shuttling of Cas to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present invention may a greatest dimension (e.g. diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

### Vectors

The systems, compositions, and/or delivery systems may comprise one or more vectors. The present disclosure also include vector systems. A vector system may comprise one or more vectors. In some embodiments, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET 11d, yeast expression vectors (e.g., pYepSecl, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC.

A vector may comprise i) Cas encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively or additionally, in a single vector, there may be a promoter controlling (e.g., driving transcription and/or expression) multiple RNA encoding sequences.

### Regulatory Elements

A vector may comprise one or more regulatory elements. The regulatory element(s) may be operably linked to coding sequences of Cas proteins, accessary proteins, guide RNAs (e.g., a single guide RNA, crRNA, and/or tracrRNA), or combination thereof. The term “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). In certain examples, a vector may comprise: a first regulatory element operably linked to a nucleotide sequence encoding a Cas protein, and a second regulatory element operably linked to a nucleotide sequence encoding a guide RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter.

### Viral Vectors

The cargos may be delivered by viruses. In some embodiments, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In some embodiments, AAV may provide a persistent source of the provided DNA, as AAV delivered genomic material can exist indefinitely in cells, e.g., either as exogenous DNA or, with some modification, be directly integrated into the host DNA. In some embodiments, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82: 5887-5911 (2008)), and shown as follows:

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

CRISPR-Cas AAV particles may be created in HEK 293 T cells. Once particles with specific tropism have been created, they are used to infect the target cell line much in the same way that native viral particles do. This may allow for persistent presence of CRISPR-Cas components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of Cas and gRNA may be packaged directly onto one DNA plasmid vector and delivered via one AAV particle. In some examples, AAVs may be used to deliver gRNAs into cells that have been previously engineered to express Cas. In some examples, coding sequences of Cas and gRNA may be made into two separate AAV particles, which are used for co-transfection of target cells. In some examples, markers, tags, and other sequences may be packaged in the same AAV particles as coding sequences of Cas and/or gRNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In certain embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In some embodiments, adenoviruses do not integrate into the genome of host cells, which may be used for limiting off-target effects of CRISPR-Cas systems in gene editing applications.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero phosphocholine, PEG-cDMA, and 1,2-dilinoleyloxy-3-(N;N-dimethyl)aminopropane (DLinDMA)

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 [symbol] (e.g., forming DNA/Ca²⁺ microcomplexes), polyethenimine (PEI) (e.g., branched PEI), and poly(L-lysine) (PLL).

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl). Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct 22; 136(42):14722-5; and Sun W et al, Angew Chem Int Ed Engl. 2015 Oct 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Gold Nanoparticles

In some embodiments, the delivery vehicles comprise gold nanoparticles (also referred to AuNPs or colloidal gold). Gold nanoparticles may form complex with cargos, e.g., Cas:gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi:10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Methods of Use in General

In another aspect, the present disclosure discloses methods of using the compositions and systems herein. In general, the methods allow for the control, modulation, and/or degradation of systems detailed herein. Such systems can be utilized for modifying a target nucleic acid by introducing in a cell or organism that comprises the target nucleic acid the engineered Cas protein, polynucleotide(s) encoding engineered Cas protein, the CRISPR-Cas system, or the vector or vector system comprising the polynucleotide(s), such that the engineered Cas protein modifies the target nucleic acid in the cell or organism. Additional applications of the systems, such as activating or repressing translation, base editing, labeling of molecules and their interactions are known in the art and can be utilized with the approaches and zinc finger systems detailed herein.

Methods of inducing degradation of a CRISPR Cas protein comprising one or more zinc finger degradation domains-RNA complex (CRISPR-Cas variant) are provided. In an aspect, the method comprises contacting the CRISPR Cas variant protein-RNA complex with a degrader, e.g. IMiD or small molecule, as detailed elsewhere herein.

Methods may comprise delivering to a cell comprising the variant Cas polypeptides of the present invention, or expressing the polynucleotide encoding the variant Cas polypeptides of the present invention, or provided a cell transfected with the vector comprising the polynucleotide, and a molecule capable of inducing degradation, for example an IMiD or other degrader of zinc finger degron.

The method may be performed in vitro, ex vivo, or in vivo. In an aspect, the method is performed in a cell. In particular embodiments, the methods are performed in a germline cell. Methods of degrading activity can be detected in a variety of ways, including measuring activity at a target molecule, via genomic disruption e.g. eGFP disruption as described in the examples herein. Varying levels of degrader agents may be utilized with eGFP disruption assayed versus an apoCas, and/or a Cas protein activity with no degrader.

The degraders herein may be used to modulate the functions and activities of RNA-guided nuclease (e.g., Cas proteins), variants thereof, and fragments thereof in animals and non-animal organisms. In some examples, the animals and non-animal organisms may have been engineered to constitutively or inducibly express an RNA-guided nuclease (e.g., Cas protein) comprising one or more functional domains. In some examples, the degraders herein may modulate the activities of the RNA-guided nucleases comprising one or more degradation domains or their interaction with other molecules, e.g., their binding with target polynucleotides.

Methods of inducing degradation of an engineered or modified Cas polypeptide are provided, and comprise delivering to a cell comprising the variant Cas polypeptides of the present invention, or expressing the polynucleotide encoding the variant Cas polypeptides of the present invention, or provided a cell transfected with the vector comprising the polynucleotide, and an IMiD, also referred to herein as a degrader. The delivery of the IMiD may occur at a time subsequent to delivery or expression of the Cas polypeptide or other programmable nuclease. In certain aspect, the exposing the cell to the IMiD is performed about 1 to 10 hours, about 10 to 24, about 24 to 36, about 24 to 48 hours after the cell is transfected with a vector, or about 2 to 8 hours, about 3 to 6 hours after transfection or expression of the variant Cas polypeptide or other programmable nuclease. In an aspect, exposing comprises incubating the cell with the IMiD or pharmaceutically acceptable salt thereof, wherein the IMiD is provided at a concentration of about 1 nM to about 10 nm, or about 10 nM to about 10 μM.

Methods of controlling Cas polypeptide editing outcomes are provided, and can comprise administering an immunomodulatory imide drug (IMiD) or a pharmaceutically acceptable salt thereof to a cell or a population of cells. The cell or population of cells comprise or express an engineered or modified Cas polypeptide as disclosed herein. In one aspect, the cell is a germline cell, in some, the cell is in an organism. In some methods, the step of exposing comprises incubating the cell with the compound or pharmaceutically acceptable salt thereof. In an aspect the exposing or administering of the IMiD is performed at a time to encourage microhomology repair or single base insertion outcomes, and/or to encourage HDR repair pathways over NHEJ repair pathways.

### Modulation of Gene Editing Mechanisms

The degraders herein may be administered to cells or organisms at doses effective to impact gene editing outcomes, e.g., to control the gene editing mechanisms via NHEJ or HDR.

The activity of NHEJ and HDR DSB repair varies significantly by cell type and cell state. NHEJ is not highly regulated by the cell cycle and is efficient across cell types, allowing for high levels of gene disruption in accessible target cell populations. In contrast, HDR acts primarily during S/G2 phase, and is therefore restricted to cells that are actively dividing, limiting treatments that require precise genome modifications to mitotic cells. Ciccia, A. & Elledge, S. J. Molecular cell 40, 179-204 (2010); Chapman, J. R., et al. Molecular cell 47, 497-510 (2012)].

The degraders may affect the gene editing mechanisms by modulating the function and activity of the RNA-guided nuclease involved in the gene editing. The efficiency of correction via HDR may be controlled by the epigenetic state or sequence of the targeted locus, or the specific repair template configuration (single vs. double stranded, long vs. short homology arms) used [Hacein-Bey-Abina, S., et al. The New England journal of medicine 346, 1185-1193 (2002); Gaspar, H. B., et al. Lancet 364, 2181-2187 (2004); Beumer, K. J., et al. G3 (2013)]. The relative activity of NHEJ and HDR machineries in target cells may also affect gene correction efficiency, as these pathways may compete to resolve DSBs [Beumer, K. J., et al. Proceedings of the National Academy of Sciences of the United States of America 105, 19821-19826 (2008)]. HDR also imposes a delivery challenge not seen with NHEJ strategies, as it requires the concurrent delivery of nucleases and repair templates. In practice, these constraints have so far led to low levels of HDR in therapeutically relevant cell types. Clinical translation has therefore largely focused on NHEJ strategies to treat disease, although proof-of-concept preclinical HDR treatments have now been described for mouse models of haemophilia B and hereditary tyrosinemia [Li, H., et al. Nature 475, 217-221 (2011); Yin, H., et al. Nature biotechnology 32, 551-553 (2014)].

The degraders herein may be used (e.g., with an RNA-guided nuclease comprising one or more degradation domains) to create a platform to model a disease or disorder of an animal, in some embodiments a mammal, in some embodiments a human. In certain embodiments, such models and platforms are rodent based, in non-limiting examples rat or mouse. Such models and platforms can take advantage of distinctions among and comparisons between inbred rodent strains. In certain embodiments, such models and platforms primate, horse, cattle, sheep, goat, swine, dog, cat or bird-based, for example to directly model diseases and disorders of such animals or to create modified and/or improved lines of such animals. Advantageously, in certain embodiments, an animal-based platform or model is created to mimic a human disease or disorder. For example, the similarities of swine to humans make swine an ideal platform for modeling human diseases. Compared to rodent models, development of swine models has been costly and time intensive. On the other hand, swine and other animals are much more similar to humans genetically, anatomically, physiologically and pathophysiologically. The degraders herein may be used to provide a high efficiency platform for targeted gene and genome editing, gene and genome modification and gene and genome regulation to be used in such animal platforms and models. Though ethical standards block development of human models and in many cases models based on non-human primates, the present invention is used with in vitro systems, including but not limited to cell culture systems, three dimensional models and systems, and organoids to mimic, model, and investigate genetics, anatomy, physiology and pathophysiology of structures, organs, and systems of humans. The platforms and models provide manipulation of single or multiple targets.

The degraders herein may be used, e.g., with an RNA-guided nuclease, to create a plant, an animal or cell that may be used to model and/or study genetic or epigenetic conditions of interest, such as a through a model of mutations of interest or a disease model. In some embodiments, the models may be generated using the RNA-guided nuclease, and the characters of the models may be further modulated and controlled using the degraders herein.

As used herein, “disease” refers to a disease, disorder, or indication in a subject. For example, a method of the invention may be used to create an animal or cell that comprises a modification in one or more nucleic acid sequences associated with a disease, or a plant, animal or cell in which the expression of one or more nucleic acid sequences associated with a disease are altered. Such a nucleic acid sequence may encode a disease associated protein sequence or may be a disease associated control sequence. Accordingly, it is understood that in embodiments of the invention, a plant, subject, patient, organism or cell can be a non-human subject, patient, organism or cell. Thus, the invention provides a plant, animal or cell, produced by the present methods, or a progeny thereof. The progeny may be a clone of the produced plant or animal, or may result from sexual reproduction by crossing with other individuals of the same species to introgress further desirable traits into their offspring. The cell may be in vivo or ex vivo in the cases of multicellular organisms, particularly animals or plants. In the instance where the cell is in cultured, a cell line may be established if appropriate culturing conditions are met and preferably if the cell is suitably adapted for this purpose (for instance a stem cell). Bacterial cell lines produced by the invention are also envisaged. Hence, cell lines are also envisaged.

In some methods, the disease model can be used to study the effects of mutations on the animal or cell and development and/or progression of the disease using measures commonly used in the study of the disease. Alternatively, such a disease model is useful for studying the effect of a pharmaceutically active compound on the disease.

In some methods, the disease model can be used to assess the efficacy of a potential gene therapy strategy. That is, a disease-associated gene or polynucleotide can be modified such that the disease development and/or progression is inhibited or reduced. In particular, the method comprises modifying a disease-associated gene or polynucleotide such that an altered protein is produced and, as a result, the animal or cell has an altered response. Accordingly, in some methods, a genetically modified animal may be compared with an animal predisposed to development of the disease such that the effect of the gene therapy event may be assessed.

In another embodiment, this invention provides a method of developing a biologically active agent that modulates a cell signaling event associated with a disease gene. The method comprises contacting a test compound with a cell comprising one or more vectors that drive expression of one or more of components of the system; and detecting a change in a readout that is indicative of a reduction or an augmentation of a cell signaling event associated with, e.g., a mutation in a disease gene contained in the cell.

A cell model or animal model can be constructed in combination with the method of the invention for screening a cellular function change. Such a model may be used to study the effects of a genome sequence modified by the systems and methods herein on a cellular function of interest. For example, a cellular function model may be used to study the effect of a modified genome sequence on intracellular signaling or extracellular signaling. Alternatively, a cellular function model may be used to study the effects of a modified genome sequence on sensory perception. In some such models, one or more genome sequences associated with a signaling biochemical pathway in the model are modified.

The degraders herein may be used for treatment in a variety of diseases and disorders. The degraders may be used to modulate the function and activity of an RNA-guided nuclease (e.g., a Cas protein) used for treating a disease. For example, the degraders may be used for regulating the strength, efficacy, timing, dosage of the therapeutic RNA-guided nuclease.

In some cases, a small molecule inhibitor herein may be administered to a subject concurrently with an RNA-guided nuclease. Alternatively, or additionally, a small molecule inhibitor herein may be administered to a subject prior to the administration of an RNA-guided nuclease. Alternatively, or additionally, a small molecule inhibitor herein may be administered to a subject after the administration of an RNA-guided nuclease. In some examples, the degraders herein are used for modulating CRISPR gene editing (e.g., by modulating Cas protein of the CRISPR system).

The degraders herein may be administered as one or more doses as needed. In some examples, the degraders may be administered as a single dose. In certain examples, the degraders may be administered as multiple doses, e.g., 2, 3, 4, 5, 6, 7, 8, 9, 10 or more doses. The multi-dose regime may be used to achieve optimal efficacy and/or temporal control of the activity and function of the RNA-guided nuclease.

### Exemplary Therapies

The degraders herein may be used for treatment in a variety of diseases and disorders. The degraders may be used to modulate the function and activity of an RNA-guided nuclease (e.g., a Cas protein) used for treating a disease.

In embodiments, the compounds can be used in method for therapy in which cells are edited ex vivo, in vivo or in vitro using CRISPR systems to modulate at least one gene. In embodiments, in vitro methods may include with subsequent administration of the edited cells to a patient in need thereof. In some embodiments, the CRISPR editing involves knocking in, knocking out or knocking down expression of at least one target gene in a cell. In particular embodiments, the degraders herein can modulate CRISPR editing when utilizing a CRIPSR protein with one or more degradation domains inserts an exogenous, gene, minigene or sequence, which may comprise one or more exons and introns or natural or synthetic introns into the locus of a target gene, a hot-spot locus, a safe harbor locus of the gene genomic locations where new genes or genetic elements can be introduced without disrupting the expression or regulation of adjacent genes, or correction by insertions or deletions one or more mutations in DNA sequences that encode regulatory elements of a target gene.

In embodiments, the treatment is for disease/disorder of an organ, including liver disease, eye disease, muscle disease, heart disease, blood disease, brain disease, kidney disease, or may comprise treatment for an autoimmune disease, central nervous system disease, cancer and other proliferative diseases, neurodegenerative disorders, inflammatory disease, metabolic disorder, musculoskeletal disorder and the like.

### Formulations

Agents described herein, including analogs thereof, and/or agents discovered to have medicinal value using the methods described herein are useful as a drug for treating diabetes. For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms.

Through this disclosure and the knowledge in the art, components of the systems and compositions herein may be delivered by a delivery system herein described both generally and in detail. The present disclosure also provides delivery systems for introduce components of the systems and compositions herein to cells, tissues, or organs. The system may comprise one or more delivery vehicles herein. The systems may further comprise one or more components of the systems herein. For examples, delivery systems may comprise vectors, polynucleotide molecules, the one or more vectors or polynucleotide molecules comprising one or more polynucleotide molecules encoding the Type II Cas protein and one or more nucleic acid components of the non-naturally occurring or engineered composition. The delivery vehicle comprising liposomes, nanoparticles, exosomes, microvesicles, nucleic acid nanoassemblies, a gene gun, an implantable device, or a vector system.

### Formulations

Agents described herein, including analogs thereof, and/or agents discovered to have medicinal value using the methods described herein are useful as a drug for treating diabetes. For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms. Generally, amounts will be in the range of those used for other agents used in the treatment of other diseases associated with diabetes.

The disclosed compounds may be administered alone (e.g., in saline or buffer) or using any delivery vehicles known in the art. For instance, the following delivery vehicles have been described: Cochleates; Emulsomes, ISCOMs; Liposomes; Live bacterial vectors (e.g., Salmonella, Escherichia coli, Bacillus calmatte-guerin, Shigella, Lactobacillus); Live viral vectors (e.g., Vaccinia, adenovirus, Herpes Simplex); Microspheres; Nucleic acid vaccines; Polymers; Polymer rings; Proteosomes; Sodium Fluoride; Transgenic plants; Virosomes; Virus-like particles. Other delivery vehicles are known in the art and some additional examples are provided below.

The disclosed compounds may be administered by any route known, such as, for example, orally, transdermally, intravenously, cutaneously, subcutaneously, nasally, intramuscularly, intraperitoneally, intracranially, and intracerebroventricularly.

In certain embodiments, disclosed compounds are administered at dosage levels greater than about 0.001 mg/kg, such as greater than about 0.01 mg/kg or greater than about 0.1 mg/kg. For example, the dosage level may be from about 0.001 mg/kg to about 50 mg/kg such as from about 0.01 mg/kg to about 25 mg/kg, from about 0.1 mg/kg to about 10 mg/kg, or from about 1 mg/kg to about 5 mg/kg of subject body weight per day, one or more times a day, to obtain the desired therapeutic effect. It will also be appreciated that dosages smaller than about 0.001 mg/kg or greater than about 50 mg/kg (for example about 50-100 mg/kg) can also be administered to a subject.

In one embodiment, the compound is administered once-daily, twice-daily, or three-times daily. In one embodiment, the compound is administered continuously (i.e., every day) or intermittently (e.g., 3-5 days a week). In another embodiment, administration could be on an intermittent schedule.

Further, administration less frequently than daily, such as, for example, every other day may be chosen. In additional embodiments, administration with at least 2 days between doses may be chosen. By way of example only, dosing may be every third day, bi-weekly or weekly. As another example, a single, acute dose may be administered. Alternatively, compounds can be administered on a non-regular basis e.g., whenever symptoms begin. For any compound described herein the effective amount can be initially determined from animal models.

Toxicity and efficacy of the compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices may have a greater effect when practicing the methods as disclosed herein. While compounds that exhibit toxic side effects may be used, care should be taken to design a delivery system that targets such compounds to the site of affected tissue in order to minimize potential damage to uninfected cells and, thereby, reduce side effects.

Data obtained from the cell culture assays and animal studies can be used in formulating a range of dosage of the compounds disclosed herein for use in humans. The dosage of such agents lies within a range of circulating concentrations that include the ED₅₀ with little or no toxicity. The dosage may vary within this range depending upon the dosage form employed and the route of administration utilized. For any compound used in the disclosed methods, the effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography. In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. Multiple doses of the compounds are also contemplated.

The formulations disclosed herein are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of one or more disclosed compounds can be administered to a subject by any mode that delivers the compound(s) to the desired surface, e.g., mucosal, systemic. Administering the pharmaceutical composition of the present disclosure may be accomplished by any means known to the skilled artisan. Disclosed compounds may be administered orally, transdermally, intravenously, cutaneously, subcutaneously, nasally, intramuscularly, intraperitoneally, intracranially, or intracerebroventricularly.

For oral administration, one or more compounds can be formulated readily by combining the active compound(s) with pharmaceutically acceptable carriers well known in the art. Such carriers enable the compounds to be formulated as tablets, pills, dragees, capsules, liquids, gels, syrups, slurries, suspensions and the like, for oral ingestion by a subject to be treated.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of one or more disclosed compounds. The compound(s) may be chemically modified so that oral delivery of the derivative is efficacious. Generally, the chemical modification contemplated is the attachment of at least one moiety to the compound itself, where said moiety permits (a) inhibition of proteolysis; and (b) uptake into the blood stream from the stomach or intestine. Also desired is the increase in overall stability of the compound(s) and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Other polymers that could be used are poly-1,3-dioxolane and poly-1,3,6-tioxocane. In some aspects for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. In some aspects, the release will avoid the deleterious effects of the stomach environment, either by protection of the compound or by release of the biologically active material beyond the stomach environment, such as in the intestine.

To ensure full gastric resistance a coating impermeable to at least pH 5.0 is important. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The disclosed compounds can be included in the formulation as fine multiparticulates in the form of granules or pellets of particle size about 1 mm. The formulation of the material for capsule administration could also be as a powder, lightly compressed plugs or even as tablets. The compound could be prepared by compression.

Colorants and flavoring agents may all be included. For example, the compound may be formulated (such as by liposome or microsphere encapsulation) and then further contained within an edible product, such as a refrigerated beverage containing colorants and flavoring agents.

One may dilute or increase the volume of compound delivered with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell. Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants is the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the compound to prevent sticking during the formulation process. Lubricants may be used as a layer between the compound and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000. Glidants that might improve the flow properties of the drug during formulation and to aid rearrangement during compression might be added. The glidants may include starch, talc, pyrogenic silica and hydrated silicoaluminate.

To aid dissolution of the compound into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the compounds of the disclosure. The compound is delivered to the lungs of a mammal while inhaling and traverses across the lung epithelial lining to the blood stream using methods well known in the art.

Contemplated for use in the practice of methods disclosed herein are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of these methods are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of compound. Typically, each formulation is specific to the type of device employed and may involve the use of an appropriate propellant material, in addition to the usual diluents, and/or carriers useful in therapy. Also, the use of liposomes, microcapsules or microspheres, inclusion complexes, or other types of carriers is contemplated. Chemically modified compound may also be prepared in different formulations depending on the type of chemical modification or the type of device employed. Formulations suitable for use with a nebulizer, either jet or ultrasonic, will typically comprise compound dissolved in water at a concentration of about 0.1 to about 25 mg of biologically active compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the compound suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifiuoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound and may also include a bulking agent, such as lactose, sorbitol, sucrose, or mannitol in amounts which facilitate dispersal of the powder from the device, e.g., about 50 to about 90% by weight of the formulation. The compound should most advantageously be prepared in particulate form with an average particle size of less than 10 mm (or microns), such as about 0.5 to about 5 mm, for an effective delivery to the distal lung.

Nasal delivery of a disclosed compound is also contemplated. Nasal delivery allows the passage of a compound to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition solution into a chamber of defined volume, which chamber has an aperture dimensioned to aerosolize and aerosol formulation by forming a spray when a liquid in the chamber is compressed. The chamber is compressed to administer the pharmaceutical composition. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. In some aspects, the nasal inhaler will provide a metered amount of the aerosol formulation, for administration of a measured dose of the drug.

The compound, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions.

Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems.

The compounds may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (about 1-2% w/v); citric acid and a salt (about 1-3% w/v); boric acid and a salt (about 0.5-2.5% w/v); and phosphoric acid and a salt (about 0.8-2% w/v). Suitable preservatives include benzalkonium chloride (about 0.003-0.03% w/v); chlorobutanol (about 0.3-0.9% w/v); parabens (about 0.01-0.25% w/v) and thimerosal (about 0.004-0.02% w/v).

The pharmaceutical compositions contain an effective amount of a disclosed compound optionally included in a pharmaceutically acceptable carrier. The term pharmaceutically acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds, and with each other, in a manner such that there is no interaction which would substantially impair the desired pharmaceutical efficiency.

The invention can be captured in the following numbered statements:

Statement 1. A hybrid zinc finger polypeptide comprising an N-terminal portion selected from SEQ ID NOs: 46, 49, 51, 53, 55, 57, 59, 61, 63, 65, 67, 69, 71, 73, 75, 77, 79, 81, 83, 85, 87; and an alpha-helix selected from SEQ ID NOs: 47, 89, 111, 133, 155, 177, 199, 221, 243, 265, 287, 309, 331, 353, 375, 397, 419, 441, 462, 484, and 506.

Statement 2. The hybrid zinc finger polypeptide of Statement 1, comprising a sequence selected from SEQ ID NOs: 48, 50, 52, 54, 56, 58, 60, 62, 64, 66, 68, 70, 72, 74, 76, 78, 80, 82, 84, 86, 88, 90, 91, 92, 93, 94, 95, 96, 97, 98, 99, 100, 101, 102, 103, 104, 105, 106, 107, 108, 109, 110, 112, 113, 114, 115, 116, 117, 118, 119, 120, 121, 122, 123, 124, 125, 126, 127, 128, 129, 130, 131, 132, 134, 135, 136, 137, 138, 139, 140, 141, 142, 143, 144, 145, 146, 147, 148, 149, 150, 151, 152, 153, 154, 156, 157, 158, 159, 160, 161, 162, 163, 164, 165, 166, 167, 168, 169, 170, 171, 172, 173, 174, 175, 176, 178, 179, 180, 181, 182, 183, 184, 185, 186, 187, 188, 189, 190, 191, 192, 193, 194, 195, 196, 197, 198, 200, 201, 202, 203, 204, 205, 206, 207, 208, 209, 210, 211, 212, 213, 214, 215, 216, 217, 218, 219, 220, 222, 223, 224, 225, 226, 227, 228, 229, 230, 231, 232, 233, 234, 235, 236, 237, 238, 239, 240, 241, 242, 244, 245, 246, 247, 248, 249, 250, 251, 252, 253, 254, 255, 256, 257, 258, 259, 260, 261, 262, 263, 264, 266, 267, 268, 269, 270, 271, 272, 273, 274, 275, 276, 277, 278, 279, 280, 281, 282, 283, 284, 285, 286, 288, 289, 290, 291, 292, 293, 294, 295, 296, 297, 298, 299, 300, 301, 302, 303, 304, 305, 306, 307, 308, 310, 311, 312, 313, 314, 315, 316, 317, 318, 319, 320, 321, 322, 323, 324, 325, 326, 327, 328, 329, 330, 332, 333, 334, 335, 336, 337, 338, 339, 340, 341, 342, 343, 344, 345, 346, 347, 348, 349, 350, 351, 352, 354, 355, 356, 357, 358, 359, 360, 361, 362, 363, 364, 365, 366, 367, 368, 369, 370, 371, 372, 373, 374, 376, 377, 378, 379, 380, 381, 382, 383, 384, 385, 386, 387, 388, 389, 390, 391, 392, 393, 394, 395, 396, 398, 399, 400, 401, 402, 403, 404, 405, 406, 407, 408, 409, 410, 411, 412, 413, 414, 415, 416, 417, 418, 420, 421, 422, 423, 424, 425, 426, 427, 428, 429, 430, 431, 432, 433, 434, 435, 436, 437, 438, 439, 440, 442, 443, 444, 445, 446, 447, 448, 449, 450, 451, 452, 453, 454, 455, 456, 457, 458, 459, 460, 461, 463, 464, 465, 466, 467, 468, 469, 470, 471, 472, 473, 474, 475, 476, 477, 478, 479, 480, 481, 482, 483, 485, 486, 487, 488, 489, 490, 491, 492, 493, 494, 495, 496, 497, 498, 499, 500, 501, 502, 503, 504, 505, 507, 508, 509, 510, 511, 512, 513, 514, 515, 516, 517, 518, 519, 520, 521, 522, 523, 524, 525, 526, or 527.

Statement 3. The hybrid zinc finger of Statement 1, wherein the hybrid zinc finger polypeptide optimized for degradation by pomalidomide, and the zinc finger polypeptide comprises a sequence selected from SEQ ID NO: XX to XX (from FIG. 17A).

Statement 4. The hybrid zinc finger of Statement 1, wherein the hybrid zinc finger polypeptide optimized for degradation by avadomide, and the zinc finger polypeptide comprises a sequence selected from SEQ ID NO: XX to XX (from FIG. 17B).

Statement 5. The hybrid zinc finger of Statement 1, wherein the hybrid zinc finger polypeptide optimized for degradation by iberomide, and the zinc finger polypeptide comprises a sequence selected from SEQ ID NO: XX to XX (from FIG. 17C).

Statement 6. The hybrid zinc finger of Statement 1, wherein the hybrid zinc finger polypeptide optimized for degradation by lenalidomide, and the zinc finger polypeptide comprises a sequence selected from SEQ ID NO: XX to XX (from FIGS. 17D/17E).

Statement 7. A programmable nuclease comprising one or more hybrid zinc finger polypeptides of Statement 2 introduced into the nuclease at one or more insertion sites.

Statement 8. The programmable nuclease of Statement 7, wherein the nuclease is a CRISPR-Cas protein, a Zinc finger nuclease, a TALEN or a meganuclease.

Statement 9. The programmable nuclease of Statement 7 that is codon optimized for expression in eukaryotes.

Statement 10. The programmable nuclease of Statement 8 wherein the CRISPR-Cas protein is a Type II, Type V or Type VI Cas protein.

Statement 11. The programmable nuclease of Statement 10, wherein the CRISPR-Cas protein is a Cas9, a Cas12a, Cas12b, Cas12c, Cas12d, Cas13a, Cas13b, Cas13c, or Cas13d protein.

Statement 12. The programmable nuclease of Statement 10, wherein the one or more insertion sites are at the N-terminal (Nt), C-terminal (Ct) or at a position corresponding to a position on the loop of a SpCas9 protein.

Statement 13. The programmable nuclease of Statement 10, wherein the sequence comprises SEQ ID NO: 45.

Statement 14. The programmable nuclease of Statement 6, wherein the CRISPR-Cas protein is a dCas9.

Statement 15. The programmable nuclease of Statement 14, wherein the dCas9 is fused to one or more functional domains.

Statement 16. The programmable nuclease of Statement 15, wherein the functional domain is a KRAB domain or a transposase domain.

Statement 17. The programmable nuclease of Statement 6, wherein the CRISPR-Cas protein is a Cas-based nickase, optionally wherein the Cas-based nickase is a Cas9 nickase which comprises a mutation in the HNH domain.

Statement 18. The programmable nuclease of Statement 17, wherein the functional component is a base editing component, optionally wherein the base editing component is fused directly or indirectly to the N terminal of the CRISPR-Cas nickase.

Statement 19. The programmable nuclease of Statement 18, wherein the base editing component comprises an adenosine deaminase.

Statement 20. The programmable nuclease of Statement 18 or 19, wherein the base editing component is fused at N-terminal or C-terminal of the adenosine deaminase, at the linker region, the N-terminal, a loop of the CRISPR-Cas nickase, or C-terminal of the CRISPR-Cas nickase.

Statement 21. A ribonucleoprotein comprising the programmable nuclease of any one of Statements 7 to 20.

Statement 22. A plasmid comprising the variant CRISPR-Cas protein of any one of Statements 7 to 20.

Statement 23. A cell transfected with the ribonucleoprotein of Statement 21 or the plasmid of Statement 22.

Statement 24. A method of inducing degradation of a programmable nuclease, comprising: exposing the cell of Statement 22 with an immunomodulatory imide drug (IMiD) or a pharmaceutically acceptable salt thereof.

Statement 25. The method of Statement 24, wherein the IMiD is selected from thalidomide, lenalidomide, pomalidomide, avadomide, iberdomide, and analogs thereof.

Statement 26. The method of Statement 25, wherein the exposing the cell with the IMiD is performed about 3 to 6 hours, about 6 to 12 hours, about 12 to 24 hours, about 24 to 48 hours after the cell is transfected.

Statement 27. The method of Statement 26, wherein the exposing comprises incubating the cell with the compound or pharmaceutically acceptable salt thereof, wherein the compound is provided at a concentration of about 10 nM to about 10 μM.

Statement 28. The method of Statement 24, wherein the cell is a germline cell.

Statement 29. The method of Statement 24, wherein the cell is in an organism.

Statement 30. The method of Statement 24, wherein the cell comprises the hybrid zinc finger comprising the sequence from FIG. 17A, and the IMiD is pomalidomide.

Statement 31. The method of Statement 22, wherein the cell comprises the hybrid zinc finger comprising the sequence from FIG. 17B, and the IMiD is avadomide.

Statement 32. The method of Statement 22, wherein the cell comprises the hybrid zinc finger comprising the sequence from FIG. 17C, and the IMiD is iberomide.

Statement 33. The method of Statement 22, wherein the cell comprises the hybrid zinc finger comprising the sequence from 17D or 17E, and the IMiD is lenalidomide.

Statement 34. A method of controlling programmable nuclease editing outcomes comprising administering an immunomodulatory imide drug (IMiD) or a pharmaceutically acceptable salt thereof to a cell or a population of cells comprising or expressing a variant CRISPR-Cas protein of any one of Statements 7 to 20.

Statement 35. The method of Statement 34, wherein the IMiD is selected from thalidomide, lenalidomide, pomalidomide, avadomide, iberomide, and analogs thereof.

Statement 36. The method of Statement 34, wherein the method is performed in vitro or in vivo.

Statement 37. The method of any of the preceding Statements wherein the exposing or administering of the IMiD is performed at a time to encourage target specificity.

The invention is further described in the following examples, which do not limit the scope of the invention described in the claims.

### EXAMPLES

### Example 1 CRISPR-Cas Editing Outcomes

Degraders targeting variant Cas9 proteins is explored in the following example. SpCas9 variants were prepared and transfected into several cell lines. The cells were incubated with dTAG, degrader compositions, and evaluated for SpCas9 activity via genomic eGFP-PEST disruption.

Control of CRIPSR-Cas degradation can control editing outcomes. 1 uM of dTAG was found sufficient for complete degradation of 2FKBP (N+L) Cas9 in multiple assays. For example, eGFP disruption assays with RNP and plasmid delivery, western blotting showing degradation of transiently expressed FKBP-Cas9, degradation kinetics of stably expressed Cas9 in 293T cells, DNA repair outcome in mouse embryonic stem cell.

Further experiments were conducted with dTAG-47 added at 6 hr, 12 hr, 24 hr, 48 hr and 120 hr-no dTAG-47, with effect on Cas9 editing explored in detail. The results indicate that the dTAG-47 degrader small molecule can be used to control the DNA repair outcome, and hence the nature of the sequence.

Regarding changes of Cas9 editing outcomes, sorting dTAG CRISPR outcome fractions by timestep confidence range, MMEJ (MH deletions, microhomology endjoining) outcomes require longer-term Cas9 treatment. NHEJ (Non-MH deletions) outcomes predominate early on and 1 bp insertions increase the longer Cas9 is present. (FIG. 1A)

Additionally, the longer the time Cas9 is present, the observed CRISPR phenotypes are increased relative in contrast to wildtype observation. (FIG. 1B)

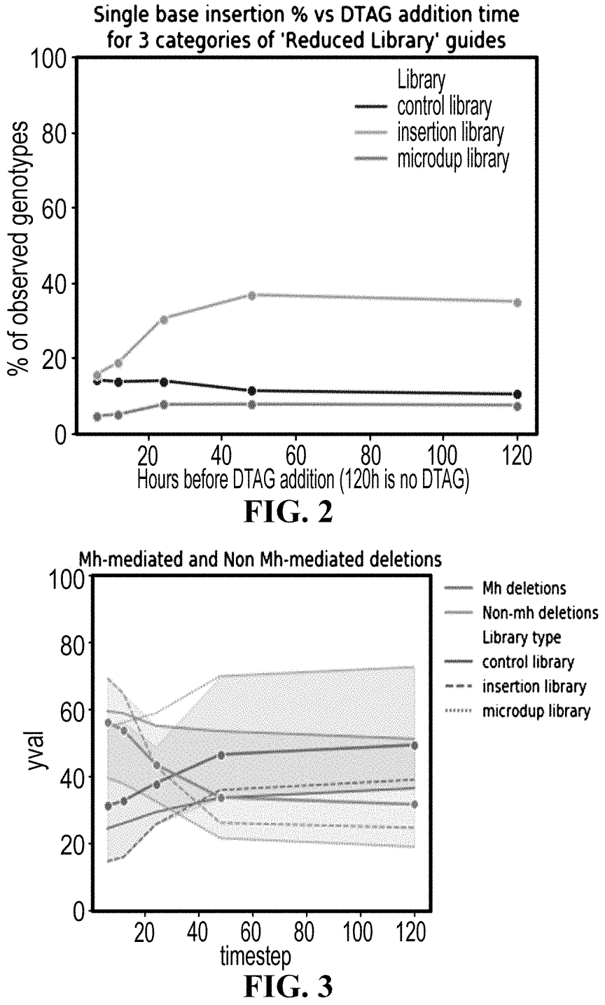

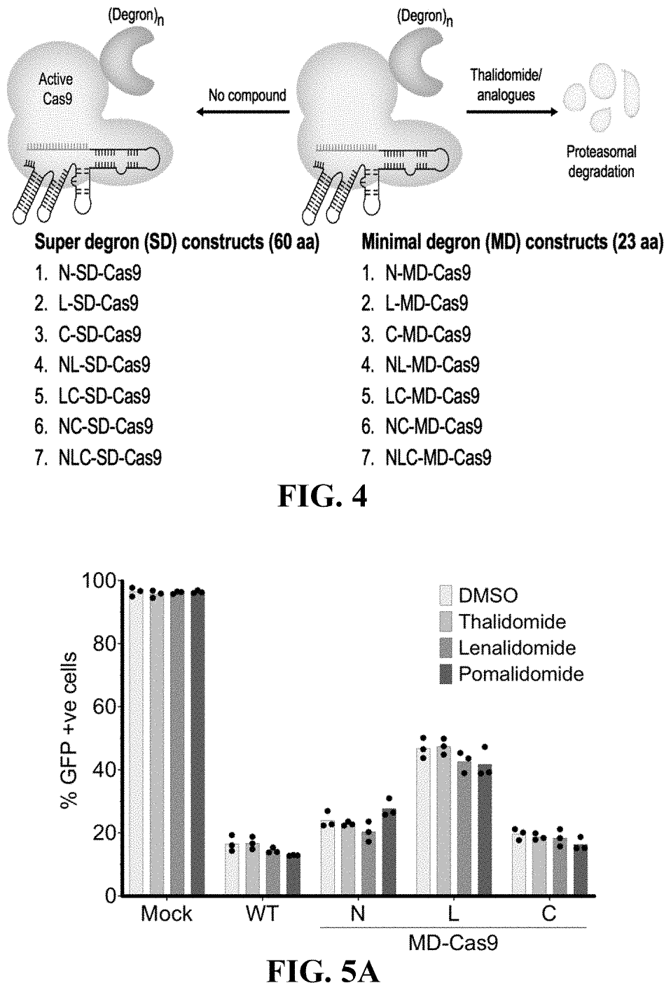

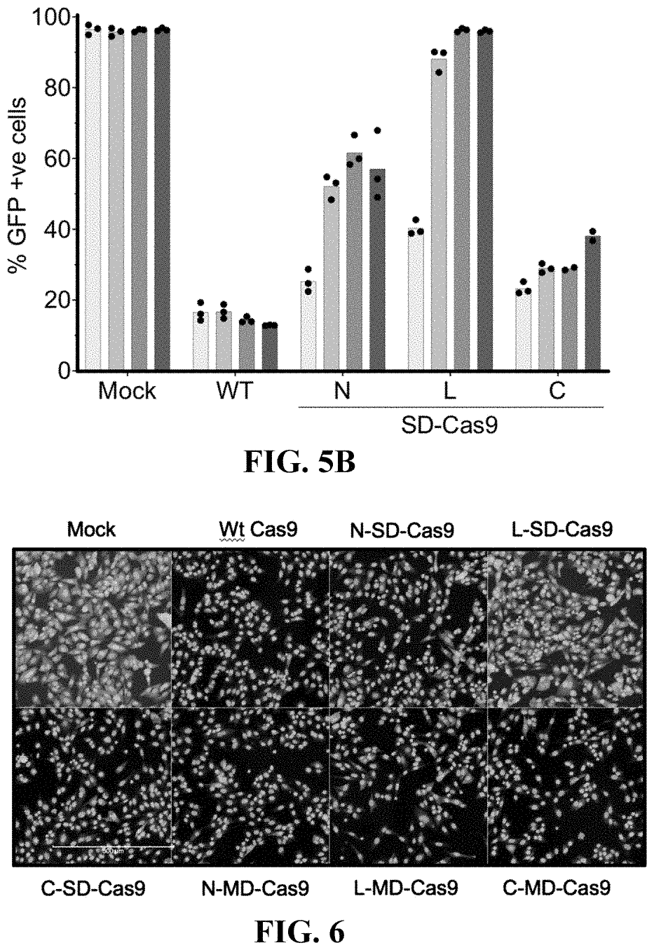

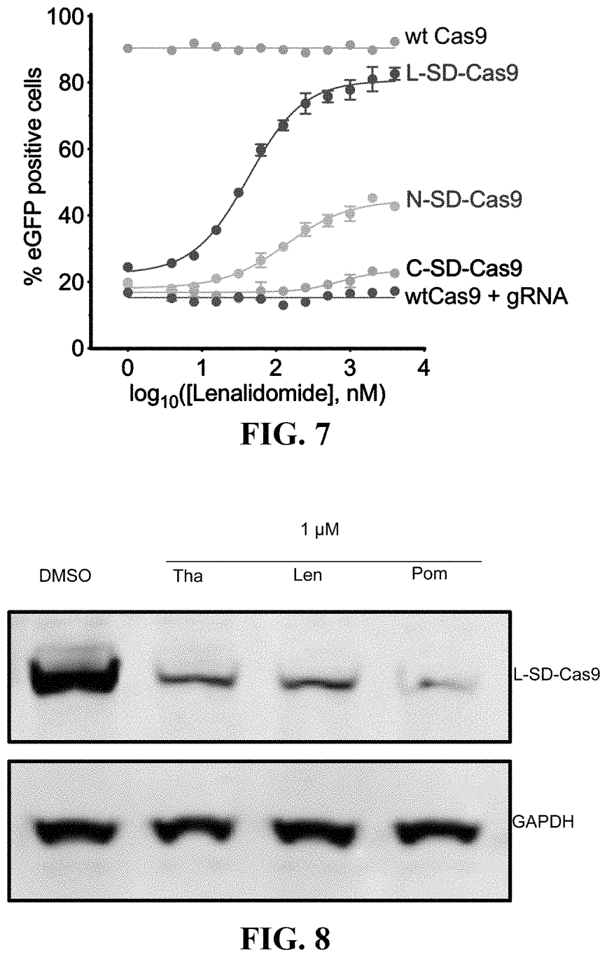

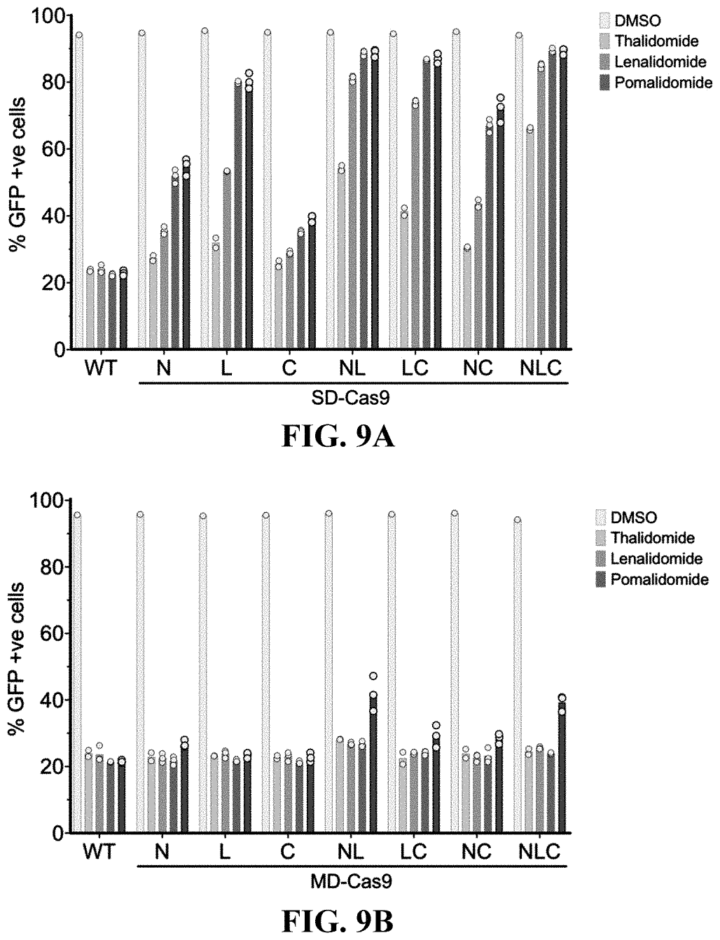

In addition, Applicants broke down the % of 1 bp insertions based on the 3 categories that the reduced 48 gRNA library contains: % of CRISPR genotypes for control gRNAs (32-47) remains the same overtime; % of CRISPR 1 bp insertions for gRNAs (0-15, insertion precision library) that favor 1 bp insertion significantly increases the longer Cas is present. (FIG. 2)

As for % MH and Non-MH mediated deletions for the grouped 3-category gRNA libraries: In both insertion and microhomology precision libraries, MH deletions events require a longer presence of Cas9. (FIG. 3)

### REFERENCES

1. Doudna, J. A. & Charpentier, E. Genome editing. The new frontier of genome engineering with CRISPR-Cas9. Science 346, 1258096 (2014).
2. Hsu, P. D., Lander, E. S. & Zhang, F. Development and applications of CRISPR-Cas9 for genome engineering. Cell 157, 1262-1278 (2014).
3. Gantz, V. M. & Bier, E. The dawn of active genetics. Bioessays 38, 50-63 (2016).
4. Chen, B. et al. Dynamic imaging of genomic loci in living human cells by an optimized CRISPR/Cas system. Cell 155, 1479-1491 (2013).
5. Hilton, I. B. et al. Epigenome editing by a CRISPR-Cas9-based acetyltransferase activates genes from promoters and enhancers. Nat. Biotechnol. 33, 510-517 (2015).
6. Dominguez, A. A., Lim, W. A. & Qi, L. S. Beyond editing: repurposing CRISPR-Cas9 for precision genome regulation and interrogation. Nat. Rev. Mol. Cell Biol. 17, 5-15 (2016).
7 Nunez, J. K., Harrington, L. B. & Doudna, J. A. Chemical and biophysical modulation of Cas9 for tunable genome engineering. ACS Chem. Biol. (2016).
8. Oakes, B. L. et al. Profiling of engineering hotspots identifies an allosteric CRISPR-Cas9 switch. Nat. Biotechnol. (2016), in press.
9. Erb et al. Transcription control by the ENL YEATS domain in acute leukemia. Nature. Mar. 9 2017. 543(7644): 270-274.
10. Huang et al. MELK is not necessary for the proliferation of basal-like breast cancer cells.
   eLife. September 2017. 6: e26693.)
11. Nabet et al. The dTAG system for immediate and target-specific protein degradation Nat Chem Biol 2018.

### Example 2—Zinc Finger Degrons

Degrons regulate protein turnover mediated by the ubiquitin-proteasome system. Guharoy, et al., Nature Communications, 5 Jan. 2016, 7:10239; doi:101038/ncomms10239. As described in Guharoy, zinc finger degrons are tripartite, comprised of a primary degron peptide motif that specifies substrate recognition by cognate E3 ubiquitin ligases, secondary sites comprising a single or multiple neighboring ubiquinated lysines and a structurally disordered segment that initiates substrate unfolding at the 26S proteasome. Thalidomide and/or its analogs lenalidomide and pomalidomide can mediate interactions between the CRL4^{CRBN} E3 ubiquitin ligase and substrate proteins such as zinc finger transcription factors, that are then degraded by the proteasome. See, e.g. Sievers, et al. Science 2018 Nov. 2: 362 (6414); doi:10.1126/science.aat0572.

### A Hybrid Zinc Finger Screen to Engineer Super Degrons

Chemical genetic control of protein stability is a cornerstone of modern molecular biology that enables rapid perturbation of biologic processes(6). Multiple orthogonal systems now exist to regulate protein degradation, including destabilization domains(7), auxin-induced degradation (8), LID (9), SMASh (10), and dTags (11). While these systems are invaluable tools and provocative models for future cell-based therapies, there is a clinical need for chemical genetic control systems that are engineered from non-immunogenic human polypeptide sequences, are controlled by clinically approved and non-immunosuppressive drugs, and afford robust ON-/OFF-switch control of protein stability. Therefore, from these first principles of clinical suitability, Applicants endeavored to create control systems gated by thalidomide derivatives for cell-based therapies.

Thalidomide, lenalidomide, and pomalidomide are effective and clinically approved therapies for multiple myeloma, subtypes of non-Hodgkin lymphoma, and myelodysplastic syndrome with chromosome 5q deletion. Thalidomide derivatives exert therapeutic properties by acting as molecular glue, bridging interactions between the CRL4^{CRBN} E3 ubiquitin ligase and disease-relevant proteins that are subsequently ubiquitinated and degraded by the proteasome (12-14). A set of Cys2-His2 (C2H2) zinc fingers have emerged as a recurrent degron motif mediating drug-dependent interactions with CRL4^{CRBN} (15-18). Applicants hypothesized that these small, modular, human polypeptide domains could be engineered and repurposed as tags to induce drug-dependent OFF-switch depletion of engineered proteins. Further, for ON-switch control, it was hypothesized that the CRBN-lenalidomide-zinc finger ternary interaction could be uncoupled from the ubiquitin-proteasome system in order to generate a stable lenalidomide-inducible dimerization system.

As proof of concept for cell-based therapies controlled by lenalidomide-gated switches, engineering systems into CARs was chosen for both clinical and biological reasons. First, while displaying remarkable efficacy culminating in clinical approvals for the treatment of B cell acute lymphoblastic leukemia and diffuse large B cell lymphoma(19), CARs pose a risk for toxic T cell hyperactivation(20). Whereas the current management of cytokine release and CAR-related encephalopathy syndromes consists of supportive care, tocilizumab, and/or high-dose corticosteroids(4), it was proposed that these hyperactivation syndromes would be more easily diagnosed and managed if clinicians could rapidly and reversibly control CAR degradation and signaling. Second, CAR regulation poses an especially difficult challenge for control by protein degradation. Because CARs transduce powerful, in some cases excessive T cell activation signals(21-23), near-complete CAR depletion would be required to prevent CAR T cell activation. A control system robust enough to completely degrade a highly expressed CAR could be a generalizable solution for the regulation of diverse cell-based therapies.

Herein is reported the engineering of two chemical genetic control systems gated by lenalidomide, with proof of concept application to CAR T cells. The use of these systems is then applied to CRISPR-Cas systems. Applicants report a systematic screen to identify “super-degrons” with enhanced sensitivity to lenalidomide-induced degradation. The degrons were used to develop lenalidomide-OFF-switch degradable CARs. After uncoupling the CRBN-lenalidomide-zinc finger interaction from the ubiquitin-proteasome system, a lenalidomide-inducible dimerization system was generated that enabled the design of lenalidomide-ON-switch split CARs. Together, these lenalidomide ON- and OFF-switches are rapid, reversible, and clinically suitable control systems that are well-positioned to improve the safety and efficacy of diverse gene- and cell-based therapies. Degron use is then shown in use for the control of CRISPR-Cas9 systems.

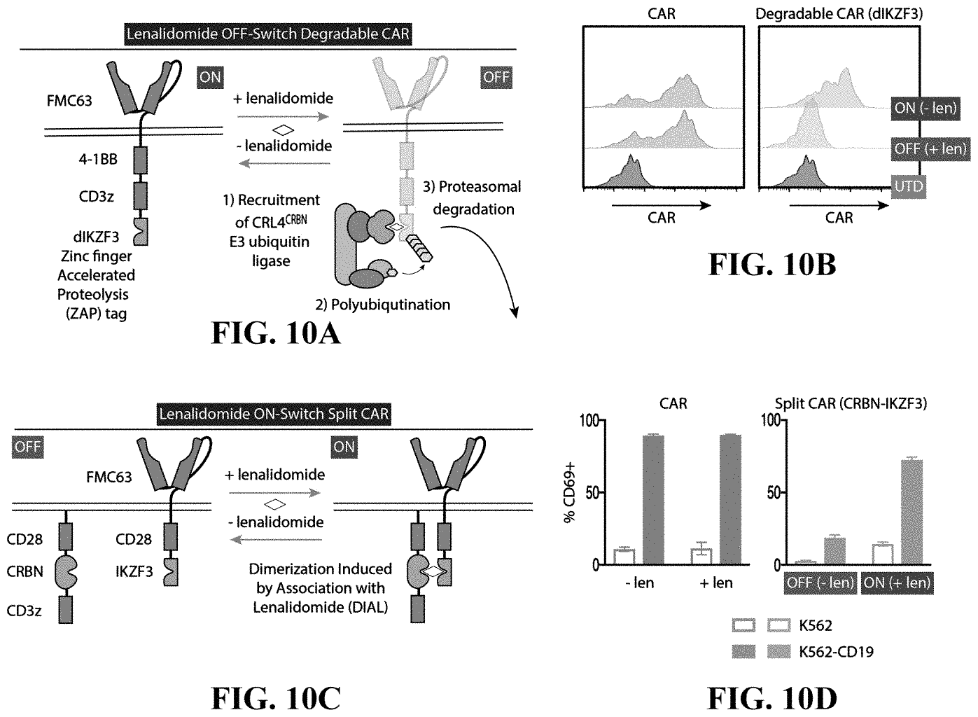

A lenalidomide-inducible proximity system was designed (FIG. 10A). Crystallographic analysis of CRL4^{CRBN} in complex with thalidomide derivatives indicate that the CRBN neosubstrate/drug binding domain is separate from the DDB1-binding domain that facilitates ubiquitin ligase recruitment (24-26). Applicants therefore hypothesized that CRBN could be derivatized to retain degron binding activity without ubiquitin ligase recruitment. Having generated a lenalidomide-inducible dimerization switch protected from degradation via endogenous CRL4^{CRBN}, these elements were incorporated into an ON-switch split CAR (27) (FIG. 10C). Lenalidomide licensed the split CAR for antigen-dependent activation (FIG. 10D). A hybrid zinc finger screen to engineer super degrons

While the IKZF3-based degradation and dimerization switches demonstrated efficacy at drug concentrations used therapeutically, engineering more robust synthetic components was desired. Inventors proposed that synthetic components could act at sub-therapeutic drug concentrations, with multiple zinc fingers found in humans individually capable of mediating drug-dependent degradation at different efficacies such that an engineered zinc finger might mediate drug dependent degradation more efficiently than any present in the human proteome (here termed “super degrons”). First, a library composed of all possible beta-hairpin and alpha-helix combinations from 22 C2H2 zinc fingers destabilized by various thalidomide derivatives was created (FIG. 11A) and encoded into a lentiviral degradation reporter vector (FIG. 11B). To screen for the synthetic zinc fingers that mediate drug-dependent degradation most efficiently, Jurkat T cells were transduced with the hybrid ZF library and then treated with vehicle control, lenalidomide, pomalidomide, avadomide, or iberdomide. Fluorescence-activated cell sorting (FACS) was used to isolate mCherry⁺eGFP^{low} cells (FIG. 11C), and the relative frequency of individual ZFs was quantified by next-generation sequencing. ZFs demonstrating drug-dependent degradation were significantly enriched in drug-treated versus control-treated mCherry⁺eGFP^{low} populations. Remarkably, with lenalidomide, the 21 most significantly depleted ZFs were hybrid forms, and 20 of these 21 candidate super degrons were composed from the matrix of 5 N-termini (ZN653, ZN827, ZFP91, ZN276, IKZF3) with 7 C-termini (ZN787, ZN517, IKZF3, ZN654, PATZ1, E4F1, and ZKSC5) (FIG. 11D). Similar findings were identified for pomalidomide, avadomide, and iberdomide (FIG. 17A-17C). The preferred N-terminal beta-hairpins converge on a similar sequence at residues with crystallographic evidence of side chain-drug interactions (15), but are otherwise molecularly diverse (FIG. 11E). These findings identify a group of ZF subdomains that can promiscuously combine to form lenalidomide-dependent hybrid super degrons more efficiently degraded than their parent ZFs.

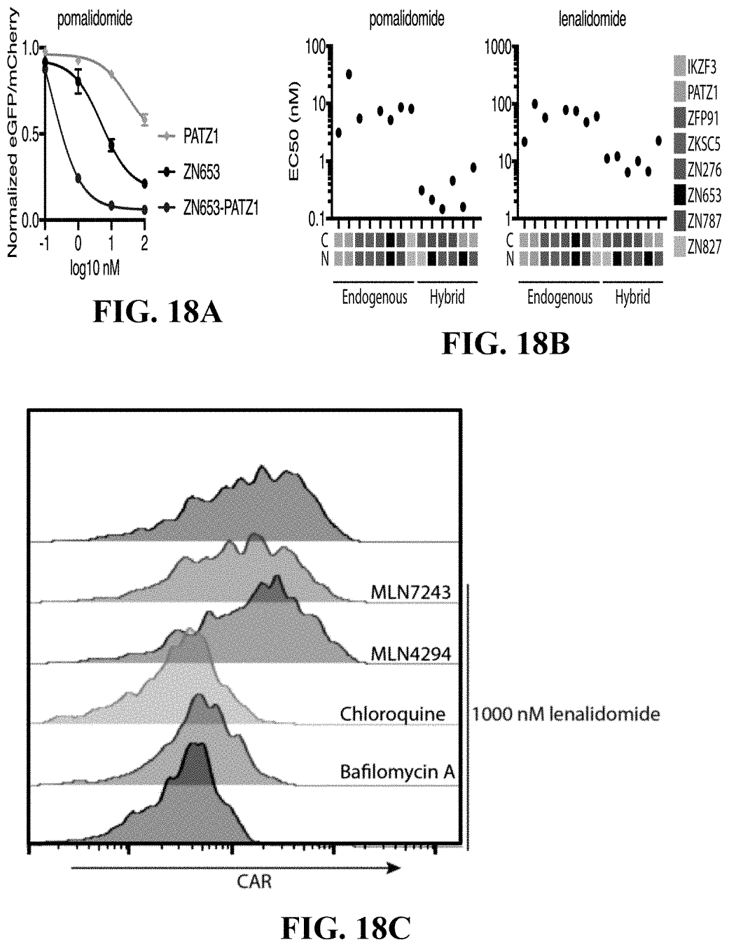

To characterize individual hybrid ZFs well-suited for synthetic biology applications such as inducible degradation tags, 6 hybrid ZFs were investigated that were more significantly degraded than all endogenous ZFs. Jurkat cells were created expressing each of the 6 hybrid and 8 associated parent ZFs and subjected them to a range of doses of lenalidomide, pomalidomide, avadomide, and iberdomide. The ZN653-PATZ1 hybrid, for example, demonstrates more efficient pomalidomide-dependent degradation than either parent ZF (FIG. 18A). The IC50 for degradation was lower for the 6 hybrid ZFs than their parent ZFs (FIG. 18B). As extended sections of the IKZF1 zinc finger array demonstrate higher affinity for CRBN-pomalidomide than the minimal 23 amino acid zinc finger degron(15), 60 amino acid extended hybrid degrons were tested to optimize the efficiency of the candidate super-degrons (FIG. 11F). One of these validated hybrids, ZFP91-IKZF3, was chosen with 1.6-6.0-times lower IC50 for degradation than IKZF3 across the tested thalidomide derivatives (FIG. 11G) hereafter termed “d91.3”, as a super degron tag for further CAR engineering, which was incorporated for evaluation of on and off-switch CARs.

### Lenalidomide-ON-Switch CAR Activation and Effector Functions

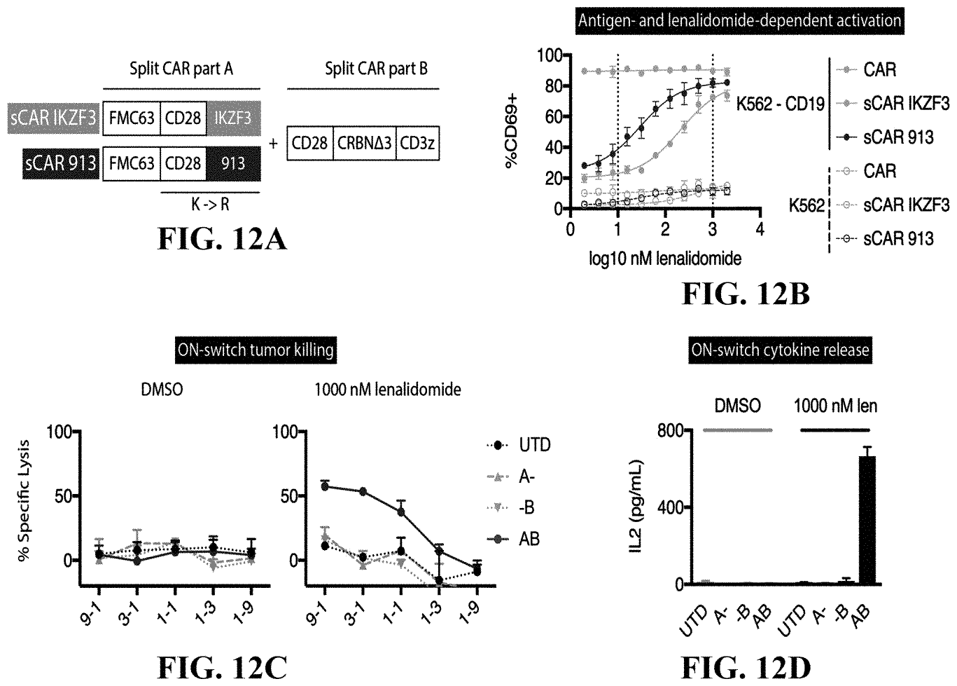

To test whether the increased sensitivity of engineered zinc finger-lenalidomide-CRBN interactions improved ON-switch CAR performance, split CARs were compared with dimerization domains engineered from IKZF3 or the hybrid d913 (sCAR IKZF3 or sCAR 913, respectively) (FIG. 12A). When Jurkat T cells expressing these split CARs were exposed to CD19+ target cells and a range of lenalidomide concentrations, the EC50 was 7-fold lower for sCAR 91.3 than for sCAR IKZF3 When Jurkat T cells expressing these split CARs were exposed to CD19+ target cells and a range of lenalidomide concentrations, the EC50 was 7-fold lower for sCAR 91.3 than for sCAR IKZF3 (FIG. 12B).

To evaluate whether effector functions of primary T cells could be gated by lenalidomide, primary sCAR 913 T cells were generated. As the two split CAR components are delivered by separate lentivectors, this gave the ability to use FACS to purify cells expressing neither, one, or both components. In a cytotoxicity assay, killing of NALM6 target cells was restricted to T cells expressing both halves of sCAR 91.3 in the presence of 1000 nM lenalidomide (FIG. 12C). Similarly, IL2 production in these co-culture experiments required the complete sCAR 91.3 and lenalidomide (FIG. 12D). In multiple myeloma patients, the maximum plasma concentration of lenalidomide with 25 mg per day dosing is 1.9 μM (29); therefore, sCAR 91.3 T cells demonstrated titratable T cell activation, tumor cell killing, and cytokine release at clinically relevant lenalidomide concentrations.

### A Super-Degron Improves Control of OFF-Switch Degradable CARs

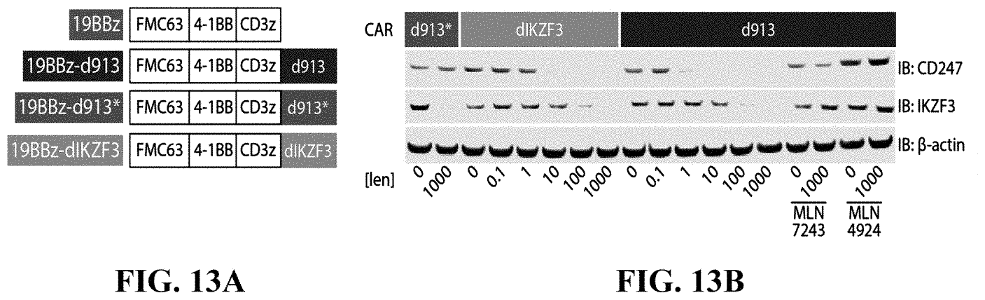

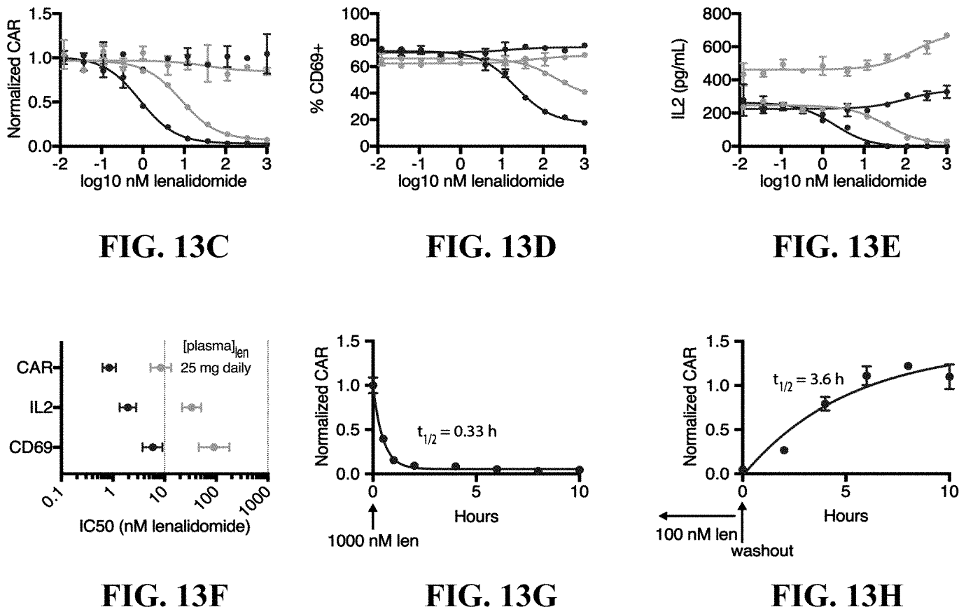

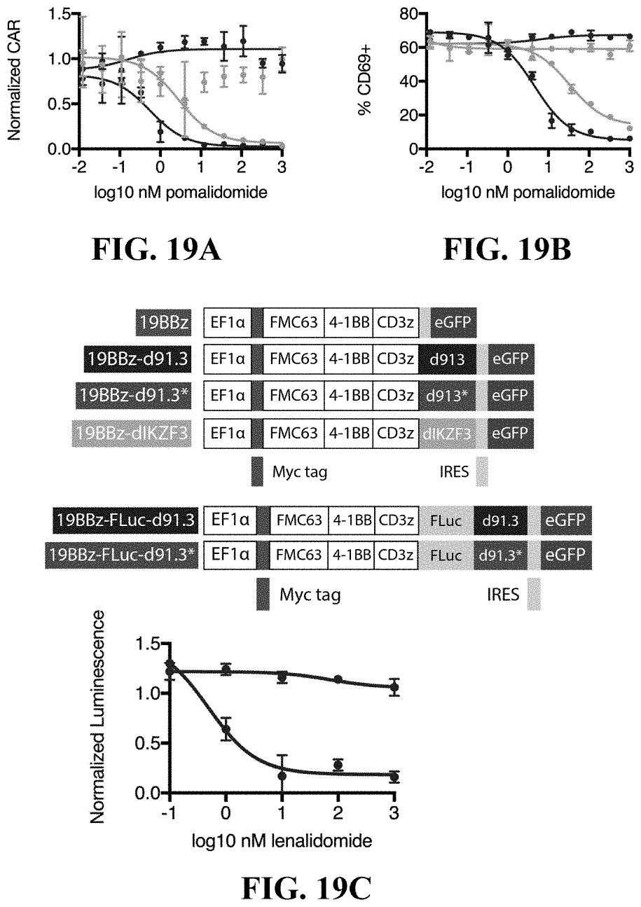

To test whether the super-degron tag also improved OFF-switch CAR control, we transduced Jurkat cells to express CARs containing no degron tag, dIKZF3, d91.3, or d91.3*, a drug-insensitive control with a cysteine to alanine substitution at the zinc-chelating position ZFP91 p.402 (FIG. 13A). Lenalidomide dose-dependent degradation of 19BBz-dIKZF3 and 19BBz-d913 were both confirmed by Western blotting and flow cytometry (FIG. 13C). The degron-tagged CARs, especially 19BBz-d913, were depleted at approximately 1/100th of the lenalidomide concentration required to deplete the canonical endogenous substrate IKZF3 (FIG. 13B—lanes 3-14). E1 and neddylation inhibitors blocked degradation (FIG. 13B—lanes 15-18), consistent with the established Cullin-RING ligase-dependent mechanism. Degron- and lenalidomide-dependent CAR depletion was also seen with pomalidomide treatment (FIG. 19).

### CAR Degradation is Rapid and Reversible

Next, the kinetics of CAR depletion was examined after the addition of lenalidomide. Half-maximal depletion of the degradable CAR, 19BBz-d91.3, occurred in ˜20 minutes (FIG. 13D). We also examined the dynamics of CAR re-synthesis after washout of lenalidomide. Half-maximal recovery of 19BBz-d91.3 expression occurred after ˜3.6 hours (FIG. 13E). In sum, we found the post-translational control of degradable CAR protein abundance to be rapid and reversible, consistent with the degradation kinetics of other thalidomide analog substrate proteins (30). These findings demonstrate reversible pharmacologic control of CAR expression.

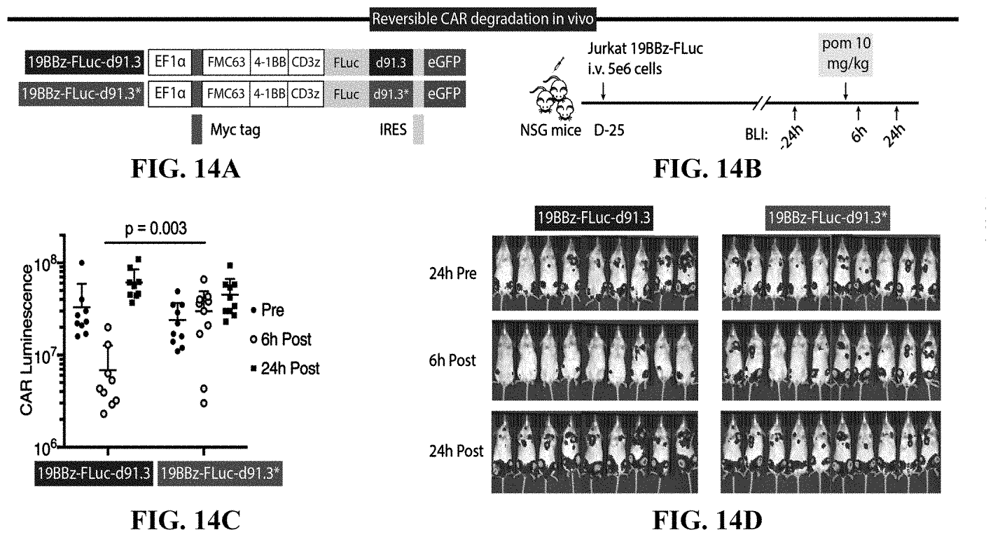

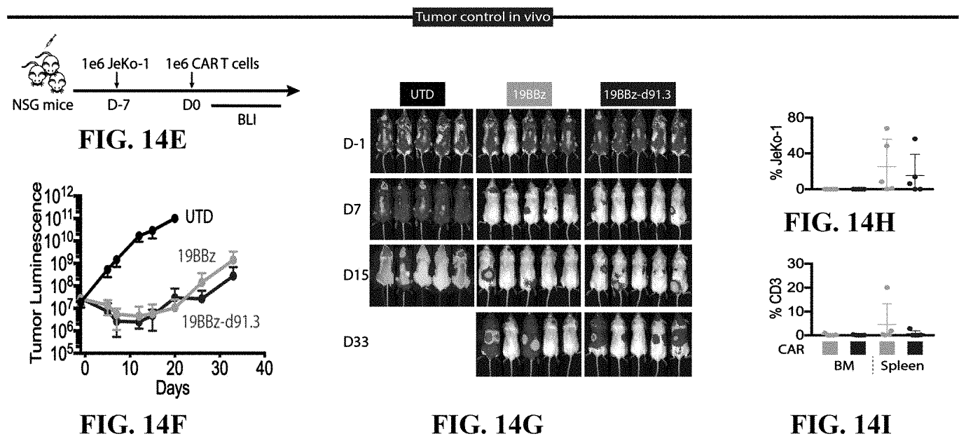

Thalidomide analogs control degradable CAR T cell activation and effector functions in vitro and in vivo. To test whether degradable CAR T cell activation could be controlled with lenalidomide, 19BBz, 19BBz-dIKZF3, 19BBz-d91.3, and 19BBz-d91.3* Jurkat CAR T cell lines were co-cultured with K562 cells engineered to express the target antigen CD19 (K562-CD19) and 11 lenalidomide concentrations or vehicle control. After overnight incubation, CD69 early activation marker expression was partially (19BBz-dIKZF3) or more completely (19BBz-d913) inhibited with higher concentrations of lenalidomide (FIG. 13F). To evaluate whether effector functions of degradable CAR T cells could be controlled with lenalidomide, primary human CAR T cells were generated and cytotoxicity assays performed comparing the conventional 19BBz CAR to the degradable 19BBz-d91.3 CAR in vitro. Whereas the specific lysis of NALM6 B-ALL target cells was similar for the two CARs without lenalidomide, target cell killing by 19BBz-d91.3 was not detected above background with 100 nM or 1000 nM lenalidomide (FIG. 14A). T cells were not pre-incubated with lenalidomide; instead, target cells and lenalidomide were pre-mixed and then added to T cells simultaneously. Complete inhibition of cytotoxicity indicates rapid kinetics of functional inhibition, consistent with the rapid kinetics of CAR depletion (FIG. 13D). Then cytokine production was analyzed in response to antigen stimulation. As expected, the 19BBz CAR demonstrated increased production of IL-2 when co-cultured with target cells in the presence of lenalidomide (FIG. 14B). Conversely, for the 19BBz-d91.3 CAR, 100 nM lenalidomide reduced the secretion of all evaluated cytokines reflective of T cell activation (FIG. 14C).

To evaluate whether degradable CAR T cell cytokine release could be controlled in vivo, a high-level tumor engraftment model was used to provoke CAR T cell cytokine release. NALM6 cells were engrafted in non-obese diabetic scid gamma (NSG) mice one week before injection of conventional 19BBz CAR T cells, degradable 19BBz-d91.3 CAR T cells, or untransduced control T cells. On days 3-5 after T cell transfer, mice were either left untreated, treated daily, or treated twice daily with pomalidomide, which was used for in vivo experiments because it has a longer in vivo half-life than lenalidomide. On the afternoon of day 5, serum plasma concentrations were measured for a panel of human T cell cytokines (FIG. 14C). IFN-gamma levels were reduced four-fold (p=0.04) with daily and six-fold (p=0.01) with twice-daily pomalidomide treatment. IL-2 levels were not significantly reduced with twice-daily treatment (p=0.06), but were significantly reduced by four-fold with daily treatment (p=0.05). Thus, pomalidomide can be used to limit cytokine release in vivo, the major driver of CAR T cell hyperactivation toxicities.

### Reversible CAR Degradation In Vivo

Having demonstrated functional inhibition of CAR T cells in vivo, we first created CAR-luciferase fusions tagged with either the d913 or the d913* control degron to monitor CAR protein abundance via bioluminescent imaging. As expected, after exposure to lenalidomide, we observed a dose-dependent decrease in luminescence from Jurkat cells expressing degradable but not control luciferase-tagged CARs (FIG. 14A). Applicants then transplanted NSG mice with the engineered T cells. After establishing detectable engraftment by luminescence imaging, we administered a single 10 mg/kg oral pomalidomide dose the following day, and measured luminescence. Six hours after drug treatment, luminescence from the degradable CAR was significantly reduced by 5-fold versus the pre-treatment timepoint (p=0.003) (FIG. 14B/14C). After 24 hours, luminescence had recovered to levels similar to that of the control CAR. Thus, the in vivo kinetics of degradation and re-expression of the degron-tagged CARs was consistent with our in vitro findings, and suggest that daily dosing of lenalidomide or pomalidomide would transiently abrogate CAR expression, with recovery of CAR expression upon drug discontinuation.

Addition of the Super-Degron Tag does not Alter CAR T Cell Anti-Tumor Efficacy In Vivo

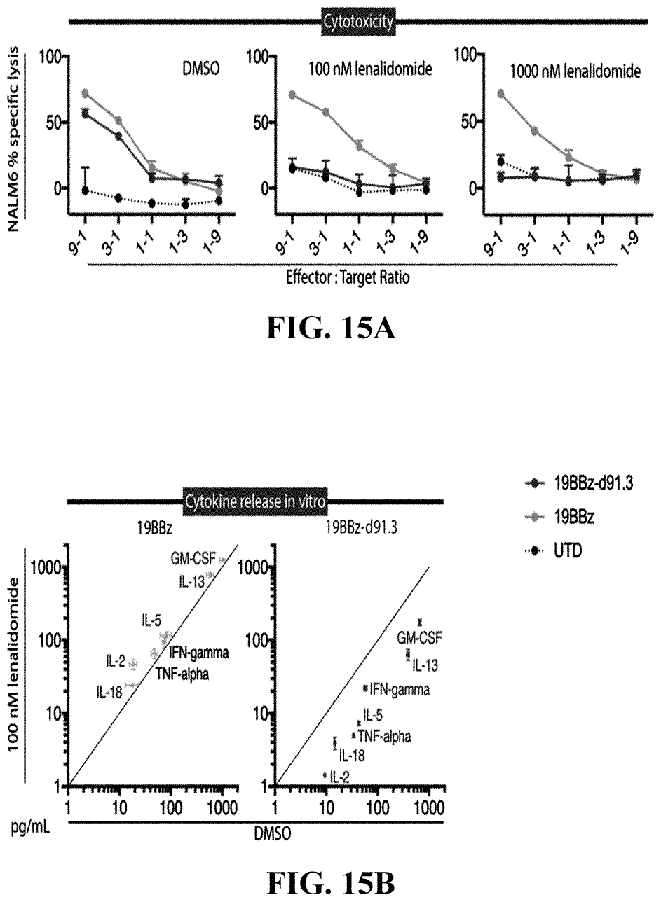

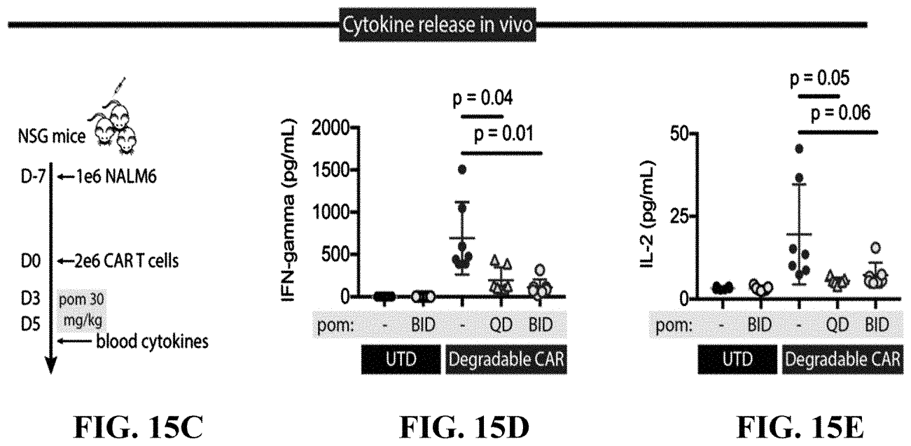

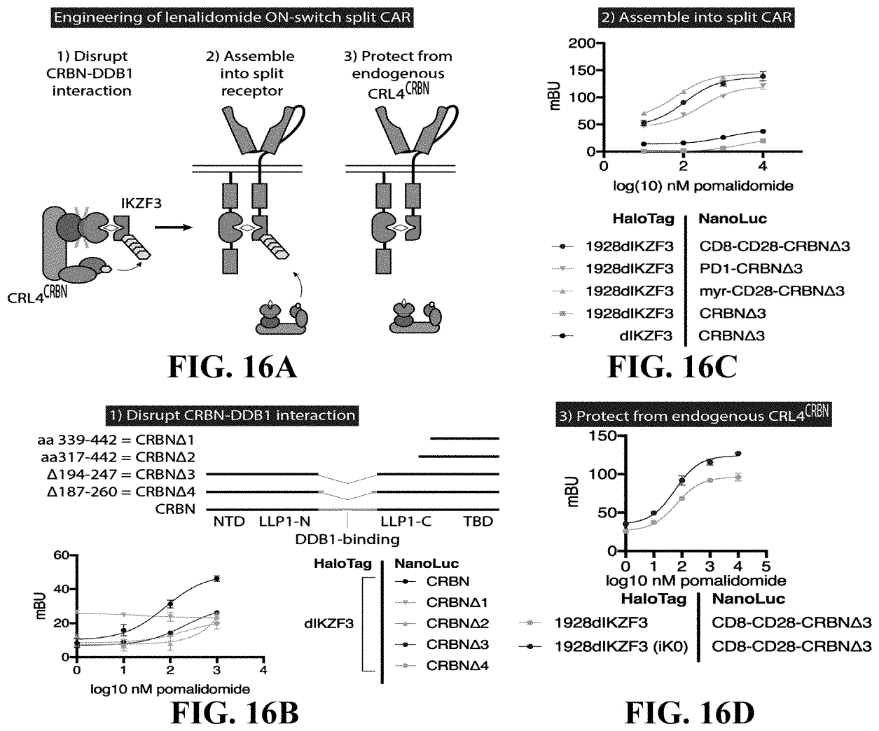

Subtle sequence changes to chimeric antigen receptors have been associated with intended and unintended consequences for CAR T cell efficacy and toxicity in clinical trials as well as pre-clinical models (23, 33, 34). Therefore, we determined whether addition of the zinc finger super-degron tag impacts CAR T cell activity in a mantle cell lymphoma xenograft model. We engrafted NSG mice with CD19+ luciferase+ JeKo-1 mantle cell lymphoma cells. One week later, we injected conventional 19BBz CART cells, degradable 19BBz-d91.3 CAR T cells, or untransduced control T cells; tumor burden was followed by BLI (FIG. 15E). Comparing the conventional and degradable CAR T cells, there were no significant differences in survival, total tumor burden assessed by BLI (FIG. 15F-15G), splenic or bone marrow tumor burden (FIG. 15H), or T cell persistence in the spleen or bone marrow (FIG. 15I). Thus, addition of the zinc finger super-degron tag did not significantly impact tumor control or CAR T cell persistence in a B cell lymphoma xenograft model.

Regulated transgene function can improve diverse gene- and cell-based therapies. User control can enable novel therapeutics conditionally deploying highly active therapeutic proteins that would be toxic if constitutively expressed (31). While many synthetic gene regulation tools have been developed (32), most use non-human components, small molecule controllers that have not been clinically validated, or immunosuppressive drugs. Simple, clinically suitable control systems are needed. Here we demonstrate chemical genetic control of CAR T cells using a 60 amino acid human protein-derived degron tag and a clinically approved, non-immunosuppressive small molecule controller. Chemical genetic ON- and OFF-switches were generated, gated by lenalidomide, a targeted protein degrader. The ternary interactions between ubiquitin ligases, small molecule degraders, and polypeptide degrons are a rich starting point to engineer novel synthetic control modules. Here it is demonstrated that 1) supraphysiologic lenalidomide-induced degrons can be engineered and 2) lenalidomide-induced dimerization events can be separated from degradation by the ubiquitin-proteasome system. As novel degraders are rapidly developed for clinical use, protein-protein interactions enforced by bifunctional molecules should be mined for new synthetic biology parts to control protein stability and dimerization. A systematic screen was developed to engineer “super-degrons” more efficiently degraded in the presence of low concentrations of lenalidomide. Whereas fundamental engineering of zinc fingers to recognize specific DNA sequences have largely focused on derivatizing known DNA-contacting residues (33), here we leveraged the modularity of beta-hairpin and alpha-helix subdomains to build a library of hybrid zinc fingers. Surprisingly, it was found that almost 5% of the hybrid zinc fingers were more efficiently degraded than all parent zinc finger degrons (FIG. 11). These findings, together with the synthetic origin of thalidomide, suggest that there has not been an evolutionary drive to optimize the ternary CRBN-drug-zinc finger degron interactions. Larger scale, molecularly diverse engineering and/or evolution approaches may uncover the sequence and structural determinants for enhanced CRBN-drug interactions, as well as even higher affinity, bio-orthogonal super-degrons that can be depleted at lenalidomide doses that spare endogenous substrates. Already, the degradable CAR 19BBz-d913 was depleted at approximately 100-fold lower lenalidomide concentrations than endogenous IKZF3 (FIG. 13B).

As proof of concept, we tested the chemical genetic switches in CARs to address 1) clinical need and 2) the challenge of regulating sensitive and highly active receptors that require near-complete control for robust switch-regulatable function. Lenalidomide-gated CARs demonstrated control of T cell activation, tumor killing, and cytokine release at or below therapeutic drug doses. In vivo, a single dose of pomalidomide induced robust degradable CAR depletion, with recovery by 24 hours. The particular robustness of the degradable CAR may be due to “event-driven” pharmacologic effects of targeted protein degraders, wherein a single molecule can induce the degradation of many target proteins via serial docking interactions with CRL4^{CRBN} and substrate proteins (34).

### Materials and Methods

### C2H2 Zinc Finger Hybrid Degron Library Screen

Jurkat cells expressing a library of 440 C2H2 zinc fingers in a eGFP/mCherry protein degradation reporter vector were treated with DMSO or thalidomide analog drug for 16 hours. mCherry⁺eGFP^{low} cell populations were isolated by FACS in triplicate, and the relative frequency of individual ZFs was quantified with next-generation sequencing. For validation, Jurkat cells were engineered to express individual zinc fingers in the protein degradation reporter; the eGFP:mCherry ratio was determined by flow cytometry after 16 hour incubation with varying concentrations of thalidomide analogs.

### Construction of Chimeric Antigen Receptors

Transgenes were synthesized and cloned into lentiviral vectors. Split CAR component A was constructed using the CSF2RA signal sequence, myc tag, anti-CD19 scFv (FMC63), CD28 hinge, transmembrane, and co-stimulatory domains, and zinc finger dimerization domain. Split CAR component B was constructed using the CD8 alpha signal sequence, hinge, and transmembrane domains, CD28 costimulatory domain, CRBNΔ3, and CD3z intracellular domain. In experiments comparing a split CAR to a conventional CAR, the conventional CAR is 1928z. The degradable CAR encodes the CD8 alpha signal sequence, myc tag, anti-CD19 scFv (FMC63), IgG4 hinge, CD28 transmembrane domain, 4-1BB costimulatory domain, and CD3z domain, followed by a degron. In experiments comparing a degradable CAR to a conventional CAR, the conventional CAR is 19BBz.

### Jurkat CAR Protein Degradation and Functional Assays

Jurkat cells transduced with lentiviral vectors encoding CARs were co-cultured for 16 hours with either K562 target cells or K562 cells engineered to express CD19 in a 5:1 ratio. Jurkat CAR-T cells were then assessed by flow cytometry for CAR (anti-Myc tag; Cell Signaling Technology, 2233) and CD69 expression (Biolegend, 310920). Normalized CAR expression was calculated via subtraction of the MFI of unstained cells and normalization to the signal intensity of vehicle control-treated cells. IL2 concentration in the co-culture supernatant was assessed by IL2 ELISA (BD Biosciences, 555190). Luciferase-tagged CAR luminescence was measured with an EnVision plate reader (PerkinElmer).

### T Cell Culture Transduction

Human T cells were purified (Stem Cell Technologies, 15061) from anonymous human healthy donor leukopacs purchased from the Massachusetts General Hospital blood bank under an Institutional Review Board-exempt protocol. Primary T cell stimulation, transduction, and expansion was performed as previously described (30089630).

### Cellular Cytotoxicity and Cytokine Assays

Primary human CAR-T effector cells were co-cultured with NALM6 target cells engineered to express click beetle green luciferase at the indicated ratios for 16 hours. Luciferase activity was measured with a Synergy Neo2 luminescence microplate reader (Biotek). Cell culture supernatant from these experiments was analyzed for soluble cytokines (Luminex).

### In Vivo Studies

All animal procedures were performed in accordance with Federal and Institutional Animal Care and Use Committee requirements under protocols approved at the Broad Institute. Bioluminescence imaging was performed using an IVIS Spectrum in vivo imaging system.

### Example 3—Use in Cas Polypeptide Systems for Temporal Control

Exemplary Zinc Finger Degrons and Cas9 proteins are provided herein.

TABLE 2
Sequences of Super degron and Minimal Degrons

| | | |
|---|---|---|
| Nucleo- | GGC TCA GGT AGC GGA AGC GGA TCA GGT | Linker |
| tide | GGA TTC AAT GTA CTG ATG GTC CAT AAA | sequence |
| Se- | CGG AGT CAC ACT GGC GAG CGC CCG CTC | itali- |
| quence | CAA TGT GAA ATC TGC GGG TTC ACG TGT | cized |
| of | CGG CAG AAG GGC AAC CTC CTC CGG CAT | |
| Super | ATC AAG CTG CAC ACG GGT GAA AAA CCG | |
| Degron | TTT AAG TGC CAT CTC TGC AAT TAC GCC | |
| | TGT CAG AGA AGA GAT GCT TTG GGT GGA | |
| | TCT GGA TCT GGC AGC GGG TCT GGC | |
| | (SEQ ID NO: 41) | |
| Amino | GSGSGSGSGG | Linker |
| Acid | FNVLMVHKRSHTGERPLQCEICGFTCRQKGNLL | sequence |
| Se- | RHIKLHTGEKPFKCHLCNYACQRRDAL | itali- |
| qunece | GGSGSGSGSG (SEQ ID NO: 42) | cized |
| of | | |
| Super | | |
| Degron | | |
| Nucleo- | GGC TCT GGG AGT GGG TCC GGC TCT GGA | Linker |
| tide | GGT CTC CAG TGC GAG ATC TGT GGC TTC | sequence |
| Se- | ACC TGT AGA CAG AAA GGT AAC TTG CTT | itali- |
| quence | CGA CAT ATC AAA CTC CAT GGG GGG TCA | cized |
| of | GGG TCT GGT AGT GGA AGC GGC | |
| Minimal | (SEQ ID NO: 43) | |
| Degron | | |
| Amino | GSGSGSGSGG LQCEICGFTCRQKGNLLRHIKLH | Linker |
| Acid | GGSGSGSGSG (SEQ ID NO: 45) | sequence |
| Se- | | itali- |
| quence | | cized |
| of | | |
| Minimal | | |
| Degron | | |
| Se- | gactataaggaccacgacggagactacaaggatcatgatattgattacaaagacg | Bold = |
| quence | atgacgataagatggccccaaagaagaagcggaaggtcggtatccacggagtcc | Super |
| of L- | cagcagccgacaagaagtacagcatcggcctggacatcggcaccaactctgtgg | degron |
| SD- | gctgggccgtgatcaccgacgagtacaaggtgcccagcaagaaattcaaggtgct | Itali- |
| Cas9 | gggcaacaccgaccggcacagcatcaagaagaacctgatcggagccctgctgtt | cize = |
| | cgacagcggcgaaacagccgaggccacccggctgaagagaaccgccagaaga | linker |
| | agatacaccagacggaagaaccggatctgctatctgcaagagatcttcagcaacg | |
| | agatggccaaggtggacgacagcttcttccacagactggaagagtccttcctggtg | |
| | gaagaggataagaagcacgagcggcaccccatcttcggcaacatcgtggacga | |
| | ggtggcctaccacgagaagtaccccaccatctaccacctgagaaagaaactggtg | |
| | gacagcaccgacaaggccgacctgcggctgatctatctggccctggcccacatg | |
| | atcaagttccggggccacttcctgatcgagggcgacctgaaccccgacaacagc | |
| | gacgtggacaagctgttcatccagctggtgcagacctacaaccagctgttcgagg | |
| | aaaaccccatcaacgccagcggcgtggacgccaaggccatcctgtctgccagac | |
| | tgagcaagagcagacggctggaaaatctgatcgcccagctgcccggcggctcag | |
| | gtagcggaagcggatcaggtggattcaatgtactgatggtccataaacggagt | |
| | cacactggcgagcgcccgctccaatgtgaaatctgcgggttcacgtgtcggca | |
| | gaagggcaacctcctccggcatatcaagctgcacacgggtgaaaaaccgttt | |
| | aagtgccatctctgcaattacgcctgtcagagaagagatgctttgggtggatct | |
| | ggatctggcagcgggtctggcgagaagaagaatggcctgttcggaaacctgattg | |
| | ccctgagcctgggcctgacccccaacttcaagagcaacttcgacctggccgagga | |
| | tgccaaactgcagctgagcaaggacacctacgacgacgacctggacaacctgct | |
| | ggcccagatcggcgaccagtacgccgacctgtttctggccgccaagaacctgtcc | |
| | gacgccatcctgctgagcgacatcctgagagtgaacaccgagatcaccaaggcc | |
| | cccctgagcgcctctatgatcaagagatacgacgagcaccaccaggacctgacc | |
| | ctgctgaaagctctcgtgcggcagcagctgcctgagaagtacaaagagattttcttc | |
| | gaccagagcaagaacggctacgccggctacattgacggcggagccagccagga | |
| | agagttctacaagttcatcaagcccatcctggaaaagatggacggcaccgaggaa | |
| | ctgctcgtgaagctgaacagagaggacctgctgcggaagcagcggaccttcgac | |
| | aacggcagcatcccccaccagatccacctgggagagctgcacgccattctgcgg | |
| | cggcaggaagatttttacccattcctgaaggacaaccgggaaaagatcgagaaga | |
| | tcctgaccttccgcatcccctactacgtgggccctctggccaggggaaacagcag | |
| | attcgcctggatgaccagaaagagcgaggaaaccatcaccccctggaacttcgag | |
| | gaagtggtggacaagggcgcttccgcccagagcttcatcgagcggatgaccaac | |
| | ttcgataagaacctgcccaacgagaaggtgctgcccaagcacagcctgctgtacg | |
| | agtacttcaccgtgtataacgagctgaccaaagtgaaatacgtgaccgagggaatg | |
| | agaaagcccgccttcctgagcggcgagcagaaaaaggccatcgtggacctgctg | |
| | ttcaagaccaaccggaaagtgaccgtgaagcagctgaaagaggactacttcaaga | |
| | aaatcgagtgcttcgactccgtggaaatctccggcgtggaagatcggttcaacgcc | |
| | tccctgggcacataccacgatctgctgaaaattatcaaggacaaggacttcctgga | |
| | caatgaggaaaacgaggacattctggaagatatcgtgctgaccctgacactgtttg | |
| | aggacagagagatgatcgaggaacggctgaaaacctatgcccacctgttcgacg | |
| | acaaagtgatgaagcagctgaagcggcggagatacaccggctggggcaggctg | |
| | agccggaagctgatcaacggcatccgggacaagcagtccggcaagacaatcctg | |
| | gatttcctgaagtccgacggcttcgccaacagaaacttcatgcagctgatccacga | |
| | cgacagcctgacctttaaagaggacatccagaaagcccaggtgtccggccaggg | |
| | cgatagcctgcacgagcacattgccaatctggccggcagccccgccattaagaag | |
| | ggcatcctgcagacagtgaaggtggtggacgagctcgtgaaagtgatgggccgg | |
| | cacaagcccgagaacatcgtgatcgaaatggccagagagaaccagaccaccca | |
| | gaagggacagaagaacagccgcgagagaatgaagcggatcgaagagggcatc | |
| | aaagagctgggcagccagatcctgaaagaacaccccgtggaaaacacccagct | |
| | gcagaacgagaagctgtacctgtactacctgcagaatgggcgggatatgtacgtg | |
| | gaccaggaactggacatcaaccggctgtccgactacgatgtggaccatatcgtgc | |
| | ctcagagctttctgaaggacgactccatcgacaacaaggtgctgaccagaagcga | |
| | caagaaccggggcaagagcgacaacgtgccctccgaagaggtcgtgaagaaga | |
| | tgaagaactactggcggcagctgctgaacgccaagctgattacccagagaaagtt | |
| | cgacaatctgaccaaggccgagagaggcggcctgagcgaactggataaggccg | |
| | gcttcatcaagagacagctggtggaaacccggcagatcacaaagcacgtggcac | |
| | agatcctggactcccggatgaacactaagtacgacgagaatgacaagctgatccg | |
| | ggaagtgaaagtgatcaccctgaagtccaagctggtgtccgatttccggaaggatt | |
| | tccagttttacaaagtgcgcgagatcaacaactaccaccacgcccacgacgcctac | |
| | ctgaacgccgtcgtgggaaccgccctgatcaaaaagtaccctaagctggaaagc | |
| | gagttcgtgtacggcgactacaaggtgtacgacgtgcggaagatgatcgccaaga | |
| | gcgagcaggaaatcggcaaggctaccgccaagtacttcttctacagcaacatcat | |
| | gaactttttcaagaccgagattaccctggccaacggcgagatccggaagcggcct | |
| | ctgatcgagacaaacggcgaaaccggggagatcgtgtgggataagggccggga | |
| | ttttgccaccgtgcggaaagtgctgagcatgccccaagtgaatatcgtgaaaaaga | |
| | ccgaggtgcagacaggcggcttcagcaaagagtctatcctgcccaagaggaaca | |
| | gcgataagctgatcgccagaaagaaggactgggaccctaagaagtacggcggct | |
| | tcgacagccccaccgtggcctattctgtgctggtggtggccaaagtggaaaaggg | |
| | caagtccaagaaactgaagagtgtgaaagagctgctggggatcaccatcatggaa | |
| | agaagcagcttcgagaagaatcccatcgactttctggaagccaagggctacaaag | |
| | aagtgaaaaaggacctgatcatcaagctgcctaagtactccctgttcgagctggaa | |
| | aacggccggaagagaatgctggcctctgccggcgaactgcagaagggaaacga | |
| | actggccctgccctccaaatatgtgaacttcctgtacctggccagccactatgagaa | |
| | gctgaagggctcccccgaggataatgagcagaaacagctgtttgtggaacagcac | |
| | aagcactacctggacgagatcatcgagcagatcagcgagttctccaagagagtga | |
| | tcctggccgacgctaatctggacaaagtgctgtccgcctacaacaagcaccggga | |
| | taagcccatcagagagcaggccgagaatatcatccacctgtttaccctgaccaatct | |
| | gggagcccctgccgccttcaagtactttgacaccaccatcgaccggaagaggtac | |
| | accagcaccaaagaggtgctggacgccaccctgatccaccagagcatcaccggc | |
| | ctgtacgagacacggatcgacctgtctcagctgggaggcgacaaaaggccggcg | |
| | gccacgaaaaaggccggccaggcaaaaaagaaaaagtaa | |
| | (SEQ ID NO: 45) | |

TABLE 3A
Zinc Finger GFPlo Enrichment TD vs. DMSO

| ZnF | N | C | Naa | Caa | NaaCaa |
|---|---|---|---|---|---|
| IKZF3_146_168- | IKZF | E4F1 | FQCNQCGA | TKGSLIRHHR | FQCNQCGASFTTKGSLIR |
| E4F1_220_242_J1 | 3 | | SFT (SEQ ID | RH (SEQ ID | HHRRH (SEQ ID NO: 48) |
| | | | NO: 46) | NO: 47) | |
| ZN628_120_142- | ZN6 | E4F1 | FICGQCGL | TKGSLIRHHR | FICGQCGLAFKTKGSLIR |
| E4F1_220_242_J1 | 28 | | AFK (SEQ | RH (SEQ ID | HHRRH (SEQ ID NO: 50) |
| | | | ID NO: 49) | NO: 47) | |
| PATZ1_383_405- | PAT | E4F1 | YSCPVCGL | TKGSLIRHHR | YSCPVCGLRFKTKGSLIR |
| E4F1_220_242_J1 | Z1 | | RFK (SEQ | RH (SEQ ID | HHRRH (SEQ ID NO: 52) |
| | | | ID NO: 51) | NO: 47) | |
| ZN398_483_505- | ZN3 | E4F1 | FSCPQCGID | TKGSLIRHHR | FSCPQCGIDFNTKGSLIR |
| E4F1_220_242_J1 | 98 | | FN (SEQ ID | RH (SEQ ID | HHRRH (SEQ ID NO: 54) |
| | | | NO: 53) | NO: 47) | |
| ZN654_25_47- | ZN6 | E4F1 | FACVICGR | TKGSLIRHHR | FACVICGRKFRTKGSLIR |
| E4F1_220_242_J1 | 54 | | KFR (SEQ | RH (SEQ ID | HHRRH (SEQ ID NO: 56) |
| | | | ID NO: 55) | NO: 47) | |
| ZN827_374_396- | ZN8 | E4F1 | FQCPICGLV | TKGSLIRHHR | FQCPICGLVIKTKGSLIRH |
| E4F1_220_242_J1 | 27 | | IK (SEQ ID | RH (SEQ ID | HRRH (SEQ ID NO: 58) |
| | | | NO: 57) | NO: 47) | |
| ZN597_341_363- | ZN5 | E4F1 | LQCPDCDM | TKGSLIRHHR | LQCPDCDMTFPTKGSLIR |
| E4F1_220_242_J1 | 97 | | TFP (SEQ ID | RH (SEQ ID | HHRRH (SEQ ID NO: 60) |
| | | | NO: 59) | NO: 47) | |
| ZNF90_481_503- | ZNF | E4F1 | YKCQECDK | TKGSLIRHHR | YKCQECDKAFKTKGSLI |
| E4F1_220_242_J1 | 90 | | AFK (SEQ | RH (SEQ ID | RHHRRH (SEQ ID NO: 62) |
| | | | ID NO: 61) | NO: 47) | |
| ZSC20_766_788- | ZSC | E4F1 | YKCLECGK | TKGSLIRHHR | YKCLECGKSFSTKGSLIR |
| E4F1_220_242_J1 | 20 | | SFS (SEQ ID | RH (SEQ ID | HHRRH (SEQ ID NO: 64) |
| | | | NO: 63) | NO: 47) | |
| ZN653_556_578- | ZN6 | E4F1 | LQCEICGY | TKGSLIRHHR | LQCEICGYQCRTKGSLIR |
| E4F1_220_242_J1 | 53 | | QCR (SEQ | RH (SEQ ID | HHRRH (SEQ ID NO: 66) |
| | | | ID NO: 65) | NO: 47) | |
| ZFP91_400_422ZN692 | ZFP9 | E4F1 | LQCEICGFT | TKGSLIRHHR | LQCEICGFTCRTKGSLIR |
| 417_439- | 1 | | CR (SEQ ID | RH (SEQ ID | HHRRH (SEQ ID NO: 68) |
| E4F1_220_242_J1 | | | NO: 67) | NO: 47) | |
| IKZF2_140_162- | IKZF | E4F1 | FHCNQCGA | TKGSLIRHHR | FHCNQCGASFTTKGSLIR |
| E4F1_220_242_J1 | 2 | | SFT (SEQ ID | RH (SEQ ID | HHRRH (SEQ ID NO: 70) |
| | | | NO: 69) | NO: 47) | |
| ZN276_524_546- | ZN2 | E4F1 | LQCEVCGF | TKGSLIRHHR | LQCEVCGFQCRTKGSLIR |
| E4F1_220_242_J1 | 76 | | QCR (SEQ | RH (SEQ ID | HHRRH (SEQ ID NO: 72) |
| | | | ID NO: 71) | NO: 47) | |
| ZKSC5_430_452- | ZKS | E4F1 | YGCNECGK | TKGSLIRHHR | YGCNECGKNFGTKGSLI |
| E4F1_220_242_J1 | C5 | | NFG (SEQ | RH (SEQ ID | RHHRRH (SEQ ID NO: 74) |
| | | | ID NO: 73) | NO: 47) | |
| ZNF74_444_466- | ZNF | E4F1 | FKCADCGK | TKGSLIRHHR | FKCADCGKGFSTKGSLIR |
| E4F1_220_242_J1 | 74 | | GFS (SEQ ID | RH (SEQ ID | HHRRH (SEQ ID NO; 76) |
| | | | NO: 75) | NO: 47) | |
| ZN582_395_417- | ZN5 | E4F1 | YQCKVCGR | TKGSLIRHHR | YQCKVCGRAFKTKGSLI |
| E4F1_220_242_J1 | 82 | | AFK (SEQ | RH (SEQ ID | RHHRRH (SEQ ID NO: 78) |
| | | | ID NO: 77) | NO: 47) | |
| ZN787_178_200- | ZN7 | E4F1 | FVCPRCGR | TKGSLIRHHR | FVCPRCGRGFSTKGSLIR |
| E4F1_220_242_J1 | 87 | | GFS (SEQ ID | RH (SEQ ID | HHRRH (SEQ ID NO: 80) |
| | | | NO: 79) | NO: 47) | |
| E4F1_220_242- | E4F1 | E4F1 | HECKLCGA | TKGSLIRHHR | HECKLCGASFRTKGSLIR |
| E4F1_220_242_J1 | | | SFR (SEQ ID | RH (SEQ ID | HHRRH (SEQ ID NO: 82) |
| | | | NO: 81) | NO: 47) | |
| ZN517_452_474- | ZN5 | E4F1 | YRCRACGR | TKGSLIRHHR | YRCRACGRACSTKGSLIR |
| E4F1_220_242_J1 | 17 | | ACS (SEQ | RH (SEQ ID | HHRRH (SEQ ID NO: 84) |
| | | | ID NO: 83) | NO: 47) | |
| ZN595_145_167- | ZN5 | E4F1 | FQCNTCVK | TKGSLIRHHR | FQCNTCVK VFSTKGSLIR |
| E4F1_220_242_J1 | 95 | | VFS (SEQ ID | RH (SEQ ID | HHRRH (SEQ ID NO: 86) |
| | | | NO: 85) | NO: 47) | |
| ZF69B_419_441- | ZF69 | E4F1 | YICNVCSK | TKGSLIRHHR | YICNVCSKTFSTKGSLIR |
| E4F1_220_242_J1 | B | | TFS (SEQ ID | RH (SEQ ID | HHRRH (SEQ ID NO: 88) |
| | | | NO: 87) | NO: 47) | |
| ZNF74_444_466- | ZNF | IKZF | FKCADCGK | QKGNLLRHI | FKCADCGKGFSQKGNLL |
| IKZF3_146_168IKZF2_ | 74 | 3 | GFS (SEQ ID | KLH (SEQ ID | RHIKLH (SEQ ID NO: 90) |
| 140_162_J1 | | | NO: 75) | NO: 89) | |
| E4F1_220_242- | E4F1 | IKZF | HECKLCGA | QKGNLLRHI | HECKLCGASFRQKGNLL |
| IKZF3_146_168IKZF2_ | | 3 | SFR (SEQ ID | KLH (SEQ ID | RHIKLH (SEQ ID NO: 91) |
| 140_162_J1 | | | NO: 81) | NO: 89) | |
| ZN582_395_417- | ZN5 | IKZF | YQCKVCGR | QKGNLLRHI | YQCKVCGRAFKQKGNL |
| IKZF3_146_168IKZF2_ | 82 | 3 | AFK (SEQ | KLH (SEQ ID | LRHIKLH (SEQ ID NO: |
| 140_162_J1 | | | ID NO: 77) | NO: 89) | 92) |
| ZNF90_481_503- | ZNF | IKZF | YKCQECDK | QKGNLLRHI | YKCQECDKAFKQKGNLL |
| IKZF3_146_168IKZF2_ | 90 | 3 | AFK (SEQ | KLH (SEQ ID | RHIKLH (SEQ ID NO: 93) |
| 140_162_J1 | | | ID NO: 61) | NO: 89) | |
| ZN653_556_578- | ZN6 | IKZF | LQCEICGY | QKGNLLRHI | LQCEICGYQCRQKGNLL |
| IKZF3_146_168IKZF2_ | 53 | 3 | QCR (SEQ | KLH (SEQ ID | RHIKLH (SEQ ID NO: 94) |
| 140_162_J1 | | | ID NO: 65) | NO: 89) | |
| ZN595_145_167- | ZN5 | IKZF | FQCNTCVK | QKGNLLRHI | FQCNTCVKVFSQKGNLL |
| IKZF3_146_168IKZF2_ | 95 | 3 | VFS (SEQ ID | KLH (SEQ ID | RHIKLH (SEQ ID NO: 95) |
| 140_162_J1 | | | NO: 85) | NO: 89) | |
| ZF69B_419_441- | ZF69 | IKZF | YICNVCSK | QKGNLLRHI | YICNVCSKTFSQKGNLLR |
| IKZF3_146_168IKZF2_ | B | 3 | TFS (SEQ ID | KLH (SEQ ID | HIKLH (SEQ ID NO: 96) |
| 140_162_J1 | | | NO: 87) | NO: 89) | |
| ZN597_341_363- | ZN5 | IKZF | LQCPDCDM | QKGNLLRHI | LQCPDCDMTFPQKGNLL |
| IKZF3_146_168IKZF2_ | 97 | 3 | TFP (SEQ ID | KLH (SEQ ID | RHIKLH (SEQ ID NO: 97) |
| 140_162_J1 | | | NO: 59) | NO: 89) | |
| IKZF2_140_162- | IKZF | IKZF | FHCNQCGA | QKGNLLRHI | FHCNQCGASFTQKGNLL |
| IKZF3_146_168IKZF2_ | 2 | 3 | SFT (SEQ ID | KLH (SEQ ID | RHIKLH (SEQ ID NO: 98) |
| 140_162_J1 | | | NO: 69) | NO: 89) | |
| ZFP91_400_422ZN692 | ZFP9 | IKZF | LQCEICGFT | QKGNLLRHI | LQCEICGFTCRQKGNLLR |
| 417_43_9- | 1 | 3 | CR (SEQ ID | KLH (SEQ ID | HIKLH (SEQ ID NO: 99) |
| IKZF3_146_168IKZF2_ | | | NO: 67) | NO: 89) | |
| 140_162_J1 | | | | | |
| ZN628_120_142- | ZN6 | IKZF | FICGQCGL | QKGNLLRHI | FICGQCGLAFKQKGNLL |
| IKZF3_146_168IKZF2_ | 28 | 3 | AFK (SEQ | KLH (SEQ ID | RHIKLH (SEQ ID NO: 100) |
| 140_162_J1 | | | ID NO: 49) | NO: 89) | |
| ZN276_524_546- | ZN2 | IKZF | LQCEVCGF | QKGNLLRHI | LQCEVCGFQCRQKGNLL |
| IKZF3_146_168IKZF2_ | 76 | 3 | QCR (SEQ | KLH (SEQ ID | RHIKLH (SEQ ID NO: 101) |
| 140_162_J1 | | | ID NO: 71) | NO: 89) | |
| IKZF3_146_168- | IKZF | IKZF | FQCNQCGA | QKGNLLRHI | FQCNQCGASFTQKGNLL |
| IKZF3_146_168IKZF2_ | 3 | 3 | SFT (SEQ ID | KLH (SEQ ID | RHIKLH (SEQ ID NO: 102) |
| 140_162_J1 | | | NO: 46 | NO: 89) | |
| ZN398_483_505- | ZN3 | IKZF | FSCPQCGID | QKGNLLRHI | FSCPQCGIDFNQKGNLLR |
| IKZF3_146_168IKZF2_ | 98 | 3 | FN (SEQ ID | KLH (SEQ ID | HIKLH (SEQ ID NO: 103) |
| 140_162_J1 | | | NO: 53) | NO: 89) | |
| ZN654_25_47- | ZN6 | IKZF | FACVICGR | QKGNLLRHI | FACVICGRKFRQKGNLL |
| IKZF3_146_168IKZF2_ | 54 | 3 | KFR (SEQ | KLH (SEQ ID | RHIKLH (SEQ ID NO: 104) |
| 140_162_J1 | | | ID NO: 55) | NO: 89) | |
| ZSC20_766_788- | ZSC | IKZF | YKCLECGK | QKGNLLRHI | YKCLECGKSFSQKGNLL |
| IKZF3_146_168IKZF2_ | 20 | 3 | SFS (SEQ ID | KLH (SEQ ID | RHIKLH (SEQ ID NO: 105) |
| 140_162_J1 | | | NO: 63) | NO: 89) | |
| ZN827_374_396- | ZN8 | IKZF | FQCPICGLV | QKGNLLRHI | FQCPICGLVIKQKGNLLR |
| IKZF3_146_168IKZF2_ | 27 | 3 | IK (SEQ ID | KLH (SEQ ID | HIKLH (SEQ ID NO: 106) |
| 140_162_J1 | | | NO: 57) | NO: 89) | |
| ZKSC5_430_452- | ZKS | IKZF | YGCNECGK | QKGNLLRHI | YGCNECGKNFGQKGNLL |
| IKZF3_146_168IKZF2_ | C5 | 3 | NFG (SEQ | KLH (SEQ ID | RHIKLH (SEQ ID NO: 107) |
| 140_162_J1 | | | ID NO: 73) | NO: 89) | |
| PATZ1_383_405- | PAT | IKZF | YSCPVCGL | QKGNLLRHI | YSCPVCGLRFKQKGNLL |
| IKZF3_146_168IKZF2_ | Z1 | 3 | RFK (SEQ | KLH (SEQ ID | RHIKLH (SEQ ID NO: 108) |
| 140_162_J1 | | | ID NO: 51) | NO: 89) | |
| ZN787_178_200- | ZN7 | IKZF | FVCPRCGR | QKGNLLRHI | FVCPRCGRGFSQKGNLL |
| IKZF3_146_168IKZF2_ | 87 | 3 | GFS (SEQ ID | KLH (SEQ ID | RHIKLH (SEQ ID NO: 109) |
| 140_162_J1 | | | NO: 79) | NO: 89) | |
| ZN517_452_474- | ZN5 | IKZF | YRCRACGR | QKGNLLRHI | YRCRACGRACSQKGNLL |
| IKZF3_146_168IKZF2_ | 17 | 3 | ACS (SEQ | KLH (SEQ ID | RHIKLH (SEQ ID NO: 110) |
| 140_162_J1 | | | ID NO: 83) | NO: 89) | |
| ZKSC5_430_452- | ZKS | PAT | YGCNECGK | RKDRMSYHV | YGCNECGKNFGRKDRM |
| PATZ1_383_405_J1 | C5 | Z1 | NFG (SEQ | RSH (SEQ ID | SYHVRSH (SEQ ID NO: |
| | | | ID NO: 73) | NO: 111) | 112) |
| ZN582_395_417- | ZN5 | PAT | YQCKVCGR | RKDRMSYHV | YQCKVCGRAFKRKDRM |
| PATZ1_383_405_J1 | 82 | Z1 | AFK (SEQ | RSH (SEQ ID | SYHVRSH (SEQ ID NO: |
| | | | ID NO: 77) | NO: 111) | 113) |
| ZFP91_400_422ZN692 | ZFP9 | PAT | LQCEICGFT | RKDRMSYHV | LQCEICGFTCRRKDRMS |
| 417_43_9- | 1 | Z1 | CR (SEQ ID | RSH (SEQ ID | YHVRSH (SEQ ID NO: |
| PATZ1_383_405_J1 | | | NO: 67) | NO: 111) | 114) |
| ZN787_178_200- | ZN7 | PAT | FVCPRCGR | RKDRMSYHV | FVCPRCGRGFSRKDRMS |
| PATZ1_383_405_J1 | 87 | Z1 | GFS (SEQ ID | RSH (SEQ ID | YHVRSH (SEQ ID NO: |
| | | | NO: 79) | NO: 111) | 115) |
| ZF69B_419_441- | ZF69 | PAT | YICNVCSK | RKDRMSYHV | YICNVCSKTFSRKDRMS |
| PATZ1_383_405_J1 | B | Z1 | TFS (SEQ ID | RSH (SEQ ID | YHVRSH (SEQ ID NO: |
| | | | NO: 87) | NO: 111) | 116) |
| ZN398_483_505- | ZN3 | PAT | FSCPQCGID | RKDRMSYHV | FSCPQCGIDFNRKDRMS |
| PATZ1_383_405_J1 | 98 | Z1 | FN (SEQ ID | RSH (SEQ ID | YHVRSH (SEQ ID NO: |
| | | | NO: 53) | NO: 111) | 117) |
| ZN517_452_474- | ZN5 | PAT | YRCRACGR | RKDRMSYHV | YRCRACGRACSRKDRMS |
| PATZ1_383_405_J1 | 17 | Z1 | ACS (SEQ | RSH (SEQ ID | YHVRSH (SEQ ID NO: |
| | | | ID NO: 83) | NO: 111) | 118) |
| PATZ1_383_405- | PAT | PAT | YSCPVCGL | RKDRMSYHV | YSCPVCGLRFKRKDRMS |
| PATZ1_383_405_J1 | Z1 | Z1 | RFK (SEQ | RSH (SEQ ID | YHVRSH (SEQ ID NO: |
| | | | ID NO: 51) | NO: 111) | 119) |
| ZN276_524_546- | ZN2 | PAT | LQCEVCGF | RKDRMSYHV | LQCEVCGFQCRRKDRMS |
| PATZ1_383_405_J1 | 76 | Z1 | QCR (SEQ | RSH (SEQ ID | YHVRSH (SEQ ID NO: |
| | | | ID NO: 71) | NO: 111) | 120) |
| ZSC20_766_788- | ZSC | PAT | YKCLECGK | RKDRMSYHV | YKCLECGKSFSRKDRMS |
| PATZ1_383_405_J1 | 20 | Z1 | SFS (SEQ ID | RSH (SEQ ID | YHVRSH (SEQ ID NO: |
| | | | NO: 63) | NO: 111) | 121) |
| ZNF74_444_466- | ZNF | PAT | FKCADCGK | RKDRMSYHV | FKCADCGKGFSRKDRMS |
| PATZ1_383_405_J1 | 74 | Z1 | GFS (SEQ ID | RSH (SEQ ID | YHVRSH (SEQ ID NO: |
| | | | NO: 75) | NO: 111) | 122) |
| IKZF2_140_162- | IKZF | PAT | FHCNQCGA | RKDRMSYHV | FHCNQCGASFTRKDRMS |
| PATZ1_383_405_J1 | 2 | Z1 | SFT (SEQ ID | RSH (SEQ ID | YHVRSH (SEQ ID NO: |
| | | | NO: 69) | NO: 111) | 123) |
| E4F1_220_242- | E4F1 | PAT | HECKLCGA | RKDRMSYHV | HECKLCGASFRRKDRMS |
| PATZ1_383_405_J1 | | Z1 | SFR (SEQ ID | RSH (SEQ ID | YHVRSH (SEQ ID NO: |
| | | | NO: 81) | NO: 111) | 124) |
| ZN628_120_142- | ZN6 | PAT | FICGQCGL | RKDRMSYHV | FICGQCGL AFKRKDRMS |
| PATZ1_383_405_J1 | 28 | Z1 | AFK (SEQ | RSH (SEQ ID | YHVRSH (SEQ ID NO: |
| | | | ID NO: 49) | NO: 111) | 125) |
| IKZF3146168- | IKZF | PAT | FQCNQCGA | RKDRMSYHV | FQCNQCGASFTRKDRMS |
| PATZ1_383_405_J1 | 3 | Z1 | SFT (SEQ ID | RSH (SEQ ID | YHVRSH (SEQ ID NO: |
| | | | NO: 46) | NO: 111) | 126) |
| ZN827_374_396- | ZN8 | PAT | FQCPICGLV | RKDRMSYHV | FQCPICGLVIKRKDRMSY |
| PATZ1_383_405_J1 | 27 | Z1 | IK (SEQ ID | RSH (SEQ ID | HVRSH |
| | | | NO: 57) | NO: 111) | (SEQ ID NO: 127) |
| ZN654_25_47- | ZN6 | PAT | FACVICGR | RKDRMSYHV | FACVICGRKFRRKDRMS |
| PATZ1_383_405_J1 | 54 | Z1 | KFR (SEQ | RSH (SEQ ID | YHVRSH (SEQ ID NO: |
| | | | ID NO: 55) | NO: 111) | 128) |
| ZN597_341_363- | ZN5 | PAT | LQCPDCDM | RKDRMSYHV | LQCPDCDMTFPRKDRMS |
| PATZ1_383_405_J1 | 97 | Z1 | TFP (SEQ ID | RSH (SEQ ID | YHVRSH (SEQ ID NO: |
| | | | NO: 59) | NO: 111) | 129) |
| ZN653_556_578- | ZN6 | PAT | LQCEICGY | RKDRMSYHV | LQCEICGYQCRRKDRMS |
| PATZ1_383_405_J1 | 53 | Z1 | QCR (SEQ | RSH (SEQ ID | YHVRSH (SEQ ID NO: |
| | | | ID NO: 65) | NO: 111) | 130) |
| ZNF90_481_503- | ZNF | PAT | YKCQECDK | RKDRMSYHV | YKCQECDKAFKRKDRM |
| PATZ1_383_405_J1 | 90 | Z1 | AFK (SEQ | RSH (SEQ ID | SYHVRSH (SEQ ID NO: |
| | | | ID NO: 61) | NO: 111) | 131) |
| ZN595_145_167- | ZN5 | PAT | FQCNTCVK | RKDRMSYHV | FQCNTCVK VFSRKDRMS |
| PATZ1_383_405_J1 | 95 | Z1 | VFS (SEQ ID | RSH (SEQ ID | YHVRSH (SEQ ID NO: |
| | | | NO: 85) | NO: 111) | 132) |
| ZN628_120_142- | ZN6 | ZF69 | FICGQCGL | HSTYLTQHQ | FICGQCGLAFKHSTYLTQ |
| ZF69B_419_441_J1 | 28 | B | AFK (SEQ | RTH (SEQ ID | HQRTH (SEQ ID NO: 134) |
| | | | ID NO: 49) | NO: 133) | |
| E4F1_220_242- | E4F1 | ZF69 | HECKLCGA | HSTYLTQHQ | HECKLCGASFRHSTYLT |
| ZF69B_419_441_J1 | | B | SFR (SEQ ID | RTH (SEQ ID | QHQRTH (SEQ ID NO: |
| | | | NO: 81) | NO: 133) | 135) |
| ZN787_178_200- | ZN7 | ZF69 | FVCPRCGR | HSTYLTQHQ | FVCPRCGRGFSHSTYLTQ |
| ZF69B_419_441_J1 | 87 | B | GFS (SEQ ID | RTH (SEQ ID | HQRTH (SEQ ID NO: 136) |
| | | | NO: 79) | NO: 133) | |
| ZN582_395_417- | ZN5 | ZF69 | YQCKVCGR | HSTYLTQHQ | YQCKVCGRAFKHSTYLT |
| ZF69B_419_441_J1 | 82 | B | AFK (SEQ | RTH (SEQ ID | QHQRTH (SEQ ID NO: |
| | | | ID NO: 77) | NO: 133) | 137) |
| ZNF90_481_503- | ZNF | ZF69 | YKCQECDK | HSTYLTQHQ | YKCQECDKAFKHSTYLT |
| ZF69B_419_441_J1 | 90 | B | AFK (SEQ | RTH (SEQ ID | QHQRTH (SEQ ID NO: |
| | | | ID NO: 61) | NO: 133) | 138) |
| IKZF3_146_168- | IKZF | ZF69 | FQCNQCGA | HSTYLTQHQ | FQCNQCGASFTHSTYLT |
| ZF69B_419_441_J1 | 3 | B | SFT (SEQ ID | RTH (SEQ ID | QHQRTH (SEQ ID NO: |
| | | | NO: 46) | NO: 133) | 139) |
| ZN276_524_546- | ZN2 | ZF69 | LQCEVCGF | HSTYLTQHQ | LQCEVCGFQCRHSTYLT |
| ZF69B_419_441_J1 | 76 | B | QCR (SEQ | RTH (SEQ ID | QHQRTH (SEQ ID NO: |
| | | | ID NO: 71) | NO: 133) | 140) |
| ZN595_145_167- | ZN5 | ZF69 | FQCNTCVK | HSTYLTQHQ | FQCNTCVK VFSHSTYLT |
| ZF69B_419_441_J1 | 95 | B | VFS (SEQ ID | RTH (SEQ ID | QHQRTH (SEQ ID NO: |
| | | | NO: 85) | NO: 133) | 141) |
| ZN398_483_505- | ZN3 | ZF69 | FSCPQCGID | HSTYLTQHQ | FSCPQCGIDFNHSTYLTQ |
| ZF69B_419_441_J1 | 98 | B | FN (SEQ ID | RTH (SEQ ID | HQRTH (SEQ ID NO: 142) |
| | | | NO: 53) | NO: 133) | |
| ZFP91_400_422ZN692 | ZFP9 | ZF69 | LQCEICGFT | HSTYLTQHQ | LQCEICGFTCRHSTYLTQ |
| 417_43_9- | 1 | B | CR (SEQ ID | RTH (SEQ ID | HQRTH (SEQ ID NO: 143) |
| ZF69B_419_441_J1 | | | NO: 67) | NO: 133) | |
| ZN654_25_47- | ZN6 | ZF69 | FACVICGR | HSTYLTQHQ | FACVICGRKFRHSTYLTQ |
| ZF69B_419_441_J1 | 54 | B | KFR (SEQ | RTH (SEQ ID | HQRTH (SEQ ID NO: 144) |
| | | | ID NO: 55) | NO: 133) | |
| IKZF2140162- | IKZF | ZF69 | FHCNQCGA | HSTYLTQHQ | FHCNQCGASFTHSTYLT |
| ZF69B_419_441_J1 | 2 | B | SFT (SEQ ID | RTH (SEQ ID | QHQRTH (SEQ ID NO: |
| | | | NO: 69) | NO: 133) | 145) |
| PATZ1_383_405- | PAT | ZF69 | YSCPVCGL | HSTYLTQHQ | YSCPVCGLRFKHSTYLT |
| ZF69B_419_441_J1 | Z1 | B | RFK (SEQ | RTH (SEQ ID | QHQRTH (SEQ ID NO: |
| | | | ID NO: 51) | NO: 133) | 146) |
| ZF69B_419_441- | ZF69 | ZF69 | YICNVCSK | HSTYLTQHQ | YICNVCSKTFSHSTYLTQ |
| ZF69B_419_441_J1 | B | B | TFS (SEQ ID | RTH (SEQ ID | HQRTH (SEQ ID NO: 147) |
| | | | NO: 87) | NO: 133) | |
| ZN653_556_578- | ZN6 | ZF69 | LQCEICGY | HSTYLTQHQ | LQCEICGYQCRHSTYLTQ |
| ZF69B_419_441_J1 | 53 | B | QCR (SEQ | RTH (SEQ ID | HQRTH (SEQ ID NO: 148) |
| | | | ID NO: 65) | NO: 133) | |
| ZKSC5_430_452- | ZKS | ZF69 | YGCNECGK | HSTYLTQHQ | YGCNECGKNFGHSTYLT |
| ZF69B_419_441_J1 | C5 | B | NFG (SEQ | RTH (SEQ ID | QHQRTH (SEQ ID NO: |
| | | | ID NO: 73) | NO: 133) | 149) |
| ZN597_341_363- | ZN5 | ZF69 | LQCPDCDM | HSTYLTQHQ | LQCPDCDMTFPHSTYLT |
| ZF69B_419_441_J1 | 97 | B | TFP (SEQ ID | RTH (SEQ ID | QHQRTH (SEQ ID NO: |
| | | | NO: 59) | NO: 133) | 150) |
| ZSC20_766_788- | ZSC | ZF69 | YKCLECGK | HSTYLTQHQ | YKCLECGKSFSHSTYLTQ |
| ZF69B_419_441_J1 | 20 | B | SFS (SEQ ID | RTH (SEQ ID | HQRTH (SEQ ID NO: 151) |
| | | | NO: 63) | NO: 133) | |
| ZNF74_444_466- | ZNF | ZF69 | FKCADCGK | HSTYLTQHQ | FKCADCGKGFSHSTYLT |
| ZF69B_419_441_J1 | 74 | B | GFS (SEQ ID | RTH (SEQ ID | QHQRTH (SEQ ID NO: |
| | | | NO: 75) | NO: 133) | 152) |
| ZN517_452_474- | ZN5 | ZF69 | YRCRACGR | HSTYLTQHQ | YRCRACGRACSHSTYLT |
| ZF69B_419_441_J1 | 17 | B | ACS (SEQ | RTH (SEQ ID | QHQRTH (SEQ ID NO: |
| | | | ID NO: 83) | NO: 133) | 153) |
| ZN827_374_396- | ZN8 | ZF69 | FQCPICGLV | HSTYLTQHQ | FQCPICGLVIKHSTYLTQ |
| ZF69B_419_441_J1 | 27 | B | IK (SEQ ID | RTH (SEQ ID | HQRTH (SEQ ID NO: 154) |
| | | | NO: 57) | NO: 133) | |
| ZN582_395_417- | ZN5 | ZFP9 | YQCKVCGR | QKASLNWH | YQCKVCGRAFKQKASLN |
| ZFP91_400_422_J1 | 82 | 1 | AFK (SEQ | MKKH (SEQ | WHMKKH (SEQ ID NO: |
| | | | ID NO: 77) | ID NO: 155) | 156) |
| ZN398_483_505- | ZN3 | ZFP9 | FSCPQCGID | QKASLNWH | FSCPQCGIDFNQKASLN |
| ZFP91_400_422_J1 | 98 | 1 | FN (SEQ ID | MKKH (SEQ | WHMKKH (SEQ ID NO: |
| | | | NO: 53) | ID NO: 155) | 157) |
| ZF69B_419_441- | ZF69 | ZFP9 | YICNVCSK | QKASLNWH | YICNVCSKTFSQKASLN |
| ZFP91_400_422_J1 | B | 1 | TFS (SEQ ID | MKKH (SEQ | WHMKKH (SEQ ID NO: |
| | | | NO: 87) | ID NO: 155) | 158) |
| ZN827_374_396- | ZN8 | ZFP9 | FQCPICGLV | QKASLNWH | FQCPICGLVIKQKASLNW |
| ZFP91_400_422_J1 | 27 | 1 | IK (SEQ ID | MKKH (SEQ | HMKKH (SEQ ID NO: 159) |
| | | | NO: 57) | ID NO: 155) | |
| PATZ1_383_405- | PAT | ZFP9 | YSCPVCGL | QKASLNWH | YSCPVCGLRFKQKASLN |
| ZFP91_400_422_J1 | Z1 | 1 | RFK (SEQ | MKKH (SEQ | WHMKKH (SEQ ID NO: |
| | | | ID NO: 51) | ID NO: 155) | 160) |
| ZN653_556_578- | ZN6 | ZFP9 | LQCEICGY | QKASLNWH | LQCEICGYQCRQKASLN |
| ZFP91_400_422_J1 | 53 | 1 | QCR (SEQ | MKKH (SEQ | WHMKKH (SEQ ID NO: |
| | | | ID NO: 65) | ID NO: 155) | 161) |
| ZN276_524_546- | ZN2 | ZFP9 | LQCEVCGF | QKASLNWH | LQCEVCGFQCRQKASLN |
| ZFP91_400_422_J1 | 76 | 1 | QCR (SEQ | MKKH (SEQ | WHMKKH (SEQ ID NO: |
| | | | ID NO: 71) | ID NO: 155) | 162) |
| ZN787_178_200- | ZN7 | ZFP9 | FVCPRCGR | QKASLNWH | FVCPRCGRGFSQKASLN |
| ZFP91_400_422_J1 | 87 | 1 | GFS (SEQ ID | MKKH (SEQ | WHMKKH (SEQ ID NO: |
| | | | NO: 79) | ID NO: 155) | 163) |
| ZN654_25_47- | ZN6 | ZFP9 | FACVICGR | QKASLNWH | FACVICGRKFRQKASLN |
| ZFP91_400_422_J1 | 54 | 1 | KFR (SEQ | MKKH (SEQ | WHMKKH (SEQ ID NO: |
| | | | ID NO: 55) | ID NO: 155) | 164) |
| ZN628_120_142- | ZN6 | ZFP9 | FICGQCGL | QKASLNWH | FICGQCGLAFKQKASLN |
| ZFP91_400_422_J1 | 28 | 1 | AFK (SEQ | MKKH (SEQ | WHMKKH (SEQ ID NO: |
| | | | ID NO: 49) | ID NO: 155) | 165) |
| IKZF2_140_162- | IKZF | ZFP9 | FHCNQCGA | QKASLNWH | FHCNQCGASFTQKASLN |
| ZFP91_400_422_J1 | 2 | 1 | SFT (SEQ ID | MKKH (SEQ | WHMKKH (SEQ ID NO: |
| | | | NO: 69) | ID NO: 155) | 166) |
| ZN597_341_363- | ZN5 | ZFP9 | LQCPDCDM | QKASLNWH | LQCPDCDMTFPQKASLN |
| ZFP91_400_422_J1 | 97 | 1 | TFP (SEQ ID | MKKH (SEQ | WHMKKH (SEQ ID NO: |
| | | | NO: 59) | ID NO: 155) | 167) |
| IKZF3_146_168- | IKZF | ZFP9 | FQCNQCGA | QKASLNWH | FQCNQCGASFTQKASLN |
| ZFP91_400_422_J1 | 3 | 1 | SFT (SEQ ID | MKKH (SEQ | WHMKKH (SEQ ID NO: |
| | | | NO: 46) | ID NO: 155) | 168) |
| ZNF74_444_466- | ZNF | ZFP9 | FKCADCGK | QKASLNWH | FKCADCGKGFSQKASLN |
| ZFP91_400_422_J1 | 74 | 1 | GFS (SEQ ID | MKKH (SEQ | WHMKKH (SEQ ID NO: |
| | | | NO: 75) | ID NO: 155) | 169) |
| E4F1_220_242- | E4F1 | ZFP9 | HECKLCGA | QKASLNWH | HECKLCGASFRQKASLN |
| ZFP91_400_422_J1 | | 1 | SFR (SEQ ID | MKKH (SEQ | WHMKKH (SEQ ID NO: |
| | | | NO: 81) | ID NO: 155) | 170) |
| ZKSC5_430_452- | ZKS | ZFP9 | YGCNECGK | QKASLNWH | YGCNECGKNFGQKASLN |
| ZFP91_400_422_J1 | C5 | 1 | NFG (SEQ | MKKH (SEQ | WHMKKH (SEQ ID NO: |
| | | | ID NO: 73) | ID NO: 155) | 171) |
| ZFP91_400_422Z | ZFP9 | ZFP9 | LQCEICGFT | QKASLNWH | LQCEICGFTCRQKASLN |
| N692_417_439- | 1 | 1 | CR (SEQ ID | MKKH (SEQ | WHMKKH (SEQ ID NO: |
| ZFP91_400_422_J1 | | | NO: 67) | ID NO: 155) | 172) |
| ZSC20_766_788- | ZSC | ZFP9 | YKCLECGK | QKASLNWH | YKCLECGKSFSQKASLN |
| ZFP91_400_422_J1 | 20 | 1 | SFS (SEQ ID | MKKH (SEQ | WHMKKH (SEQ ID NO: |
| | | | NO: 63) | ID NO: 155) | 173) |
| ZNF90_481_503- | ZNF | ZFP9 | YKCQECDK | QKASLNWH | YKCQECDKAFKQKASLN |
| ZFP91_400_422_J1 | 90 | 1 | AFK (SEQ | MKKH (SEQ | WHMKKH (SEQ ID NO: |
| | | | ID NO: 61) | ID NO: 155) | 174) |
| ZN517_452_474- | ZN5 | ZFP9 | YRCRACGR | QKASLNWH | YRCRACGRACSQKASLN |
| ZFP91_400_422_J1 | 17 | 1 | ACS (SEQ | MKKH (SEQ | WHMKKH (SEQ ID NO: |
| | | | ID NO: 83) | ID NO: 155) | 175) |
| ZN595_145_167- | ZN5 | ZFP9 | FQCNTCVK | QKASLNWH | FQCNTCVK VFSQKASLN |
| ZFP91_400_422_J1 | 95 | 1 | VFS (SEQ ID | MKKH (SEQ | WHMKKH (SEQ ID NO: |
| | | | NO: 85) | ID NO: 155) | 176) |
| ZN597_341_363- | ZN5 | ZKS | LQCPDCDM | RHSHLIEHLK | LQCPDCDMTFPRHSHLIE |
| ZKSC5_430_452_J1 | 97 | C5 | TFP (SEQ ID | RH (SEQ ID | HLKRH (SEQ ID NO: 178) |
| | | | NO: 59) | NO: 177) | |
| ZNF90_481_503- | ZNF | ZKS | YKCQECDK | RHSHLIEHLK | YKCQECDKAFKRHSHLI |
| ZKSC5_430_452_J1 | 90 | C5 | AFK (SEQ | RH (SEQ ID | EHLKRH (SEQ ID NO: |
| | | | ID NO: 61) | NO: 177) | 179) |
| ZN398_483_505- | ZN3 | ZKS | FSCPQCGID | RHSHLIEHLK | FSCPQCGIDFNRHSHLIE |
| ZKSC5_430_452_J1 | 98 | C5 | FN (SEQ ID | RH (SEQ ID | HLKRH (SEQ ID NO: 180) |
| | | | NO: 53) | NO: 177) | |
| IKZF2_140_162- | IKZF | ZKS | FHCNQCGA | RHSHLIEHLK | FHCNQCGASFTRHSHLIE |
| ZKSC5_430_452_J1 | 2 | C5 | SFT (SEQ ID | RH (SEQ ID | HLKRH (SEQ ID NO: 181) |
| | | | NO: 69) | NO: 177) | |
| ZKSC5_430_452- | ZKS | ZKS | YGCNECGK | RHSHLIEHLK | YGCNECGKNFGRHSHLI |
| ZKSC5_430_452_J1 | C5 | C5 | NFG (SEQ | RH (SEQ ID | EHLKRH (SEQ ID NO: |
| | | | ID NO: 73) | NO: 177) | 182) |
| ZN628_120_142- | ZN6 | ZKS | FICGQCGL | RHSHLIEHLK | FICGQCGL AFKRHSHL IE |
| ZKSC5_430_452_J1 | 28 | C5 | AFK (SEQ | RH (SEQ ID | HLKRH (SEQ ID NO: 183) |
| | | | ID NO: 49) | NO: 177) | |
| PATZ1_383_405- | PAT | ZKS | YSCPVCGL | RHSHLIEHLK | YSCPVCGLRFKRHSHLIE |
| ZKSC5_430_452_J1 | Z1 | C5 | RFK (SEQ | RH (SEQ ID | HLKRH (SEQ ID NO: 184) |
| | | | ID NO: 51) | NO: 177) | |
| ZN654_25_47- | ZN6 | ZKS | FACVICGR | RHSHLIEHLK | FACVICGRKFRRHSHLIE |
| ZKSC5_430_452_J1 | 54 | C5 | KFR (SEQ | RH (SEQ ID | HLKRH (SEQ ID NO: 185) |
| | | | ID NO: 55) | NO: 177) | |
| ZSC20_766_788- | ZSC | ZKS | YKCLECGK | RHSHLIEHLK | YKCLECGKSFSRHSHLIE |
| ZKSC5_430_452_J1 | 20 | C5 | SFS (SEQ ID | RH (SEQ ID | HLKRH (SEQ ID NO: 186) |
| | | | NO: 63) | NO: 177) | |
| ZN582_395_417- | ZN5 | ZKS | YQCKVCGR | RHSHLIEHLK | YQCKVCGRAFKRHSHLI |
| ZKSC5_430_452_J1 | 82 | C5 | AFK (SEQ | RH (SEQ ID | EHLKRH (SEQ ID NO: |
| | | | ID NO: 77) | NO: 177) | 187) |
| ZN787_178_200- | ZN7 | ZKS | FVCPRCGR | RHSHLIEHLK | FVCPRCGRGFSRHSHLIE |
| ZKSC5_430_452_J1 | 87 | C5 | GFS (SEQ ID | RH (SEQ ID | HLKRH (SEQ ID NO: 188) |
| | | | NO: 79) | NO: 177) | |
| ZN276_524_546- | ZN2 | ZKS | LQCEVCGF | RHSHLIEHLK | LQCEVCGFQCRRHSHLIE |
| ZKSC5_430_452_J1 | 76 | C5 | QCR (SEQ | RH (SEQ ID | HLKRH (SEQ ID NO: 189) |
| | | | ID NO: 71) | NO: 177) | |
| ZF69B_419_441- | ZF69 | ZKS | YICNVCSK | RHSHLIEHLK | YICNVCSKTFSRHSHLIE |
| ZKSC5_430_452_J1 | B | C5 | TFS (SEQ ID | RH (SEQ ID | HLKRH (SEQ ID NO: 190) |
| | | | NO: 87) | NO: 177) | |
| ZN595_145_167- | ZN5 | ZKS | FQCNTCVK | RHSHLIEHLK | FQCNTCVK VFSRHSHLIE |
| ZKSC5_430_452_J1 | 95 | C5 | VFS (SEQ ID | RH (SEQ ID | HLKRH (SEQ ID NO: 191) |
| | | | NO: 85) | NO: 177) | |
| ZN653_556_578- | ZN6 | ZKS | LQCEICGY | RHSHLIEHLK | LQCEICGYQCRRHSHLIE |
| ZKSC5_430_452_J1 | 53 | C5 | QCR (SEQ | RH (SEQ ID | HLKRH (SEQ ID NO: 192) |
| | | | ID NO: 65) | NO: 177) | |
| E4F1_220_242- | E4F1 | ZKS | HECKLCGA | RHSHLIEHLK | HECKLCGASFRRHSHLIE |
| ZKSC5_430_452_J1 | | C5 | SFR (SEQ ID | RH (SEQ ID | HLKRH (SEQ ID NO: 193) |
| | | | NO: 81) | NO: 177) | |
| ZN517_452_474- | ZN5 | ZKS | YRCRACGR | RHSHLIEHLK | YRCRACGRACSRHSHLIE |
| ZKSC5_430_452_J1 | 17 | C5 | ACS (SEQ | RH (SEQ ID | HLKRH (SEQ ID NO: 194) |
| | | | ID NO: 83) | NO: 177) | |
| ZNF74_444_466- | ZNF | ZKS | FKCADCGK | RHSHLIEHLK | FKCADCGKGFSRHSHLIE |
| ZKSC5_430_452_J1 | 74 | C5 | GFS (SEQ ID | RH (SEQ ID | HLKRH (SEQ ID NO: 195) |
| | | | NO: 75) | NO: 177) | |
| ZFP91_400_422ZN692 | ZFP9 | ZKS | LQCEICGFT | RHSHLIEHLK | LQCEICGFTCRRHSHLIE |
| 417_43_9- | 1 | C5 | CR (SEQ ID | RH (SEQ ID | HLKRH (SEQ ID NO: 196) |
| ZKSC5_430_452_J1 | | | NO: 67) | NO: 177) | |
| IKZF3_146_168- | IKZF | ZKS | FQCNQCGA | RHSHLIEHLK | FQCNQCGASFTRHSHLIE |
| ZKSC5_430_452_J1 | 3 | C5 | SFT (SEQ ID | RH (SEQ ID | HLKRH (SEQ ID NO: 197) |
| | | | NO: 46) | NO: 177) | |
| ZN827_374_396- | ZN8 | ZKS | FQCPICGLV | RHSHLIEHLK | FQCPICGLVIKRHSHLIEH |
| ZKSC5_430_452_J1 | 27 | C5 | IK (SEQ ID | RH (SEQ ID | LKRH (SEQ ID NO: 198) |
| | | | NO: 57) | NO: 177) | |
| ZF69B_419_441- | ZF69 | ZN2 | YICNVCSK | QRASLKYHM | YICNVCSKTFSQRASLKY |
| ZN276_524_546_J1 | B | 76 | TFS (SEQ ID | TKH (SEQ ID | HMTKH (SEQ ID NO: 200) |
| | | | NO: 87) | NO: 199) | |
| ZN517_452_474- | ZN5 | ZN2 | YRCRACGR | QRASLKYHM | YRCRACGRACSQRASLK |
| ZN276_524_546_J1 | 17 | 76 | ACS (SEQ | TKH (SEQ ID | YHMTKH (SEQ ID NO: |
| | | | ID NO: 83) | NO: 199) | 201) |
| IKZF2_140_162- | IKZF | ZN2 | FHCNQCGA | QRASLKYHM | FHCNQCGASFTQRASLK |
| ZN276_524_546_J1 | 2 | 76 | SFT (SEQ ID | TKH (SEQ ID | YHMTKH (SEQ ID NO: |
| | | | NO: 69) | NO: 199) | 202) |
| IKZF3_146_168- | IKZF | ZN2 | FQCNQCGA | QRASLKYHM | FQCNQCGASFTQRASLK |
| ZN276_524_546_J1 | 3 | 76 | SFT (SEQ ID | TKH (SEQ ID | YHMTKH (SEQ ID NO: |
| | | | NO: 46) | NO: 199) | 203 |
| ZN628_120_142- | ZN6 | ZN2 | FICGQCGL | QRASLKYHM | FICGQCGLAFKQRASLK |
| ZN276_524_546_J1 | 28 | 76 | AFK (SEQ | TKH (SEQ ID | YHMTKH (SEQ ID NO: |
| | | | ID NO: 49) | NO: 199) | 204) |
| ZN398_483_505- | ZN3 | ZN2 | FSCPQCGID | QRASLKYHM | FSCPQCGIDFNQRASLKY |
| ZN276_524_546_J1 | 98 | 76 | FN (SEQ ID | TKH (SEQ ID | HMTKH (SEQ ID NO: 205) |
| | | | NO: 53) | NO: 199) | |
| ZN597_341_363- | ZN5 | ZN2 | LQCPDCDM | QRASLKYHM | LQCPDCDMTFPQRASLK |
| ZN276_524_546_J1 | 97 | 76 | TFP (SEQ ID | TKH (SEQ ID | YHMTKH (SEQ ID NO: |
| | | | NO: 59) | NO: 199) | 206) |
| E4F1_220_242- | E4F1 | ZN2 | HECKLCGA | QRASLKYHM | HECKLCGASFRQRASLK |
| ZN276_524_546_J1 | | 76 | SFR (SEQ ID | TKH (SEQ ID | YHMTKH (SEQ ID NO: |
| | | | NO: 81) | NO: 199) | 207) |
| ZN827_374_396- | ZN8 | ZN2 | FQCPICGLV | QRASLKYHM | FQCPICGLVIKQRASLKY |
| ZN276_524_546_J1 | 27 | 76 | IK (SEQ ID | TKH (SEQ ID | HMTKH (SEQ ID NO: 208) |
| | | | NO: 57) | NO: 199) | |
| ZN787_178_200- | ZN7 | ZN2 | FVCPRCGR | QRASLKYHM | FVCPRCGRGFSQRASLK |
| ZN276_524_546_J1 | 87 | 76 | GFS (SEQ ID | TKH (SEQ ID | YHMTKH (SEQ ID NO: |
| | | | NO: 79) | NO: 199) | 209) |
| ZNF90_481_503- | ZNF | ZN2 | YKCQECDK | QRASLKYHM | YKCQECDKAFKQRASLK |
| ZN276_524_546_J1 | 90 | 76 | AFK (SEQ | TKH (SEQ ID | YHMTKH (SEQ ID NO: |
| | | | ID NO: 61) | NO: 199) | 210) |
| ZSC20_766_788- | ZSC | ZN2 | YKCLECGK | QRASLKYHM | YKCLECGKSFSQRASLK |
| ZN276_524_546_J1 | 20 | 76 | SFS (SEQ ID | TKH (SEQ ID | YHMTKH (SEQ ID NO: |
| | | | NO: 63) | NO: 199) | 211) |
| PATZ1_383_405- | PAT | ZN2 | YSCPVCGL | QRASLKYHM | YSCPVCGLRFKQRASLK |
| ZN276_524_546_J1 | Z1 | 76 | RFK (SEQ | TKH (SEQ ID | YHMTKH (SEQ ID NO: |
| | | | ID NO: 51) | NO: 199) | 212) |
| ZN582_395_417- | ZN5 | ZN2 | YQCKVCGR | QRASLKYHM | YQCKVCGRAFKQRASLK |
| ZN276_524_546_J1 | 82 | 76 | AFK (SEQ | TKH (SEQ ID | YHMTKH (SEQ ID NO: |
| | | | ID NO: 77) | NO: 199) | 213) |
| ZN653_556_578- | ZN6 | ZN2 | LQCEICGY | QRASLKYHM | LQCEICGYQCRQRASLK |
| ZN276_524_546_J1 | 53 | 76 | QCR (SEQ | TKH (SEQ ID | YHMTKH (SEQ ID NO: |
| | | | ID NO: 65) | NO: 199) | 214) |
| ZN276_524_546- | ZN2 | ZN2 | LQCEVCGF | QRASLKYHM | LQCEVCGFQCRQRASLK |
| ZN276_524_546_J1 | 76 | 76 | QCR (SEQ | TKH (SEQ ID | YHMTKH (SEQ ID NO: |
| | | | ID NO: 71) | NO: 199) | 215) |
| ZFP91_400_422Z | ZFP9 | ZN2 | LQCEICGFT | QRASLKYHM | LQCEICGFTCRQRASLKY |
| N692 417_439- | 1 | 76 | CR (SEQ ID | TKH (SEQ ID | HMTKH (SEQ ID NO: 216) |
| ZN276_524_546_J1 | | | NO: 67) | NO: 199) | |
| ZNF74_444_466- | ZNF | ZN2 | FKCADCGK | QRASLKYHM | FKCADCGKGFSQRASLK |
| ZN276_524_546_J1 | 74 | 76 | GFS (SEQ ID | TKH (SEQ ID | YHMTKH (SEQ ID NO: |
| | | | NO: 75) | NO: 199) | 217) |
| ZKSC5_430_452- | ZKS | ZN2 | YGCNECGK | QRASLKYHM | YGCNECGKNFGQRASLK |
| ZN276_524_546_J1 | C5 | 76 | NFG (SEQ | TKH (SEQ ID | YHMTKH (SEQ ID NO: |
| | | | ID NO: 73) | NO: 199) | 218) |
| ZN654_25_47- | ZN6 | ZN2 | FACVICGR | QRASLKYHM | FACVICGRKFRQRASLK |
| ZN276_524_546_J1 | 54 | 76 | KFR (SEQ | TKH (SEQ ID | YHMTKH (SEQ ID NO: |
| | | | ID NO: 55) | NO: 199) | 219) |
| ZN595_145_167- | ZN5 | ZN2 | FQCNTCVK | QRASLKYHM | FQCNTCVK VFSQRASLK |
| ZN276_524_546_J1 | 95 | 76 | VFS (SEQ ID | TKH (SEQ ID | YHMTKH (SEQ ID NO: |
| | | | NO: 85) | NO: 199) | 220) |
| PATZ1_383_405- | PAT | ZN3 | YSCPVCGL | GHSALIRHQ | YSCPVCGLRFKGHSALIR |
| ZN398_483_505_J1 | Z1 | 98 | RFK (SEQ | MIH (SEQ ID | HQMIH (SEQ ID NO: 222) |
| | | | ID NO: 51) | NO: 221) | |
| ZNF74_444_466- | ZNF | ZN3 | FKCADCGK | GHSALIRHQ | FKCADCGKGFSGHSALIR |
| ZN398_483_505_J1 | 74 | 98 | GFS (SEQ ID | MIH (SEQ ID | HQMIH (SEQ ID NO: 223) |
| | | | NO: 75) | NO: 221) | |
| ZKSC5_430_452- | ZKS | ZN3 | YGCNECGK | GHSALIRHQ | YGCNECGKNFGGHSALI |
| ZN398_483_505_J1 | C5 | 98 | NFG (SEQ | MIH (SEQ ID | RHQMIH (SEQ ID NO: |
| | | | ID NO: 73) | NO: 221) | 224) |
| ZN276_524_546- | ZN2 | ZN3 | LQCEVCGF | GHSALIRHQ | LQCEVCGFQCRGHSALIR |
| ZN398_483_505_J1 | 76 | 98 | QCR (SEQ | MIH (SEQ ID | HQMIH (SEQ ID NO: 225) |
| | | | ID NO: 71) | NO: 221) | |
| ZN517_452_474- | ZN5 | ZN3 | YRCRACGR | GHSALIRHQ | YRCRACGRACSGHSALI |
| ZN398_483_505_J1 | 17 | 98 | ACS (SEQ | MIH (SEQ ID | RHQMIH (SEQ ID NO: |
| | | | ID NO: 83) | NO: 221) | 226) |
| ZN827_374_396- | ZN8 | ZN3 | FQCPICGLV | GHSALIRHQ | FQCPICGLVIKGHSALIRH |
| ZN398_483_505_J1 | 27 | 98 | IK (SEQ ID | MIH (SEQ ID | QMIH (SEQ ID NO: 227) |
| | | | NO: 57) | NO: 221) | |
| IKZF2_140_162- | IKZF | ZN3 | FHCNQCGA | GHSALIRHQ | FHCNQCGASFTGHSALIR |
| ZN398_483_505_J1 | 2 | 98 | SFT (SEQ ID | MIH (SEQ ID | HQMIH (SEQ ID NO: 228) |
| | | | NO: 69) | NO: 221) | |
| ZN398_483_505- | ZN3 | ZN3 | FSCPQCGID | GHSALIRHQ | FSCPQCGIDFNGHSALIR |
| ZN398_483_505_J1 | 98 | 98 | FN (SEQ ID | MIH (SEQ ID | HQMIH (SEQ ID NO: 229) |
| | | | NO: 53) | NO: 221) | |
| ZF69B_419_441- | ZF69 | ZN3 | YICNVCSK | GHSALIRHQ | YICNVCSKTFSGHSALIR |
| ZN398_483_505_J1 | B | 98 | TFS (SEQ ID | MIH (SEQ ID | HQMIH (SEQ ID NO: 230) |
| | | | NO: 87) | NO: 221) | |
| E4F1_220_242- | E4F1 | ZN3 | HECKLCGA | GHSALIRHQ | HECKLCGASFRGHSALIR |
| ZN398_483_505_J1 | | 98 | SFR (SEQ ID | MIH (SEQ ID | HQMIH (SEQ ID NO: 231) |
| | | | NO: 81) | NO: 221) | |
| ZN654_25_47- | ZN6 | ZN3 | FACVICGR | GHSALIRHQ | FACVICGRKFRGHSALIR |
| ZN398_483_505_J1 | 54 | 98 | KFR (SEQ | MIH (SEQ ID | HQMIH (SEQ ID NO: 232) |
| | | | ID NO: 55) | NO: 221) | |
| ZN628_120_142- | ZN6 | ZN3 | FICGQCGL | GHSALIRHQ | FICGQCGLAFKGHSALIR |
| ZN398_483_505_J1 | 28 | 98 | AFK (SEQ | MIH (SEQ ID | HQMIH (SEQ ID NO: 233) |
| | | | ID NO: 49) | NO: 221) | |
| ZN595_145_167- | ZN5 | ZN3 | FQCNTCVK | GHSALIRHQ | FQCNTCVK VFSGHSALIR |
| ZN398_483_505_J1 | 95 | 98 | VFS (SEQ ID | MIH (SEQ ID | HQMIH (SEQ ID NO: 234) |
| | | | NO: 85) | NO: 221) | |
| IKZF3_146_168- | IKZF | ZN3 | FQCNQCGA | GHSALIRHQ | FQCNQCGASFTGHSALIR |
| ZN398_483_505_J1 | 3 | 98 | SFT (SEQ ID | MIH (SEQ ID | HQMIH (SEQ ID NO: 235) |
| | | | NO: 46) | NO: 221) | |
| ZN582_395_417- | ZN5 | ZN3 | YQCKVCGR | GHSALIRHQ | YQCKVCGRAFKGHSALI |
| ZN398_483_505_J1 | 82 | 98 | AFK (SEQ | MIH (SEQ ID | RHQMIH (SEQ ID NO: |
| | | | ID NO: 77) | NO: 221) | 236) |
| ZNF90_481_503- | ZNF | ZN3 | YKCQECDK | GHSALIRHQ | YKCQECDKAFKGHSALI |
| ZN398_483_505_J1 | 90 | 98 | AFK (SEQ | MIH (SEQ ID | RHQMIH (SEQ ID NO: |
| | | | ID NO: 61) | NO: 221) | 237) |
| ZN787_178_200- | ZN7 | ZN3 | FVCPRCGR | GHSALIRHQ | FVCPRCGRGFSGHSALIR |
| ZN398_483_505_J1 | 87 | 98 | GFS (SEQ ID | MIH (SEQ ID | HQMIH (SEQ ID NO: 238) |
| | | | NO: 79) | NO: 221) | |
| ZSC20_766_788- | ZSC | ZN3 | YKCLECGK | GHSALIRHQ | YKCLECGKSFSGHSALIR |
| ZN398_483_505_J1 | 20 | 98 | SFS (SEQ ID | MIH (SEQ ID | HQMIH (SEQ ID NO: 239) |
| | | | NO: 63) | NO: 221) | |
| ZFP91_400_422Z | ZFP9 | ZN3 | LQCEICGFT | GHSALIRHQ | LQCEICGFTCRGHSALIR |
| N692 417_43_9- | 1 | 98 | CR (SEQ ID | MIH (SEQ ID | HQMIH (SEQ ID NO: 240) |
| ZN398_483_505_J1 | | | NO: 67) | NO: 221) | |
| ZN597_341_363- | ZN5 | ZN3 | LQCPDCDM | GHSALIRHQ | LQCPDCDMTFPGHSALIR |
| ZN398_483_505_J1 | 97 | 98 | TFP (SEQ ID | MIH (SEQ ID | HQMIH (SEQ ID NO: 241) |
| | | | NO: 59) | NO: 221) | |
| ZN653_556_578- | ZN6 | ZN3 | LQCEICGY | GHSALIRHQ | LQCEICGYQCRGHSALIR |
| ZN398_483_505_J1 | 53 | 98 | QCR (SEQ | MIH (SEQ ID | HQMIH (SEQ ID NO: 242) |
| | | | ID NO: 65) | NO: 221) | |
| ZN628_120_142- | ZN6 | ZN5 | FICGQCGL | RLSTLIQHQK | FICGQCGLAFKRLSTLIQ |
| ZN517_452_474_J1 | 28 | 17 | AFK (SEQ | VH (SEQ ID | HQKVH (SEQ ID NO: 244) |
| | | | ID NO: 49) | NO: 243) | |
| IKZF3_146_168- | IKZF | ZN5 | FQCNQCGA | RLSTLIQHQK | FQCNQCGASFTRLSTLIQ |
| ZN517_452_474_J1 | 3 | 17 | SFT (SEQ ID | VH (SEQ ID | HQKVH (SEQ ID NO: 245) |
| | | | NO: 46) | NO: 243) | |
| ZN517_452_474- | ZN5 | ZN5 | YRCRACGR | RLSTLIQHQK | YRCRACGRACSRLSTLIQ |
| ZN517_452_474_J1 | 17 | 17 | ACS (SEQ | VH (SEQ ID | HQKVH (SEQ ID NO: 246) |
| | | | ID NO: 83) | NO: 243) | |
| ZN653_556_578- | ZN6 | ZN5 | LQCEICGY | RLSTLIQHQK | LQCEICGYQCRRLSTLIQ |
| ZN517_452_474_J1 | 53 | 17 | QCR (SEQ | VH (SEQ ID | HQKVH (SEQ ID NO: 247) |
| | | | ID NO: 65) | NO: 243) | |
| PATZ1_383_405- | PAT | ZN5 | YSCPVCGL | RLSTLIQHQK | YSCPVCGLRFKRLSTLIQ |
| ZN517_452_474_J1 | Z1 | 17 | RFK (SEQ | VH (SEQ ID | HQKVH (SEQ ID NO: 248) |
| | | | ID NO: 51) | NO: 243) | |
| ZN595_145_167- | ZN5 | ZN5 | FQCNTCVK | RLSTLIQHQK | FQCNTCVK VFSRLSTLIQ |
| ZN517_452_474_J1 | 95 | 17 | VFS (SEQ ID | VH (SEQ ID | HQKVH (SEQ ID NO: 249) |
| | | | NO: 85) | NO: 243) | |
| ZN597_341_363- | ZN5 | ZN5 | LQCPDCDM | RLSTLIQHQK | LQCPDCDMTFPRLSTLIQ |
| ZN517_452_474_J1 | 97 | 17 | TFP (SEQ ID | VH (SEQ ID | HQKVH (SEQ ID NO: 250) |
| | | | NO: 59) | NO: 243) | |
| ZSC20_766_788- | ZSC | ZN5 | YKCLECGK | RLSTLIQHQK | YKCLECGKSFSRLSTLIQ |
| ZN517_452_474_J1 | 20 | 17 | SFS (SEQ ID | VH (SEQ ID | HQKVH (SEQ ID NO: 251) |
| | | | NO: 63) | NO: 243) | |
| ZFP91_400_422ZN692 | ZFP9 | ZN5 | LQCEICGFT | RLSTLIQHQK | LQCEICGFTCRRLSTLIQH |
| 417_439- | 1 | 17 | CR (SEQ ID | VH (SEQ ID | QKVH (SEQ ID NO: 252) |
| ZN517_452_474_J1 | | | NO: 67) | NO: 243) | |
| ZNF90_481_503- | ZNF | ZN5 | YKCQECDK | RLSTLIQHQK | YKCQECDKAFKRLSTLIQ |
| ZN517_452_474_J1 | 90 | 17 | AFK (SEQ | VH (SEQ ID | HQKVH (SEQ ID NO: 253) |
| | | | ID NO: 61) | NO: 243) | |
| ZN654_25_47- | ZN6 | ZN5 | FACVICGR | RLSTLIQHQK | FACVICGRKFRRLSTLIQ |
| ZN517_452_474_J1 | 54 | 17 | KFR (SEQ | VH (SEQ ID | HQKVH (SEQ ID NO: 254) |
| | | | ID NO: 55) | NO: 243) | |
| ZN398_483_505- | ZN3 | ZN5 | FSCPQCGID | RLSTLIQHQK | FSCPQCGIDFNRLSTLIQH |
| ZN517_452_474_J1 | 98 | 17 | FN (SEQ ID | VH (SEQ ID | QKVH (SEQ ID NO: 255) |
| | | | NO: 53) | NO: 243) | |
| ZN276_524_546- | ZN2 | ZN5 | LQCEVCGF | RLSTLIQHQK | LQCEVCGFQCRRLSTLIQ |
| ZN517_452_474_J1 | 76 | 17 | QCR (SEQ | VH (SEQ ID | HQKVH (SEQ ID NO: 256) |
| | | | ID NO: 71) | NO: 243) | |
| IKZF2_140_162- | IKZF | ZN5 | FHCNQCGA | RLSTLIQHQK | FHCNQCGASFTRLSTLIQ |
| ZN517_452_474_J1 | 2 | 17 | SFT (SEQ ID | VH (SEQ ID | HQKVH (SEQ ID NO: 257) |
| | | | NO: 69) | NO: 243) | |
| ZKSC5_430_452- | ZKS | ZN5 | YGCNECGK | RLSTLIQHQK | YGCNECGKNFGRLSTLIQ |
| ZN517_452_474_J1 | C5 | 17 | NFG (SEQ | VH (SEQ ID | HQKVH (SEQ ID NO: 258) |
| | | | ID NO: 73) | NO: 243) | |
| ZN787_178_200- | ZN7 | ZN5 | FVCPRCGR | RLSTLIQHQK | FVCPRCGRGFSRLSTLIQ |
| ZN517_452_474_J1 | 87 | 17 | GFS (SEQ ID | VH (SEQ ID | HQKVH (SEQ ID NO: 259) |
| | | | NO: 79) | NO: 243) | |
| ZF69B_419_441- | ZF69 | ZN5 | YICNVCSK | RLSTLIQHQK | YICNVCSKTFSRLSTLIQH |
| ZN517_452_474_J1 | B | 17 | TFS (SEQ ID | VH (SEQ ID | QKVH (SEQ ID NO: 260) |
| | | | NO: 87) | NO: 243) | |
| ZN827_374_396- | ZN8 | ZN5 | FQCPICGLV | RLSTLIQHQK | FQCPICGLVIKRLSTLIQH |
| ZN517_452_474_J1 | 27 | 17 | IK (SEQ ID | VH (SEQ ID | QKVH (SEQ ID NO: 261) |
| | | | NO: 57) | NO: 243) | |
| ZNF74_444_466- | ZNF | ZN5 | FKCADCGK | RLSTLIQHQK | FKCADCGKGFSRLSTLIQ |
| ZN517_452_474_J1 | 74 | 17 | GFS (SEQ ID | VH (SEQ ID | HQKVH (SEQ ID NO: 262) |
| | | | NO: 75) | NO: 243) | |
| ZN582_395_417- | ZN5 | ZN5 | YQCKVCGR | RLSTLIQHQK | YQCKVCGRAFKRLSTLI |
| ZN517_452_474_J1 | 82 | 17 | AFK (SEQ | VH (SEQ ID | QHQKVH (SEQ ID NO: |
| | | | ID NO: 77) | NO: 243) | 263) |
| E4F1_220_242- | E4F1 | ZN5 | HECKLCGA | RLSTLIQHQK | HECKLCGASFRRLSTLIQ |
| ZN517_452_474_J1 | | 17 | SFR (SEQ ID | VH (SEQ ID | HQKVH (SEQ ID NO: 264) |
| | | | NO: 81) | NO: 243) | |
| ZN595_145_167- | ZN5 | ZN5 | FQCNTCVK | RVSHLTVHY | FQCNTCVKVFSRVSHLT |
| ZN582_395_417_J1 | 95 | 82 | VFS (SEQ ID | RIH (SEQ ID | VHYRIH (SEQ ID NO: |
| | | | NO: 85) | NO: 265) | 266) |
| IKZF2_140_162- | IKZF | ZN5 | FHCNQCGA | RVSHLTVHY | FHCNQCGASFTRVSHLT |
| ZN582_395_417_J1 | 2 | 82 | SFT (SEQ ID | RIH (SEQ ID | VHYRIH (SEQ ID NO: |
| | | | NO: 69) | NO: 265) | 267) |
| ZN582_395_417- | ZN5 | ZN5 | YQCKVCGR | RVSHLTVHY | YQCKVCGRAFKRVSHLT |
| ZN582_395_417_J1 | 82 | 82 | AFK (SEQ | RIH (SEQ ID | VHYRIH (SEQ ID NO: |
| | | | ID NO: 77) | NO: 265) | 268) |
| ZN517_452_474- | ZN5 | ZN5 | YRCRACGR | RVSHLTVHY | YRCRACGRACSRVSHLT |
| ZN582_395_417_J1 | 17 | 82 | ACS (SEQ | RIH (SEQ ID | VHYRIH (SEQ ID NO: |
| | | | ID NO: 83) | NO: 265) | 269) |
| ZN628_120_142- | ZN6 | ZN5 | FICGQCGL | RVSHLTVHY | FICGQCGLAFKRVSHLTV |
| ZN582_395_417_J1 | 28 | 82 | AFK (SEQ | RIH (SEQ ID | HYRIH (SEQ ID NO: 270) |
| | | | ID NO: 49) | NO: 265) | |
| ZN654_25_47- | ZN6 | ZN5 | FACVICGR | RVSHLTVHY | FACVICGRKFRRVSHLTV |
| ZN582_395_417_J1 | 54 | 82 | KFR (SEQ | RIH (SEQ ID | HYRIH (SEQ ID NO: 271) |
| | | | ID NO: 55) | NO: 265) | |
| ZN597_341_363- | ZN5 | ZN5 | LQCPDCDM | RVSHLTVHY | LQCPDCDMTFPRVSHLT |
| ZN582_395_417_J1 | 97 | 82 | TFP (SEQ ID | RIH (SEQ ID | VHYRIH (SEQ ID NO: |
| | | | NO: 59) | NO: 265) | 272) |
| ZF69B_419_441- | ZF69 | ZN5 | YICNVCSK | RVSHLTVHY | YICNVCSKTFSRVSHLTV |
| ZN582_395_417_J1 | B | 82 | TFS (SEQ ID | RIH (SEQ ID | HYRIH (SEQ ID NO: 273) |
| | | | NO: 87) | NO: 265) | |
| ZNF74_444_466- | ZNF | ZN5 | FKCADCGK | RVSHLTVHY | FKCADCGKGFSRVSHLT |
| ZN582_395_417_J1 | 74 | 82 | GFS (SEQ ID | RIH (SEQ ID | VHYRIH (SEQ ID NO: |
| | | | NO: 75) | NO: 265) | 274) |
| ZNF90_481_503- | ZNF | ZN5 | YKCQECDK | RVSHLTVHY | YKCQECDKAFKRVSHLT |
| ZN582_395_417_J1 | 90 | 82 | AFK (SEQ | RIH (SEQ ID | VHYRIH (SEQ ID NO: |
| | | | ID NO: 61) | NO: 265) | 275) |
| ZN398_483_505- | ZN3 | ZN5 | FSCPQCGID | RVSHLTVHY | FSCPQCGIDFNRVSHLTV |
| ZN582_395_417_J1 | 98 | 82 | FN (SEQ ID | RIH (SEQ ID | HYRIH (SEQ ID NO: 276) |
| | | | NO: 53) | NO: 265) | |
| ZKSC5_430_452- | ZKS | ZN5 | YGCNECGK | RVSHLTVHY | YGCNECGKNFGRVSHLT |
| ZN582_395_417_J1 | C5 | 82 | NFG (SEQ | RIH (SEQ ID | VHYRIH (SEQ ID NO: |
| | | | ID NO: 73) | NO: 265) | 277) |
| IKZF3_146_168- | IKZF | ZN5 | FQCNQCGA | RVSHLTVHY | FQCNQCGASFTRVSHLT |
| ZN582_395_417_J1 | 3 | 82 | SFT (SEQ ID | RIH (SEQ ID | VHYRIH (SEQ ID NO: |
| | | | NO: 46) | NO: 265) | 278) |
| ZN276_524_546- | ZN2 | ZN5 | LQCEVCGF | RVSHLTVHY | LQCEVCGFQCRRVSHLT |
| ZN582_395_417_J1 | 76 | 82 | QCR (SEQ | RIH (SEQ ID | VHYRIH (SEQ ID NO: |
| | | | ID NO: 71) | NO: 265) | 279) |
| ZSC20_766_788- | ZSC | ZN5 | YKCLECGK | RVSHLTVHY | YKCLECGKSFSRVSHLT |
| ZN582_395_417_J1 | 20 | 82 | SFS (SEQ ID | RIH (SEQ ID | VHYRIH (SEQ ID NO: |
| | | | NO: 63) | NO: 265) | 280) |
| E4F1_220_242- | E4F1 | ZN5 | HECKLCGA | RVSHLTVHY | HECKLCGASFRRVSHLT |
| ZN582_395_417_J1 | | 82 | SFR (SEQ ID | RIH (SEQ ID | VHYRIH (SEQ ID NO: |
| | | | NO: 81) | NO: 265) | 281) |
| PATZ1_383_405- | PAT | ZN5 | YSCPVCGL | RVSHLTVHY | YSCPVCGLRFKRVSHLT |
| ZN582_395_417_J1 | Z1 | 82 | RFK (SEQ | RIH (SEQ ID | VHYRIH (SEQ ID NO: |
| | | | ID NO: 51) | NO: 265) | 282) |
| ZN653_556_578- | ZN6 | ZN5 | LQCEICGY | RVSHLTVHY | LQCEICGYQCRRVSHLT |
| ZN582_395_417_J1 | 53 | 82 | QCR (SEQ | RIH (SEQ ID | VHYRIH (SEQ ID NO: |
| | | | ID NO: 65) | NO: 265) | 283) |
| ZFP91_400_422ZN692 | ZFP9 | ZN5 | LQCEICGFT | RVSHLTVHY | LQCEICGFTCRRVSHLTV |
| 417_43_9- | 1 | 82 | CR (SEQ ID | RIH (SEQ ID | HYRIH (SEQ ID NO: 284) |
| ZN582_395_417_J1 | | | NO: 67) | NO: 265) | |
| ZN787_178_200- | ZN7 | ZN5 | FVCPRCGR | RVSHLTVHY | FVCPRCGRGFSRVSHLTV |
| ZN582_395_417_J1 | 87 | 82 | GFS (SEQ ID | RIH (SEQ ID | HYRIH (SEQ ID NO: 285) |
| | | | NO: 79) | NO: 265) | |
| ZN827_374_396- | ZN8 | ZN5 | FQCPICGLV | RVSHLTVHY | FQCPICGLVIKRVSHLTV |
| ZN582_395_417_J1 | 27 | 82 | IK (SEQ ID | RIH (SEQ ID | HYRIH (SEQ ID NO: 286) |
| | | | NO: 57) | NO: 265) | |
| ZSC20_766_788- | ZSC | ZN5 | YKCLECGK | KFSNSNKHKI | YKCLECGKSFSKFSNSNK |
| ZN595_145_167_J1 | 20 | 95 | SFS (SEQ ID | RH (SEQ ID | HKIRH (SEQ ID NO: 288) |
| | | | NO: 63) | NO: 287) | |
| ZN582_395_417- | ZN5 | ZN5 | YQCKVCGR | KFSNSNKHKI | YQCKVCGRAFKKFSNSN |
| ZN595_145_167_J1 | 82 | 95 | AFK (SEQ | RH (SEQ ID | KHKIRH (SEQ ID NO: |
| | | | ID NO: 77) | NO: 287) | 289) |
| ZN398_483_505- | ZN3 | ZN5 | FSCPQCGID | KFSNSNKHKI | FSCPQCGIDFNKFSNSNK |
| ZN595_145_167_J1 | 98 | 95 | FN (SEQ ID | RH (SEQ ID | HKIRH (SEQ ID NO: 290) |
| | | | NO: 53) | NO: 287) | |
| PATZ1_383_405- | PAT | ZN5 | YSCPVCGL | KFSNSNKHKI | YSCPVCGLRFKKFSNSN |
| ZN595_145_167_J1 | Z1 | 95 | RFK (SEQ | RH (SEQ ID | KHKIRH (SEQ ID NO: |
| | | | ID NO: 51) | NO: 287) | 291) |
| ZN787_178_200- | ZN7 | ZN5 | FVCPRCGR | KFSNSNKHKI | FVCPRCGRGFSKFSNSNK |
| ZN595_145_167_J1 | 87 | 95 | GFS (SEQ ID | RH (SEQ ID | HKIRH SEQ ID NO: 292) |
| | | | NO: 79) | NO: 287) | |
| ZKSC5_430_452- | ZKS | ZN5 | YGCNECGK | KFSNSNKHKI | YGCNECGKNFGKFSNSN |
| ZN595_145_167_J1 | C5 | 95 | NFG (SEQ | RH (SEQ ID | KHKIRH (SEQ ID NO: |
| | | | ID NO: 73) | NO: 287) | 293) |
| ZNF90_481_503- | ZNF | ZN5 | YKCQECDK | KFSNSNKHKI | YKCQECDKAFKKFSNSN |
| ZN595_145_167_J1 | 90 | 95 | AFK (SEQ | RH (SEQ ID | KHKIRH (SEQ ID NO: |
| | | | ID NO: 61) | NO: 287) | 294) |
| ZN597_341_363- | ZN5 | ZN5 | LQCPDCDM | KFSNSNKHKI | LQCPDCDMTFPKFSNSN |
| ZN595_145_167_J1 | 97 | 95 | TFP (SEQ ID | RH (SEQ ID | KHKIRH (SEQ ID NO: |
| | | | NO: 59) | NO: 287) | 295) |
| ZN827_374_396- | ZN8 | ZN5 | FQCPICGLV | KFSNSNKHKI | FQCPICGLVIKKFSNSNK |
| ZN595_145_167_J1 | 27 | 95 | IK (SEQ ID | RH (SEQ ID | HKIRH (SEQ ID NO: 296) |
| | | | NO: 57) | NO: 287) | |
| IKZF3_146_168- | IKZF | ZN5 | FQCNQCGA | KFSNSNKHKI | FQCNQCGASFTKFSNSN |
| ZN595_145_167_J1 | 3 | 95 | SFT (SEQ ID | RH (SEQ ID | KHKIRH (SEQ ID NO: |
| | | | NO: 46) | NO: 287) | 297) |
| ZN595_145_167- | ZN5 | ZN5 | FQCNTCVK | KFSNSNKHKI | FQCNTCVKVFSKFSNSN |
| ZN595_145_167_J1 | 95 | 95 | VFS (SEQ ID | RH (SEQ ID | KHKIRH (SEQ ID NO: |
| | | | NO: 85) | NO: 287) | 298) |
| ZN276_524_546- | ZN2 | ZN5 | LQCEVCGF | KFSNSNKHKI | LQCEVCGFQCRKFSNSN |
| ZN595_145_167_J1 | 76 | 95 | QCR (SEQ | RH (SEQ ID | KHKIRH (SEQ ID NO: |
| | | | ID NO: 71) | NO: 287) | 299) |
| ZNF74_444_466- | ZNF | ZN5 | FKCADCGK | KFSNSNKHKI | FKCADCGKGFSKFSNSN |
| ZN595_145_167_J1 | 74 | 95 | GFS (SEQ ID | RH (SEQ ID | KHKIRH (SEQ ID NO: |
| | | | NO: 75) | NO: 287) | 300) |
| ZN628_120_142- | ZN6 | ZN5 | FICGQCGL | KFSNSNKHKI | FICGQCGLAFKKFSNSNK |
| ZN595_145_167_J1 | 28 | 95 | AFK (SEQ | RH (SEQ ID | HKIRH (SEQ ID NO: 301) |
| | | | ID NO: 49) | NO: 287) | |
| ZF69B_419_441- | ZF69 | ZN5 | YICNVCSK | KFSNSNKHKI | YICNVCSKTFSKFSNSNK |
| ZN595_145_167_J1 | B | 95 | TFS (SEQ ID | RH (SEQ ID | HKIRH (SEQ ID NO: 302) |
| | | | NO: 87) | NO: 287) | |
| ZFP91_400_422ZN692 | ZFP9 | ZN5 | LQCEICGFT | KFSNSNKHKI | LQCEICGFTCRKFSNSNK |
| 417_43_9- | 1 | 95 | CR (SEQ ID | RH (SEQ ID | HKIRH (SEQ ID NO: 303) |
| ZN595_145_167_J1 | | | NO: 67) | NO: 287) | |
| ZN654_25_47- | ZN6 | ZN5 | FACVICGR | KFSNSNKHKI | FACVICGRKFRKFSNSNK |
| ZN595_145_167_J1 | 54 | 95 | KFR (SEQ | RH (SEQ ID | HKIRH (SEQ ID NO: 304) |
| | | | ID NO: 55) | NO: 287) | |
| ZN653_556_578- | ZN6 | ZN5 | LQCEICGY | KFSNSNKHKI | LQCEICGYQCRKFSNSNK |
| ZN595_145_167_J1 | 53 | 95 | QCR (SEQ | RH (SEQ ID | HKIRH (SEQ ID NO: 305) |
| | | | ID NO: 65) | NO: 287) | |
| ZN517_452_474- | ZN5 | ZN5 | YRCRACGR | KFSNSNKHKI | YRCRACGRACSKFSNSN |
| ZN595_145_167_J1 | 17 | 95 | ACS (SEQ | RH (SEQ ID | KHKIRH (SEQ ID NO: |
| | | | ID NO: 83) | NO: 287) | 306) |
| E4F1_220_242- | E4F1 | ZN5 | HECKLCGA | KFSNSNKHKI | HECKLCGASFRKFSNSN |
| ZN595_145_167_J1 | | 95 | SFR (SEQ ID | RH (SEQ ID | KHKIRH (SEQ ID NO: |
| | | | NO: 81) | NO: 287) | 307) |
| IKZF2_140_162- | IKZF | ZN5 | FHCNQCGA | KFSNSNKHKI | FHCNQCGASFTKFSNSN |
| ZN595_145_167_J1 | 2 | 95 | SFT (SEQ ID | RH (SEQ ID | KHKIRH (SEQ ID NO: |
| | | | NO: 69) | NO: 287) | 308) |
| E4F1_220_242- | E4F1 | ZN5 | HECKLCGA | CFSELISHQNI | HECKLCGASFRCFSELIS |
| ZN597_341_363_J1 | | 97 | SFR (SEQ ID | H (SEQ ID NO: | HQNIH (SEQ ID NO: 310) |
| | | | NO: 81) | 309) | |
| ZN827_374_396- | ZN8 | ZN5 | FQCPICGLV | CFSELISHQNI | FQCPICGLVIKCFSELISH |
| ZN597_341_363_J1 | 27 | 97 | IK (SEQ ID | H (SEQ ID NO: | QNIH (SEQ ID NO: 311) |
| | | | NO: 57) | 309) | |
| ZNF74_444_466- | ZNF | ZN5 | FKCADCGK | CFSELISHQNI | FKCADCGKGFSCFSELIS |
| ZN597_341_363_J1 | 74 | 97 | GFS (SEQ ID | H (SEQ ID NO: | HQNIH (SEQ ID NO: 312) |
| | | | NO: 75) | 309) | |
| ZNF90_481_503- | ZNF | ZN5 | YKCQECDK | CFSELISHQNI | YKCQECDKAFKCFSELIS |
| ZN597_341_363_J1 | 90 | 97 | AFK (SEQ | H (SEQ ID NO: | HQNIH (SEQ ID NO: 313) |
| | | | ID NO: 61) | 309) | |
| ZN787_178_200- | ZN7 | ZN5 | FVCPRCGR | CFSELISHQNI | FVCPRCGRGFSCFSELISH |
| ZN597_341_363_J1 | 87 | 97 | GFS (SEQ ID | H (SEQ ID NO: | QNIH (SEQ ID NO: 314) |
| | | | NO: 79) | 309) | |
| IKZF3_146_168- | IKZF | ZN5 | FQCNQCGA | CFSELISHQNI | FQCNQCGASFTCFSELIS |
| ZN597_341_363_J1 | 3 | 97 | SFT (SEQ ID | H (SEQ ID NO: | HQNIH (SEQ ID NO: 315) |
| | | | NO: 46) | 309) | |
| ZN582_395_417- | ZN5 | ZN5 | YQCKVCGR | CFSELISHQNI | YQCKVCGRAFKCFSELIS |
| ZN597_341_363_J1 | 82 | 97 | AFK (SEQ | H (SEQ ID NO: | HQNIH (SEQ ID NO: 316) |
| | | | ID NO: 77) | 309) | |
| ZN654_25_47- | ZN6 | ZN5 | FACVICGR | CFSELISHQNI | FACVICGRKFRCFSELISH |
| ZN597_341_363_J1 | 54 | 97 | KFR (SEQ | H (SEQ ID NO: | QNIH (SEQ ID NO: 317) |
| | | | ID NO: 55) | 309) | |
| ZN597_341_363- | ZN5 | ZN5 | LQCPDCDM | CFSELISHQNI | LQCPDCDMTFPCFSELIS |
| ZN597_341_363_J1 | 97 | 97 | TFP (SEQ ID | H (SEQ ID NO: | HQNIH (SEQ ID NO: 318) |
| | | | NO: 59) | 309) | |
| ZN595_145_167- | ZN5 | ZN5 | FQCNTCVK | CFSELISHQNI | FQCNTCVK VFSCFSELIS |
| ZN597_341_363_J1 | 95 | 97 | VFS (SEQ ID | H (SEQ ID NO: | HQNIH (SEQ ID NO: 319) |
| | | | NO: 85) | 309) | |
| ZN628_120_142- | ZN6 | ZN5 | FICGQCGL | CFSELISHQNI | FICGQCGL AFK CFSELISH |
| ZN597_341_363_J1 | 28 | 97 | AFK (SEQ | H (SEQ ID NO: | QNIH (SEQ ID NO: 320) |
| | | | ID NO: 49) | 309) | |
| ZN398_483_505- | ZN3 | ZN5 | FSCPQCGID | CFSELISHQNI | FSCPQCGIDFNCFSELISH |
| ZN597_341_363_J1 | 98 | 97 | FN (SEQ ID | H (SEQ ID NO: | QNIH (SEQ ID NO: 321) |
| | | | NO: 53) | 309) | |
| ZN517_452_474- | ZN5 | ZN5 | YRCRACGR | CFSELISHQNI | YRCRACGRACSCFSELIS |
| ZN597_341_363_J1 | 17 | 97 | ACS (SEQ | H (SEQ ID NO: | HQNIH (SEQ ID NO: 322) |
| | | | ID NO: 83) | 309) | |
| ZF69B_419_441- | ZF69 | ZN5 | YICNVCSK | CFSELISHQNI | YICNVCSKTFSCFSELISH |
| ZN597_341_363_J1 | B | 97 | TFS (SEQ ID | H (SEQ ID NO: | QNIH (SEQ ID NO: 323) |
| | | | NO: 87) | 309) | |
| PATZ1_383_405- | PAT | ZN5 | YSCPVCGL | CFSELISHQNI | YSCPVCGLRFKCFSELIS |
| ZN597_341_363_J1 | Z1 | 97 | RFK (SEQ | H (SEQ ID NO: | HQNIH (SEQ ID NO: 324) |
| | | | ID NO: 51) | 309) | |
| ZN653_556_578- | ZN6 | ZN5 | LQCEICGY | CFSELISHQNI | LQCEICGYQCRCFSELIS |
| ZN597_341_363_J1 | 53 | 97 | QCR (SEQ | H (SEQ ID NO: | HQNIH (SEQ ID NO: 325) |
| | | | ID NO: 65) | 309) | |
| ZKSC5_430_452- | ZKS | ZN5 | YGCNECGK | CFSELISHQNI | YGCNECGKNFGCFSELIS |
| ZN597_341_363_J1 | C5 | 97 | NFG (SEQ | H (SEQ ID NO: | HQNIH (SEQ ID NO: 326) |
| | | | ID NO: 73) | 309) | |
| IKZF2_140_162- | IKZF | ZN5 | FHCNQCGA | CFSELISHQNI | FHCNQCGASFTCFSELIS |
| ZN597_341_363_J1 | 2 | 97 | SFT (SEQ ID | H (SEQ ID NO: | HQNIH (SEQ ID NO: 327) |
| | | | NO: 69) | 309) | |
| ZSC20_766_788- | zsc | ZN5 | YKCLECGK | CFSELISHQNI | YKCLECGKSFSCFSELIS |
| ZN597_341_363_J1 | 20 | 97 | SFS (SEQ ID | H (SEQ ID NO: | HQNIH (SEQ ID NO: 328) |
| | | | NO: 63) | 309) | |
| ZFP91_400_422ZN692 | ZFP9 | ZN5 | LQCEICGFT | CFSELISHQNI | LQCEICGFTCRCFSELISH |
| 417_43_9- | 1 | 97 | CR (SEQ ID | H (SEQ ID NO: | QNIH (SEQ ID NO: 329) |
| ZN597_341_363_J1 | | | NO: 67) | 309) | |
| ZN276_524_546- | ZN2 | ZN5 | LQCEVCGF | CFSELISHQNI | LQCEVCGFQCRCFSELIS |
| ZN597_341_363_J1 | 76 | 97 | QCR (SEQ | H (SEQ ID NO: | HQNIH (SEQ ID NO: 330) |
| | | | ID NO: 71) | 309) | |
| PATZ1_383_405- | PAT | ZN6 | YSCPVCGL | WSSHYQYHL | YSCPVCGLRFKWSSHYQ |
| ZN628_120_142_J1 | Z1 | 28 | RFK (SEQ | RQH (SEQ ID | YHLRQH (SEQ ID NO: |
| | | | ID NO: 51) | NO: 331) | 332) |
| ZN398_483_505- | ZN3 | ZN6 | FSCPQCGID | WSSHYQYHL | FSCPQCGIDFNWSSHYQ |
| ZN628_120_142_J1 | 98 | 28 | FN (SEQ ID | RQH (SEQ ID | YHLRQH (SEQ ID NO: |
| | | | NO: 53) | NO: 331) | 333) |
| ZN827_374_396- | ZN8 | ZN6 | FQCPICGLV | WSSHYQYHL | FQCPICGLVIKWSSHYQY |
| ZN628_120_142_J1 | 27 | 28 | IK (SEQ ID | RQH (SEQ ID | HLRQH (SEQ ID NO: 334) |
| | | | NO: 57) | NO: 331) | |
| ZN787_178_200- | ZN7 | ZN6 | FVCPRCGR | WSSHYQYHL | FVCPRCGRGFSWSSHYQ |
| ZN628_120_142_J1 | 87 | 28 | GFS (SEQ ID | RQH (SEQ ID | YHLRQH (SEQ ID NO: |
| | | | NO: 79) | NO: 331) | 335) |
| ZN276_524_546- | ZN2 | ZN6 | LQCEVCGF | WSSHYQYHL | LQCEVCGFQCRWSSHYQ |
| ZN628_120_142_J1 | 76 | 28 | QCR (SEQ | RQH (SEQ ID | YHLRQH (SEQ ID NO: |
| | | | ID NO: 71) | NO: 331) | 336) |
| ZFP91_400_422ZN692 | ZFP9 | ZN6 | LQCEICGFT | WSSHYQYHL | LQCEICGFTCRWSSHYQ |
| 417_43_9- | 1 | 28 | CR (SEQ ID | RQH (SEQ ID | YHLRQH (SEQ ID NO: |
| ZN628_120_142_J1 | | | NO: 67) | NO: 331) | 337) |
| ZNF74_444_466- | ZNF | ZN6 | FKCADCGK | WSSHYQYHL | FKCADCGKGFSWSSHYQ |
| ZN628_120_142_J1 | 74 | 28 | GFS (SEQ ID | RQH (SEQ ID | YHLRQH (SEQ ID NO: |
| | | | NO: 75) | NO: 331) | 338) |
| ZN595_145_167- | ZN5 | ZN6 | FQCNTCVK | WSSHYQYHL | FQCNTCVKVFSWSSHYQ |
| ZN628_120_142_J1 | 95 | 28 | VFS (SEQ ID | RQH (SEQ ID | YHLRQH (SEQ ID NO: |
| | | | NO: 85) | NO: 331) | 339) |
| ZN653_556_578- | ZN6 | ZN6 | LQCEICGY | WSSHYQYHL | LQCEICGYQCRWSSHYQ |
| ZN628_120_142_J1 | 53 | 28 | QCR (SEQ | RQH (SEQ ID | YHLRQH (SEQ ID NO: |
| | | | ID NO: 65) | NO: 331) | 340) |
| ZKSC5_430_452- | ZKS | ZN6 | YGCNECGK | WSSHYQYHL | YGCNECGKNFGWSSHY |
| ZN628_120_142_J1 | C5 | 28 | NFG (SEQ | RQH (SEQ ID | QYHLRQH (SEQ ID NO: |
| | | | ID NO: 73) | NO: 331) | 341) |
| E4F1_220_242- | E4F1 | ZN6 | HECKLCGA | WSSHYQYHL | HECKLCGASFRWSSHYQ |
| ZN628_120_142_J1 | | 28 | SFR (SEQ ID | RQH (SEQ ID | YHLRQH (SEQ ID NO: |
| | | | NO: 81) | NO: 331) | 342) |
| ZNF90_481_503- | ZNF | ZN6 | YKCQECDK | WSSHYQYHL | YKCQECDKAFKWSSHY |
| ZN628_120_142_J1 | 90 | 28 | AFK (SEQ | RQH (SEQ ID | QYHLRQH (SEQ ID NO: |
| | | | ID NO: 61) | NO: 331) | 343) |
| ZN628_120_142- | ZN6 | ZN6 | FICGQCGL | WSSHYQYHL | FICGQCGLAFKWSSHYQ |
| ZN628_120_142_J1 | 28 | 28 | AFK (SEQ | RQH (SEQ ID | YHLRQH (SEQ ID NO: |
| | | | ID NO: 49) | NO: 331) | 344) |
| ZSC20_766_788- | ZSC | ZN6 | YKCLECGK | WSSHYQYHL | YKCLECGKSFSWSSHYQ |
| ZN628_120_142_J1 | 20 | 28 | SFS (SEQ ID | RQH (SEQ ID | YHLRQH (SEQ ID NO: |
| | | | NO: 63) | NO: 331) | 345) |
| ZN597_341_363- | ZN5 | ZN6 | LQCPDCDM | WSSHYQYHL | LQCPDCDMTFPWSSHYQ |
| ZN628_120_142_J1 | 97 | 28 | TFP (SEQ ID | RQH (SEQ ID | YHLRQH (SEQ ID NO: |
| | | | NO: 59) | NO: 331) | 346) |
| ZN654_25_47- | ZN6 | ZN6 | FACVICGR | WSSHYQYHL | FACVICGRKFRWSSHYQ |
| ZN628_120_142_J1 | 54 | 28 | KFR (SEQ | RQH (SEQ ID | YHLRQH (SEQ ID NO: |
| | | | ID NO: 55) | NO: 331) | 347) |
| ZN517_452_474- | ZN5 | ZN6 | YRCRACGR | WSSHYQYHL | YRCRACGRACSWSSHYQ |
| ZN628_120_142_J1 | 17 | 28 | ACS (SEQ | RQH (SEQ ID | YHLRQH (SEQ ID NO: |
| | | | ID NO: 83) | NO: 331) | 348) |
| IKZF3_146_168- | IKZF | ZN6 | FQCNQCGA | WSSHYQYHL | FQCNQCGASFTWSSHYQ |
| ZN628_120_142_J1 | 3 | 28 | SFT (SEQ ID | RQH (SEQ ID | YHLRQH (SEQ ID NO: |
| | | | NO: 46) | NO: 331) | 349) |
| IKZF2_140_162- | IKZF | ZN6 | FHCNQCGA | WSSHYQYHL | FHCNQCGASFTWSSHYQ |
| ZN628_120_142_J1 | 2 | 28 | SFT (SEQ ID | RQH (SEQ ID | YHLRQH (SEQ ID NO: |
| | | | NO: 69) | NO: 331) | 350) |
| ZF69B_419_441- | ZF69 | ZN6 | YICNVCSK | WSSHYQYHL | YICNVCSKTFSWSSHYQ |
| ZN628_120_142_J1 | B | 28 | TFS (SEQ ID | RQH (SEQ ID | YHLRQH (SEQ ID NO: |
| | | | NO: 87) | NO: 331) | 351) |
| ZN582_395_417- | ZN5 | ZN6 | YQCKVCGR | WSSHYQYHL | YQCKVCGRAFKWSSHY |
| ZN628_120_142_J1 | 82 | 28 | AFK (SEQ | RQH (SEQ ID | QYHLRQH (SEQ ID NO: |
| | | | ID NO: 77) | NO: 331) | 352) |
| ZN654_25_47- | ZN6 | ZN6 | FACVICGR | QRASLNWH | FACVICGRKFRQRASLN |
| ZN653_556_578_J1 | 54 | 53 | KFR (SEQ | MKKH (SEQ | WHMKKH (SEQ ID NO: |
| | | | ID NO: 55) | ID NO: 353) | 354) |
| ZNF90_481_503- | ZNF | ZN6 | YKCQECDK | QRASLNWH | YKCQECDKAFKQRASLN |
| ZN653_556_578_J1 | 90 | 53 | AFK (SEQ | MKKH (SEQ | WHMKKH (SEQ ID NO: |
| | | | ID NO: 61) | ID NO: 353) | 355) |
| ZN595_145_167- | ZN5 | ZN6 | FQCNTCVK | QRASLNWH | FQCNTCVKVFSQRASLN |
| ZN653_556_578_J1 | 95 | 53 | VFS (SEQ ID | MKKH (SEQ | WHMKKH (SEQ ID NO: |
| | | | NO: 85) | ID NO: 353) | 356) |
| ZN582_395_417- | ZN5 | ZN6 | YQCKVCGR | QRASLNWH | YQCKVCGRAFKQRASLN |
| ZN653_556_578_J1 | 82 | 53 | AFK (SEQ | MKKH (SEQ | WHMKKH (SEQ ID NO: |
| | | | ID NO: 77) | ID NO: 353) | 357) |
| ZN827_374_396- | ZN8 | ZN6 | FQCPICGLV | QRASLNWH | FQCPICGLVIKQRASLNW |
| ZN653_556_578_J1 | 27 | 53 | IK (SEQ ID | MKKH (SEQ | HMKKH (SEQ ID NO: 358) |
| | | | NO: 57) | ID NO: 353) | |
| IKZF3_146_168- | IKZF | ZN6 | FQCNQCGA | QRASLNWH | FQCNQCGASFTQRASLN |
| ZN653_556_578_J1 | 3 | 53 | SFT (SEQ ID | MKKH (SEQ | WHMKKH (SEQ ID NO: |
| | | | NO: 46) | ID NO: 353) | 359) |
| ZN787_178_200- | ZN7 | ZN6 | FVCPRCGR | QRASLNWH | FVCPRCGRGFSQRASLN |
| ZN653_556_578_J1 | 87 | 53 | GFS (SEQ ID | MKKH (SEQ | WHMKKH (SEQ ID NO: |
| | | | NO: 79) | ID NO: 353) | 360) |
| ZN517_452_474- | ZN5 | ZN6 | YRCRACGR | QRASLNWH | YRCRACGRACSQRASLN |
| ZN653_556_578_J1 | 17 | 53 | ACS (SEQ | MKKH (SEQ | WHMKKH (SEQ ID NO: |
| | | | ID NO: 83) | ID NO: 353) | 361) |
| IKZF2_140_162- | IKZF | ZN6 | FHCNQCGA | QRASLNWH | FHCNQCGASFTQRASLN |
| ZN653_556_578_J1 | 2 | 53 | SFT (SEQ ID | MKKH (SEQ | WHMKKH (SEQ ID NO: |
| | | | NO: 69) | ID NO: 353) | 362) |
| ZNF74_444_466- | ZNF | ZN6 | FKCADCGK | QRASLNWH | FKCADCGKGFSQRASLN |
| ZN653_556_578_J1 | 74 | 53 | GFS (SEQ ID | MKKH (SEQ | WHMKKH (SEQ ID NO: |
| | | | NO: 75) | ID NO: 353) | 363) |
| ZFP91_400_422ZN692 | ZFP9 | ZN6 | LQCEICGFT | QRASLNWH | LQCEICGFTCRQRASLN |
| 417_43_9- | 1 | 53 | CR (SEQ ID | MKKH (SEQ | WHMKKH (SEQ ID NO: |
| ZN653_556_578_J1 | | | NO: 67) | ID NO: 353) | 364) |
| E4F1_220_242- | E4F1 | ZN6 | HECKLCGA | QRASLNWH | HECKLCGASFRQRASLN |
| ZN653_556_578_J1 | | 53 | SFR (SEQ ID | MKKH (SEQ | WHMKKH (SEQ ID NO: |
| | | | NO: 81) | ID NO: 353) | 365) |
| ZN653_556_578- | ZN6 | ZN6 | LQCEICGY | QRASLNWH | LQCEICGYQCRQRASLN |
| ZN653_556_578_J1 | 53 | 53 | QCR (SEQ | MKKH (SEQ | WHMKKH (SEQ ID NO: |
| | | | ID NO: 65) | ID NO: 353) | 366) |
| ZKSC5_430_452- | ZKS | ZN6 | YGCNECGK | QRASLNWH | YGCNECGKNFGQRASLN |
| ZN653_556_578_J1 | C5 | 53 | NFG (SEQ | MKKH (SEQ | WHMKKH (SEQ ID NO: |
| | | | ID NO: 73) | ID NO: 353) | 367) |
| ZN398_483_505- | ZN3 | ZN6 | FSCPQCGID | QRASLNWH | FSCPQCGIDFNQRASLN |
| ZN653_556_578_J1 | 98 | 53 | FN (SEQ ID | MKKH (SEQ | WHMKKH (SEQ ID NO: |
| | | | NO: 53) | ID NO: 353) | 368) |
| ZN597_341_363- | ZN5 | ZN6 | LQCPDCDM | QRASLNWH | LQCPDCDMTFPQRASLN |
| ZN653_556_578_J1 | 97 | 53 | TFP (SEQ ID | MKKH (SEQ | WHMKKH (SEQ ID NO: |
| | | | NO: 59) | ID NO: 353) | 369) |
| ZN628_120_142- | ZN6 | ZN6 | FICGQCGL | QRASLNWH | FICGQCGLAFKQRASLN |
| ZN653_556_578_J1 | 28 | 53 | AFK (SEQ | MKKH (SEQ | WHMKKH (SEQ ID NO: |
| | | | ID NO: 49) | ID NO: 353) | 370) |
| ZN276_524_546- | ZN2 | ZN6 | LQCEVCGF | QRASLNWH | LQCEVCGFQCRQRASLN |
| ZN653_556_578_J1 | 76 | 53 | QCR (SEQ | MKKH (SEQ | WHMKKH (SEQ ID NO: |
| | | | ID NO: 71) | ID NO: 353) | 371) |
| ZF69B_419_441- | ZF69 | ZN6 | YICNVCSK | QRASLNWH | YICNVCSKTFSQRASLN |
| ZN653_556_578_J1 | B | 53 | TFS (SEQ ID | MKKH (SEQ | WHMKKH (SEQ ID NO: |
| | | | NO: 87) | ID NO: 353) | 372) |
| PATZ1_383_405- | PAT | ZN6 | YSCPVCGL | QRASLNWH | YSCPVCGLRFKQRASLN |
| ZN653_556_578_J1 | Z1 | 53 | RFK (SEQ | MKKH (SEQ | WHMKKH (SEQ ID NO: |
| | | | ID NO: 51) | ID NO: 353) | 373) |
| ZSC20_766_788- | ZSC | ZN6 | YKCLECGK | QRASLNWH | YKCLECGKSFSQRASLN |
| ZN653_556_578_J1 | 20 | 53 | SFS (SEQ ID | MKKH (SEQ | WHMKKH (SEQ ID NO: |
| | | | NO: 63) | ID NO: 353) | 374) |
| ZN276_524_546- | ZN2 | ZN6 | LQCEVCGF | NRGLMQKHL | LQCEVCGFQCRNRGLMQ |
| ZN654_25_47_J1 | 76 | 54 | QCR (SEQ | KNH (SEQ ID | KHLKNH (SEQ ID NO: |
| | | | ID NO: 71) | NO: 375) | 376) |
| ZN595_145_167- | ZN5 | ZN6 | FQCNTCVK | NRGLMQKHL | FQCNTCVK VFSNRGLMQ |
| ZN654_25_47_J1 | 95 | 54 | VFS (SEQ ID | KNH (SEQ ID | KHLKNH (SEQ ID NO: |
| | | | NO: 85) | NO: 375) | 377) |
| ZFP91_400_422Z | ZFP9 | ZN6 | LQCEICGFT | NRGLMQKHL | LQCEICGFTCRNRGLMQ |
| N692417_43_9- | 1 | 54 | CR (SEQ ID | KNH (SEQ ID | KHLKNH (SEQ ID NO: |
| ZN654_25_47_J1 | | | NO: 67) | NO: 375) | 378) |
| ZN597_341_363- | ZN5 | ZN6 | LQCPDCDM | NRGLMQKHL | LQCPDCDMTFPNRGLMQ |
| ZN654_25_47_J1 | 97 | 54 | TFP (SEQ ID | KNH (SEQ ID | KHLKNH (SEQ ID NO: |
| | | | NO: 59) | NO: 375) | 379) |
| E4F1_220_242- | E4F1 | ZN6 | HECKLCGA | NRGLMQKHL | HECKLCGASFRNRGLMQ |
| ZN654_25_47_J1 | | 54 | SFR (SEQ ID | KNH (SEQ ID | KHLKNH (SEQ ID NO: |
| | | | NO: 81) | NO: 375) | 380) |
| ZN628_120_142- | ZN6 | ZN6 | FICGQCGL | NRGLMQKHL | FICGQCGLAFKNRGLMQ |
| ZN654_25_47_J1 | 28 | 54 | AFK (SEQ | KNH (SEQ ID | KHLKNH (SEQ ID NO: |
| | | | ID NO: 49) | NO: 375) | 381) |
| ZN398_483_505- | ZN3 | ZN6 | FSCPQCGID | NRGLMQKHL | FSCPQCGIDFNNRGLMQ |
| ZN654_25_47_J1 | 98 | 54 | FN (SEQ ID | KNH (SEQ ID | KHLKNH (SEQ ID NO: |
| | | | NO: 53) | NO: 375) | 382) |
| ZKSC5_430_452- | ZKS | ZN6 | YGCNECGK | NRGLMQKHL | YGCNECGKNFGNRGLM |
| ZN654_25_47_J1 | C5 | 54 | NFG (SEQ | KNH (SEQ ID | QKHLKNH (SEQ ID NO: |
| | | | ID NO: 73) | NO: 375) | 383) |
| ZN517_452_474- | ZN5 | ZN6 | YRCRACGR | NRGLMQKHL | YRCRACGRACSNRGLM |
| ZN654_25_47_J1 | 17 | 54 | ACS (SEQ | KNH (SEQ ID | QKHLKNH (SEQ ID NO: |
| | | | ID NO: 83) | NO: 375) | 384) |
| ZSC20_766_788- | ZSC | ZN6 | YKCLECGK | NRGLMQKHL | YKCLECGKSFSNRGLMQ |
| ZN654_25_47_J1 | 20 | 54 | SFS (SEQ ID | KNH (SEQ ID | KHLKNH (SEQ ID NO: |
| | | | NO: 63) | NO: 375) | 385) |
| ZNF74_444_466- | ZNF | ZN6 | FKCADCGK | NRGLMQKHL | FKCADCGKGFSNRGLMQ |
| ZN654_25_47_J1 | 74 | 54 | GFS (SEQ ID | KNH (SEQ ID | KHLKNH (SEQ ID NO: |
| | | | NO: 75) | NO: 375) | 386) |
| ZN827_374_396- | ZN8 | ZN6 | FQCPICGLV | NRGLMQKHL | FQCPICGLVIKNRGLMQK |
| ZN654_25_47_J1 | 27 | 54 | IK (SEQ ID | KNH (SEQ ID | HLKNH (SEQ ID NO: 387) |
| | | | NO: 57) | NO: 375) | |
| ZN582_395_417- | ZN5 | ZN6 | YQCKVCGR | NRGLMQKHL | YQCKVCGRAFKNRGLM |
| ZN654_25_47_J1 | 82 | 54 | AFK (SEQ | KNH (SEQ ID | QKHLKNH (SEQ ID NO: |
| | | | ID NO: 77) | NO: 375) | 388) |
| ZN787_178_200- | ZN7 | ZN6 | FVCPRCGR | NRGLMQKHL | FVCPRCGRGFSNRGLMQ |
| ZN654_25_47_J1 | 87 | 54 | GFS (SEQ ID | KNH (SEQ ID | KHLKNH (SEQ ID NO: |
| | | | NO: 79) | NO: 375) | 389) |
| IKZF3_146_168- | IKZF | ZN6 | FQCNQCGA | NRGLMQKHL | FQCNQCGASFTNRGLMQ |
| ZN654_25_47_J1 | 3 | 54 | SFT (SEQ ID | KNH (SEQ ID | KHLKNH (SEQ ID NO: |
| | | | NO: 46) | NO: 375) | 390) |
| IKZF2_140_162- | IKZF | ZN6 | FHCNQCGA | NRGLMQKHL | FHCNQCGASFTNRGLMQ |
| ZN654_25_47_J1 | 2 | 54 | SFT (SEQ ID | KNH (SEQ ID | KHLKNH (SEQ ID NO: |
| | | | NO: 69) | NO: 375) | 391) |
| ZN653_556_578- | ZN6 | ZN6 | LQCEICGY | NRGLMQKHL | LQCEICGYQCRNRGLMQ |
| ZN654_25_47_J1 | 53 | 54 | QCR (SEQ | KNH (SEQ ID | KHLKNH (SEQ ID NO: |
| | | | ID NO: 65) | NO: 375) | 392) |
| ZF69B_419_441- | ZF69 | ZN6 | YICNVCSK | NRGLMQKHL | YICNVCSKTFSNRGLMQ |
| ZN654_25_47_J1 | B | 54 | TFS (SEQ ID | KNH (SEQ ID | KHLKNH (SEQ ID NO: |
| | | | NO: 87) | NO: 375) | 393) |
| ZN654_25_47- | ZN6 | ZN6 | FACVICGR | NRGLMQKHL | FACVICGRKFRNRGLMQ |
| ZN654_25_47_J1 | 54 | 54 | KFR (SEQ | KNH (SEQ ID | KHLKNH (SEQ ID NO: |
| | | | ID NO: 55) | NO: 375) | 394) |
| PATZ1_383_405- | PAT | ZN6 | YSCPVCGL | NRGLMQKHL | YSCPVCGLRFKNRGLMQ |
| ZN654_25_47_J1 | Z1 | 54 | RFK (SEQ | KNH (SEQ ID | KHLKNH (SEQ ID NO: |
| | | | ID NO: 51) | NO: 375) | 395) |
| ZNF90_481_503- | ZNF | ZN6 | YKCQECDK | NRGLMQKHL | YKCQECDKAFKNRGLM |
| ZN654_25_47_J1 | 90 | 54 | AFK (SEQ | KNH (SEQ ID | QKHLKNH (SEQ ID NO: |
| | | | ID NO: 61) | NO: 375) | 396) |
| IKZF3_146_168- | IKZF | ZN6 | FQCNQCGA | QKASLNWHQ | FQCNQCGASFTQKASLN |
| ZN692_417_439_J1 | 3 | 92 | SFT (SEQ ID | RKH (SEQ ID | WHQRKH (SEQ ID NO: |
| | | | NO: 46) | NO: 397) | 398) |
| ZN276_524_546- | ZN2 | ZN6 | LQCEVCGF | QKASLNWHQ | LQCEVCGFQCRQKASLN |
| ZN692_417_439_J1 | 76 | 92 | QCR (SEQ | RKH (SEQ ID | WHQRKH (SEQ ID NO: |
| | | | ID NO: 71) | NO: 397) | 399) |
| ZNF74_444_466- | ZNF | ZN6 | FKCADCGK | QKASLNWHQ | FKCADCGKGFSQKASLN |
| ZN692_417_439_J1 | 74 | 92 | GFS (SEQ ID | RKH (SEQ ID | WHQRKH (SEQ ID NO: |
| | | | NO: 75) | NO: 397) | 400) |
| ZN654_25_47- | ZN6 | ZN6 | FACVICGR | QKASLNWHQ | FACVICGRKFRQKASLN |
| ZN692_417_439_J1 | 54 | 92 | KFR (SEQ | RKH (SEQ ID | WHQRKH (SEQ ID NO; |
| | | | ID NO: 55) | NO: 397) | 401) |
| ZN787_178_200- | ZN7 | ZN6 | FVCPRCGR | QKASLNWHQ | FVCPRCGRGFSQKASLN |
| ZN692_417_439_J1 | 87 | 92 | GFS (SEQ ID | RKH (SEQ ID | WHQRKH (SEQ ID NO: |
| | | | NO: 79) | NO: 397) | 402) |
| ZFP91_400_422ZN692 | ZFP9 | ZN6 | LQCEICGFT | QKASLNWHQ | LQCEICGFTCRQKASLN |
| 417_43_9- | 1 | 92 | CR (SEQ ID | RKH (SEQ ID | WHQRKH (SEQ ID NO: |
| ZN692_417_439_J1 | | | NO: 67) | NO: 397) | 403) |
| ZN628_120_142- | ZN6 | ZN6 | FICGQCGL | QKASLNWHQ | FICGQCGLAFKQKASLN |
| ZN692_417_439_J1 | 28 | 92 | AFK (SEQ | RKH (SEQ ID | WHQRKH (SEQ ID NO: |
| | | | ID NO: 49) | NO: 397) | 404) |
| ZN653_556_578- | ZN6 | ZN6 | LQCEICGY | QKASLNWHQ | LQCEICGYQCRQKASLN |
| ZN692_417_439_J1 | 53 | 92 | QCR (SEQ | RKH (SEQ ID | WHQRKH (SEQ ID NO: |
| | | | ID NO: 65) | NO: 397) | 405) |
| ZF69B_419_441- | ZF69 | ZN6 | YICNVCSK | QKASLNWHQ | YICNVCSKTFSQKASLN |
| ZN692_417_439_J1 | B | 92 | TFS (SEQ ID | RKH (SEQ ID | WHQRKH (SEQ ID NO: |
| | | | NO: 87) | NO: 397) | 406) |
| E4F1_220_242- | E4F1 | ZN6 | HECKLCGA | QKASLNWHQ | HECKLCGASFRQKASLN |
| ZN692_417_439_J1 | | 92 | SFR (SEQ ID | RKH (SEQ ID | WHQRKH (SEQ ID NO: |
| | | | NO: 81) | NO: 397) | 407) |
| ZN597_341_363- | ZN5 | ZN6 | LQCPDCDM | QKASLNWHQ | LQCPDCDMTFPQKASLN |
| ZN692_417_439_J1 | 97 | 92 | TFP (SEQ ID | RKH (SEQ ID | WHQRKH (SEQ ID NO: |
| | | | NO: 59) | NO: 397) | 408) |
| ZSC20_766_788- | ZSC | ZN6 | YKCLECGK | QKASLNWHQ | YKCLECGKSFSQKASLN |
| ZN692_417_439_J1 | 20 | 92 | SFS (SEQ ID | RKH (SEQ ID | WHQRKH (SEQ ID NO: |
| | | | NO: 63) | NO: 397) | 409) |
| ZKSC5_430_452- | ZKS | ZN6 | YGCNECGK | QKASLNWHQ | YGCNECGKNFGQKASLN |
| ZN692_417_439_J1 | C5 | 92 | NFG (SEQ | RKH (SEQ ID | WHQRKH (SEQ ID NO: |
| | | | ID NO: 73) | NO: 397) | 410) |
| ZNF90_481_503- | ZNF | ZN6 | YKCQECDK | QKASLNWHQ | YKCQECDKAFKQKASLN |
| ZN692_417_439_J1 | 90 | 92 | AFK (SEQ | RKH (SEQ ID | WHQRKH (SEQ ID NO: |
| | | | ID NO: 61) | NO: 397) | 411) |
| PATZ1_383_405- | PAT | ZN6 | YSCPVCGL | QKASLNWHQ | YSCPVCGLRFKQKASLN |
| ZN692_417_439_J1 | Z1 | 92 | RFK (SEQ | RKH (SEQ ID | WHQRKH (SEQ ID NO: |
| | | | ID NO: 51) | NO: 397) | 412) |
| ZN595_145_167- | ZN5 | ZN6 | FQCNTCVK | QKASLNWHQ | FQCNTCVK VFSQKASLN |
| ZN692_417_439_J1 | 95 | 92 | VFS (SEQ ID | RKH (SEQ ID | WHQRKH (SEQ ID NO: |
| | | | NO: 85) | NO: 397) | 413) |
| ZN517_452_474- | ZN5 | ZN6 | YRCRACGR | QKASLNWHQ | YRCRACGRACSQKASLN |
| ZN692_417_439_J1 | 17 | 92 | ACS (SEQ | RKH (SEQ ID | WHQRKH (SEQ ID NO: |
| | | | ID NO: 83) | NO: 397) | 414) |
| ZN582_395_417- | ZN5 | ZN6 | YQCKVCGR | QKASLNWHQ | YQCKVCGRAFKQKASLN |
| ZN692_417_439_J1 | 82 | 92 | AFK (SEQ | RKH (SEQ ID | WHQRKH (SEQ ID NO: |
| | | | ID NO: 77) | NO: 397) | 415) |
| ZN398_483_505- | ZN3 | ZN6 | FSCPQCGID | QKASLNWHQ | FSCPQCGIDFNQKASLN |
| ZN692_417_439_J1 | 98 | 92 | FN (SEQ ID | RKH (SEQ ID | WHQRKH (SEQ ID NO: |
| | | | NO: 53) | NO: 397) | 416) |
| ZN827_374_396- | ZN8 | ZN6 | FQCPICGLV | QKASLNWHQ | FQCPICGLVIKQKASLNW |
| ZN692_417_439_J1 | 27 | 92 | IK (SEQ ID | RKH (SEQ ID | HQRKH (SEQ ID NO: 417) |
| | | | NO: 57) | NO: 397) | |
| IKZF2_140_162- | IKZF | ZN6 | FHCNQCGA | QKASLNWHQ | FHCNQCGASFTQKASLN |
| ZN692_417_439_J1 | 2 | 92 | SFT (SEQ ID | RKH (SEQ ID | WHQRKH (SEQ ID NO: |
| | | | NO: 69) | NO: 397) | 418) |
| ZN582_395_417- | ZN5 | ZN7 | YQCKVCGR | QPKSLARHL | YQCKVCGRAFKQPKSLA |
| ZN787_178_200_J1 | 82 | 87 | AFK (SEQ | RLH (SEQ ID | RHLRLH (SEQ ID NO: |
| | | | ID NO: 77) | NO: 419) | 420) |
| IKZF3_146_168- | IKZF | ZN7 | FQCNQCGA | QPKSLARHL | FQCNQCGASFTQPKSLA |
| ZN787_178_200_J1 | 3 | 87 | SFT (SEQ ID | RLH (SEQ ID | RHLRLH (SEQ ID NO: |
| | | | NO: 46) | NO: 419) | 421) |
| ZN628_120_142- | ZN6 | ZN7 | FICGQCGL | QPKSLARHL | FICGQCGLAFKQPKSLAR |
| ZN787_178_200_J1 | 28 | 87 | AFK (SEQ | RLH (SEQ ID | HLRLH (SEQ ID NO: 422) |
| | | | ID NO: 49) | NO: 419) | |
| ZN517_452_474- | ZN5 | ZN7 | YRCRACGR | QPKSLARHL | YRCRACGRACSQPKSLA |
| ZN787_178_200_J1 | 17 | 87 | ACS (SEQ | RLH (SEQ ID | RHLRLH (SEQ ID NO: |
| | | | ID NO: 83) | NO: 419) | 423) |
| ZN827_374_396- | ZN8 | ZN7 | FQCPICGLV | QPKSLARHL | FQCPICGLVIKQPKSLAR |
| ZN787_178_200_J1 | 27 | 87 | IK (SEQ ID | RLH (SEQ ID | HLRLH (SEQ ID NO: 424) |
| | | | NO: 57) | NO: 419) | |
| ZN398_483_505- | ZN3 | ZN7 | FSCPQCGID | QPKSLARHL | FSCPQCGIDFNQPKSLAR |
| ZN787_178_200_J1 | 98 | 87 | FN (SEQ ID | RLH (SEQ ID | HLRLH (SEQ ID NO: 425) |
| | | | NO: 53) | NO: 419) | |
| ZFP91_400_422ZN692 | ZFP9 | ZN7 | LQCEICGFT | QPKSLARHL | LQCEICGFTCRQPKSLAR |
| 417_43_9- | 1 | 87 | CR (SEQ ID | RLH (SEQ ID | HLRLH (SEQ ID NO: 426) |
| ZN787_178_200_J1 | | | NO: 67) | NO: 419) | |
| IKZF2_140_162- | IKZF | ZN7 | FHCNQCGA | QPKSLARHL | FHCNQCGASFTQPKSLA |
| ZN787_178_200_J1 | 2 | 87 | SFT (SEQ ID | RLH (SEQ ID | RHLRLH (SEQ ID NO: |
| | | | NO: 69) | NO: 419) | 427) |
| PATZ1_383_405- | PAT | ZN7 | YSCPVCGL | QPKSLARHL | YSCPVCGLRFKQPKSLA |
| ZN787_178_200_J1 | Z1 | 87 | RFK (SEQ | RLH (SEQ ID | RHLRLH (SEQ ID NO: |
| | | | ID NO: 51) | NO: 419) | 428) |
| E4F1_220_242- | E4F1 | ZN7 | HECKLCGA | QPKSLARHL | HECKLCGASFRQPKSLA |
| ZN787_178_200_J1 | | 87 | SFR (SEQ ID | RLH (SEQ ID | RHLRLH (SEQ ID NO: |
| | | | NO: 81) | NO: 419) | 429) |
| ZSC20_766_788- | ZSC | ZN7 | YKCLECGK | QPKSLARHL | YKCLECGKSFSQPKSLAR |
| ZN787_178_200_J1 | 20 | 87 | SFS (SEQ ID | RLH (SEQ ID | HLRLH (SEQ ID NO: 430) |
| | | | NO: 63) | NO: 419) | |
| ZN653_556_578- | ZN6 | ZN7 | LQCEICGY | QPKSLARHL | LQCEICGYQCRQPKSLAR |
| ZN787_178_200_J1 | 53 | 87 | QCR (SEQ | RLH (SEQ ID | HLRLH (SEQ ID NO: 431) |
| | | | ID NO: 65) | NO: 419) | |
| ZNF74_444_466- | ZNF | ZN7 | FKCADCGK | QPKSLARHL | FKCADCGKGFSQPKSLA |
| ZN787_178_200_J1 | 74 | 87 | GFS (SEQ ID | RLH (SEQ ID | RHLRLH (SEQ ID NO: |
| | | | NO: 75) | NO: 419) | 432) |
| ZF69B_419_441- | ZF69 | ZN7 | YICNVCSK | QPKSLARHL | YICNVCSKTFSQPKSLAR |
| ZN787_178_200_J1 | B | 87 | TFS (SEQ ID | RLH (SEQ ID | HLRLH (SEQ ID NO: 433) |
| | | | NO: 87) | NO: 419) | |
| ZN595_145_167- | ZN5 | ZN7 | FQCNTCVK | QPKSLARHL | FQCNTCVK VFSQPKSLA |
| ZN787_178_200_J1 | 95 | 87 | VFS (SEQ ID | RLH (SEQ ID | RHLRLH (SEQ ID NO: |
| | | | NO: 85) | NO: 419) | 434) |
| ZN276_524_546- | ZN2 | ZN7 | LQCEVCGF | QPKSLARHL | LQCEVCGFQCRQPKSLA |
| ZN787_178_200_J1 | 76 | 87 | QCR (SEQ | RLH (SEQ ID | RHLRLH (SEQ ID NO: |
| | | | ID NO: 71) | NO: 419) | 435) |
| ZKSC5_430_452- | ZKS | ZN7 | YGCNECGK | QPKSLARHL | YGCNECGKNFGQPKSLA |
| ZN787_178_200_J1 | C5 | 87 | NFG (SEQ | RLH (SEQ ID | RHLRLH (SEQ ID NO: |
| | | | ID NO: 73) | NO: 419) | 436) |
| ZNF90_481_503- | ZNF | ZN7 | YKCQECDK | QPKSLARHL | YKCQECDKAFKQPKSLA |
| ZN787_178_200_J1 | 90 | 87 | AFK (SEQ | RLH (SEQ ID | RHLRLH (SEQ ID NO: |
| | | | ID NO: 61) | NO: 419) | 437) |
| ZN597_341_363- | ZN5 | ZN7 | LQCPDCDM | QPKSLARHL | LQCPDCDMTFPQPKSLA |
| ZN787_178_200_J1 | 97 | 87 | TFP (SEQ ID | RLH (SEQ ID | RHLRLH (SEQ ID NO: |
| | | | NO: 59) | NO: 419) | 438) |
| ZN654_25_47- | ZN6 | ZN7 | FACVICGR | QPKSLARHL | FACVICGRKFRQPKSLAR |
| ZN787_178_200_J1 | 54 | 87 | KFR (SEQ | RLH (SEQ ID | HLRLH (SEQ ID NO: 439) |
| | | | ID NO: 55) | NO: 419) | |
| ZN787_178_200- | ZN7 | ZN7 | FVCPRCGR | QPKSLARHL | FVCPRCGRGFSQPKSLAR |
| ZN787_178_200_J1 | 87 | 87 | GFS (SEQ ID | RLH (SEQ ID | HLRLH (SEQ ID NO: 440) |
| | | | NO: 79) | NO: 419) | |
| ZSC20_766_788- | ZSC | ZN8 | YKCLECGK | RKSYWKRH | YKCLECGKSFSRKSYWK |
| ZN827_374_396_J1 | 20 | 27 | SFS (SEQ ID | MVIH (SEQ ID | RHMVIH (SEQ ID NO: |
| | | | NO: 63) | NO: 441) | 442) |
| ZN653_556_578- | ZN6 | ZN8 | LQCEICGY | RKSYWKRH | LQCEICGYQCRRKSYWK |
| ZN827_374_396_J1 | 53 | 27 | QCR (SEQ | MVIH (SEQ ID | RHMVIH (SEQ ID NO: |
| | | | ID NO: 65) | NO: 441) | 443) |
| ZN628_120_142- | ZN6 | ZN8 | FICGQCGL | RKSYWKRH | FICGQCGL AFKRKSYWK |
| ZN827_374_396_J1 | 28 | 27 | AFK (SEQ | MVIH (SEQ ID | RHMVIH (SEQ ID NO: |
| | | | ID NO: 49) | NO: 441) | 444) |
| ZKSC5_430_452- | ZKS | ZN8 | YGCNECGK | RKSYWKRH | YGCNECGKNFGRKSYW |
| ZN827_374_396_J1 | C5 | 27 | NFG (SEQ | MVIH (SEQ ID | KRHMVIH (SEQ ID NO: |
| | | | ID NO: 73) | NO: 441) | 445) |
| ZN276_524_546- | ZN2 | ZN8 | LQCEVCGF | RKSYWKRH | LQCEVCGFQCRRKSYWK |
| ZN827_374_396_J1 | 76 | 27 | QCR (SEQ | MVIH (SEQ ID | RHMVIH (SEQ ID NO: |
| | | | ID NO: 71) | NO: 441) | 446) |
| ZN398_483_505- | ZN3 | ZN8 | FSCPQCGID | RKSYWKRH | FSCPQCGIDFNRKSYWK |
| ZN827_374_396_J1 | 98 | 27 | FN (SEQ ID | MVIH (SEQ ID | RHMVIH (SEQ ID NO: |
| | | | NO: 53) | NO: 441) | 447) |
| IKZF3_146_168- | IKZF | ZN8 | FQCNQCGA | RKSYWKRH | FQCNQCGASFTRKSYWK |
| ZN827_374_396_J1 | 3 | 27 | SFT (SEQ ID | MVIH (SEQ ID | RHMVIH (SEQ ID NO: |
| | | | NO: 46) | NO: 441) | 448) |
| PATZ1_383_405- | PAT | ZN8 | YSCPVCGL | RKSYWKRH | YSCPVCGLRFKRKSYWK |
| ZN827_374_396_J1 | Z1 | 27 | RFK (SEQ | MVIH (SEQ ID | RHMVIH (SEQ ID NO: |
| | | | ID NO: 51) | NO: 441) | 449) |
| ZN787_178_200- | ZN7 | ZN8 | FVCPRCGR | RKSYWKRH | FVCPRCGRGFSRKSYWK |
| ZN827_374_396_J1 | 87 | 27 | GFS (SEQ ID | MVIH (SEQ ID | RHMVIH (SEQ ID NO: |
| | | | NO: 79) | NO: 441) | 450) |
| ZFP91_400_422ZN692 | ZFP9 | ZN8 | LQCEICGFT | RKSYWKRH | LQCEICGFTCRRKSYWK |
| 417_43_9- | 1 | 27 | CR (SEQ ID | MVIH (SEQ ID | RHMVIH (SEQ ID NO: |
| ZN827_374_396_J1 | | | NO: 67) | NO: 441) | 451) |
| ZN654_25_47- | ZN6 | ZN8 | FACVICGR | RKSYWKRH | FACVICGRKFRRKSYWK |
| ZN827_374_396_J1 | 54 | 27 | KFR (SEQ | MVIH (SEQ ID | RHMVIH (SEQ ID NO: |
| | | | ID NO: 55) | NO: 441) | 452) |
| ZNF74_444_466- | ZNF | ZN8 | FKCADCGK | RKSYWKRH | FKCADCGKGFSRKSYWK |
| ZN827_374_396_J1 | 74 | 27 | GFS (SEQ ID | MVIH (SEQ ID | RHMVIH (SEQ ID NO: |
| | | | NO: 75) | NO: 441) | 453) |
| ZF69B_419_441- | ZF69 | ZN8 | YICNVCSK | RKSYWKRH | YICNVCSKTFSRKSYWK |
| ZN827_374_396_J1 | B | 27 | TFS (SEQ ID | MVIH (SEQ ID | RHMVIH (SEQ ID NO: |
| | | | NO: 87) | NO: 441) | 454) |
| E4F1_220_242- | E4F1 | ZN8 | HECKLCGA | RKSYWKRH | HECKLCGASFRRKSYWK |
| ZN827_374_396_J1 | | 27 | SFR (SEQ ID | MVIH (SEQ ID | RHMVIH (SEQ ID NO: |
| | | | NO: 81) | NO: 441) | 455) |
| ZN827_374_396- | ZN8 | ZN8 | FQCPICGLV | RKSYWKRH | FQCPICGLVIKRKSYWKR |
| ZN827_374_396_J1 | 27 | 27 | IK (SEQ ID | MVIH (SEQ ID | HMVIH (SEQ ID NO: 456) |
| | | | NO: 57) | NO: 441) | |
| ZN517_452_474- | ZN5 | ZN8 | YRCRACGR | RKSYWKRH | YRCRACGRACSRKSYW |
| ZN827_374_396_J1 | 17 | 27 | ACS (SEQ | MVIH (SEQ ID | KRHMVIH (SEQ ID NO: |
| | | | ID NO: 83) | NO: 441) | 457) |
| ZN582_395_417- | ZN5 | ZN8 | YQCKVCGR | RKSYWKRH | YQCKVCGRAFKRKSYW |
| ZN827_374_396_J1 | 82 | 27 | AFK (SEQ | MVIH (SEQ ID | KRHMVIH (SEQ ID NO: |
| | | | ID NO: 77) | NO: 441) | 458) |
| IKZF2_140_162- | IKZF | ZN8 | FHCNQCGA | RKSYWKRH | FHCNQCGASFTRKSYWK |
| ZN827_374_396_J1 | 2 | 27 | SFT (SEQ ID | MVIH (SEQ ID | RHMVIH (SEQ ID NO: |
| | | | NO: 69) | NO: 441) | 459) |
| ZN597_341_363- | ZN5 | ZN8 | LQCPDCDM | RKSYWKRH | LQCPDCDMTFPRKSYWK |
| ZN827_374_396_J1 | 97 | 27 | TFP (SEQ ID | MVIH (SEQ ID | RHMVIH (SEQ ID NO: |
| | | | NO: 59) | NO: 441) | 460) |
| ZN595_145_167- | ZN5 | ZN8 | FQCNTCVK | RKSYWKRH | FQCNTCVK VFSRKSYWK |
| ZN827_374_396_J1 | 95 | 27 | VFS (SEQ ID | MVIH (SEQ ID | RHMVIH (SEQ ID NO: |
| | | | NO: 85) | NO: 441) | 461) |
| E4F1_220_242- | E4F1 | ZNF | HECKLCGA | CHAYLLVHR | HECKLCGASFRCHAYLL |
| ZNF74_444_466_J1 | | 74 | SFR (SEQ ID | RIH (SEQ ID | VHRRIH (SEQ ID NO: 463) |
| | | | NO: 81) | NO: 462) | |
| ZN827_374_396- | ZN8 | ZNF | FQCPICGLV | CHAYLLVHR | FQCPICGLVIKCHAYLLV |
| ZNF74_444_466_J1 | 27 | 74 | IK (SEQ ID | RIH (SEQ ID | HRRIH (SEQ ID NO: 464) |
| | | | NO: 57) | NO: 462) | |
| ZN628_120_142- | ZN6 | ZNF | FICGQCGL | CHAYLLVHR | FICGQCGL AFK CHAYLL |
| ZNF74_444_466_J1 | 28 | 74 | AFK (SEQ | RIH (SEQ ID | VHRRIH (SEQ ID NO: 465) |
| | | | ID NO: 49) | NO: 462) | |
| ZN595_145_167- | ZN5 | ZNF | FQCNTCVK | CHAYLLVHR | FQCNTCVK VFSCHAYLL |
| ZNF74_444_466_J1 | 95 | 74 | VFS (SEQ ID | RIH (SEQ ID | VHRRIH (SEQ ID NO: 466) |
| | | | NO: 85) | NO: 462) | |
| ZN398_483_505- | ZN3 | ZNF | FSCPQCGID | CHAYLLVHR | FSCPQCGIDFNCHAYLLV |
| ZNF74_444_466_J1 | 98 | 74 | FN (SEQ ID | RIH (SEQ ID | HRRIH (SEQ ID NO: 467) |
| | | | NO: 53) | NO: 462) | |
| ZN653_556_578- | ZN6 | ZNF | LQCEICGY | CHAYLLVHR | LQCEICGYQCRCHAYLL |
| ZNF74_444_466_J1 | 53 | 74 | QCR (SEQ | RIH (SEQ ID | VHRRIH (SEQ ID NO: 468) |
| | | | ID NO: 65) | NO: 462) | |
| IKZF3_146_168- | IKZF | ZNF | FQCNQCGA | CHAYLLVHR | FQCNQCGASFTCHAYLL |
| ZNF74_444_466_J1 | 3 | 74 | SFT (SEQ ID | RIH (SEQ ID | VHRRIH (SEQ ID NO: 469) |
| | | | NO: 46) | NO: 462) | |
| ZF69B_419_441- | ZF69 | ZNF | YICNVCSK | CHAYLLVHR | YICNVCSKTFSCHAYLLV |
| ZNF74_444_466_J1 | B | 74 | TFS (SEQ ID | RIH (SEQ ID | HRRIH (SEQ ID NO: 470) |
| | | | NO: 87) | NO: 462) | |
| PATZ1_383_405- | PAT | ZNF | YSCPVCGL | CHAYLLVHR | YSCPVCGLRFKCHAYLL |
| ZNF74_444_466_J1 | Z1 | 74 | RFK (SEQ | RIH (SEQ ID | VHRRIH (SEQ ID NO: 471) |
| | | | ID NO: 51) | NO: 462) | |
| ZNF74_444_466- | ZNF | ZNF | FKCADCGK | CHAYLLVHR | FKCADCGKGFSCHAYLL |
| ZNF74_444_466_J1 | 74 | 74 | GFS (SEQ ID | RIH (SEQ ID | VHRRIH (SEQ ID NO: 472) |
| | | | NO: 75) | NO: 462) | |
| ZSC20_766_788- | ZSC | ZNF | YKCLECGK | CHAYLLVHR | YKCLECGKSFSCHAYLL |
| ZNF74_444_466_J1 | 20 | 74 | SFS (SEQ ID | RIH (SEQ ID | VHRRIH (SEQ ID NO: 473) |
| | | | NO: 63) | NO: 462) | |
| ZNF90_481_503- | ZNF | ZNF | YKCQECDK | CHAYLLVHR | YKCQECDKAFKCHAYLL |
| ZNF74_444_466_J1 | 90 | 74 | AFK (SEQ | RIH (SEQ ID | VHRRIH (SEQ ID NO: 474) |
| | | | ID NO: 61) | NO: 462) | |
| ZKSC5_430_452- | ZKS | ZNF | YGCNECGK | CHAYLLVHR | YGCNECGKNFGCHAYLL |
| ZNF74_444_466_J1 | C5 | 74 | NFG (SEQ | RIH (SEQ ID | VHRRIH (SEQ ID NO: 475) |
| | | | ID NO: 73) | NO: 462) | |
| IKZF2_140_162- | IKZF | ZNF | FHCNQCGA | CHAYLLVHR | FHCNQCGASFTCHAYLL |
| ZNF74_444_466_J1 | 2 | 74 | SFT (SEQ ID | RIH (SEQ ID | VHRRIH (SEQ ID NO: 476) |
| | | | NO: 69) | NO: 462) | |
| ZN597_341_363- | ZN5 | ZNF | LQCPDCDM | CHAYLLVHR | LQCPDCDMTFPCHAYLL |
| ZNF74_444_466_J1 | 97 | 74 | TFP (SEQ ID | RIH (SEQ ID | VHRRIH (SEQ ID NO: 477) |
| | | | NO: 59) | NO: 462) | |
| ZN276_524_546- | ZN2 | ZNF | LQCEVCGF | CHAYLLVHR | LQCEVCGFQCRCHAYLL |
| ZNF74_444_466_J1 | 76 | 74 | QCR (SEQ | RIH (SEQ ID | VHRRIH (SEQ ID NO: 478) |
| | | | ID NO: 71) | NO: 462) | |
| ZN582_395_417- | ZN5 | ZNF | YQCKVCGR | CHAYLLVHR | YQCKVCGRAFKCHAYLL |
| ZNF74_444_466_J1 | 82 | 74 | AFK (SEQ | RIH (SEQ ID | VHRRIH (SEQ ID NO: 479) |
| | | | ID NO: 77) | NO: 462) | |
| ZN517_452_474- | ZN5 | ZNF | YRCRACGR | CHAYLLVHR | YRCRACGRACSCHAYLL |
| ZNF74_444_466_J1 | 17 | 74 | ACS (SEQ | RIH (SEQ ID | VHRRIH (SEQ ID NO: 480) |
| | | | ID NO: 83) | NO: 462) | |
| ZN787_178_200- | ZN7 | ZNF | FVCPRCGR | CHAYLLVHR | FVCPRCGRGFSCHAYLL |
| ZNF74_444_466_J1 | 87 | 74 | GFS (SEQ ID | RIH (SEQ ID | VHRRIH (SEQ ID NO: 481) |
| | | | NO: 79) | NO: 462) | |
| ZFP91_400_422ZN692 | ZFP9 | ZNF | LQCEICGFT | CHAYLLVHR | LQCEICGFTCRCHAYLLV |
| 417_43_9- | 1 | 74 | CR (SEQ ID | RIH (SEQ ID | HRRIH (SEQ ID NO: 482) |
| ZNF74_444_466_J1 | | | NO: 67) | NO: 462) | |
| ZN654_25_47- | ZN6 | ZNF | FACVICGR | CHAYLLVHR | FACVICGRKFRCHAYLL |
| ZNF74_444_466_J1 | 54 | 74 | KFR (SEQ | RIH (SEQ ID | VHRRIH (SEQ ID NO: 483) |
| | | | ID NO: 55) | NO: 462) | |
| ZF69B_419_441- | ZF69 | ZNF | YICNVCSK | YSSALSTHKII | YICNVCSKTFSYSSALST |
| ZNF90_481_503_J1 | B | 90 | TFS (SEQ ID | H (SEQ ID NO: | HKIIH (SEQ ID NO: 485) |
| | | | NO: 87) | 484) | |
| ZKSC5_430_452- | ZKS | ZNF | YGCNECGK | YSSALSTHKII | YGCNECGKNFGYSSALS |
| ZNF90_481_503_J1 | C5 | 90 | NFG (SEQ | H (SEQ ID NO: | THKIIH (SEQ ID NO: 486) |
| | | | ID NO: 73) | 484) | |
| ZFP91_400_422ZN692 | ZFP9 | ZNF | LQCEICGFT | YSSALSTHKII | LQCEICGFTCRYSSALST |
| 417_43_9- | 1 | 90 | CR (SEQ ID | H (SEQ ID NO: | HKIIH (SEQ ID NO: 487) |
| ZNF90_481_503_J1 | | | NO: 67) | 484) | |
| ZN595_145_167- | ZN5 | ZNF | FQCNTCVK | YSSALSTHKII | FQCNTCVKVFSYSSALST |
| ZNF90_481_503_J1 | 95 | 90 | VFS (SEQ ID | H (SEQ ID NO: | HKIIH (SEQ ID NO; 488) |
| | | | NO: 85) | 484) | |
| ZN597_341_363- | ZN5 | ZNF | LQCPDCDM | YSSALSTHKII | LQCPDCDMTFPYSSALST |
| ZNF90_481_503_J1 | 97 | 90 | TFP (SEQ ID | H (SEQ ID NO: | HKIIH (SEQ ID NO: 489) |
| | | | NO: 59) | 484) | |
| ZN653_556_578- | ZN6 | ZNF | LQCEICGY | YSSALSTHKII | LQCEICGYQCRYSSALST |
| ZNF90_481_503_J1 | 53 | 90 | QCR (SEQ | H (SEQ ID NO: | HKIIH (SEQ ID NO: 490) |
| | | | ID NO: 65) | 484) | |
| ZN787_178_200- | ZN7 | ZNF | FVCPRCGR | YSSALSTHKII | FVCPRCGRGFSYSSALST |
| ZNF90_481_503_J1 | 87 | 90 | GFS (SEQ ID | H (SEQ ID NO: | HKIIH (SEQ ID NO: 491) |
| | | | NO: 79) | 484) | |
| ZN827_374_396- | ZN8 | ZNF | FQCPICGLV | YSSALSTHKII | FQCPICGLVIKYSSALSTH |
| ZNF90_481_503_J1 | 27 | 90 | IK (SEQ ID | H (SEQ ID NO: | KIIH (SEQ ID NO: 492) |
| | | | NO: 57) | 484) | |
| ZN582_395_417- | ZN5 | ZNF | YQCKVCGR | YSSALSTHKII | YQCKVCGRAFKYSSALS |
| ZNF90_481_503_J1 | 82 | 90 | AFK (SEQ | H (SEQ ID NO: | THKIIH (SEQ ID NO: 493) |
| | | | ID NO: 77) | 484) | |
| ZN276_524_546- | ZN2 | ZNF | LQCEVCGF | YSSALSTHKII | LQCEVCGFQCRYSSALST |
| ZNF90_481_503_J1 | 76 | 90 | QCR (SEQ | H (SEQ ID NO: | HKIIH (SEQ ID NO: 494) |
| | | | ID NO: 71) | 484) | |
| IKZF3_146_168- | IKZF | ZNF | FQCNQCGA | YSSALSTHKII | FQCNQCGASFTYSSALST |
| ZNF90_481_503_J1 | 3 | 90 | SFT (SEQ ID | H (SEQ ID NO: | HKIIH (SEQ ID NO: 495) |
| | | | NO: 46) | 484) | |
| ZN654_25_47- | ZN6 | ZNF | FACVICGR | YSSALSTHKII | FACVICGRKFRYSSALST |
| ZNF90_481_503_J1 | 54 | 90 | KFR (SEQ | H (SEQ ID NO: | HKIIH (SEQ ID NO: 496) |
| | | | ID NO: 55) | 484) | |
| ZN628_120_142- | ZN6 | ZNF | FICGQCGL | YSSALSTHKII | FICGQCGLAFKYSSALST |
| ZNF90_481_503_J1 | 28 | 90 | AFK (SEQ | H (SEQ ID NO: | HKIIH (SEQ ID NO: 497) |
| | | | ID NO: 49) | 484) | |
| ZNF74_444_466- | ZNF | ZNF | FKCADCGK | YSSALSTHKII | FKCADCGKGFSYSSALST |
| ZNF90_481_503_J1 | 74 | 90 | GFS (SEQ ID | H (SEQ ID NO: | HKIIH (SEQ ID NO: 498) |
| | | | NO: 75) | 484) | |
| ZSC20_766_788- | zsc | ZNF | YKCLECGK | YSSALSTHKII | YKCLECGKSFSYSSALST |
| ZNF90_481_503_J1 | 20 | 90 | SFS (SEQ ID | H (SEQ ID NO: | HKIIH (SEQ ID NO: 499) |
| | | | NO: 63) | 484) | |
| ZN517_452_474- | ZN5 | ZNF | YRCRACGR | YSSALSTHKII | YRCRACGRACSYSSALS |
| ZNF90_481_503_J1 | 17 | 90 | ACS (SEQ | H (SEQ ID NO: | THKIIH (SEQ ID NO: 500) |
| | | | ID NO: 83) | 484) | |
| ZNF90_481_503- | ZNF | ZNF | YKCQECDK | YSSALSTHKII | YKCQECDKAFKYSSALS |
| ZNF90_481_503_J1 | 90 | 90 | AFK (SEQ | H (SEQ ID NO: | THKIIH (SEQ ID NO: 501) |
| | | | ID NO: 61) | 484) | |
| E4F1_220_242- | E4F1 | ZNF | HECKLCGA | YSSALSTHKII | HECKLCGASFRYSSALST |
| ZNF90_481_503_J1 | | 90 | SFR (SEQ ID | H (SEQ ID NO: | HKIIH (SEQ ID NO: 502) |
| | | | NO: 81) | 484) | |
| ZN398_483_505- | ZN3 | ZNF | FSCPQCGID | YSSALSTHKII | FSCPQCGIDFNYSSALST |
| ZNF90_481_503_J1 | 98 | 90 | FN (SEQ ID | H (SEQ ID NO: | HKIIH (SEQ ID NO: 503) |
| | | | NO: 53) | 484) | |
| IKZF2_140_162- | IKZF | ZNF | FHCNQCGA | YSSALSTHKII | FHCNQCGASFTYSSALST |
| ZNF90_481_503_J1 | 2 | 90 | SFT (SEQ ID | H (SEQ ID NO: | HKIIH (SEQ ID NO: 504) |
| | | | NO: 69) | 484) | |
| PATZ1_383_405- | PAT | ZNF | YSCPVCGL | YSSALSTHKII | YSCPVCGLRFKYSSALST |
| ZNF90_481_503_J1 | Z1 | 90 | RFK (SEQ | H (SEQ ID NO: | HKIIH (SEQ ID NO: 505) |
| | | | ID NO: 51) | 484) | |
| ZNF90_481_503- | ZNF | ZSC | YKCQECDK | DHSNLITHQR | YKCQECDKAFKDHSNLI |
| ZSC20_766_788_J1 | 90 | 20 | AFK (SEQ | IH (SEQ ID | THQRIH (SEQ ID NO: 507) |
| | | | ID NO: 61) | NO: 506) | |
| ZN595_145_167- | ZN5 | ZSC | FQCNTCVK | DHSNLITHQR | FQCNTCVK VFSDHSNLIT |
| ZSC20_766_788_J1 | 95 | 20 | VFS (SEQ ID | IH (SEQ ID | HQRIH (SEQ ID NO: 508) |
| | | | NO: 85) | NO: 506) | |
| IKZF3_146_168- | IKZF | ZSC | FQCNQCGA | DHSNLITHQR | FQCNQCGASFTDHSNLIT |
| ZSC20_766_788_J1 | 3 | 20 | SFT (SEQ ID | IH (SEQ ID | HQRIH (SEQ ID NO: 509) |
| | | | NO: 46) | NO: 506) | |
| ZN827_374_396- | ZN8 | ZSC | FQCPICGLV | DHSNLITHQR | FQCPICGLVIKDHSNLITH |
| ZSC20_766_788_J1 | 27 | 20 | IK (SEQ ID | IH (SEQ ID | QRIH (SEQ ID NO: 510) |
| | | | NO: 57) | NO: 506) | |
| ZN276_524_546- | ZN2 | ZSC | LQCEVCGF | DHSNLITHQR | LQCEVCGFQCRDHSNLIT |
| ZSC20_766_788_J1 | 76 | 20 | QCR (SEQ | IH (SEQ ID | HQRIH (SEQ ID NO: 511) |
| | | | ID NO: 71) | NO: 506) | |
| ZKSC5_430_452- | ZKS | ZSC | YGCNECGK | DHSNLITHQR | YGCNECGKNFGDHSNLI |
| ZSC20_766_788_J1 | C5 | 20 | NFG (SEQ | IH (SEQ ID | THQRIH (SEQ ID NO: 512) |
| | | | ID NO: 73) | NO: 506) | |
| ZN628_120_142- | ZN6 | ZSC | FICGQCGL | DHSNLITHQR | FICGQCGLAFKDHSNLIT |
| ZSC20_766_788_J1 | 28 | 20 | AFK (SEQ | IH (SEQ ID | HQRIH (SEQ ID NO: 513) |
| | | | ID NO: 49) | NO: 506) | |
| ZN653_556_578- | ZN6 | ZSC | LQCEICGY | DHSNLITHQR | LQCEICGYQCRDHSNLIT |
| ZSC20_766_788_J1 | 53 | 20 | QCR (SEQ | IH (SEQ ID | HQRIH (SEQ ID NO: 514) |
| | | | ID NO: 65) | NO: 506) | |
| ZN517_452_474- | ZN5 | ZSC | YRCRACGR | DHSNLITHQR | YRCRACGRACSDHSNLI |
| ZSC20_766_788_J1 | 17 | 20 | ACS (SEQ | IH (SEQ ID | THQRIH (SEQ ID NO: 515) |
| | | | ID NO: 83) | NO: 506) | |
| ZN398_483_505- | ZN3 | ZSC | FSCPQCGID | DHSNLITHQR | FSCPQCGIDFNDHSNLIT |
| ZSC20_766_788_J1 | 98 | 20 | FN (SEQ ID | IH (SEQ ID | HQRIH (SEQ ID NO: 516) |
| | | | NO: 53) | NO: 506) | |
| ZN582_395_417- | ZN5 | ZSC | YQCKVCGR | DHSNLITHQR | YQCKVCGRAFKDHSNLI |
| ZSC20_766_788_J1 | 82 | 20 | AFK (SEQ | IH (SEQ ID | THQRIH (SEQ ID NO: 517) |
| | | | ID NO: 77) | NO: 506) | |
| ZF69B_419_441- | ZF69 | ZSC | YICNVCSK | DHSNLITHQR | YICNVCSKTFSDHSNLIT |
| ZSC20_766_788_J1 | B | 20 | TFS (SEQ ID | IH (SEQ ID | HQRIH (SEQ ID NO: 518) |
| | | | NO: 87) | NO: 506) | |
| ZN787_178_200- | ZN7 | ZSC | FVCPRCGR | DHSNLITHQR | FVCPRCGRGFSDHSNLIT |
| ZSC20_766_788_J1 | 87 | 20 | GFS (SEQ ID | IH (SEQ ID | HQRIH (SEQ ID NO: 519) |
| | | | NO: 79) | NO: 506) | |
| ZN654_25_47- | ZN6 | ZSC | FACVICGR | DHSNLITHQR | FACVICGRKFRDHSNLIT |
| ZSC20_766_788_J1 | 54 | 20 | KFR (SEQ | IH (SEQ ID | HQRIH (SEQ ID NO: 520) |
| | | | ID NO: 55) | NO: 506) | |
| E4F1_220_242- | E4F1 | ZSC | HECKLCGA | DHSNLITHQR | HECKLCGASFRDHSNLIT |
| ZSC20_766_788_J1 | | 20 | SFR (SEQ ID | IH (SEQ ID | HQRIH (SEQ ID NO: 521) |
| | | | NO: 81) | NO: 506) | |
| IKZF2_140_162- | IKZF | ZSC | FHCNQCGA | DHSNLITHQR | FHCNQCGASFTDHSNLIT |
| ZSC20_766_788_J1 | 2 | 20 | SFT (SEQ ID | IH (SEQ ID | HQRIH (SEQ ID NO: 522) |
| | | | NO: 69) | NO: 506) | |
| ZNF74_444_466- | ZNF | ZSC | FKCADCGK | DHSNLITHQR | FKCADCGKGFSDHSNLIT |
| ZSC20_766_788_J1 | 74 | 20 | GFS (SEQ ID | IH (SEQ ID | HQRIH (SEQ ID NO: 523) |
| | | | NO: 75) | NO: 506) | |
| ZSC20_766_788- | zsc | ZSC | YKCLECGK | DHSNLITHQR | YKCLECGKSFSDHSNLIT |
| ZSC20_766_788_J1 | 20 | 20 | SFS (SEQ ID | IH (SEQ ID | HQRIH (SEQ ID NO: 524) |
| | | | NO: 63) | NO: 506) | |
| ZN597_341_363- | ZN5 | ZSC | LQCPDCDM | DHSNLITHQR | LQCPDCDMTFPDHSNLIT |
| ZSC20_766_788_J1 | 97 | 20 | TFP (SEQ ID | IH (SEQ ID | HQRIH (SEQ ID NO: 525) |
| | | | NO: 59) | NO: 506) | |
| PATZ1_383_405- | PAT | ZSC | YSCPVCGL | DHSNLITHQR | YSCPVCGLRFKDHSNLIT |
| ZSC20_766_788_J1 | Z1 | 20 | RFK (SEQ | IH (SEQ ID | HQRIH (SEQ ID NO: 526) |
| | | | ID NO: 51) | NO: 506) | |
| ZFP91_400_422ZN692 | ZFP9 | ZSC | LQCEICGFT | DHSNLITHQR | LQCEICGFTCRDHSNLIT |
| 417_43_9- | 1 | 20 | CR (SEQ ID | IH (SEQ ID | HQRIH (SEQ ID NO: 527) |
| ZSC20_766_788_J1 | | | NO: 67) | NO: 506) | |

TABLE 3B
Validation of Hybrid Zinc Fingers

| | | | | | | | | | | | EC50 |
|---|---|---|---|---|---|---|---|---|---|---|---|
| Valida- | | | | | | HZnF | EC50 | EC50 | EC50 | EC50 | len |
| tion | ZnF | N | C | Naa | Caa | aa | len | pom | cc122 | cc220 | repeat |
| HZnF_ | ZN276_ | ZN | ZN276 | LQCEV | QRAS | LQCE | 78.56 | 7.405 | 73.62 | 0.000815 | |
| 01 | 524546- | 276 | | CGFQCR | LKYH | VCGF | | | | | |
| | ZN276_ | | | (SEQ | MTKH | QCRQ | | | | | |
| | J1 | | | ID NO: | (SEQ | RASL | | | | | |
| | | | | 71) | ID NO: | KYHM | | | | | |
| | | | | | 199) | TKH | | | | | |
| | | | | | | (SEQ | | | | | |
| | | | | | | ID NO: | | | | | |
| | | | | | | 215) | | | | | |
| HZnF_ | PATZ1_ | PATZ1 | PATZ1 | YSCPVCG | RKDRMSYH | YSCPVCGL | 100 | 32.36 | 176 | 6.57E−05 | |
| 02 | 383405- | | | LRFK | VRSH (SEQ | RFKRKDR | | | | | |
| | PATZ1_ | | | (SEQ ID | ID NO: | MSYHVRSH | | | | | |
| | 383_ | | | NO: 51) | 111) | (SEQ ID | | | | | |
| | 405_ | | | | | NO: 119) | | | | | |
| | J1 | | | | | | | | | | |
| HZnF_ | ZN517_ | ZN517 | ZN517 | YRCRAC | RLSTLIQHQ | YRCRACGR | 55.16 | 4.47 | 29.5 | 0.000999 | |
| 03 | 452_ | | | GRACS | KVH (SEQ | ACSRLSTLI | | | | | |
| | 474- | | | (SEQ ID | ID NO: | QHQKVH | | | | | |
| | ZN517_ | | | NO: 83) | 243) | (SEQ ID | | | | | |
| | 452_ | | | | | NO: | | | | | |
| | 474_ | | | | | 246) | | | | | |
| | J1 | | | | | | | | | | |
| HZnF_ | ZFP91_ | ZFP91 | ZN628 | LQCEIC | WSSHYQYH | LQCEICGFT | 323.7 | 14.07 | 101.4 | 0.001282 | |
| 04 | 400_ | | | GFTCR | LRQH (SEQ | CRWSSHYQ | | | | | |
| | 422Z | | | (SEQ ID | ID NO: | YHLRQH | | | | | |
| | N692_ | | | NO: 67) | 331) | (SEQ ID | | | | | |
| | 417_ | | | | | NO: | | | | | |
| | 439- | | | | | 337) | | | | | |
| | ZN628_ | | | | | | | | | | |
| | 120142_ | | | | | | | | | | |
| | J1 | | | | | | | | | | |
| HZnF_ | ZN787_ | ZN787 | ZN787 | FVCPR | QPKSLARH | FVCPRCGR | 47.95 | 8.555 | 2.928 | 0.000493 | |
| 05 | 178_ | | | CGRG | LRLH (SEQ | GFSQPKSL | | | | | |
| | 200- | | | FS | ID NO: | ARHLRLH | | | | | |
| | ZN787_ | | | (SEQ | 419) | (SEQ ID | | | | | |
| | 178_ | | | ID NO: | | NO: | | | | | |
| | 200_ | | | 79) | | 440) | | | | | |
| | J1 | | | | | | | | | | |
| HZnF_ | IKZF3_ | IKZF3 | ZN517 | FQCN | RLST | FQCN | 433.1 | 21.91 | 56.11 | 0.004382 | |
| 06 | 146168- | | | QCGA | LIQH | QCGA | | | | | |
| | ZN517_ | | | SFT(SEQ | QKVH | SFTR | | | | | |
| | 452474_ | | | ID | (SEQ | LSTL | | | | | |
| | J1 | | | NO: | ID | IQHQ | | | | | |
| | | | | 46) | NO: | KVH | | | | | |
| | | | | | 243) | (SEQ | | | | | |
| | | | | | | ID | | | | | |
| | | | | | | NO: | | | | | |
| | | | | | | 245) | | | | | |
| HZnF_ | E4F1_ | E4F1 | E | HECK | TKGS | HECK | 34.77 | 11.35 | 58.55 | 0.001201 | |
| 07 | 220_ | | | LCGA | LIRH | LCGA | | | | | |
| | 242- | | | SFR(SEQ | HRRH | SFRT | | | | | |
| | E4F1_ | | | ID | (SEQ | KGSL | | | | | |
| | 220_ | | | NO: | ID | IRHH | | | | | |
| | 242_ | | | 81) | NO: | RRH | | | | | |
| | J1 | | | | 47) | (SEQ | | | | | |
| | | | | | | ID | | | | | |
| | | | | | | NO: | | | | | |
| | | | | | | 82) | | | | | |
| HZnF_ | IKZF3_ | IKZF3 | E4F1 | FQCN | TKGS | FQCN | 18.32 | 3.613 | 10.1 | 0.00036 | |
| 08 | 146168- | | | QCGA | LIRH | QCGA | | | | | |
| | E4F1_ | | | SFT(SEQ | HRRH | SFTT | | | | | |
| | 220_ | | | ID | (SEQ | KGSL | | | | | |
| | 242_ | | | NO: | ID | IRHH | | | | | |
| | J1 | | | 46) | NO: | RRH | | | | | |
| | | | | | 47) | (SEQ | | | | | |
| | | | | | | ID | | | | | |
| | | | | | | NO: | | | | | |
| | | | | | | 48) | | | | | |
| HZnF_ | ZKSC5_ | ZKSC5 | ZKSC5 | YGCN | RHSH | YGCN | | 0.2241 | 68.15 | 0.000198 | |
| 09 | 430452- | | | ECGK | LIEH | ECGK | | | | | |
| | ZKSC5_ | | | NFG(SEQ | LKRH | NFGR | | | | | |
| | 430452_ | | | ID | (SEQ | HSHL | | | | | |
| | J1 | | | NO: | ID | IEHL | | | | | |
| | | | | 73) | NO: | KRH | | | | | |
| | | | | | 177) | (SEQ | | | | | |
| | | | | | | ID | | | | | |
| | | | | | | NO: | | | | | |
| | | | | | | 182) | | | | | |
| HZnF_ | IKZF3_ | 1KZF3 | ZKSC5 | FQCN | RHSH | FQCN | 55.37 | 9.051 | 32.42 | 0.000608 | |
| 10 | 146168- | | | QCGA | LIEH | QCGA | | | | | |
| | ZKSC5_ | | | SFT(SEQ | LKRH | SFTR | | | | | |
| | 430452_ | | | ID | (SEQ | HSHL | | | | | |
| | J1 | | | NO: | ID | IEHL | | | | | |
| | | | | 46) | NO: | KRH | | | | | |
| | | | | | 177) | (SEQ | | | | | |
| | | | | | | ID | | | | | |
| | | | | | | NO: | | | | | |
| | | | | | | 197) | | | | | |
| HZnF_ | ZN654_ | ZN654 | ZN654 | FACV | NRGL | FACV | 59.85 | 18.84 | 178.9 | 0.00031 | |
| 11 | 2547- | | | ICGR | MQKH | ICGR | | | | | |
| | ZN654_ | | | KFR(SEQ | LKNH | KFRN | | | | | |
| | 2547_ | | | ID | (SEQ | RGLM | | | | | |
| | J1 | | | NO: | ID | QKHL | | | | | |
| | | | | 55)L | NO: | KNH | | | | | |
| | | | | | 375) | (SEQ | | | | | |
| | | | | | | ID | | | | | |
| | | | | | | NO: | | | | | |
| | | | | | | 394) | | | | | |
| HZnF_ | ZN653_ | ZN653 | ZN653 | QCE | QRAS | LQCE | 75.06 | 5.167 | 36.98 | 0.000582 | |
| 12 | 556578- | | | ICGY | LNWH | ICGY | | | | | |
| | ZN653_ | | | QCR | MKKH | QCRQ | | | | | |
| | 556578_ | | | (SEQ | (SEQ | RASL | | | | | |
| | J1 | | | ID | ID | NWHM | | | | | |
| | | | | NO: | NO: | KKH | | | | | |
| | | | | 65) | 353) | (SEQ | | | | | |
| | | | | | | ID | | | | | |
| | | | | | | NO: | | | | | |
| | | | | | | 366) | | | | | |
| HZnF_ | ZFP91_ | ZFP91 | ZFP91 | LQCE | QKAS | LQCE | 57.81 | 5.471 | 33 | 0.000282 | |
| 13 | 400422 | | | ICGF | LNWH | ICGF | | | | | |
| | ZN692417_ | | | TCR(SEQ | MKKH | TCRQ | | | | | |
| | 439- | | | ID | (SEQ | KASL | | | | | |
| | ZFP91_ | | | NO: | ID | NWHM | | | | | |
| | 400422_ | | | 67) | NO: | KKH | | | | | |
| | J1 | | | | 155) | (SEQ | | | | | |
| | | | | | | ID | | | | | |
| | | | | | | NO: | | | | | |
| | | | | | | 172) | | | | | |
| HZnF_ | ZN582_ | ZN582 | IKZF3 | YQCK | QKGN | YQCK | 83.12 | 5.192 | 26.04 | 0.000579 | |
| 14 | 395417- | | | VCGR | LLRH | VCGR | | | | | |
| | IKZF3_ | | | AFK(SEQ | IKLH | AFKQ | | | | | |
| | 146168 | | | ID | (SEQ | KGNL | | | | | |
| | IKZF2_ | | | NO: | ID | LRHI | | | | | |
| | 140_ | | | 77) | NO: | KLH | | | | | |
| | 162_ | | | | 89) | (SEQ | | | | | |
| | J1 | | | | | ID | | | | | |
| | | | | | | NO: | | | | | |
| | | | | | | 92) | | | | | |
| HZnF_ | ZN582_ | ZN582 | ZN517 | YQCK | RLST | YQCK | 71.86 | 5.118 | 35.16 | 0.001521 | |
| 15 | 395_ | | | VCGR | LIQH | VCGR | | | | | |
| | 417- | | | AFK(SEQ | QKVH | AFKR | | | | | |
| | ZN517_ | | | ID | (SEQ | LSTL | | | | | |
| | 452474_ | | | NO: | ID | IQHQ | | | | | |
| | J1 | | | 77) | NO: | KVH | | | | | |
| | | | | | 243) | (SEQ | | | | | |
| | | | | | | ID | | | | | |
| | | | | | | NO: | | | | | |
| | | | | | | 263) | | | | | |
| HZnF_ | ZN827_ | ZN827 | ZN827 | FQCP | RKSY | FQCP | 60.53 | 8.058 | 50.18 | 0.01022 | |
| 16 | 374396- | | | ICGL | WKRH | ICGL | | | | | |
| | ZN827_ | | | VIK(SEQ | MVIH | VIKR | | | | | |
| | 374396_ | | | ID | (SEQ | KSYW | | | | | |
| | J1 | | | NO: | ID | KRHM | | | | | |
| | | | | 57) | NO: | VIH | | | | | |
| | | | | | 441) | (SEQ ID | | | | | |
| | | | | | | NO: | | | | | |
| | | | | | | 456) | | | | | |
| HZnF_ | ZFP91_ | ZFP91 | ZKSC5 | LQCE | RHSH | LQCE | 149.3 | 5.288 | 73.43 | 0.000384 | |
| 17 | 400_ | | | ICGF | LIEH | ICGF | | | | | |
| | 422ZN692_ | | | TCR(SEQ | LKRH | TCRR | | | | | |
| | 417_ | | | ID | (SEQ | HSHL | | | | | |
| | 439- | | | NO: | ID | IEHL | | | | | |
| | ZKSC5_ | | | 67) | NO: | KRH | | | | | |
| | 430452_ | | | | 177) | (SEQ ID | | | | | |
| | J1 | | | | | NO: | | | | | |
| | | | | | | 196) | | | | | |
| HZnF_ | ZN653_ | ZN653 | ZN517 | LQCE | RLST | LQCE | 22.51 | 1.864 | 7.071 | 0.000545 | |
| 1 | 556_ | | | ICGY | LIQH | ICGY | | | | | |
| | 578- | | | QCR(SEQ | QKVH | QCRR | | | | | |
| | ZN517_ | | | ID | (SEQ | LSTL | | | | | |
| | 452 | | | NO: | ID | IQHQ | | | | | |
| | | | | 65) | NO: | KVH | | | | | |
| | | | | | 243) | (SEQ ID | | | | | |
| | | | | | | NO: | | | | | |
| | | | | | | 247) | | | | | |
| 8HZnF_ | 474_ | ZN582 | ZN582 | YQCK | RVSH | YQCK | 247.6 | 16.93 | 125 | ~2.571 | |
| _ | J1ZN582_ | | | VCGR | LTVH | VCGR | | | | | |
| 19 | 395_ | | | AFK(SEQ | YRIH | AFKR | | | | | |
| | 417- | | | ID | (SEQ | VSHL | | | | | |
| | ZN582_ | | | NO: | ID | TVHY | | | | | |
| | 395417_ | | | 77) | NO: | RIH | | | | | |
| | J1 | | | | 265) | (SEQ ID | | | | | |
| | | | | | | NO: | | | | | |
| | | | | | | 268) | | | | | |
| HZnF_ | IKZF3_ | IKZF3 | ZN787 | FQCN | QPKS | FQCN | 4.593 | 1.054 | 5.22 | 3.97E−05 | 6.09 |
| 20 | 146_ | | | QCGA | LARH | QCGA | | | | | |
| | 168- | | | SFT(SEQ | LRLH | SFTQ | | | | | |
| | ZN787_ | | | ID | (SEQ | PKSL | | | | | |
| | 178200_ | | | NO: | ID | ARHL | | | | | |
| | J1 | | | 46) | NO: | RLH | | | | | |
| | | | | | 419) | (SEQ ID | | | | | |
| | | | | | | NO: | | | | | |
| | | | | | | 421) | | | | | |
| HZnF_ | ZN827_ | ZN827 | ZKSC5 | FQCP | RHSH | FQCP | 11.17 | 0.3106 | 2.46 | 0.000107 | 8.23 |
| 21 | 374396- | | | I | LIEH | ICGL | | | | | |
| | ZKSC5_ | | | CGLV | LKRH | VIKR | | | | | |
| | 430452_ | | | I | (SEQ | HSHL | | | | | |
| | J1 | | | K | ID | IEHL | | | | | |
| | | | | (SEQ | NO: | KRH | | | | | |
| | | | | ID | 177) | (SEQ ID | | | | | |
| | | | | NO: | | NO: | | | | | |
| | | | | 57) | | 198) | | | | | |
| HZnF_ | ZN653_ | ZN653 | ZN787 | LQCE | QPKS | LQCE | 12.17 | 0.2124 | 2.417 | 0.000037 | 6.59 |
| 22 | 556_ | | | ICGY | LARH | ICGY | | | | | |
| | 578- | | | QCR(SEQ | LRLH | QCRQ | | | | | |
| | ZN787_ | | | ID | (SEQ | PKSL | | | | | |
| | 178200_ | | | NO: | ID | ARHL | | | | | |
| | J1 | | | 65) | NO: | RLH | | | | | |
| | | | | | 419) | (SEQ ID | | | | | |
| | | | | | | NO: | | | | | |
| | | | | | | 431) | | | | | |
| HZnF_ | ZFP91_ | ZFP91 | ZN787 | LQCE | QPKS | LQCE | 6.452 | 0.1463 | 0.9833 | 1.06E−05 | 4.66 |
| 23 | 400_ | | | ICGF | LARH | ICGF | | | | | |
| | 422ZN692_ | | | TCR(SEQ | LRLH | TCRQ | | | | | |
| | 417_ | | | ID | (SEQ | PKSL | | | | | |
| | 439- | | | NO: | ID | ARHL | | | | | |
| | ZN787_ | | | 67) | NO: | RLH | | | | | |
| | 178200_ | | | | 419) | (SEQ ID | | | | | |
| | J1 | | | | | NO: | | | | | |
| | | | | | | 426) | | | | | |
| HZnF_ | ZN276_ | ZN276 | ZN787 | LQCE | QPKS | LQCE | 10.05 | 0.4597 | 5.672 | 5.69E−05 | 14.96 |
| 24 | 524546- | | | VCGF | LARH | VCGF | | | | | |
| | ZN787_ | | | QCR | LRLH | QCRQ | | | | | |
| | 178200_ | | | (SEQ | (SEQ | PKSL | | | | | |
| | J1 | | | ID | ID | ARHL | | | | | |
| | | | | NO: | NO: | RLH | | | | | |
| | | | | 71) | 419) | (SEQ | | | | | |
| | | | | | | ID | | | | | |
| | | | | | | NO: | | | | | |
| | | | | | | 435) | | | | | |
| HZnF_ | ZN653_ | ZN653 | PATZ1 | LQCE | RKDR | LQCE | 6.641 | 0.1604 | 0.6928 | 4.97E−05 | 3.53 |
| 25 | 556578- | | | ICGY | MSYH | ICGY | | | | | |
| | PATZ1_ | | | QCR(SEQ | VRSH | QCRR | | | | | |
| | 383405_ | | | ID | (SEQ | KDRM | | | | | |
| | J1 | | | NO: | ID | SYHV | | | | | |
| | | | | 65) | NO: | RSH | | | | | |
| | | | | | 111) | (SEQ | | | | | |
| | | | | | | ID | | | | | |
| | | | | | | NO: | | | | | |
| | | | | | | 130) | | | | | |
| HZnF_ | ZFP91_ | ZFP91 | IKZF3 | LQCE | QKGN | LQCE | 22.87 | 0.78 | 13.36 | 2.08E−05 | 29.44 |
| 26 | 400_ | | | ICGF | LLRH | ICGF | | | | | |
| | 422ZN692_ | | | TCR(SEQ | IKLH | TCRQ | | | | | |
| | 417_ | | | ID | (SEQ | KGNL | | | | | |
| | 439- | | | NO: | ID | LRHI | | | | | |
| | IKZF3_ | | | 67) | NO: | KLH | | | | | |
| | 146_168 | | | | 89) | (SEQ | | | | | |
| | IKZF2_ | | | | | ID | | | | | |
| | 140_ | | | | | NO: | | | | | |
| | 162_ | | | | | 99) | | | | | |
| | J1 | | | | | | | | | | |
| HZnF_ | IKZF3_ | IKZF3 | IKZF3 | FQCN | QKGN | FQCN | 21.86 | 3.105 | 33.46 | 6.82E−05 | 28.2 |
| 27 | 146168- | | | QCGA | LLRH | QCGA | | | | | |
| | IKZF3_ | | | SFT(SEQ | IKLH | SFTQ | | | | | |
| | 146_168 | | | ID | (SEQ | KGNL | | | | | |
| | IKZF2_ | | | NO: | ID | LRHI | | | | | |
| | 140_ | | | 46) | NO: | KLH | | | | | |
| | 162_ | | | | 89) | (SEQ | | | | | |
| | J1 | | | | | ID | | | | | |
| | | | | | | NO: | | | | | |
| | | | | | | 102) | | | | | |

In Table 3B, italicized N and C indicate endogenous ZF controls.

### Example 4

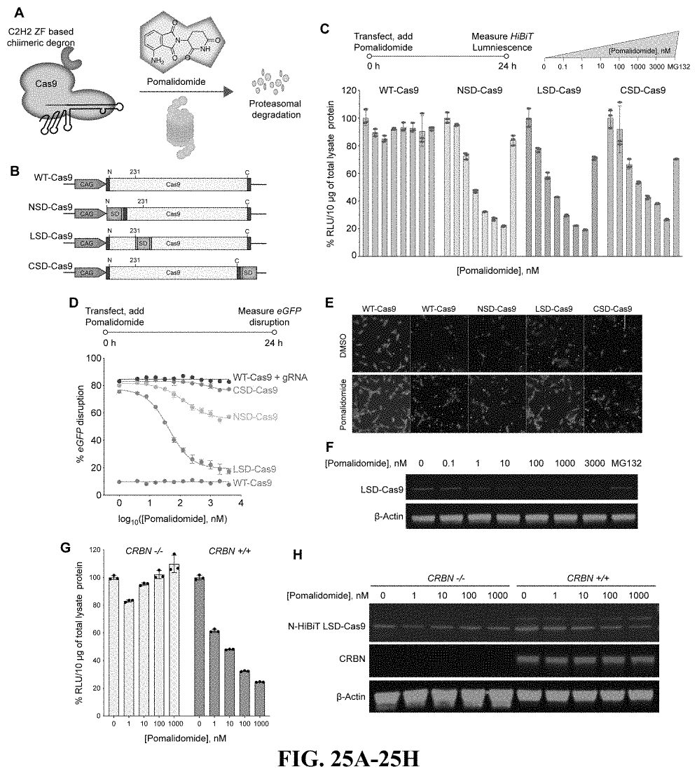

Exemplary Cas9 degradation using exemplary zinc finger degrons was conducted. (FIG. 25A-25H). Fusion of Cas9 at N-terminal Loop-231 and C-terminal fusions (FIG. 25B) were investigated for pomalidomide-induced degradation, and dose-dependent degradation measured in U2OS cells. (FIG. 25D). Cells were transfected and pomalidomide added with HiBiT luminescence measured at 24 hours. (FIG. 25D) measured by eGFP disruption assay images (FIG. 25E). Pomalidomide induced degradation of an N-HiBIT fused LSD-Cas9 protein of transiently transfected HEK293T cells, FIG. 25G, 25H.

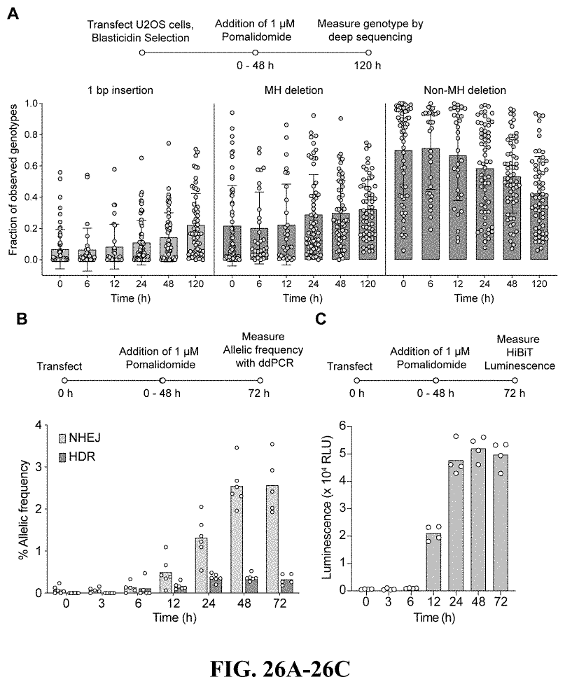

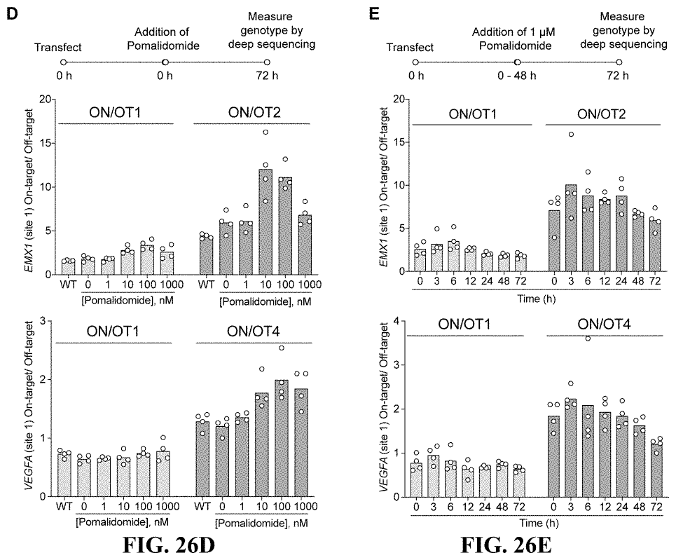

Targeting specificity and DNA repair outcome is explored with respect to an LSD-Cas9 transposon and pomalidomide degradation treated at different tine points after transfection in U2OS cells. (FIG. 26A). FIG. 26B details NHEJ versus HDR DNA repair. An example embodiment LSD-Cas9 plasmid, GAPDH gRNA plasmid, and ssODN template were transfected in HEK293T cells followed by addition of pomalidomide at different time points after transfection with luminescence-based quantification measured. (FIG. 26C). Cas9 lifetime can impact Cas9 targeting specificity, as exemplified by pomalidomide dose-dependent control of on-target activity (FIG. 26D, 26E).

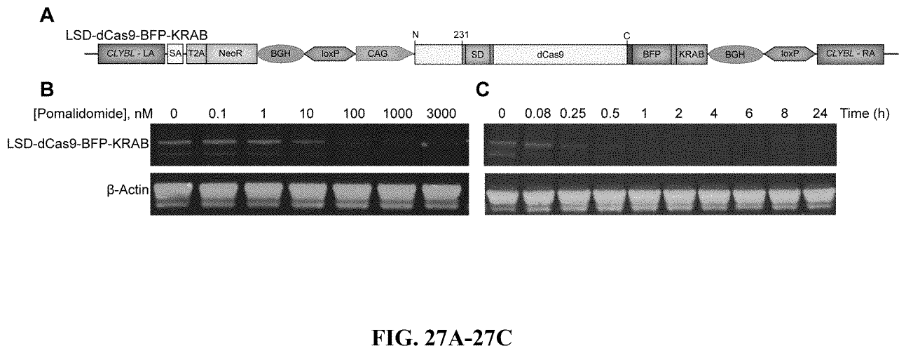

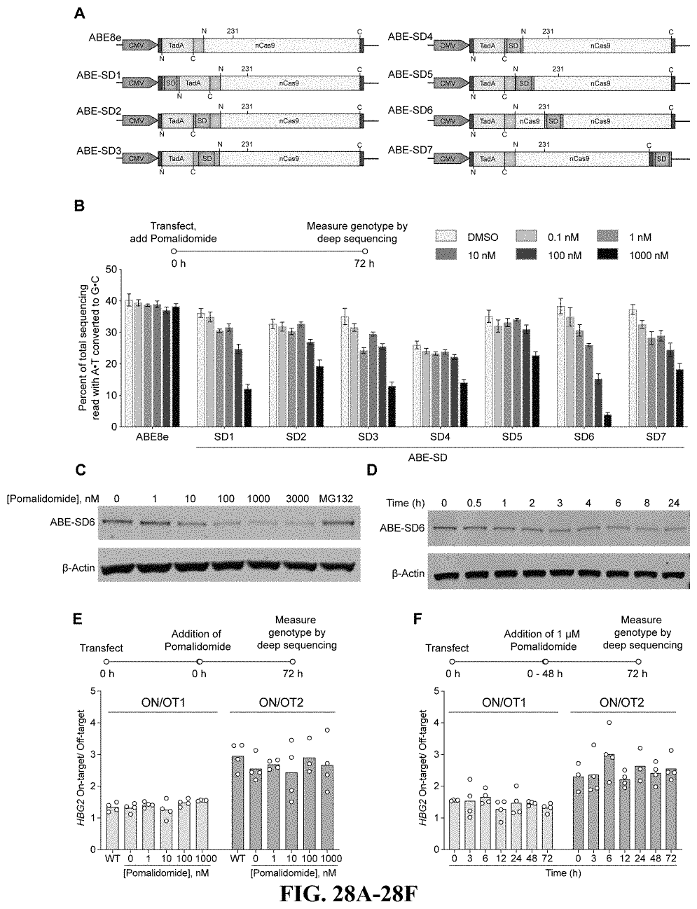

Exemplary dCas9-KRAB fusion with exemplary zinc finger degron CRISPR system knock-in in human iPSCs and pomalidomide dose induced degradation was monitored by immunoblots. (FIG. 27B-FIG. 27C). Base editors fused with an exemplary super degron tag at N-terminal (ABE-SD1), C-terminal (ABE-SD2) of TadA deaminase, at the linker region (ABE-SD3, ABE-SD4), and N-terminal (ABE-SD5), Loop-231 (ABE-SD6), and C-terminal (ABE-SD7) of Cas9 nickase regions. (FIG. 28A). Pomalidomide dose-induced and time-dependent degradation in I-1E1(293T cells as shown in immunoblots (FIG. 28E, 28F), As shown in FIG. 28F, Pomalidomide induced lifetime-dependent control of on-target versus off-target activity of an ABE-SD6 targeting HBG in cells.

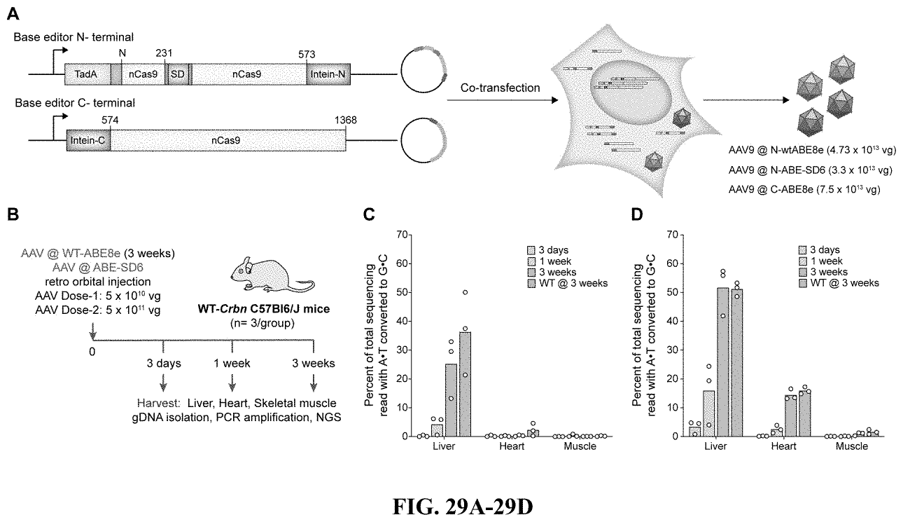

An AAV split ABE-S6 zinc finger mice model was utilized to explore kinetics of base editing activity. As depicted in FIG. 29A, an intein reconstitution strategy was used to reconstitute a full length protein following expression in host cells, SD represents super degron fused at Loop 231 of the nCas9. Retro-orbital injection of the split ABE-S6 zinc finger system AAVs were performed in C57Bl6/J mice, harvested at 3 days, 1 week, or 3 weeks post-injection to measure editing efficiency in liver, heart and skeletal muscle. (FIG. 29C, 29D).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A hybrid zinc finger polypeptide comprising an N-terminal beta hairpin subdomain selected from SEQ ID NOs: 46, 49, 51, 53, 55, 57, 59, 61, 63, 65, 67, 69, 71, 73, 75, 77, 79, 81, 83, 85, 87; and an alpha-helix C-terminal subdomain selected from SEQ ID NOs: 47, 89, 111, 133, 155, 177, 199, 221, 243, 265, 287, 309, 331, 353, 375, 397, 419, 441, 462, 484, and 506.

2. The hybrid zinc finger polypeptide of claim 1, comprising a sequence selected from SEQ ID NOs: 48, 50, 52, 54, 56, 58, 60, 62, 64, 66, 68, 70, 72, 74, 76, 78, 80, 82, 84, 86, 88, 90, 91, 92, 93, 94, 95, 96, 97, 98, 99, 100, 101, 102, 103, 104, 105, 106, 107, 108, 109, 110, 112, 113, 114, 115, 116, 117, 118, 119, 120, 121, 122, 123, 124, 125, 126, 127, 128, 129, 130, 131, 132, 134, 135, 136, 137, 138, 139, 140, 141, 142, 143, 144, 145, 146, 147, 148, 149, 150, 151, 152, 153, 154, 156, 157, 158, 159, 160, 161, 162, 163, 164, 165, 166, 167, 168, 169, 170, 171, 172, 173, 174, 175, 176, 178, 179, 180, 181, 182, 183, 184, 185, 186, 187, 188, 189, 190, 191, 192, 193, 194, 195, 196, 197, 198, 200, 201, 202, 203, 204, 205, 206, 207, 208, 209, 210, 211, 212, 213, 214, 215, 216, 217, 218, 219, 220, 222, 223, 224, 225, 226, 227, 228, 229, 230, 231, 232, 233, 234, 235, 236, 237, 238, 239, 240, 241, 242, 244, 245, 246, 247, 248, 249, 250, 251, 252, 253, 254, 255, 256, 257, 258, 259, 260, 261, 262, 263, 264, 266, 267, 268, 269, 270, 271, 272, 273, 274, 275, 276, 277, 278, 279, 280, 281, 282, 283, 284, 285, 286, 288, 289, 290, 291, 292, 293, 294, 295, 296, 297, 298, 299, 300, 301, 302, 303, 304, 305, 306, 307, 308, 310, 311, 312, 313, 314, 315, 316, 317, 318, 319, 320, 321, 322, 323, 324, 325, 326, 327, 328, 329, 330, 332, 333, 334, 335, 336, 337, 338, 339, 340, 341, 342, 343, 344, 345, 346, 347, 348, 349, 350, 351, 352, 354, 355, 356, 357, 358, 359, 360, 361, 362, 363, 364, 365, 366, 367, 368, 369, 370, 371, 372, 373, 374, 376, 377, 378, 379, 380, 381, 382, 383, 384, 385, 386, 387, 388, 389, 390, 391, 392, 393, 394, 395, 396, 398, 399, 400, 401, 402, 403, 404, 405, 406, 407, 408, 409, 410, 411, 412, 413, 414, 415, 416, 417, 418, 420, 421, 422, 423, 424, 425, 426, 427, 428, 429, 430, 431, 432, 433, 434, 435, 436, 437, 438, 439, 440, 442, 443, 444, 445, 446, 447, 448, 449, 450, 451, 452, 453, 454, 455, 456, 457, 458, 459, 460, 461, 463, 464, 465, 466, 467, 468, 469, 470, 471, 472, 473, 474, 475, 476, 477, 478, 479, 480, 481, 482, 483, 485, 486, 487, 488, 489, 490, 491, 492, 493, 494, 495, 496, 497, 498, 499, 500, 501, 502, 503, 504, 505, 507, 508, 509, 510, 511, 512, 513, 514, 515, 516, 517, 518, 519, 520, 521, 522, 523, 524, 525, 526, or 527.

3. The hybrid zinc finger of claim 1, wherein the hybrid zinc finger polypeptide is optimized for degradation by pomalidomide, and the zinc finger polypeptide comprises a sequence selected from SEQ ID NOs: 175, 361, 201, 457, 269, 110, 84, 246, 168, 359, 203, 448, 278, 102, 48, 209, 450, 285, 109, 440, 171, 367, 218, 277, 107, 161, 366, 214, 443, 283, 172, 364, 216, 451, 284, 162, 371, 165, 370, 444, 452, 170, 91, 82, 373, and 156.

4. The hybrid zinc finger of claim 1, wherein the hybrid zinc finger polypeptide is optimized for degradation by avadomide, and the zinc finger polypeptide comprises a sequence selected from SEQ ID NOs: 175, 361, 457, 201, 269, 110, 84, 246, 168, 359, 448, 203, 278, 102, 171, 367, 445, 277, 107, 182, 163, 360, 450, 209, 109, 164, 354, 452, 219, 271, 161, 366, 443, 283, 162, 371, 446, 170, 365, 91, 172, 364, 451, 373, 156, 357, and 444.

5. The hybrid zinc finger of claim 1, wherein the hybrid zinc finger polypeptide is optimized for degradation by iberomide, and the zinc finger polypeptide comprises a sequence selected from SEQ ID NOs: 360, 209, 405, 109, 440, 359, 203, 448, 48, 102, 278, 367, 171, 218, 445, 74, 107, 361, 175, 201, 84, 371, 162, 215, 446, 443, 354, 164, 219, 452, 170, 82, 91, 364, 172, 216, 373, 212, 165, and 156.

6. The hybrid zinc finger of claim 1, wherein the hybrid zinc finger polypeptide is optimized for degradation by lenalidomide, and the zinc finger polypeptide comprises a sequence selected from SEQ ID NOs: 445, 455, 91, 373, 449, 160, 212, 354, 452, 164, 219, 359, 448, 168, 102, 361, 457, 175, 201, 360, 450, 163, 209, and 109.

7. A programmable nuclease comprising one or more hybrid zinc finger polypeptides of claim 2 introduced into the nuclease at one or more insertion sites.

8. The programmable nuclease of claim 7, wherein the nuclease is a CRISPR-Cas protein, a Zinc finger nuclease, a TALEN or a meganuclease,
optionally wherein, the programmable nuclease is codon optimized for expression in eukaryotes;
optionally wherein, the CRISPR-Cas protein is a Type II, Type V or Type VI Cas protein;
optionally wherein, the CRISPR-Cas protein is a Cas9, a Cas12a, Cas12b, Cas12c, Cas12d, Cas13a, Cas13b, Cas13c, or Cas13d protein;
optionally wherein, the one or more insertion sites are at the N-terminal (Nt), C-terminal (Ct) or at a position corresponding to a position on the loop of a SpCas9 protein; and
optionally wherein the sequence comprises SEQ ID NO: 45.

9.-13. (canceled)

14. The programmable nuclease of claim 8, wherein the CRISPR-Cas protein is a dCas9, optionally wherein the dCas9 is fused to one or more functional domains and optionally wherein the functional domain is a KRAB domain or a transposase domain.

15. (canceled)

16. (canceled)

17. The programmable nuclease of claim 6, wherein the CRISPR-Cas protein is a Cas-based nickase, optionally wherein the Cas-based nickase is a Cas9 nickase which comprises a mutation in the HNH domain,
optionally wherein, the functional component is a base editing component, optionally wherein the base editing component is fused directly or indirectly to the N terminal of the CRISPR-Cas nickase;
optionally wherein, the base editing component comprises an adenosine deaminase; and
optionally wherein, the base editing component is fused at N-terminal or C-terminal of the adenosine deaminase, at the linker region, the N-terminal, a loop of the CRISPR-Cas nickase, or C-terminal of the CRISPR-Cas nickase.

18.-20. (canceled)

21. A ribonucleoprotein comprising the programmable nuclease of any one of claim 7.

22. A plasmid comprising the variant CRISPR-Cas protein of any one of claim 7.

23. A cell transfected with the ribonucleoprotein of claim 21 or the plasmid of claim 22.

24. A method of inducing degradation of a programmable nuclease, comprising: exposing the cell of claim 23 with an immunomodulatory imide drug (IMiD) or a pharmaceutically acceptable salt thereof,
optionally wherein, the IMiD is selected from thalidomide, lenalidomide, pomalidomide, avadomide, iberdomide, and analogs thereof;
optionally wherein, the exposing the cell with the IMiD is performed about 3 to 6 hours, about 6 to 12 hours, about 12 to 24 hours, or about 24 to 48 hours after the cell is transfected;
optionally wherein, the exposing comprises incubating the cell with the compound or pharmaceutically acceptable salt thereof, wherein the compound is provided at a concentration of about 10 nM to about 10 μM;
optionally wherein, the cell is a germline cell; and
optionally wherein, the cell is in an organism.

25.-29. (canceled)

30. The method of claim 24, wherein the cell comprises the hybrid zinc finger comprising the selected from: SEQ ID NOs: 175, 361, 201, 457, 269, 110, 84, 246, 168, 359, 203, 448, 278, 102, 48, 209, 450, 285, 109, 440, 171, 367, 218, 277, 107, 161, 366, 214, 443, 283, 172, 364, 216, 451, 284, 162, 371, 165, 370, 444, 452, 170, 91, 82, 373, and 156, and the IMiD is pomalidomide;
SEQ ID NOs: 175, 361, 457, 201, 269, 110, 84, 246, 168, 359, 448, 203, 278, 102, 171, 367, 445, 277, 107, 182, 163, 360, 450, 209, 109, 164, 354, 452, 219, 271, 161, 366, 443, 283, 162, 371, 446, 170, 365, 91, 172, 364, 451, 373, 156, 357, and 444, and the IMiD is avadomide;
SEQ ID NOs: 360, 209, 405, 109, 440, 359, 203, 448, 48, 102, 278, 367, 171, 218, 445, 74, 107, 361, 175, 201, 84, 371, 162, 215, 446, 443, 354, 164, 219, 452, 170, 82, 91, 364, 172, 216, 373, 212, 165, and 156, and the IMiD is iberomide; and
SEQ ID NOs: 445, 455, 91, 373, 449, 160, 212, 354, 452, 164, 219, 359, 448, 168, 102, 361, 457, 175, 201, 360, 450, 163, 209, and 109, and the IMiD is lenalidomide.

31.-33. (canceled)

34. A method of controlling programmable nuclease editing outcomes comprising administering an immunomodulatory imide drug (IMiD) or a pharmaceutically acceptable salt thereof to a cell or a population of cells comprising or expressing a variant CRISPR-Cas protein of claim 7,
optionally wherein, the IMiD is selected from thalidomide, lenalidomide, pomalidomide, avadomide, iberomide, and analogs thereof and
optionally wherein the method is performed in vitro or in vivo.

35. (canceled)

36. (canceled)

37. The method of claim 1, wherein the exposing or administering of the IMiD is performed at a time to encourage target specificity.
